(19) United States
(12) Patent Application Publication
(10) Pub. No.: US 2026/0115280 A1
(43) Pub. Date: Apr. 30, 2026

(54) VACCINES

(71) Applicant: ADJUVANCE TECHNOLOGIES, INC., Lincoln, NE (US)

(72) Inventors: J. Tyler MARTIN, Roca, NE (US); Jared William WENGER, Lincoln, NE (US); Eric Jon FARRIS, Lincoln, NE (US); Patrick J. FRENCHICK (Deceased); Anna Therese LAMPE, Lincoln, NE (US)

(21) Appl. No.: 18/552,863

(22) PCT Filed: Apr. 13, 2022
(86) PCT No.: PCT/US2022/024594

§ 371 Date: Sep. 27, 2023

Related U.S. Application Data
(60) Provisional application No. 63/174,910, filed on Apr. 14, 2021.
(60) Provisional application No. 63/221,878, filed on Jul. 14, 2021.

(51) Int. Cl. A61K 39/39; A61K 31/704; A61K 39/00; A61K 39/215; A61K 39/25; A61P 31/22; C12N 7/00

(52) U.S. Cl. CPC A61K 39/39; A61K 31/704; A61K 39/215; A61K 39/25; A61P 31/22; C12N 7/00; A61K 2039/55577; C12N 2710/16734; C12N 2770/20034

(57) ABSTRACT

The present application relates to triterpene glycoside saponin-derived adjuvants, TLR4 agonists and antagonists, TLR9 agonists and antagonists, and combinations thereof, as well as pharmaceutical compositions comprising the foregoing and methods of making and of using the foregoing in the treatment of certain diseases.

### INCORPORATION BY REFERENCE OF RELATED APPLICATIONS

This application is based upon and claims priority under 35 U.S.C. § 119 (e) to U.S. Provisional Application U.S. Ser. No. 63/221,878, filed Jul. 14, 2021, and to U.S. Provisional Application U.S. Ser. No. 63/174,910, filed Apr. 14, 2021, the entire contents of which are incorporated herein by reference in their entirety.

This application is related to U.S. provisional application U.S. Ser. No. 62/930,727 filed Nov. 5, 2019, and International application PCT/US2020/058859, the entire contents of all of which are incorporated herein by reference in their entirety.

### FIELD OF THE INVENTION

The present application relates to triterpene glycoside saponin-derived adjuvants, TLR4 (toll-like receptor 4) agonists and antagonists, and combinations thereof, as well as pharmaceutical compositions capable of inducing an immune response, methods of administering such compositions, and methods of producing such compositions.

### STATEMENT

To comply with 37 C.F.R. § 1.821, this application contains sequence listings included in an ASCII text file submitted via EFS-WEB. The ASCII text file has the following attributes: (1) Name: Sequences Prov_ST25.txt, (2) Date of creation: Aug. 3, 2021, (3) File size in bytes: 50,719. Pursuant to MPEP § 2422.03 (a), Applicant hereby incorporates by reference the foregoing ASCII text file and all material disclosed therein into this application.

### BACKGROUND

Vaccines against infectious diseases continue to improve public health across the world. With increased knowledge of etiologic pathogens and necessary immune responses have come increasingly defined or targeted vaccines. Hepatitis B, DTaP, HPV, pneumococcal and other widely used vaccines require the use of the immunological adjuvant alum. However, alum, which was introduced over 80 years ago, is a poor adjuvant restricting the potency of some of these vaccines and requiring higher or more doses of others. A far more potent adjuvant employed more recently is the natural saponin adjuvant QS-21, used widely despite 3 major liabilities: dose-limiting toxicity, poor stability, and limited availability of quality products.

Saponins are glycosidic compounds that are produced as secondary metabolites of steroids and triterpenes. They are widely distributed among plant species and in some marine invertebrates. The chemical structure of saponins imparts a wide range of pharmacological and biological activities, including some potent and efficacious immunological activity. Semi-purified saponin extracts from the bark of the South American Quillaja saponaria Molina tree (Quillaja saponins) exhibit remarkable immunoadjuvant activity. Because the Quillaja saponins are found as a mixture of at least one hundred structurally related saponin glycosides, their separation and isolation is often difficult if not prohibitive. The most active fraction of these extracts, designated QS-21, has been found to include a mixture of two principal isomeric triterpene glycoside saponins, each incorporating a quillaic acid triterpene core, flanked on either side by complex oligosaccharides and a stereochemically rich glycosylated fatty acyl chain.

The potency of QS-21 and its acceptable toxicity profile in dozens of recent and ongoing vaccine clinical trials (melanoma, breast cancer, small cell lung cancer, prostate cancer, HIV-1, malaria) have established it as a promising new adjuvant for immune response potentiation and dose-sparing. However, the tolerated dose of QS-21 in cancer patients does not exceed 100-150 μg, above which significant local and systemic side effects arise. The highest practical tolerable dose in well (non-cancer) adult and child recipients is 25-50 mcg, an immunologically suboptimal dose. As a result, the clinical success of non-cancer vaccines continues to critically depend on the identification of, and access to, novel, potent adjuvants that are more tolerable.

In view of the drawbacks associated with QS-21, synthetic saponin molecules have been explored in an effort to reduce toxicity, improve stability, and increase availability. Several such saponins are described in PCT/US2009/039954, PCT/US2015/33567, PCT/US2016/67530, and PCT/US2016/60564. In particular, the saponin adjuvant TQL-1055 (Compound I-9) discussed in PCT/US2016/60564 has emerged as a leading QS-21 analogue, exhibiting reduced toxicity and improved efficacy as compared to the natural product. However, little remains known regarding the mechanism of cellular activation induced by saponin adjuvants, including QS-21 and TQL-1055.

Toll-like receptor 4 (TLR4) is a protein in humans that recognizes and is activated by a core component of the outer membrane of Gram-negative bacteria known as lipopolysaccharide (LPS). TLR4 activation leads to an intracellular signaling pathway and inflammatory cytokine production, which ultimately influences activation of the immune system. In particular, the production of cytokines is believed to be necessary for the development of effective immunity against certain pathogens. Additionally, treatment with naturally occurring or synthetic TLR4 agonists, e.g. Lipid A or MPL, has also been shown to augment the host response to infection and to serve as a potential vaccine adjuvant.

Toll-like receptor 9 (TLR9) is a protein in humans that recognizes and is activated by DNA present in bacteria and viruses. TLR9 activation triggers signaling cascades that lead to a pro-inflammatory cytokine response. TLR9 may be activated by certain unmethylated CpG sequences in DNA molecules, which are relatively rare in vertebrate genomes in comparison to bacterial genomes or viral DNA.

The human immune system consists of the ancient innate immune system passed on along the evolution from invertebrates and the recently acquired adaptive immune system uniquely present in vertebrates. The principal functions of the immune system are the recognition with subsequent elimination of foreign antigens, formation of immunologic memory, and development of tolerance to self-antigens. The lymphocyte population is mainly made up of the thymus-derived lymphocytes (T-lymphocytes), bone-marrow-derived (B-lymphocytes), and the natural-killer cells (NK cells). T-lymphocytes mediating the cellular immunity, along with B lymphocytes mediating humoral immunity, provide adaptive immunity, which work in close collaboration with the innate immune system. B-lymphocytes mature in the bone marrow itself, while the T-lymphocytes require the thymus to mature, before being deployed to the peripheral lymphoid organs for further antigen-mediated differentiation. A small subset of the CD4+ cells, including natural regulatory cells and natural killer T cells (NKT cells), are already distinct differentiated cells on release from the thymus. CD4+ T cells along with CD8+ T cells make up the majority of T-lymphocytes. CD4+ T cells after being activated and differentiated into distinct effector subtypes play a major role in mediating immune response through the secretion of specific cytokines. The CD4+ T cells carry out multiple functions, ranging from activation of the cells of the innate immune system, B-lymphocytes, cytotoxic T cells, as well as nonimmune cells, and also play critical role in the suppression of immune reaction.

### SUMMARY

The inventors of the present application have invented combinations of certain saponin adjuvants, including preferably TQL-1055, and certain TLR4 and/or TLR9 agonists, which together will produce surprising, beneficial, and/or synergistic effects. The inventors of the present application also provide pharmaceutical compositions comprising the foregoing and methods of making and of using the foregoing in the treatment and prevention of certain diseases.

The inventors of the present application have also invented compositions which improve the magnitude and duration of an induced CD4 immune response, and thus are capable of vaccinating against certain pathogens where CD4 responses are thought to control infection directly, including, for example, HIV, all herpes viruses (e.g. HSV-1/2, varicella zoster virus (VZV), Epstein Barr virus (EBV), Cytomeglovirus (CMV), and Human herpesvirus (HHV6/7/8), and their related disorders such as herpes zoster and post herpetic neuralgia (PHN)), Plasmodium falciparum and other Plasmodium species, Mycobacterium tuberculosis, M. leprae other Mycobacteria species, Salmonella typhi and other Salmonella species, Leishmania major and other Leishmania species, Necator americanus, Ancylostoma duodenale, Giardia lamblia and other Giardia species, Cryptosporidium parvum and other Cryptosporidium species, Coccidioides immitis and other Coccidiodes species, and Histoplasma capsulatum and other Histoplasma species.

The inventors of the present application have also invented compositions which are capable of vaccinating against certain pathogens where the antibody response may be improved in quality or duration by a helper CD4 response, including, for example, Influenza A and B viruses, Bordetella pertussis, Coronaviruses (including SARS-COV1, MERS-COV, and SARS-COV2), vesicular stomatitis virus (VSV), Vaccinia viruses, respiratory syncytial virus (RSV), Dengue virus, Zika virus, West Nile Virus, Yellow Fever virus (YFV), and alphaviruses including equine encephalitis (VEE/EEE/WEE), Chikungunya, Ross River, and O'Nyong-nyong viruses. The inventors of the present application have also invented methods of administering such compositions and methods of producing such compositions.

In one aspect, the present application provides TQL-1055:

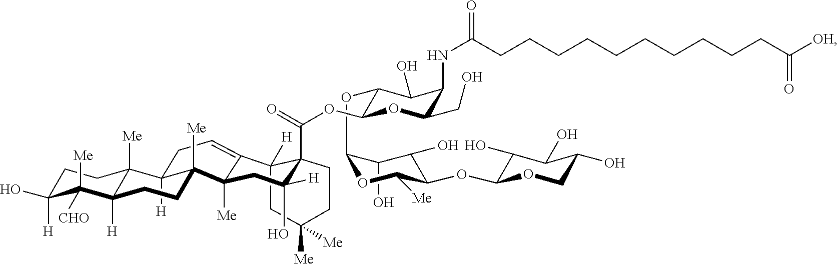

or a pharmaceutically acceptable salt thereof.

In another aspect, the present application provides compounds of Formula I.

In an embodiment of the present application, a composition which induces an immune response against Varicella zoster virus comprises an optionally truncated VZV glycoprotein or a fragment thereof in combination with a triterpene glycoside saponin-derived adjuvant. The optionally truncated VZV glycoprotein may be VZV glycoprotein E or a fragment thereof, and may have the sequence of SEQ ID No. 1.

In an embodiment of the present application, the saponin-derived adjuvant may be a compound according to Formula I:

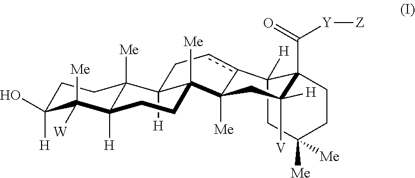

or a pharmaceutically acceptable salt thereof, wherein
[symbol] is a single or double bond;
W is —CHO;
V is —OH;
Y is —O—;
wherein Z is a carbohydrate domain having the structure:

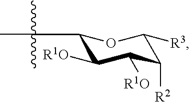

wherein:
R¹ is independently H or

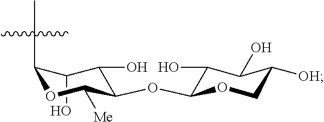

R² is NHR⁴;
R³ is CH₂OH; and
R⁴ is -T-R^{z}, —C(O)-T-R^{z}, —NH-T-R^{z}, —O-T-R^{z}, —S-T-R^{z}, —C(O)NH-T-R^{z}, C(O)O-T-R^{z}, C(O)S-T-R^{z}, C(O)NH-T-O-T-R^{z}, —O-T-R^{z}, -T-O-T-R^{z}, -T-S-T-R^{z}, or

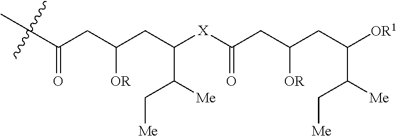

wherein:
X is —O—, —NR—, or T-R^{z};
T is a covalent bond or a bivalent C₁₋₂₆ saturated or unsaturated, straight or branched, aliphatic or heteroaliphatic chain;
R^{z} is hydrogen, halogen, —OR, —OR^{x}, —OR^{1′}, —SR, NR₂, —C(O)OR, —C(O)R, —NHC(O)R, —NHC(O)OR, NC(O)OR, or an optionally substituted group selected from acyl, arylalkyl, heteroarylalkyl, C₁₋₆ aliphatic, 6-10-membered aryl, 5-10-membered heteroaryl having 1-4 heteroatoms independently selected from nitrogen, oxygen, or sulfur, 4-7-membered heterocyclyl having 1-2 heteroatoms independently selected from the group consisting of nitrogen, oxygen, and sulfur;
R^{x} is independently hydrogen or an oxygen protecting group selected from the group consisting of alkyl ethers, benzyl ethers, silyl ethers, acetals, ketals, esters, carbamates, and carbonates; and
R is independently hydrogen, an optionally substituted group selected from acyl, arylalkyl, 6-10-membered aryl, C₁₋₆ aliphatic, or C₁₋₆ heteroaliphatic having 1-2 heteroatoms independently selected from the group consisting of nitrogen, oxygen, and sulfur, or:
   two R on the same nitrogen atom are taken with the nitrogen atom to form a 4-7-membered heterocyclic ring having 1-2 heteroatoms independently selected from the group consisting of nitrogen, oxygen, and sulfur;
R^{1′} is R^{x} or a carbohydrate domain having the structure:

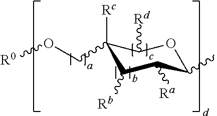

wherein:
each occurrence of a, b, and c is independently 0, 1, or 2;
d is an integer from 1-5, wherein each d bracketed structure may be the same or different; with the proviso that the d bracketed structure represents a furanose or a pyranose moiety, and the sum of b and c is 1 or 2;
R⁰ is hydrogen; an oxygen protecting group selected from the group consisting of alkyl ethers, benzyl ethers, silyl ethers, acetals, ketals, esters, carbamates, and carbonates; or an optionally substituted moiety selected from the group consisting of acyl, C₁₋₁₀ aliphatic, C₁₋₆ heteroaliphatic, 6-10-membered aryl, arylalkyl, 5-10 membered heteroaryl having 1-4 heteroatoms independently selected from nitrogen, oxygen, or sulfur, 4-7 membered heterocyclyl having 1-2 heteroatoms independently selected from the group consisting of nitrogen, oxygen, and sulfur;
each occurrence of R^{a}, R^{b}, R^{c}, and R^{d} is independently hydrogen, halogen, OH, OR, OR^{x}, NR₂, NHCOR, or an optionally substituted group selected from acyl, C₁₋₁₀ aliphatic, C₁₋₆ heteroaliphatic, 6-10-membered aryl, arylalkyl, 5-10-membered heteroaryl having 1-4 heteroatoms independently selected from nitrogen, oxygen, sulfur; 4-7-membered heterocyclyl having 1-2 heteroatoms independently selected from the group consisting of nitrogen, oxygen, and sulfur.

In one aspect, the present application provides compounds of Formula II:

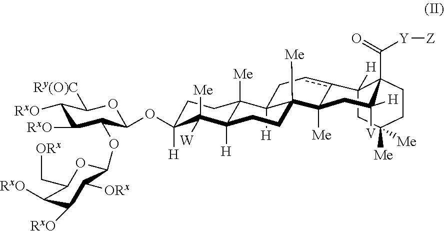

or a pharmaceutically acceptable salt thereof, wherein
[symbol] is a single or double bond;
W is Me, —CHO, or

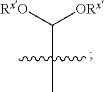

V is hydrogen or OR^{x};
Y is CH₂, —O—, —NR—, or —NH—;
Z is hydrogen; a cyclic or acyclic, optionally substituted moiety selected from the group consisting of acyl, aliphatic, heteroaliphatic, aryl, arylalkyl, heteroacyl, and heteroaryl; or a carbohydrate domain having the structure:

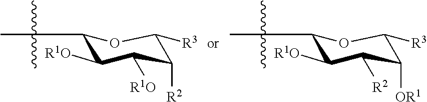

wherein each occurrence of R¹ is R^{x} or a carbohydrate domain having the structure:

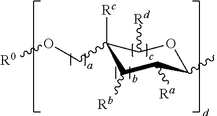

wherein:
each occurrence of a, b, and c is independently 0, 1, or 2;
d is an integer from 1-5, wherein each d bracketed structure may be the same or different; with the proviso that the d bracketed structure represents a furanose or a pyranose moiety, and the sum of b and c is 1 or 2;
R⁰ is hydrogen; an oxygen protecting group selected from the group consisting of alkyl ethers, benzyl ethers, silyl ethers, acetals, ketals, esters, carbamates, and carbonates; or an optionally substituted moiety selected from the group consisting of acyl, C₁₋₁₀ aliphatic, C₁₋₆ heteroaliphatic, 6-10-membered aryl, arylalkyl, 5-10 membered heteroaryl having 1-4 heteroatoms independently selected from nitrogen, oxygen, or sulfur, 4-7 membered heterocyclyl having 1-2 heteroatoms independently selected from the group consisting of nitrogen, oxygen, and sulfur;
   each occurrence of R^{a}, R^{b}, R^{c}, and R^{d} is independently hydrogen, halogen, OH, OR, OR^{x}, NR₂, NHCOR, or an optionally substituted group selected from acyl, C₁₋₁₀ aliphatic, C₁₋₆ heteroaliphatic, 6-10-membered aryl, arylalkyl, 5-10-membered heteroaryl having 1-4 heteroatoms independently selected from nitrogen, oxygen, sulfur; 4-7-membered heterocyclyl having 1-2 heteroatoms independently selected from the group consisting of nitrogen, oxygen, and sulfur;
R² is hydrogen, halogen, OH, OR, OC(O)R⁴, OC(O)OR⁴, OC(O)NHR⁴, OC(O)NRR⁴, OC(O)SR⁴, NHC(O)R⁴, NRC(O)R⁴, NHC(O)OR⁴, NHC(O)NHR⁴, NHC(O)NRR⁴, NHR⁴, N(R⁴)₂, NHR⁴, NRR⁴, N₃, or an optionally substituted group selected from C₁₋₁₀ aliphatic, C₁₋₆ heteroaliphatic, 6-10-membered aryl, arylalkyl, 5-10 membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur;
R³ is hydrogen, halogen, CH₂OR¹, or an optionally substituted group selected from the group consisting of acyl, C₁₋₁₀ aliphatic, C₁₋₆ heteroaliphatic, 6-10-membered aryl, arylalkyl, 5-10-membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur,
R⁴ is -T-R^{z}, —C(O)-T-R^{z}, —NH-T-R^{z}, —O-T-R^{z}, —S-T-R^{z}, —C(O)NH-T-R^{z}, C(O)O-T-R^{z}, C(O)S-T-R^{z}, C(O)NH-T-O-T-R^{z}, —O-T-R^{z}, -T-O-T-R^{z}, -T-S-T-R^{z}, or

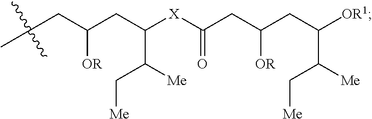

wherein
X is —O—, —NR—, or T-R^{z};
T is a covalent bond or a bivalent C₁₋₂₆ saturated or unsaturated, straight or branched, aliphatic or heteroaliphatic chain; and
R^{z} is hydrogen, halogen, —OR, —OR^{x}, —OR¹, —SR, NR₂, —C(O)OR, —C(O)R, —NHC(O)R, —NHC(O)OR, NC(O)OR, or an optionally substituted group selected from acyl, arylalkyl, heteroarylalkyl, C₁₋₆ aliphatic, 6-10-membered aryl, 5-10-membered heteroaryl having 1-4 heteroatoms independently selected from nitrogen, oxygen, or sulfur, 4-7-membered heterocyclyl having 1-2 heteroatoms independently selected from the group consisting of nitrogen, oxygen, and sulfur;
each occurrence of R^{x} is independently hydrogen or an oxygen protecting group selected from the group consisting of alkyl ethers, benzyl ethers, silyl ethers, acetals, ketals, esters, carbamates, and carbonates;
R^{y} is —OH, —OR, or a carboxyl protecting group selected from the group consisting of ester, amides, and hydrazides;
R^{s} is

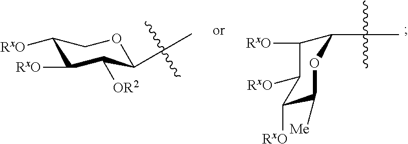

each occurrence of R^{x′} is independently an optionally substituted group selected from 6-10-membered aryl, C₁₋₆ aliphatic, or C₁₋₆ heteroaliphatic having 1-2 heteroatoms independently selected from the group consisting of nitrogen, oxygen, and sulfur; or:
   two R^{x′} are taken together to form a 5-7-membered heterocyclic ring having 1-2 heteroatoms independently selected from the group consisting of nitrogen, oxygen, and sulfur;
each occurrence of R is independently hydrogen, an optionally substituted group selected from acyl, arylalkyl, 6-10-membered aryl, C₁₋₆ aliphatic, or C₁₋₆ heteroaliphatic having 1-2 heteroatoms independently selected from the group consisting of nitrogen, oxygen, and sulfur, or:
   two R on the same nitrogen atom are taken with the nitrogen atom to form a 4-7-membered heterocyclic ring having 1-2 heteroatoms independently selected from the group consisting of nitrogen, oxygen, and sulfur.

In a preferred embodiment of the present application, a composition which induces an immune response against Varicella zoster virus comprises a VZV glycoprotein E truncated to remove the carboxy terminal anchor region, which gE is not in the form of a fusion protein, in combination with an adjuvant comprising a compound shown below (or a pharmaceutically acceptable salt thereof), a TLR4 agonist, and a liposome-forming compound, such as DOPC, DMPC, DMPG, cholesterol, and/or combinations of the foregoing.

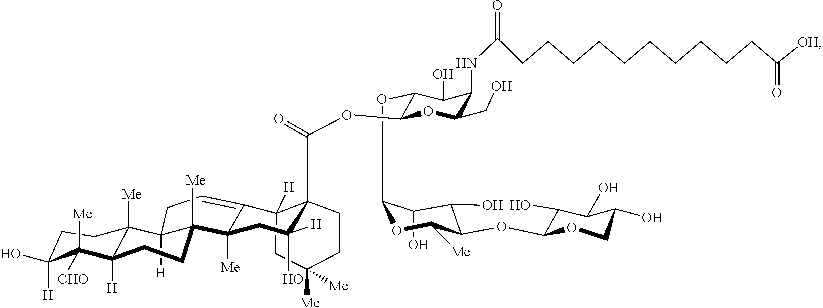

In another aspect, the present application provides for other saponin adjuvants for combination with a TLR4 and/or TLR9 agonist and an antigen. In some embodiments, the saponin is Matrix M, VSA-1, or VSA-2. In another aspect, the present application provides natural saponins for combination with a TLR4 and/or TLR9 agonist and an antigen. In some embodiments, the natural saponin is QS-21, QS-9, QS-7, or Quil A.

In another aspect, the present application provides TLR9 agonists. In some embodiments, the TLR9 agonist is an immunostimulatory sequence (“ISS”). In some embodiments, the ISS is selected from the group consisting of CpG oligodeoxynucleotides class A, B and C, or an immunostimulatory peptide mimicking any of the CpG oligodeoxynucleotides. In some embodiments, the ISS is CpG DNA or a synthetic oligonucleotide containing CpG motifs. In some embodiments, the ISS is CpG 1018, SD-101, or CpG 2006. In some embodiments, the ISS is ODN 7909, AZD1419, ODN 1585, ODN 1668, ODN 1826, ODN 2006, ODN 2007, ODN 2216, ODN 2336, ODN 2395, or ODN M362.

In some embodiments, the ISS comprises the sequence 5′-purine, purine, C, G, pyrimidine, pyrimidine, C. G-3′ or 5′-purine, purine, C, G, pyrimidine, pyrimidine, C, C-3′. In some embodiments, the ISS comprises a sequence selected from the group consisting of AACGTTCC, AACGTTCG, GACGTTCC and GACGTTCG. In some embodiments, the ISS comprises the sequence 5′-T, C, G-3′. In some embodiments of the methods and kits of the invention, the ISS comprises the sequence 5′-TGACTGTGAACGTTCGAGATGA-3′ (SEQ ID NO: 10). In some embodiments, the ISS comprises the sequence 5′-TCGAACGTTCGAACGTTCGAACGTTCGAAT-3′ (SEQ ID NO: 11).

In some embodiments, the TLR9 agonist is a CpG-C ON or a CpG-C type oligonucleotide. “CpG-C ONs” or “CpG-C type oligonucleotides” are oligonucleotides from 12 to 100 bases in length, which have one or more 5′-TCG trinucleotides wherein the 5′-T is positioned 0, 1, 2, or 3 bases from the 5′-end of the oligonucleotide, and at least one palindromic sequence of at least 8 bases in length comprising one or more unmethylated CG dinucleotides. The one or more 5′-TCG trinucleotide sequence may be separated from the 5′-end of the palindromic sequence by 0, 1, or 2 bases or the palindromic sequence may contain all or part of the one or more 5′-TCG trinucleotide sequence. In one embodiment, the oligonucleotide is an oligodeoxynucleotide (ODN). In one embodiment, the oligonucleotide is a 2′-oligodeoxynucleotide. CpG-C ODNs have the ability to stimulate B cells, induce plasmacytoid dendritic cell (PDC) maturation and cause secretion of high levels of type I interferons (e.g., IFN-α, IFN-γ, etc.). In some embodiments, the CpG-C ODNs are 12 to 100 bases in length, preferably 12 to 50 bases in length, preferably 12 to 40 bases in length, or preferably 12-30 bases in length. In some embodiments, the ODN is at least (lower limit) 12, 13, 14, 15, 16, 17, 18, 19, 20, 21, 22, 23, 24, 25, 26, 27, 28, 29, 30, 32, 34, 36, 38, 40, 50, 60, 70, 80, or 90 bases in length. In some embodiments, the ODN is at most (upper limit) 100, 90, 80, 70, 60, 50, 49, 48, 47, 46, 45, 44, 43, 42, 41, 40, 39, 38, 37, 36, 35, 34, 33, 32, 31, or 30 bases in length. In some embodiments, the at least one palindromic sequence is 8 to 97 bases in length, preferably 8 to 50 bases in length, or preferably 8 to 32 bases in length. In some embodiments, the at least one palindromic sequence is at least (lower limit) 8, 10, 12, 14, 16, 18, 20, 22, 24, 26, 28, or 30 bases in length. In some embodiments, the at least one palindromic sequence is at most (upper limit) 50, 48, 46, 44, 42, 40, 38, 36, 34, 32, 30, 28, 26, 24, 22, 20, 18, 16, 14, 12 or 10 bases in length. In one embodiment, the oligonucleotide is an oligodeoxynucleotide. In one embodiment, one or more of the internucleotide linkages of the CpG-C ODN are modified linkages. In one embodiment, one or more of the internucleotide linkages of CpG-C ODN are phosphorothioate (PS) linkages. In one embodiment, all of the internucleotide linkages of CpG-C ODN are phosphorothioate (PS) linkages. A phosphorothioate backbone refers to all of the internucleotide linkages of CpG-C ODN being phosphorothioate (PS) linkages.

In one embodiment, the CpG-C ODNs of the present disclosure comprise:
(a) 5′-Nₓ(TCG(N_{q}))_{y}N_{w}(X₁X₂CGX₂′X₁′(CG)ₚ)_{z},Nᵥ (SEQ ID NO:22) wherein N are nucleosides, x=0, 1, 2 or 3, y=1, 2, 3 or 4, w=0, 1 or 2, p=0 or 1, q=0, 1 or 2, v=0 to 89 and z=1 to 20. X₁ and X₁′ are self-complementary nucleosides. X₂ and X₂′ are self-complementary nucleosides, and wherein the 5′-T of the (TCG(N_{q}))_{y}, sequence is 0-3 bases from the 5′ end of the oligonucleotide; and
(b) a palindromic sequence at least 8 bases in length wherein the palindromic sequence comprises the first (X₁X₂CGX₂′X₁′) (SEQ ID NO:25) of the (X₁X₂CGX₂′X₁′(CG)ₚ)_{z} (SEQ ID NO:26) sequences, wherein the ODN is from 12 to 100 bases in length. In some embodiments, x=0, y=1, w=0, p=0 or 1, q=0, 1 or 2, v=0 to 20 and z=1, 2, 3 or 4. In some embodiments, X₁ and X₂ are each either A or T. In some embodiments, the palindromic sequence has a base composition of more than one-third As and Ts. In some embodiments, the CpG-C ODN comprises a sequence selected from the group consisting of SEQ ID NOs: 10-24.

In some embodiments, the CpG-C ODNs of the present disclosure consist of TCGN_{q}(X₁X₂CGX₂′X₁′CG)_{z}Nᵥ (SEQ ID NO:23), wherein N are nucleosides, q=0, 1, 2, 3, 4, or 5, v=0 to 20, z=1 to 4, X₁ and X₁′ are self-complementary nucleosides, X₂ and X₂′ are self-complementary nucleosides, and wherein the ODN is at least 12 bases in length. In some embodiments, the CpG-C ODN consists of a sequence selected from the group consisting of SEQ ID NOs: 10-24.

In some embodiments, the CpG-C ODNs of the present disclosure consist of 5′-TCGN_{q}TTCGAACGTTCGAACGTTNₛ-3′ (SEQ ID NO:24), wherein N are nucleosides, q=0, 1, 2, 3, 4, or 5, s=0 to 20, and wherein the ODN is at least 12 bases in length. In one embodiment, s=0, 1, 2, 3, 4, or 5. In some embodiments, the CpG-C ODN consists of a sequence selected from the group consisting of

(SEQ ID NO: 13)
5′-TCGTTCGAACGTTCGAACGTTCGAA-3′
q = 0 and s = 4,
(SEQ ID NO: 14)
5′-TCGAACGTTCGAACGTTCGAACGTT-3′
q = 4 and s = 0,
(SEQ ID NO: 11)
5′-TCGAACGTTCGAACGTTCGAACGTTCGAAT-3′
q = 4 and s = 5,
(SEQ ID NO: 16)
5′-TCGTAACGTTCGAACGTTCGAACGTTA-3′
q = 5 and s = 1,
and
(SEQ ID NO: 17)
5′-TCGTAACGTTCGAACGTTCGAACGTT-3′
q = 5 and s = 0.

In one embodiment, the TLR9 agonist is a CpG-C ODN consisting of the sequence 5′-TCGAACGTTCGAACGTTCGAACGTTCGAAT-3′ (SEQ ID NO:11). In another embodiment, the CpG-C ODN is the sodium salt of 5′-TCGAACGTTCGAACGTTCGAACGTTCGAAT-3′ (SEQ ID NO:11). In a further embodiment, the CpG-C type oligonucleotide has a sequence that consists of 5′-TCGTTCGAACGTTCGAACGTTCGAA-3′ (SEQ ID NO:13). In a further embodiment, the CpG-C type oligonucleotide is a sodium salt of 5′-TCGTTCGAACGTTCGAACGTTCGAA-3′ (SEQ ID NO:13).

In another embodiment, the TLR9 agonist CpG-C type oligonucleotide is selected from the group consisting of:

(SEQ ID NO: 12)
5′-TCGTCGAACGTTCGAGATGAT-3′;
(SEQ ID NO: 13)
5′-TCGTTCGAACGTTCGAACGTTCGAA-3′;
(SEQ ID NO: 14)
5′-TCGAACGTTCGAACGTTCGAACGTT-3′;
(SEQ ID NO: 15)
5′-TCGAACGTTCGAACGTTCGAATTTT-3′;
(SEQ ID NO: 11)
5′-TCGAACGTTCGAACGTTCGAACGTTCGAAT-3′;
(SEQ ID NO: 16)
5′-TCGTAACGTTCGAACGTTCGAACGTTA-3′;
(SEQ ID NO: 17)
5′-TCGTAACGTTCGAACGTTCGAACGTT-3′;
(SEQ ID NO: 18)
5′-TCGTAACGTTCGAACGTTCGAACGT-3′;
(SEQ ID NO: 19)
5′-TCGTAACGTTCGAACGTTCGAACG-3′;
(SEQ ID NO: 20)
5′-TCGTAACGTTCGAACGTTCGAAC-3′;
and
(SEQ ID NO: 21)
5′-TCGTAACGTTCGAACGTTCGAA-3′.

In another aspect, the present application provides TLR4 agonists. In one aspect, the present application provides the following compounds: Lipid A, MPL, 3D-MPL, GLA, LPS, RC-529, E6020, ONO-4007, CRX-527, CRX-547, GSK1795091, CRX-601, SLA, PET-lipid A, PHAD, 3D-PHAD, 3D-(6-acyl)-PHAD, OM-294, OM-174, OK-432, 2B182C.

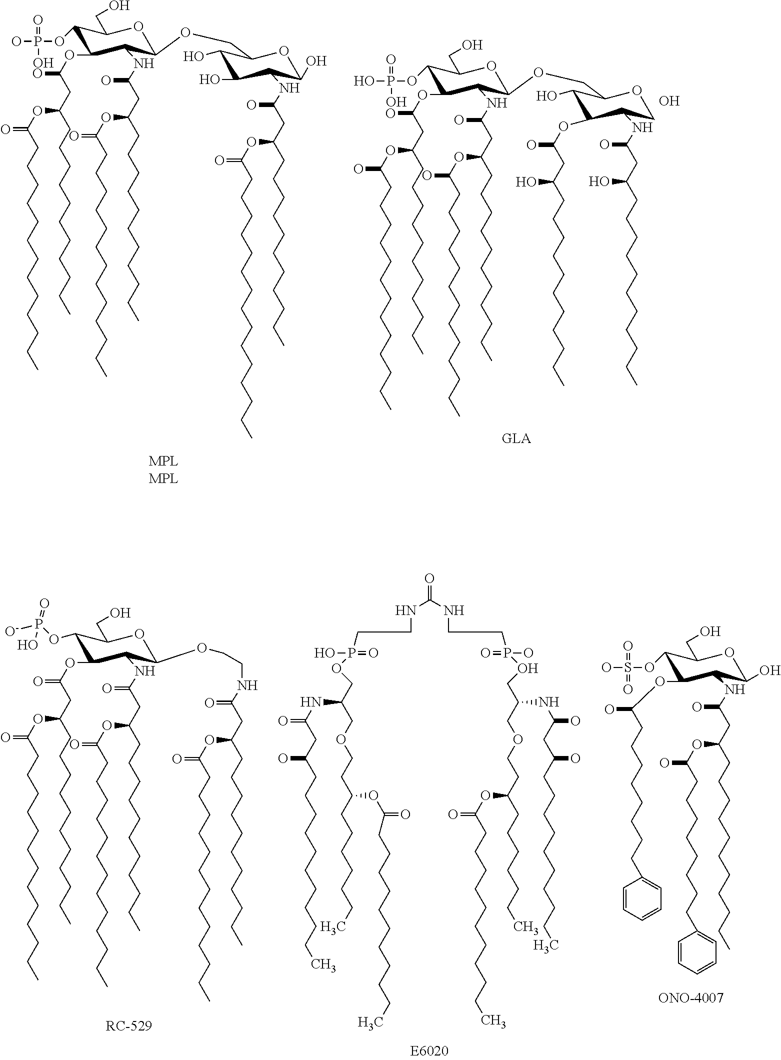

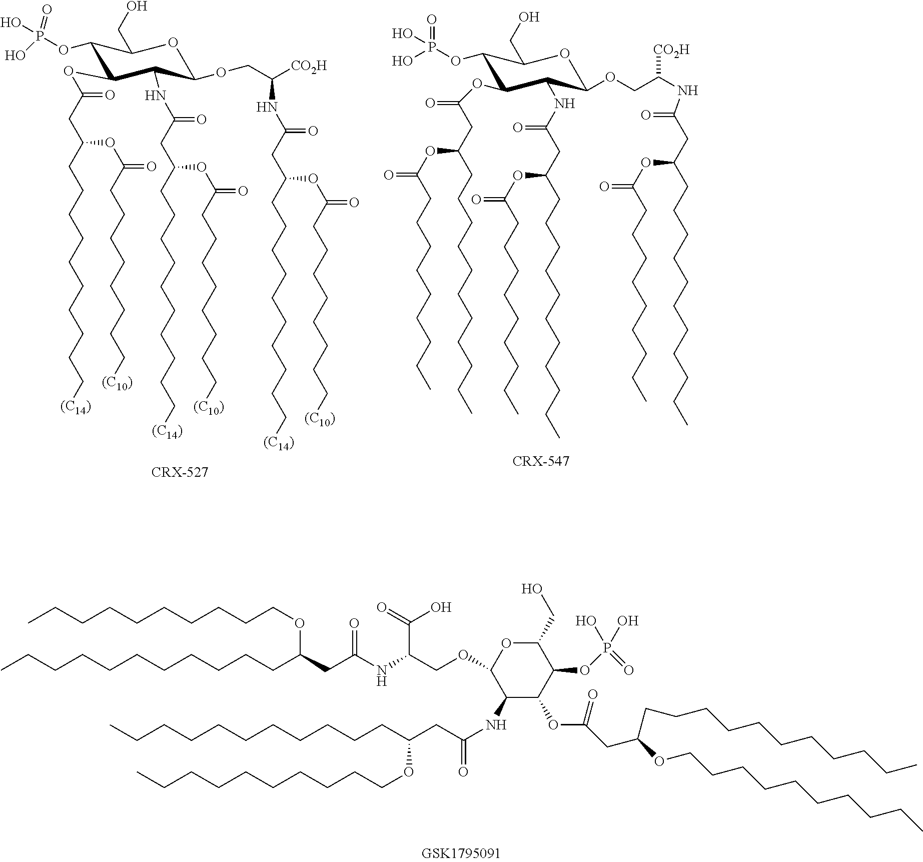

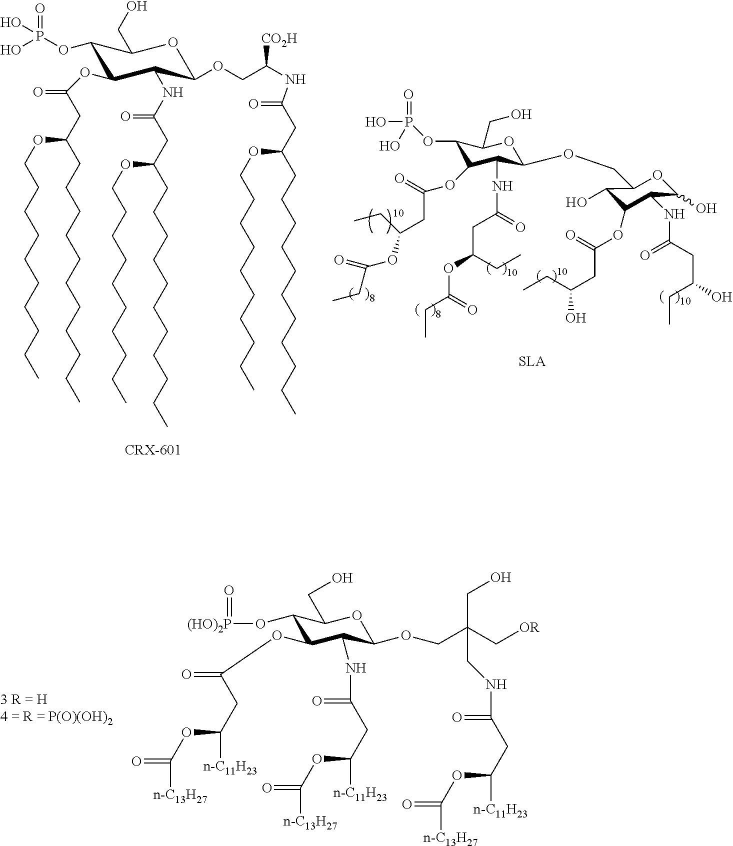

### PET-Lipid A (Containing an Amine-Substituted Pentaerythritol Instead of a Glucosamine Unit Found in Natural Lipid A and Retains Only One Phosphoryl Group)

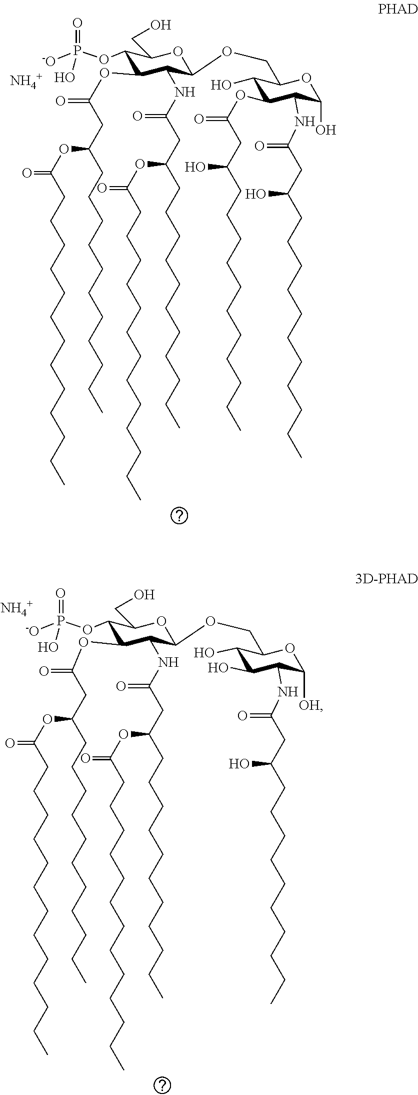

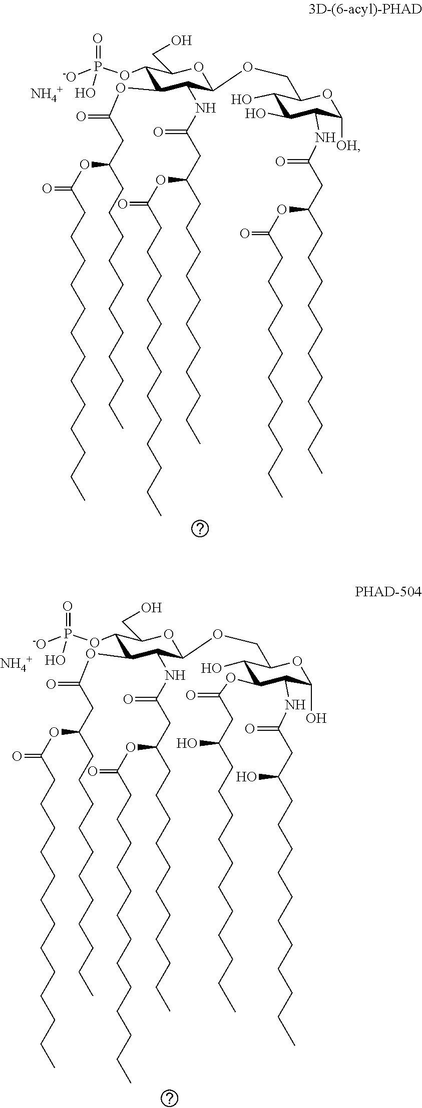

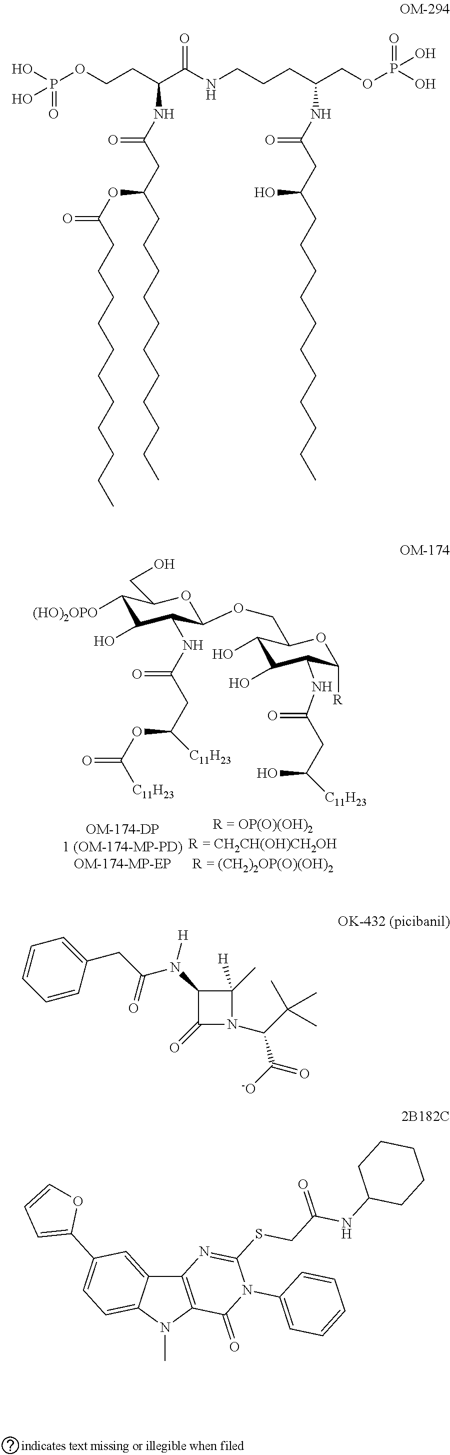

In another aspect, the present application provides E. coli derived monophosphoryl lipid A (EcML). EcML is a mixture of 4′-monophosphoryl lipids A (MPLAs) produced directly by an engineered Escherichia coli strain. In some embodiments, EcML comprises a mixture of hexa-acylated and penta-acylated MPLA congeners. In some embodiments, EcML comprises a mixture of hexa-acylated, penta-acylated, and tetra-acylated MPLA

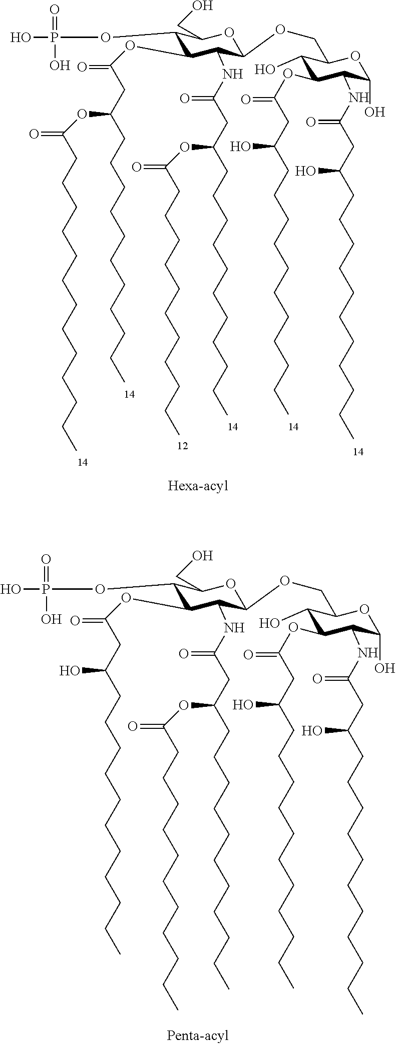

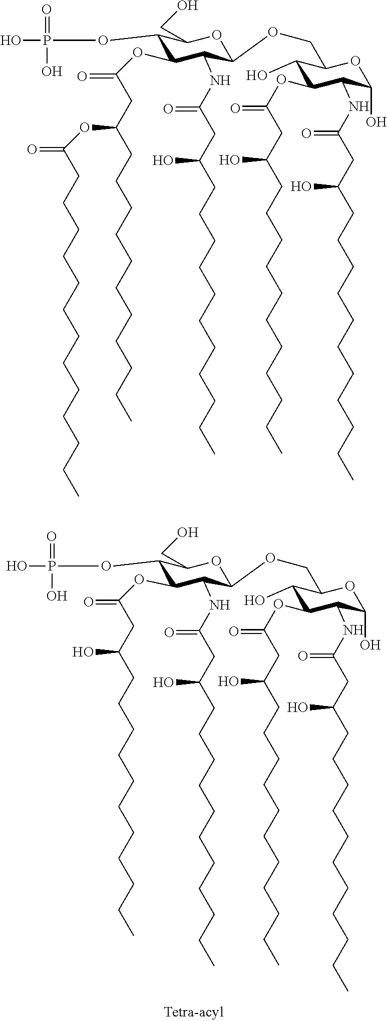

In some embodiments, EcML comprises at least 60% of the hexa-acyl congener and up to 40% of the penta-acyl congeners. In some embodiments, EcML comprises approximately 70% of the hexa-acyl congener, approximately 22% of the penta-acyl congeners, and approximately 8% of the tetra acyl congeners.

In another aspect, the present application provides the following compounds:

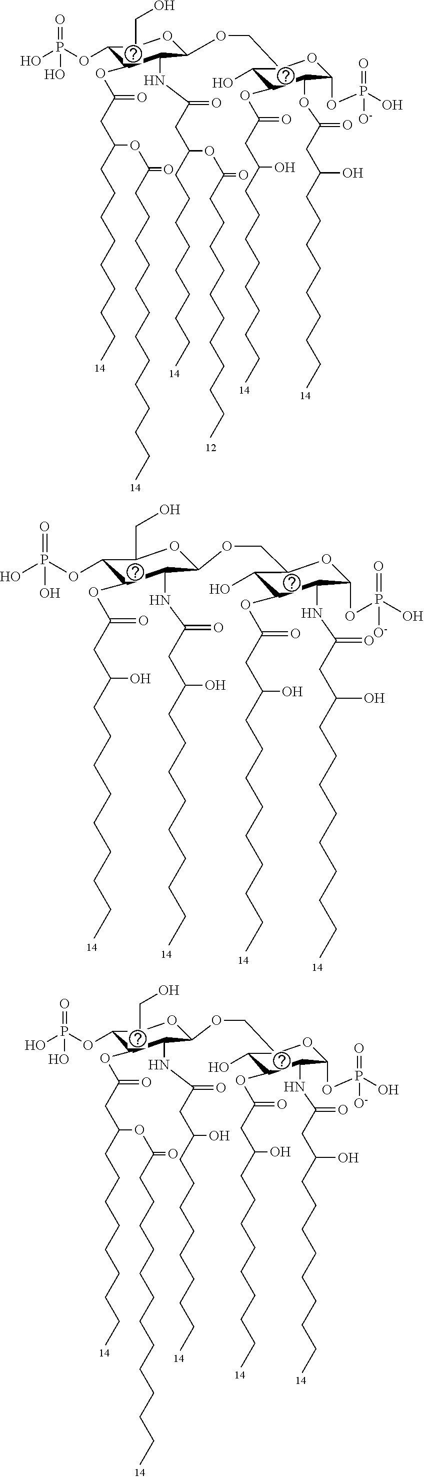

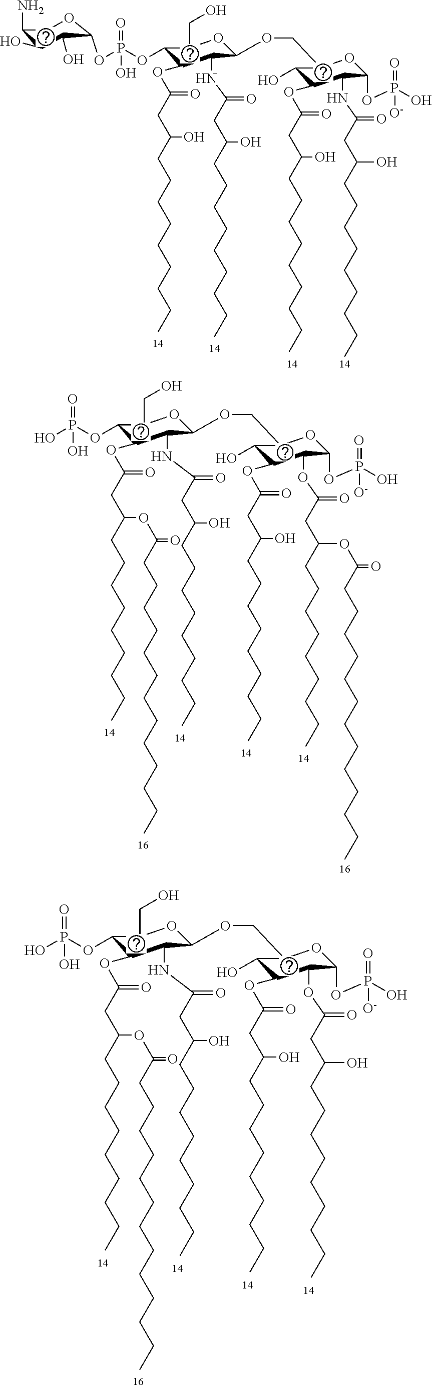

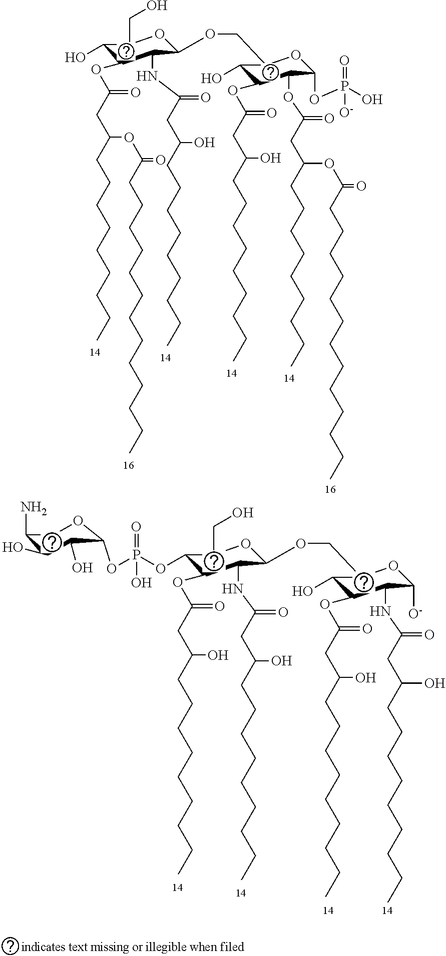

In preferred embodiments, the compound of Formula I is in free acid form, choline salt form, or formulated with cyclodextrin (e.g. HPBCD, (2-Hydroxypropyl)-β-cyclodextrin). In preferred embodiments, the compound of Formula I is TQL-1055. In preferred embodiments, the TLR4 agonist is PHAD, MPL, EcML, CRX-527, or MPLA or variants thereof. In preferred embodiments, the TLR9 agonist is CPG-1018 or SD-101.

In some of the preferred embodiments, the VZV glycoprotein contains a non-native signal peptide on the N-terminus to improve cleavage. In the context of the present application, such non-native signal peptides together with truncated or untruncated glycoproteins are not considered fusion proteins.

In another preferred embodiment, the present application utilizes an emulsion-based technology instead of or in conjunction with a liposome-forming compound. In some embodiments, the present application provides a combination having liposomes containing a TLR4 and/or TLR9 agonist and an emulsion containing the compound of Formula I or Formula II (or a pharmaceutically acceptable salt thereof). In some embodiments, the present application provides liposomes containing a TLR4 and/or TLR9 agonist and the compound of Formula I or Formula II (or a pharmaceutically acceptable salt thereof). In some embodiments, the present application provides an emulsion containing a TLR4 and/or TLR9 agonist and the compound of Formula I or Formula II (or a pharmaceutically acceptable salt thereof).

The present application also relates to methods of using a composition which induces an immune response against Varicella zoster virus, in the preparation of a medicament for the prevention or amelioration of varicella, herpes zoster, and/or post herpetic neuralgia.

The present application also relates to a method for the prevention or amelioration of varicella, herpes zoster, and/or post herpetic neuralgia, the method comprising administering to a human in need thereof an immunogenic composition or vaccine comprising a composition which induces an immune response against Varicella zoster virus as described above.

It will be appreciated by one of ordinary skill in the art that the compounds of the present application include, but are not necessarily limited to, those compounds encompassed in the genus set forth herein. The compounds encompassed by this application include at least all of the compounds disclosed in the entire specification as a whole, including all individual species within each genus.

According to another aspect of the present subject matter, the compounds disclosed in this application are useful as adjuvants. In another aspect, the present application provides a method for preparing compounds according to the embodiments of this application. In another aspect, the present invention provides a method of potentiating an immune response to an antigen, comprising administering to a subject a provided vaccine in an effective amount to potentiate the immune response of said subject to said antigen.

In another aspect, the present invention provides methods of vaccinating a subject, comprising administering a provided vaccine to said subject. In some embodiments, the subject is human. In some embodiments, the vaccine is administered as an injectable.

In another aspect, the invention provides pharmaceutical compositions comprising compounds of the invention and pharmaceutically acceptable excipients. In certain embodiments, the pharmaceutical composition is a vaccine comprising an antigen and an inventive adjuvant.

In another aspect, the invention provides kits comprising pharmaceutical compositions of inventive compounds. In some embodiments, the kits comprise prescribing information. In some embodiments, such kits include the combination of an inventive adjuvant compound and another immunotherapeutic agent. The agents may be packaged separately or together. The kit optionally includes instructions for prescribing the medication. In certain embodiments, the kit includes multiple doses of each agent. The kit may include sufficient quantities of each component to treat a subject for a week, two weeks, three weeks, four weeks, or multiple months. In certain embodiments, the kit includes one cycle of immunotherapy. In certain embodiments, the kit includes a sufficient quantity of a pharmaceutical composition to immunize a subject against an antigen long-term.

In another aspect, the application provides formulations of compositions according to the present application in an adjuvant system. In some embodiments, the adjuvant system utilizes a carrier. In some embodiments, the carrier is a particulate carrier such as metallic salt particles, emulsions, polymers, liposomes, or immune-stimulating complexes (ISCOMs). In some embodiments, the adjuvant system includes GLA, MPL, 3D-MPL, LPS, cholesterol, CpG (e.g. CpG 7907 or CpG 1018), PolyIC:LC, aluminum hydroxide, aluminum phosphate, tocopherol, acylated monosaccharides, other saponin derivatives (e.g. Quil-A, ISCOM, QS-21, and saponins contained in AS02 and AS01), soluble triterpene glycosides, Toll-like receptor 4 (TLR4) agonists, Toll-like receptor 3 (TLR3) agonists, montanides (ISA51, ISA720), immunostimulatory oligonucleotides, and imidazoquinolines. In some embodiments, the adjuvant system includes known immunostimulants. In some embodiments, the adjuvant system utilizes common adjuvants such as alum, Freund's adjuvant (an oil-in-water emulsion with dead mycobacteria), Freund's adjuvant with MDP (an oil-in-water emulsion with muramyl dipeptide, MDP, a constituent of mycobacteria), alum plus Bordetella pertussis (aluminum hydroxide gel with killed B. pertussis), enterobacteria, FU glycosides, synthetic or derived outer membrane vesicles, chitosan microparticles, and microcarrier parties, or other known adjuvants.

As used herein, the following definitions shall apply unless otherwise indicated.

“Liposomes” as used herein refer to closed bilayer membranes containing an entrapped aqueous volume. Liposomes may also be uni-lamellar vesicles possessing a single membrane bilayer or multi-lamellar vesicles with multiple membrane bilayers, each separated from the next by an aqueous layer. The structure of the resulting membrane bilayer is such that the hydrophobic (non-polar) tails of the lipid are oriented toward the center of the bilayer while the hydrophilic (polar) heads orient towards the aqueous phase. Liposomes, as they are ordinarily used, consist of smectic mesophases, and can consist of either phospholipid or nonphospholipid smectic mesophases. Smectic mesophase is most accurately described by Small, HANDBOOK OF LIPID RESEARCH, Vol. 4, Plenum, NY, 1986, pp. 49-50. According to Small, “[w]hen a given molecule is heated, instead of melting directly into an isotropic liquid, it may instead pass through intermediate states called mesophases or liquid crystals, characterized by residual order in some directions but by lack of order in others . . . . In general, the molecules of liquid crystals are somewhat longer than they are wide and have a polar or aromatic part somewhere along the length of the molecule. The molecular shape and the polar-polar, or aromatic, interaction permits the molecules to align in partially ordered arrays . . . . These structures characteristically occur in molecules that possess a polar group at one end. Liquid crystals with long-range order in the direction of the long axis of the molecule are called smectic, layered, or lamellar liquid crystals . . . . In the smectic states the molecules may be in single or double layers, normal or tilted to the plane of the layer, and with frozen or melted aliphatic chains.”

The term “aliphatic” or “aliphatic group,” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-12 aliphatic carbon atoms. In some embodiments, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups, and hybrids thereof such as (cycloalkyl)alkyl, (cycloalkenyl)alkyl or (cycloalkyl)alkenyl.

The term “lower alkyl” refers to a C₁₋₄ straight or branched alkyl group. Exemplary lower alkyl groups are methyl, ethyl, propyl, isopropyl, butyl, isobutyl, and tert-butyl. The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example, N (as in 3,4-dihydro-2H-pyrrolyl), NH (as in pyrrolidinyl) or NR+ (as in N-substituted pyrrolidinyl)).

The term “unsaturated,” as used herein, means that a moiety has one or more units of unsaturation.

As used herein, the term “bivalent C₁₋₁₂ (or C₁₋₂₆, C₁₋₁₆, C₁₋₈) or saturated or unsaturated, straight or branched, hydrocarbon chain,” refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)n-, wherein n is a positive integer, preferably from 1 to 30, from 1 to 28, from 1 to 26, from 1 to 24, from 1 to 22, from 1 to 20, from 1 to 18, from 1 to 16, from 1 to 14, from 1 to 12, from 1 to 10, from 1 to 8, from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “alkynylene” refers to a bivalent alkynyl group. A substituted alkynylene chain is a polymethylene group containing at least one double bond in which one or more hydrogen atoms are replaced with a substituent. Suitable substituents include those described below for a substituted aliphatic group.

The term “acyl,” used alone or as a part of a larger moiety, refers to groups formed by removing a hydroxy group from a carboxylic acid.

The term “halogen” means F, Cl, Br, or I.

The terms “aralkyl” and “arylalkyl” are used interchangeably and refer to alkyl groups in which a hydrogen atom has been replaced with an aryl group. Such groups include, without limitation, benzyl, cinnamyl, and dihyrocinnamyl.

The term “aryl” used alone or as part of a larger moiety as in “aralkyl.” “aralkoxy.” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.”

In certain embodiments of the present invention, “aryl” refers to an aromatic ring system which includes, but is not limited to, phenyl, biphenyl, naphthyl, anthracyl, and the like, which may bear one or more substituents. Also, included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The terms “heteroaralkyl” and “heteroarylalkyl” refer to an alkyl group substituted by a heteroaryl moiety, wherein the alkyl and heteroaryl portions independently are optionally substituted.

The term “heteroaliphatic,” as used herein, means aliphatic groups wherein one or two carbon atoms are independently replaced by one or more of oxygen, sulfur, nitrogen, or phosphorus. Heteroaliphatic groups may be substituted or unsubstituted, branched or unbranched, cyclic or acyclic, and include “heterocycle,” “heterocyclyl,” “heterocycloaliphatic,” or “heterocyclic” groups.

As used herein, the terms “heterocycle,” “heterocyclyl.” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur, or nitrogen, the nitrogen may be N (as in 3,4-dihydro-2H-pyrrolyl), NH (as in pyrrolidinyl), or +NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group.” “heterocyclic moiety.” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

In another aspect, the present invention provides “pharmaceutically acceptable” compositions, which comprise a therapeutically effective amount of one or more of the compounds described herein, formulated together with one or more pharmaceutically acceptable carriers (additives) and/or diluents. As described in detail, the pharmaceutical compositions of the present invention may be specially formulated for administration by injection.

The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically-acceptable material, composition, or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials that can serve as pharmaceutically-acceptable carriers include sugars, such as lactose, glucose, and sucrose; starches, such as corn starch and potato starch; cellulose, and its derivatives, such as sodium carboxymethyl cellulose, ethyl cellulose, and cellulose acetate; powdered tragacanth; malt; gelatin; talc; excipients, such as cocoa butter and suppository waxes: oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

As used herein, the term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio.

Unless otherwise stated, structures depicted herein are also meant to include all isomeric (e.g., enantiomeric, diastereomeric, and geometric (or conformational)) forms of the structure; for example, the R and S configurations for each stereocenter, Z and E double bond isomers, and Z and E conformational isomers. Therefore, single stereochemical isomers, as well as enantiomeric, diastereomeric, and geometric (or conformational) mixtures of the present compounds, are within the scope of the invention. Unless otherwise stated, all tautomeric forms of the compounds of the invention are within the scope of the invention.

Provided compounds may comprise one or more saccharide moieties. Unless otherwise specified, both D- and L-configurations, and mixtures thereof, are within the scope of the invention. Unless otherwise specified, both α- and β-linked embodiments, and mixtures thereof, are contemplated by the present invention.

If, for instance, a particular enantiomer of a compound of the present invention is desired, it may be prepared by asymmetric synthesis, chiral chromatography, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a 13C- or 14C-enriched carbon are within the scope of this invention. Such compounds are useful, for example, as analytical tools, as probes in biological assays, or as therapeutic agents in accordance with the present invention.

One of ordinary skill in the art will appreciate that the synthetic methods, as described herein, utilize a variety of protecting groups. By the term “protecting group,” as used herein, it is meant that a particular functional moiety, e.g., O, S, or N, is masked or blocked, permitting, if desired, a reaction to be carried out selectively at another reactive site in a multifunctional compound. In preferred embodiments, a protecting group reacts selectively in good yield to give a protected substrate that is stable to the projected reactions; the protecting group is preferably selectively removable by readily available, preferably non-toxic reagents that do not attack the other functional groups; the protecting group forms a separable derivative (more preferably without the generation of new stereogenic centers); and the protecting group will preferably have a minimum of additional functionality to avoid further sites of reaction. As detailed herein, oxygen, sulfur, nitrogen, and carbon protecting groups may be utilized. By way of non-limiting example, hydroxyl protecting groups include methyl, methoxylmethyl (MOM), methylthiomethyl (MTM), t-butylthiomethyl, (phenyldimethylsilyl) methoxymethyl (SMOM), benzyloxymethyl (BOM): p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl) xanthenyl, 9-(9-phenyl-10-oxo) anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio) pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio)ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy) butyrate, 2-(methylthiomethoxymethyl)benzoate, 2,6-dichloro-4-methylphenoxyacetate, 2,6-dichloro-4-(1,1,3,3-tetramethylbutyl) phenoxyacetate, 2,4-bis(1,1-dimethylpropyl) phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate. Amino-protecting groups include methyl carbamate, ethyl carbamate, 9-fluorenylmethyl carbamate (Fmoc), 9-(2-sulfo) fluorenylmethyl carbamate, 9-(2,7-dibromo) fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido) ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl) ethyl carbamate. [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino) acetamide, 3-(p-hydroxyphenyl) propanamide, 3-(0-nitrophenyl) propanamide, 2-methyl-2-(o-nitrophenoxy) propanamide, 2-methyl-2-(o-phenylazophenoxy) propanamide, 4-chlorobutanamide, 3-methyl-3-nitrobutanamide, o-nitrocinnamide, N-acetylmethionine derivative, o-nitrobenzamide, 0-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)-amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N′,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6, -trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide. Exemplary protecting groups are detailed herein, however, it will be appreciated that the present invention is not intended to be limited to these protecting groups; rather, a variety of additional equivalent protecting groups can be readily identified using the above criteria and utilized in the method of the present invention. Additionally, a variety of protecting groups are described by Greene and Wuts (supra).

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen: —(CH₂)₀₋₄R^{o}; —(CH₂)₀₋₄OR^{o}; —O(CH₂)₀₋₄R^{o}, —O—(CH₂)₀₋₄C(O)OR^{o}; —(CH₂)₀₋₄CH(OR^{o})₂; —(CH₂)₀₋₄SR^{o}; —(CH₂)₀₋₄Ph, which may be substituted with R^{o}: —(CH₂)₀₋₄O(CH₂)₀₋₁Ph, which may be substituted with R^{o}; —CH═CHPh, which may be substituted with R^{o}; —(CH₂)₀₋₄O(CH₂)₀₋₁-pyridyl which may be substituted with R^{o}: —NO₂; —CN: —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}₂; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o}C(O)NR^{o}₂; —N(R^{o})N(R^{o}) C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR, —SC(S)SR^{o}; —(CH₂)₀₋₄SC(O)R^{o}; —(CH₂)₀₋₄C(O)NR^{o}₂; —C(S)NR^{o}₂; —C(S)SR^{o}; —SC(S)SR^{o}, —(CH₂)₀₋₄OC(O)NR^{o}₂; —C(O)N(OR^{o}) R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}: —(CH₂)₀₋₄S(O)₂OR^{o}; —(CH₂)₀₋₄OS(O)₂R^{o}; —S(O)₂NR^{o}₂; —(CH₂)₀₋₄S(O)R^{o}; —N(R^{o})S(O)₂NR^{o}₂; —N(R^{o})S(O)₂R^{o}; —N(OR^{o})R^{o}; —C(NH)NR^{o}₂; —P(O)₂R^{o}; —P(O)R^{o}₂; —OP(O)R^{o}₂; —OP(O)(OR^{o})₂; SiR^{o}₃; —(C₁₋₄ straight or branched)alkylene)O—N(R^{o})₂; or —(C₁₋₄ straight or branched)alkylene)C(O)O—N(R^{o})₂, wherein each R^{o} may be substituted as defined below and is independently hydrogen, C₁₋₆ aliphatic, —CH₂Ph, —O(CH₂)₀₋₁Ph, —CH₂-(5-6-membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms) are independently halogen, —(CH₂)₀₋₂R^{Δ}, -(haloR^{Δ}), —(CH₂)₀₋₂OH, —(CH₂)₀₋₂OR^{Δ}, —(CH₂)₀₋₂CH(OR^{Δ})₂; —O(haloR^{Δ}), —CN, —N₃, —(CH₂)₀₋₂C(O)R^{Δ}, —(CH₂)₀₋₂C(O)OH, —(CH₂)₀₋₂C(O)OR^{Δ}, —(CH₂)₆₋₂SR^{Δ}, —(CH₂)₀₋₂SH, —(CH₂)₀₋₂NH₂, —(CH₂)₀₋₂NHR^{Δ}, —(CH₂)₀₋₂NR^{Δ}₂, —NO₂, —SiR^{Δ}₃, —OSiR^{Δ}₃, —C(O)SR^{Δ}, —(C₁₋₄ straight or branched alkylene)C(O)OR^{Δ}, or —SSR, wherein each R^{Δ} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{o} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R′ include halogen, —R^{Δ}, -(haloR^{Δ}), —OH, —OR^{Δ}, —O(haloR^{Δ}), —CN, —C(O)OH, —C(O)OR^{Δ}, —NH₂, —NHR^{Δ}, —NR^{Δ}₂, or —NO₂, wherein each R^{Δ} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}. —S(O)₂R^{†}, —S(O)₂NR^{†}₂, —C(S)NR^{†}₂, —C(NH)NR^{†}₂, or —N(R^{†})S(O)₂R^{†}; wherein each Rt is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of Rt, taken together with their intervening atom(s) form an unsubstituted 3-12-membered saturated, partially unsaturated, or aryl mono- or bicyclic ring having 0-4 heteroatoms independently selected from nitrogen, oxygen, or sulfur. Suitable substituents on the aliphatic group of Rt are independently halogen, —R^{Δ}, -(haloR^{Δ}), —OH, —OR^{Δ}, —O(haloR^{Δ}), —CN, —C(O)OH, —C(O)OR^{Δ}, —NH₂, —NHR^{Δ}, —NR^{Δ}₂, or —NO₂, wherein each R^{Δ} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug, or other material other than directly into the central nervous system, such that it enters the patient's system and, thus, is subject to metabolism and other like processes, for example, subcutaneous administration.

The term “enriched” as used herein refers to a mixture having an increased proportion of one or more species. In some embodiments, the mixture is “enriched” following a process that increases the proportion of one or more desired species in the mixture. In some embodiments, the desired species comprise(s) greater than 10% of the mixture. In some embodiments, the desired species comprise(s) greater than 25% of the mixture. In some embodiments, the desired species comprise(s) greater than 40% of the mixture. In some embodiments, the desired species comprise(s) greater than 60% of the mixture. In some embodiments, the desired species comprise(s) greater than 75% of the mixture. In some embodiments, the desired species comprise(s) greater than 85% of the mixture. In some embodiments, the desired species comprise(s) greater than 90% of the mixture. In some embodiments, the desired species comprise(s) greater than 95% of the mixture. Such proportions can be measured in any number of ways, for example, as a molar ratio, volume to volume, or weight to weight.

The term “pure” refers to compounds that are substantially free of compounds of related non-target structure or chemical precursors (when chemically synthesized). This quality may be measured or expressed as “purity.” In some embodiments, a target compound has less than about 30%, 20%, 10%, 5%, 2%, 1%, 0.5%, and 0.1% of non-target structures or chemical precursors. In certain embodiments, a pure compound of the present invention is only one prosapogenin compound (i.e., separation of target prosapogenin from other prosapogenins).

The term “carbohydrate” refers to a sugar or polymer of sugars. The terms “saccharide”, “polysaccharide”. “carbohydrate”, and “oligosaccharide”, may be used interchangeably. Most carbohydrates are aldehydes or ketones with many hydroxyl groups, usually one on each carbon atom of the molecule. Carbohydrates generally have the molecular formula CₙH₂ₙOₙ. A carbohydrate may be a monosaccharide, a disaccharide, trisaccharide, oligosaccharide, or polysaccharide. The most basic carbohydrate is a monosaccharide, such as glucose, sucrose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates may contain modified saccharide units such as 2′-deoxyribose wherein a hydroxyl group is removed, 2′-fluororibose wherein a hydroxyl group is replaced with a fluorine, or N-acetylglucosamine, a nitrogen-containing form of glucose. (e.g., 2′-fluororibose, deoxyribose, and hexose). Carbohydrates may exist in many different forms, for example, conformers, cyclic forms, acyclic forms, stereoisomers, tautomers, anomers, and isomers.

Further objects, features, and advantages of the present application will become apparent from the detailed description which is set forth below when considered together with the figures of drawing.

### BRIEF DESCRIPTION OF THE DRAWINGS

FIG. 1 depicts the sequence of a truncated VZV gE.

FIG. 2 depicts the sequence of a truncated VZV gE.

FIG. 3 depicts the sequence of a VZV gE.

FIG. 4 depicts the sequence of a VZV gE.

FIG. 5 depicts the sequence of a VZV gB.

FIG. 6 depicts the sequence of a VZV gH.

FIG. 7 depicts the sequence of a VZV gI.

FIG. 8 depicts the sequence of a VZV gC.

FIG. 9 depicts the sequence of a truncated VZV gE.

FIGS. 10-15 depict results and data from Example 1 discussed below.

FIGS. 16-19 depict results and data from Example 2 discussed below.

FIGS. 20-21 depict results and data from Example 3 discussed below.

FIGS. 22-27 depict results and data from Example 4 discussed below.

FIGS. 28-39 depict results and data from Example 5 discussed below.

FIGS. 40-75 depict results and data from Example 6 discussed below.

### DETAILED DESCRIPTION OF CERTAIN EMBODIMENTS

In its broadest aspect the present application relates to compositions and regimes for provoking an immune response. In one aspect the immune response generated by exposure to such compositions is higher and statistically significant when compared to that obtained in individuals who have received no exposure to the compositions of the present application. The immune response may be assessed by analysis of any one or more aspects of cell-mediated immune (CMI) response and/or antibody responses using any of the techniques outlined below or those familiar to a person of ordinary skill in the art.

The inventors of the present application have invented combinations of certain saponin adjuvants, including preferably TQL-1055, and certain TLR4 and/or TLR9 agonists, which together will produce surprising, beneficial, and/or synergistic effects. The inventors of the present application also provide pharmaceutical compositions comprising the foregoing and methods of making and of using the foregoing in the treatment of certain diseases.

In another aspect, the present application discloses methods for preventing and/or decreasing the severity of infection of certain pathogens where CD4 or CD8 responses are thought to control infection directly, including, for example, HIV, all herpes viruses (e.g. HSV-1/2, varicella zoster virus (VZV), EBV, CMV, and HHV6/7/8, and their related disorders such as herpes zoster and post herpetic neuralgia (PHN)), Plasmodium falciparum and other Plasmodium species, Mycobacterium tuberculosis, M. leprae other Mycobacteria species, Salmonella typhi and other Salmonella species, Leishmania major and other Leishmania species, Necator americanus, Ancylostoma duodenale, Giardia lamblia and other Giardia species, Cryptosporidium parvum and other Cryptosporidium species, Coccidioides immitis and other Coccidiodes species, and Histoplasma capsulatum and other Histoplasma species.

In another aspect, the present application discloses methods for preventing and/or decreasing the severity of infection of certain pathogens where the antibody response may be improved in quality or duration by a helper CD4 response, including, for example, Influenza A and B viruses, Bordetella pertussis, Coronaviruses (including SARS-COV1, MERS-COV, and SARS-COV2), vesicular stomatitis virus (VSV), Vaccinia viruses, respiratory syncytial virus (RSV), Dengue virus, Zika virus, West Nile Virus, Yellow Fever virus (YFV), and alphaviruses including equine encephalitis (VEE/EEE/WEE), Chikungunya, Ross River, and O'Nyong-nyong viruses.

In another aspect, the present application discloses methods for preventing and/or decreasing the severity of varicella, herpes zoster, and post herpetic neuralgia (PHN). Thus, in one aspect, the present application discloses methods of prevention of the incidence of varicella.

Where infection in one of the foregoing does occur, the severity is reduced compared with an unvaccinated control (amelioration of disease). In another aspect, the present application discloses methods of prevention of the incidence of infection with one of the foregoing. Where infection does occur, the severity of the reactivation of certain pathogens is reduced compared with an unvaccinated control (amelioration of zoster). In a further aspect, where zoster does occur, the present application discloses methods of prevention of the incidence of PHN. In a further aspect where PHN does occur then the severity of the PHN is suitably reduced compared with an unvaccinated control (amelioration of PHN). Reduction in severity can suitably be assessed by a reduction in the pain caused by varicella, herpes zoster, or PHN, for example, using known measures of burden of pain (e.g. Coplan et al J Pain 2004; 5 (6) 344-56). Reduction in severity can also be assessed by other criteria such as duration of varicella, herpes zoster, or PHN, proportion of body area affected by varicella, herpes zoster, or PHN, or the site of varicella, herpes zoster, or PHN.

The present application provides pharmaceutical compositions comprising the compounds of the present application together with an immunologically effective amount of an antigen associated with certain pathogens where CD4 responses are thought to control infection directly, including, for example, HIV, all herpes viruses (e.g. HSV-1/2, varicella zoster virus (VZV), EBV, CMV, and HHV6/7/8, and their related disorders such as herpes zoster and post herpetic neuralgia (PHN)), Plasmodium falciparum and other Plasmodium species, Mycobacterium tuberculosis, M. leprae other Mycobacteria species, Salmonella typhi and other Salmonella species, Leishmania major and other Leishmania species, Necator americanus, Ancylostoma duodenale, Giardia lamblia and other Giardia species, Cryptosporidium parvum and other Cryptosporidium species, Coccidioides immitis and other Coccidiodes species, and Histoplasma capsulatum and other Histoplasma species.

The present application provides pharmaceutical compositions comprising the compounds of the present application together with an immunologically effective amount of an antigen associated with certain pathogens where the antibody response may be improved in quality or duration by a helper CD4 response, including, for example, Influenza A and B viruses, Bordetella pertussis, Coronaviruses (including SARS-COV1, MERS-COV, and SARS-Cov2), vesicular stomatitis virus (VSV), Vaccinia viruses, respiratory syncytial virus (RSV), Dengue virus, Zika virus, West Nile Virus, Yellow Fever virus (YFV), and alphaviruses including equine encephalitis (VEE/EEE/WEE), Chikungunya, Ross River, and O'Nyong-nyong viruses.

The present application provides pharmaceutical compositions comprising the compounds of the present application together with an immunologically effective amount of an antigen associated with Varicella zoster virus (VZV).

The application also includes methods of vaccinating a human patient comprising administering an immunologically effective amount of a pharmaceutical compositions or of the compounds of the present application. The application also includes methods for increasing the immune response to a vaccine comprising administering an immunologically effective amount of a pharmaceutical compositions or of the compounds of the present application.

### Compounds

Compounds of this invention include those described generally above, and are further illustrated by the classes, subclasses, and species disclosed herein. In some embodiments, provided compounds are analogs of naturally occurring triterpene glycoside saponins and intermediates thereto. For purposes of this invention, the chemical elements are identified in accordance with the Periodic Table of the Elements, CAS version, Handbook of Chemistry and Physics, 75th Ed. Additionally, general principles of organic chemistry are described in Organic Chemistry, Thomas Sorrell, University Science Books, Sausalito: 1999, and March's Advanced Organic Chemistry, 5th Ed., Ed.: Smith, M. B. and March, J., John Wiley & Sons, New York: 2001, the entire contents of which are hereby incorporated by reference.

### Saponins

In some embodiments, provided compounds are analogs of Quillaja saponins. In some embodiments, provided compounds are prosapogenins. In certain embodiments, provided compounds are analogs of QS-7 and QS-21 and possess potent adjuvant activity.

In one aspect, the present application provides compounds of Formula I:

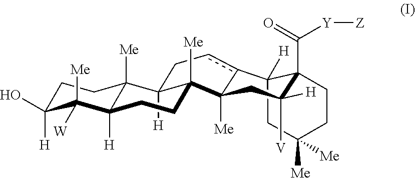

or a pharmaceutically acceptable salt thereof, wherein
[symbol] is a single or double bond;
W is —CHO;
V is hydrogen or OR^{x};
Y is CH₂, —O—, —NR—, or —NH—;
Z is hydrogen; a cyclic or acyclic, optionally substituted moiety selected from the group consisting of acyl, aliphatic, heteroaliphatic, aryl, arylalkyl, heteroacyl, and heteroaryl; or a carbohydrate domain having the structure:

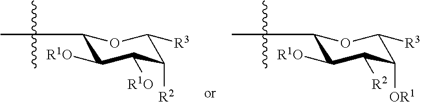

wherein each occurrence of R¹ is R^{x} or a carbohydrate domain having the structure:

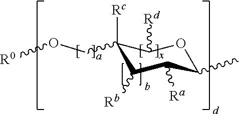

wherein:
each occurrence of a, b, and c is independently 0, 1, or 2;
d is an integer from 1-5, wherein each d bracketed structure may be the same or different; with the proviso that the d bracketed structure represents a furanose or a pyranose moiety, and the sum of b and c is 1 or 2;
R⁰ is hydrogen; an oxygen protecting group selected from the group consisting of alkyl ethers, benzyl ethers, silyl ethers, acetals, ketals, esters, carbamates, and carbonates; or an optionally substituted moiety selected from the group consisting of acyl, C₁₋₁₀ aliphatic, C₁₋₆ heteroaliphatic, 6-10-membered aryl, arylalkyl, 5-10 membered heteroaryl having 1-4 heteroatoms independently selected from nitrogen, oxygen, or sulfur, 4-7 membered heterocyclyl having 1-2 heteroatoms independently selected from the group consisting of nitrogen, oxygen, and sulfur;
each occurrence of R^{a}, R^{b}, R^{c}, and R^{d} is independently hydrogen, halogen, OH, OR, OR^{x}, NR₂, NHCOR, or an optionally substituted group selected from acyl, C₁₋₁₀ aliphatic, C₁₋₆ heteroaliphatic, 6-10-membered aryl, arylalkyl, 5-10-membered heteroaryl having 1-4 heteroatoms independently selected from nitrogen, oxygen, sulfur; 4-7-membered heterocyclyl having 1-2 heteroatoms independently selected from the group consisting of nitrogen, oxygen, and sulfur;
R² is hydrogen, halogen, OH, OR, OC(O)R⁴, OC(O)OR⁴, OC(O)NHR⁴, OC(O)NRR⁴, OC(O)SR⁴, NHC(O)R⁴, NRC(O)R⁴, NHC(O)OR⁴, NHC(O)NHR⁴, NHC(O)NRR⁴, NHR⁴, N(R⁴)₂, NHR⁴, NRR⁴, N₃, or an optionally substituted group selected from C₁₋₁₀ aliphatic, C₁₋₆ heteroaliphatic, 6-10-membered aryl, arylalkyl, 5-10 membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur;
R³ is hydrogen, halogen, CH₂OR¹, or an optionally substituted group selected from the group consisting of acyl, C₁₋₁₀ aliphatic, C₁₋₆ heteroaliphatic, 6-10-membered aryl, arylalkyl, 5-10-membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur,
R⁴ is -T-R^{z}, —C(O)-T-R^{z}, —NH-T-R^{z}, —O-T-R^{z}, —S-T-R^{z}, —C(O)NH-T-R^{z}, C(O)O-T-R^{z}, C(O)S-T-R^{z}, C(O)NH-T-O-T-R^{z}, —O-T-R^{z}, -T-O-T-R^{z}, -T-S-T-R^{z}, or

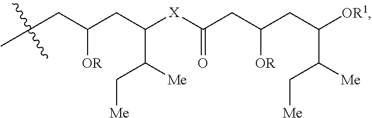

wherein
X is —O—, —NR—, or T-R^{z};
T is a covalent bond or a bivalent C₁₋₂₆ saturated or unsaturated, straight or branched, aliphatic or heteroaliphatic chain; and
R^{z} is hydrogen, halogen, —OR, —OR^{x}, —OR¹, —SR, NR₂, —C(O)OR, —C(O)R, —NHC(O)R, —NHC(O)OR, NC(O)OR, or an optionally substituted group selected from acyl, arylalkyl, heteroarylalkyl, C₁₋₆ aliphatic, 6-10-membered aryl, 5-10-membered heteroaryl having 1-4 heteroatoms independently selected from nitrogen, oxygen, or sulfur, 4-7-membered heterocyclyl having 1-2 heteroatoms independently selected from the group consisting of nitrogen, oxygen, and sulfur;
each occurrence of R^{x} is independently hydrogen or an oxygen protecting group selected from the group consisting of alkyl ethers, benzyl ethers, silyl ethers, acetals, ketals, esters, carbamates, and carbonates;
each occurrence of R is independently hydrogen, an optionally substituted group selected from acyl, arylalkyl, 6-10-membered aryl, C₁₋₆ aliphatic, or C₁₋₆ heteroaliphatic having 1-2 heteroatoms independently selected from the group consisting of nitrogen, oxygen, and sulfur, or:
   two R on the same nitrogen atom are taken with the nitrogen atom to form a 4-7-membered heterocyclic ring having 1-2 heteroatoms independently selected from the group consisting of nitrogen, oxygen, and sulfur.

In one aspect, the present application provides compounds of Formula II:

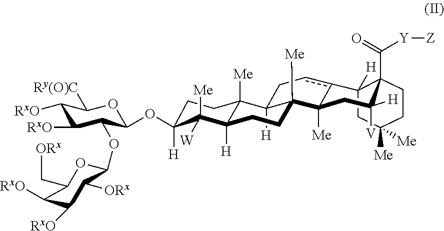

or a pharmaceutically acceptable salt thereof, wherein
[symbol] is a single or double bond;
W is Me, —CHO, or

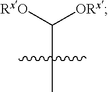

V is hydrogen or OR^{x};
Y is CH₂, —O—, —NR—, or —NH—;
Z is hydrogen; a cyclic or acyclic, optionally substituted moiety selected from the group consisting of acyl, aliphatic, heteroaliphatic, aryl, arylalkyl, heteroacyl, and heteroaryl; or a carbohydrate domain having the structure:

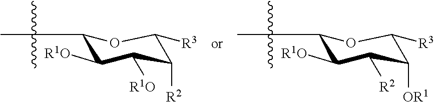

wherein each occurrence of R¹ is R^{x} or a carbohydrate domain having the structure:

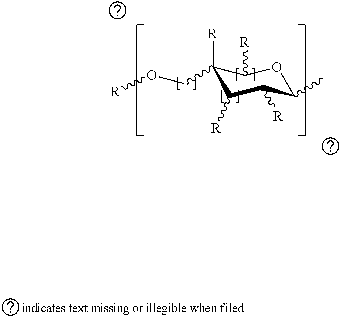

wherein:
each occurrence of a, b, and c is independently 0, 1, or 2;
d is an integer from 1-5, wherein each d bracketed structure may be the same or different; with the proviso that the d bracketed structure represents a furanose or a pyranose moiety, and the sum of b and c is 1 or 2;
R⁰ is hydrogen; an oxygen protecting group selected from the group consisting of alkyl ethers, benzyl ethers, silyl ethers, acetals, ketals, esters, carbamates, and carbonates; or an optionally substituted moiety selected from the group consisting of acyl, C₁₋₁₀ aliphatic, C₁₋₆ heteroaliphatic, 6-10-membered aryl, arylalkyl, 5-10 membered heteroaryl having 1-4 heteroatoms independently selected from nitrogen, oxygen, or sulfur, 4-7 membered heterocyclyl having 1-2 heteroatoms independently selected from the group consisting of nitrogen, oxygen, and sulfur;
each occurrence of R^{a}, R^{b}, R^{c}, and R^{d} is independently hydrogen, halogen, OH, OR, OR^{x}, NR₂, NHCOR, or an optionally substituted group selected from acyl, C₁₋₁₀ aliphatic, C₁₋₆ heteroaliphatic, 6-10-membered aryl, arylalkyl, 5-10-membered heteroaryl having 1-4 heteroatoms independently selected from nitrogen, oxygen, sulfur; 4-7-membered heterocyclyl having 1-2 heteroatoms independently selected from the group consisting of nitrogen, oxygen, and sulfur;
R² is hydrogen, halogen, OH, OR, OC(O)R⁴, OC(O)OR⁴, OC(O)NHR⁴, OC(O)NRR⁴, OC(O)SR⁴, NHC(O)R⁴, NRC(O)R⁴, NHC(O)OR⁴, NHC(O)NHR⁴, NHC(O)NRR⁴, NHR⁴, N(R⁴)₂, NHR⁴, NRR⁴, N₃, or an optionally substituted group selected from C₁₋₁₀ aliphatic, C₁₋₆ heteroaliphatic, 6-10-membered aryl, arylalkyl, 5-10 membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur;
R³ is hydrogen, halogen, CH₂OR¹, or an optionally substituted group selected from the group consisting of acyl, C₁₋₁₀ aliphatic, C₁₋₆ heteroaliphatic, 6-10-membered aryl, arylalkyl, 5-10-membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur,
R⁴ is -T-R^{z}, —C(O)-T-R^{z}, —NH-T-R^{z}, —O-T-R^{z}, —S-T-R^{z}, —C(O)NH-T-R^{z}, C(O)O-T-R^{z}, C(O)S-T-R^{z}, C(O)NH-T-O-T-R^{z}, —O-T-R^{z}, -T-O-T-R^{z}, -T-S-T-R^{z}, or

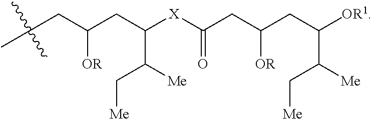

wherein
X is —O—, —NR—, or T-R^{z};
T is a covalent bond or a bivalent C₁₋₂₆ saturated or unsaturated, straight or branched, aliphatic or heteroaliphatic chain; and
R^{z} is hydrogen, halogen, —OR, —OR^{x}, —OR¹, —SR, NR₂, —C(O)OR, —C(O)R, —NHC(O)R, —NHC(O)OR, NC(O)OR, or an optionally substituted group selected from acyl, arylalkyl, heteroarylalkyl, C₁₋₆ aliphatic, 6-10-membered aryl, 5-10-membered heteroaryl having 1-4 heteroatoms independently selected from nitrogen, oxygen, or sulfur, 4-7-membered heterocyclyl having 1-2 heteroatoms independently selected from the group consisting of nitrogen, oxygen, and sulfur;
each occurrence of R^{x} is independently hydrogen or an oxygen protecting group selected from the group consisting of alkyl ethers, benzyl ethers, silyl ethers, acetals, ketals, esters, carbamates, and carbonates;
R^{y} is —OH, —OR, or a carboxyl protecting group selected from the group consisting of ester, amides, and hydrazides;
R^{s} is

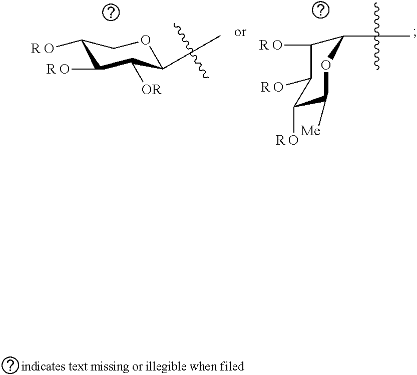

each occurrence of R^{x′} is independently an optionally substituted group selected from 6-10-membered aryl, C₁₋₆ aliphatic, or C₁₋₆ heteroaliphatic having 1-2 heteroatoms independently selected from the group consisting of nitrogen, oxygen, and sulfur; or:
   two R^{x′} are taken together to form a 5-7-membered heterocyclic ring having 1-2 heteroatoms independently selected from the group consisting of nitrogen, oxygen, and sulfur;
each occurrence of R is independently hydrogen, an optionally substituted group selected from acyl, arylalkyl, 6-10-membered aryl, C₁₋₆ aliphatic, or C₁₋₆ heteroaliphatic having 1-2 heteroatoms independently selected from the group consisting of nitrogen, oxygen, and sulfur, or:
   two R on the same nitrogen atom are taken with the nitrogen atom to form a 4-7-membered heterocyclic ring having 1-2 heteroatoms independently selected from the group consisting of nitrogen, oxygen, and sulfur.

In one aspect, the present application provides compounds of Formula I:

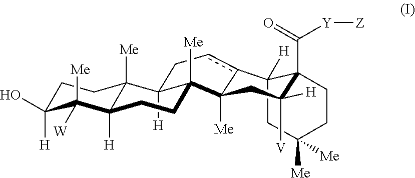

or a pharmaceutically acceptable salt thereof, wherein
[symbol] is a single or double bond;
W is —CHO;
V is ˜OH;
Y is —O—;
wherein Z is a carbohydrate domain having the structure:

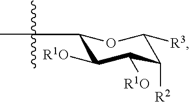

wherein:
   R¹ is independently H or

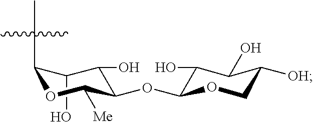

R² is NHR⁴;
R³ is CH₂OH; and
R⁴ is -T-R^{z}, —C(O)-T-R^{z}, —NH-T-R^{z}, —O-T-R^{z}, —S-T-R^{z}, —C(O)NH-T-R^{z}, C(O)O-T-R^{z}, C(O)S-T-R^{z}, C(O)NH-T-O-T-R^{z}, —O-T-R^{z}, -T-O-T-R^{z}, -T-S-T-R^{z}, or

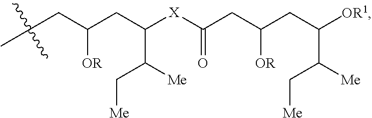

wherein:
X is —O—, —NR—, or T-R^{z};
T is a covalent bond or a bivalent C₁₋₂₆ saturated or unsaturated, straight or branched, aliphatic or heteroaliphatic chain; and
R^{z} is hydrogen, halogen, —OR, —OR^{x}, —OR¹, —SR, NR₂, —C(O)OR, —C(O)R, —NHC(O)R, —NHC(O)OR, NC(O)OR, or an optionally substituted group selected from acyl, arylalkyl, heteroarylalkyl, C₁₋₆ aliphatic, 6-10-membered aryl, 5-10-membered heteroaryl having 1-4 heteroatoms independently selected from nitrogen, oxygen, or sulfur, 4-7-membered heterocyclyl having 1-2 heteroatoms independently selected from the group consisting of nitrogen, oxygen, and sulfur.

It will be appreciated by one of ordinary skill in the art that the compounds of the present application include but are not necessarily limited to those compounds encompassed in the genus definitions set forth as part of the present section. The compounds encompassed by this application include at least all of the compounds disclosed in the entire specification as a whole, including all individual species within each genus.

In certain embodiments, V is OR^{x}. In certain embodiments, V is OH. In certain embodiments, V is H.

In certain embodiments, Y is —O—. In certain embodiments, Y is —NH—. In certain embodiments, Y is —NR—. In certain embodiments, Y is CH₂.

In certain embodiments, Z is hydrogen. In certain embodiments, Z is a cyclic or acyclic, optionally substituted moiety. In certain embodiments, Z is an acyl. In certain embodiments, Z is an aliphatic. In certain embodiments, Z is a heteroaliphatic. In certain embodiments, Z is aryl. In certain embodiments, Z is arylalkyl. In certain embodiments, Z is heteroacyl. In certain embodiments, Z is heteroaryl. In certain embodiments, Z is a carbohydrate domain having the structure:

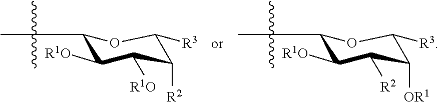

In some embodiments Z is a carbohydrate domain having the structure:

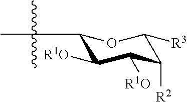

wherein:
   R¹ is independently H or

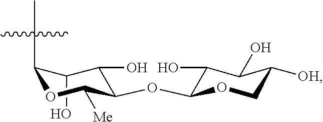

R² is NHR⁴,
R³ is CH₂OH, and
R⁴ is selected from:

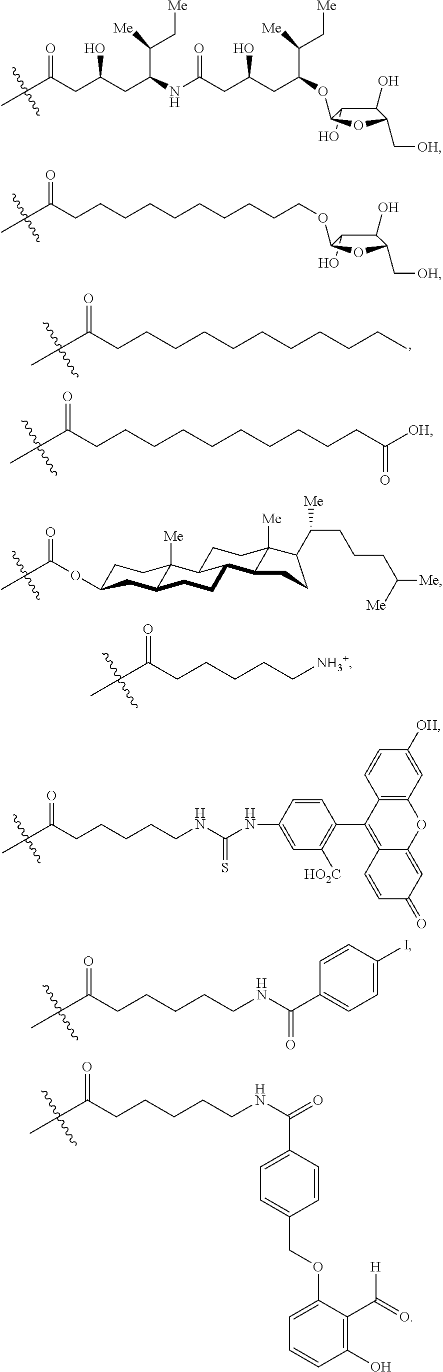

In some embodiments, R¹ is R^{x}. In other embodiments, R¹ is a carbohydrate domain having the structure:

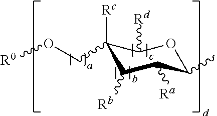

In some aspects, each occurrence of a, b, and c is independently 0, 1, or 2. In some embodiments, d is an integer from 1-5. In some embodiments, each d bracketed structure may be the same. In some embodiments, each d bracketed structure may be different. In some embodiments, the d bracketed structure represents a furanose or a pyranose moiety. In some embodiments, the sum of b and c is 1 or 2.

In some embodiments, R⁰ is hydrogen. In some embodiments, R⁰ is an oxygen protecting group selected from the group. In some embodiments, R⁰ is an alkyl ether. In some embodiments, R⁰ is a benzyl ether. In some embodiments, R⁰ is a silyl ether. In some embodiments, R⁰ is an acetal. In some embodiments, R⁰ is ketal. In some embodiments, R⁰ is an ester. In some embodiments, R⁰ is a carbamate. In some embodiments, R⁰ is a carbonate. In some embodiments, R⁰ is an optionally substituted moiety. In some embodiments, R⁰ is an acyl. In some embodiments, R⁰ is a C₁₋₁₀ aliphatic. In some embodiments, R⁰ is a C₁₋₆ heteroaliphatic. In some embodiments, R⁰ is a 6-10-membered aryl. In some embodiments, R⁰ is an arylalkyl. In some embodiments, R⁰ is a 5-10 membered heteroaryl having 1-4 heteroatoms independently selected from nitrogen, oxygen, or sulfur. In some embodiments, R⁰ is a 4-7 membered heterocyclyl having 1-2 heteroatoms independently selected from the group consisting of nitrogen, oxygen, and sulfur.

In some embodiments, R^{a} is hydrogen. In some embodiments, R^{a} is a halogen. In some embodiments, R^{a} is OH. In some embodiments, R^{a} is OR. In some embodiments, R^{a} is OR^{x}. In some embodiments. R^{a} is NR₂. In some embodiments, R^{a} is NHCOR. In some embodiments, R^{a} an acyl. In some embodiments, R^{a} is C₁₋₁₀ aliphatic. In some embodiments. R^{a} is C₁₋₆ heteroaliphatic. In some embodiments, R^{a} is 6-10-membered aryl. In some embodiments, R^{a} is arylalkyl. In some embodiments, R^{a} is a 5-10-membered heteroaryl having 1-4 heteroatoms independently selected from nitrogen, oxygen, and sulfur. In some embodiments, R^{a} is a 4-7-membered heterocyclyl having 1-2 heteroatoms independently selected from the group consisting of nitrogen, oxygen, and sulfur.

In some embodiments, R^{b} is hydrogen. In some embodiments, R^{b} is a halogen. In some embodiments, R^{b} is OH. In some embodiments, R^{b} is OR. In some embodiments, R^{b} is OR^{x}. In some embodiments. R^{s} is NR₂. In some embodiments, R^{b} is NHCOR. In some embodiments, R^{b} an acyl. In some embodiments, R^{b} is C₁₋₁₀ aliphatic. In some embodiments. R^{b} is C₁₋₆ heteroaliphatic. In some embodiments, R^{b} is 6-10-membered aryl. In some embodiments, R^{b} is arylalkyl. In some embodiments, R^{b} is a 5-10-membered heteroaryl having 1-4 heteroatoms independently selected from nitrogen, oxygen, and sulfur. In some embodiments, R^{b} is 4-7-membered heterocyclyl having 1-2 heteroatoms independently selected from the group consisting of nitrogen, oxygen, and sulfur.

In some embodiments, R^{b} is hydrogen. In some embodiments, R^{b} is a halogen. In some embodiments, R⁰ is OH. In some embodiments, R^{b} is OR. In some embodiments, R⁰ is OR^{x}. In some embodiments. R^{b} is NR₂. In some embodiments, R^{b} is NHCOR. In some embodiments, R^{b} an acyl. In some embodiments, R^{b} is C₁₋₁₀ aliphatic. In some embodiments. R^{b} is C₁₋₆ heteroaliphatic. In some embodiments, R^{b} is 6-10-membered aryl. In some embodiments, R^{b} is arylalkyl. In some embodiments, R^{b} is a 5-10-membered heteroaryl having 1-4 heteroatoms independently selected from nitrogen, oxygen, and sulfur. In some embodiments, R^{b} is 4-7-membered heterocyclyl having 1-2 heteroatoms independently selected from the group consisting of nitrogen, oxygen, and sulfur.

In some embodiments, R^{c} is hydrogen. In some embodiments, R^{c} is a halogen. In some embodiments, R^{c} is OH. In some embodiments, R^{c} is OR. In some embodiments, R^{c} is OR^{x}. In some embodiments, R^{c} is NR₂. In some embodiments, R^{c} is NHCOR. In some embodiments, R^{c} an acyl. In some embodiments, R^{c} is C₁₋₁₀ aliphatic. In some embodiments, R^{c} is C₁₋₆ heteroaliphatic. In some embodiments, R^{c} is 6-10-membered aryl. In some embodiments, R^{c} is arylalkyl. In some embodiments, R^{c} is a 5-10-membered heteroaryl having 1-4 heteroatoms independently selected from nitrogen, oxygen, and sulfur. In some embodiments, R^{c} is 4-7-membered heterocyclyl having 1-2 heteroatoms independently selected from the group consisting of nitrogen, oxygen, and sulfur.

In some embodiments, R^{d} is hydrogen. In some embodiments, R^{d} is a halogen. In some embodiments, R^{d} is OH. In some embodiments, R^{d} is OR. In some embodiments. R^{d} is OR^{x}. In some embodiments, R^{d} is NR₂. In some embodiments, R^{d} is NHCOR. In some embodiments, R^{d} an acyl. In some embodiments, R^{d} is C₁₋₁₀ aliphatic. In some embodiments, R^{d} is C₁₋₆ heteroaliphatic. In some embodiments, R^{d} is 6-10-membered aryl. In some embodiments, R^{d} is arylalkyl. In some embodiments, R^{d} is a 5-10-membered heteroaryl having 1-4 heteroatoms independently selected from nitrogen, oxygen, and sulfur. In some embodiments, R^{d} is 4-7-membered heterocyclyl having 1-2 heteroatoms independently selected from the group consisting of nitrogen, oxygen, and sulfur.

In some embodiments, R² is hydrogen. In some embodiments, R² is a halogen. In some embodiments, R² is OH. In some embodiments, R² is OR. In some embodiments. R² is OC(O)R⁴. In some embodiments, R² is OC(O)OR⁴. In some embodiments, R² is OC(O)NHR⁴. In some embodiments, R² is OC(O)NRR⁴. In some embodiments, R² is OC(O)SR⁴. In some embodiments, R² is NHC(O)R⁴. In some embodiments, R² is NRC(O)R⁴. In some embodiments, R² is NHC(O)OR⁴. In some embodiments, R² is NHC(O)NHR⁴. In some embodiments, R² is NHC(O)NRR⁴. In some embodiments, R² is NHR⁴. In some embodiments, R² is N(R⁴)₂. In some embodiments, R² is NHR⁴. In some embodiments. R² is NRR⁴. In some embodiments, R² is N₃. In some embodiments, R² is C₁₋₁₀ aliphatic. In some embodiments, R² is C₁₋₆ heteroaliphatic. In some embodiments, R² is 6-10-membered aryl. In some embodiments. R² is arylalkyl. In some embodiments, R² is 5-10 membered heteroaryl having 1-4 heteroatoms independently selected from the group consisting of nitrogen, oxygen, and sulfur. In some embodiments, R² is 4-7-membered heterocyclyl having 1-2 heteroatoms independently selected from the group consisting of nitrogen, oxygen, and sulfur.

In some embodiments, R³ is hydrogen. In some embodiments, R³ is a halogen. In some embodiments, R³ is CH₂OR¹. In some embodiments, R³ is an acyl. In some embodiments, R³ is C₁-10 aliphatic. In some embodiments, R³ is C₁₋₆ heteroaliphatic. In some embodiments, R³ is 6-10-membered aryl. In some embodiments. R³ is arylalkyl. In some embodiments, R³ is a 5-10-membered heteroaryl having 1-4 heteroatoms independently selected from the group consisting of nitrogen, oxygen, and sulfur. In some embodiments, R³ is a 4-7-membered heterocyclyl having 1-2 heteroatoms independently selected from the group consisting of nitrogen, oxygen, and sulfur.

In some embodiments, R⁴ is -T-R^{z}. In some embodiments, R⁴ is —C(O)-T-R^{z}. In some embodiments, R⁴ is —NH-T-R^{z}. In some embodiments, R⁴ is —O-T-R^{z}. In some embodiments. R⁴ is —S-T-R^{z}. In some embodiments, R⁴ is —C(O)NH-T-R^{z}. In some embodiments, R⁴ is C(O)O-T-R^{z}. In some embodiments, R⁴ is C(O)S-T-R^{z}. In some embodiments, R⁴ is C(O)NH-T-O-T-R^{z}. In some embodiments, R⁴ is —O-T-R^{z}. In some embodiments. R⁴ is -T-O-T-R^{z}. In some embodiments, R⁴ is -T-S-T-R^{z}. In some embodiments. R⁴ is

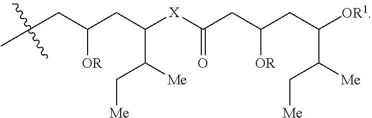

In some embodiments, X is —O—. In some embodiments, X is —NR—. In some embodiments, X is T-R^{z}.

In some embodiments, T is a covalent bond or a bivalent C₁₋₂₆ saturated or unsaturated, straight or branched, aliphatic or heteroaliphatic chain.

In some embodiments, R^{z} is hydrogen. In some embodiments, R^{z} is a halogen. In some embodiments. R^{z} is —OR. In some embodiments. R^{z} is —OR^{x}. In some embodiments, R^{z} is —OR¹. In some embodiments, R^{z} is —OR¹. In some embodiments, R^{z} is —SR. In some embodiments, R^{z} is NR₂. In some embodiments, R^{z} is —C(O)OR. In some embodiments, R^{z} is —C(O)R. In some embodiments, R^{z} is —NHC(O)R. In some embodiments, R^{z} is —NHC(O)OR. In some embodiments, R^{z} is NC(O)OR. In some embodiments, R^{z} is an acyl. In some embodiments, R^{z} is arylalkyl. In some embodiments, R^{z} is heteroarylalkyl. In some embodiments, R^{z} is C₁₋₆ aliphatic. In some embodiments, R^{z} is 6-10-membered aryl. In some embodiments, R^{z} is a 5-10-membered heteroaryl having 1-4 heteroatoms independently selected from nitrogen, oxygen, or sulfur. In some embodiments, R^{z} is a 4-7-membered heterocyclyl having 1-2 heteroatoms independently selected from the group consisting of nitrogen, oxygen, and sulfur.

In some embodiments, R^{x} is hydrogen. In some embodiments, R^{x} is an oxygen protecting group. In some embodiments, R^{x} is an alkyl ether. In some embodiments, R^{x} is a benzyl ether. In some embodiments, R^{x} is silyl ether. In some embodiments, R^{x} is an acetal. In some embodiments, R^{x} is ketal. In some embodiments. R is ester. In some embodiments, R^{x} is carbamate. In some embodiments, R^{x} is carbonate.

In some embodiments, R^{y} is —OH. In some embodiments, R^{y} is —OR. In some embodiments, R^{y} is a carboxyl protecting group. In some embodiments, R^{y} is an ester. In some embodiments, R^{y} is an amide. In some embodiments, R^{y} is a hydrazide.

In some embodiments, R^{s} is

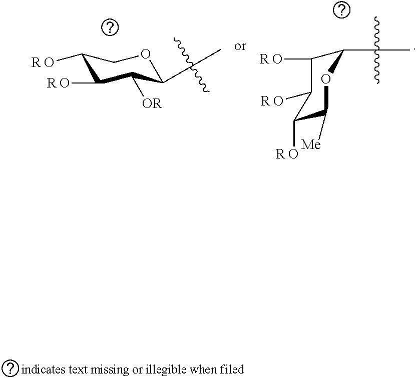

In some embodiments, R^{x′} is optionally substituted 6-10-membered aryl. In some embodiments, R^{x′} is optionally substituted C₁₋₆ aliphatic. In some embodiments, R^{x} is optionally substituted or C₁₋₆ heteroaliphatic having 1-2 heteroatoms independently selected from the group consisting of nitrogen, oxygen, and sulfur. In some embodiments, two R^{x′} are taken together to form a 5-7-membered heterocyclic ring having 1-2 heteroatoms independently selected from the group consisting of nitrogen, oxygen, and sulfur.

In some embodiments, R is hydrogen. In some embodiments, R is an acyl. In some embodiments, R is arylalkyl. In some embodiments, R is 6-10-membered aryl. In some embodiments, R is C₁₋₆ aliphatic. In some embodiments, R is C₁₋₆ heteroaliphatic having 1-2 heteroatoms independently selected from the group consisting of nitrogen, oxygen, and sulfur. In some embodiments, two R on the same nitrogen atom are taken with the nitrogen atom to form a 4-7-membered heterocyclic ring having 1-2 heteroatoms independently selected from the group consisting of nitrogen, oxygen, and sulfur.

In some embodiments, R^{1′} has the same embodiments as R¹.

Exemplary compounds of Formula I are set forth in Table 1 below:

TABLE 1
EXEMPLARY COMPOUNDS OF FORMULA I

| | |
|---|---|
| 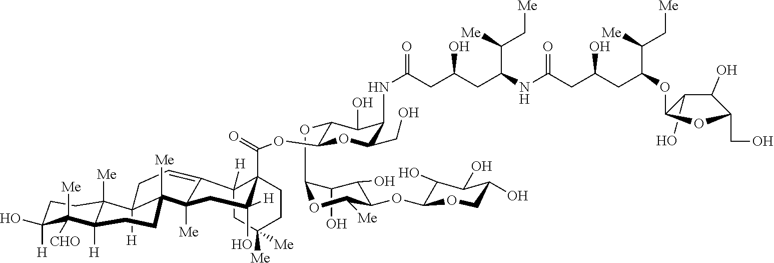 | I-1 |
| 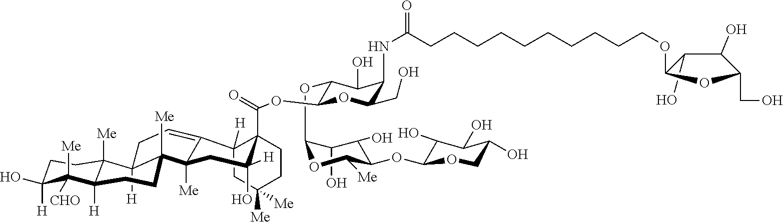 | I-2 |
| 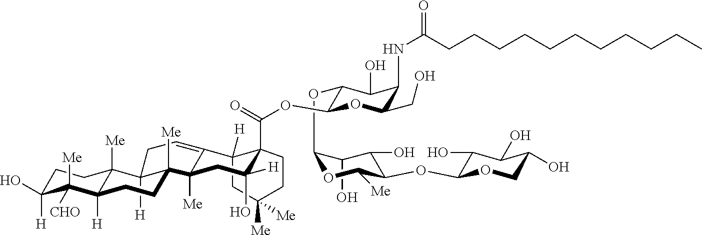 | I-3 |
| 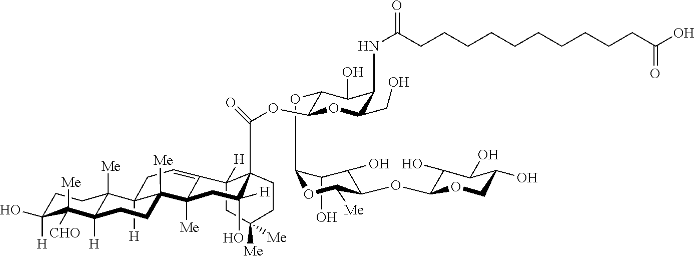 | I-4 |
| 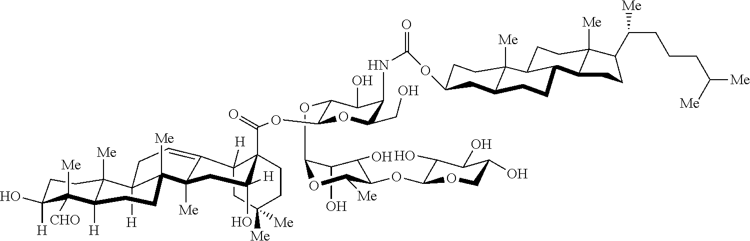 | I-5 |
| 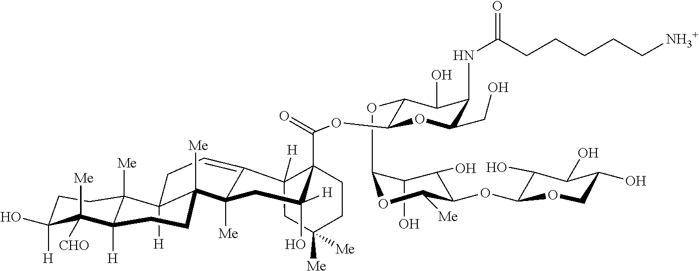 | I-6 |
| 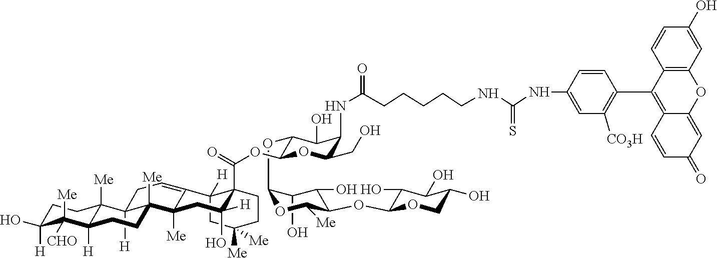 | I-7 |
| 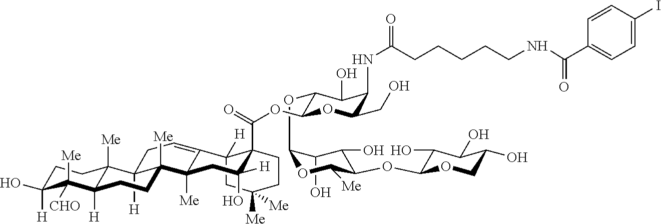 | I-8 |
| 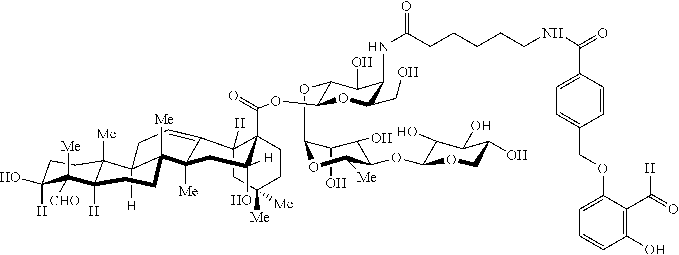 | I-9 |
| 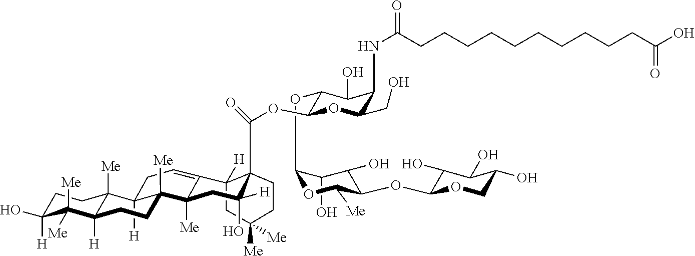 | I-10 |

### TLR4 Agonists

In another aspect, the present application provides TLR4 agonists for use in combination with a saponin derivative.

In one aspect, the present application provides the following compounds: Lipid A, MPL, 3D-MPL, GLA, LPS, RC-529, E6020, ONO-4007, CRX-527, CRX-547, GSK1795091, CRX-601, SLA, PET-lipid A, PHAD, 3D-PHAD, 3D-(6-acyl)-PHAD, OM-294, OM-174, OK-432, 2B182C.

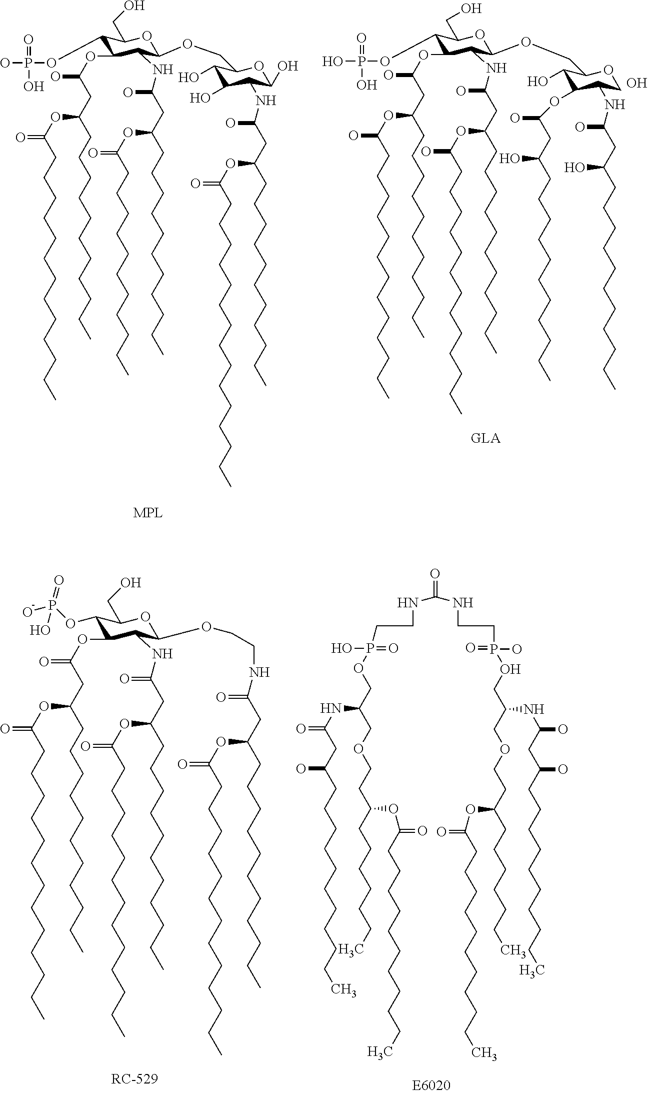

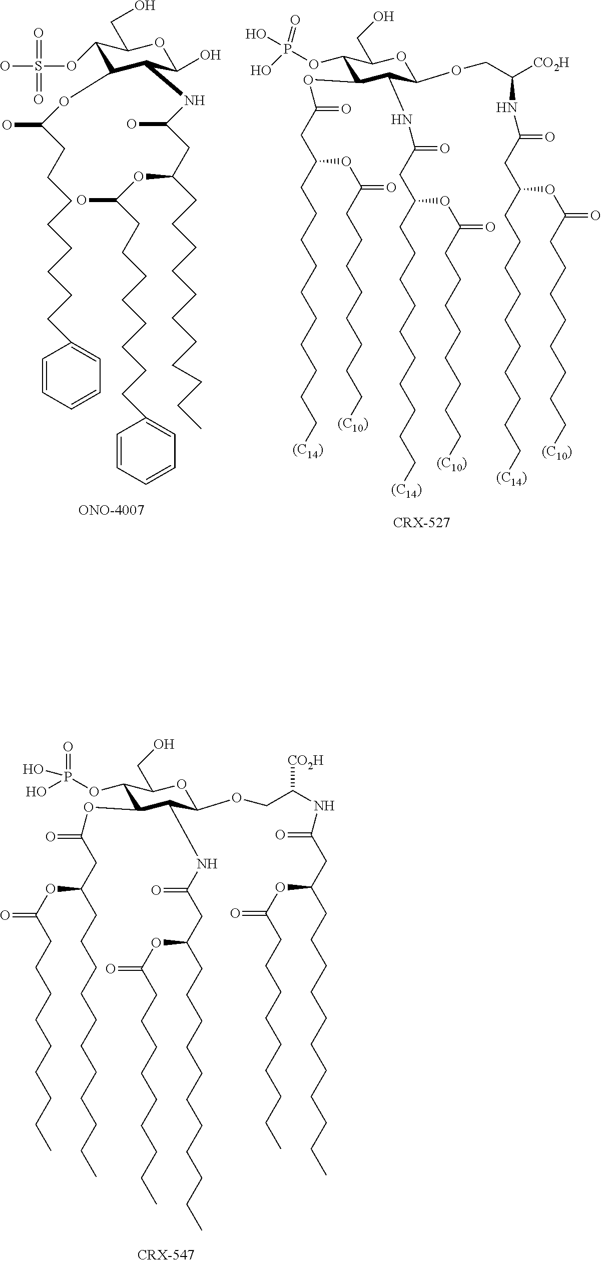

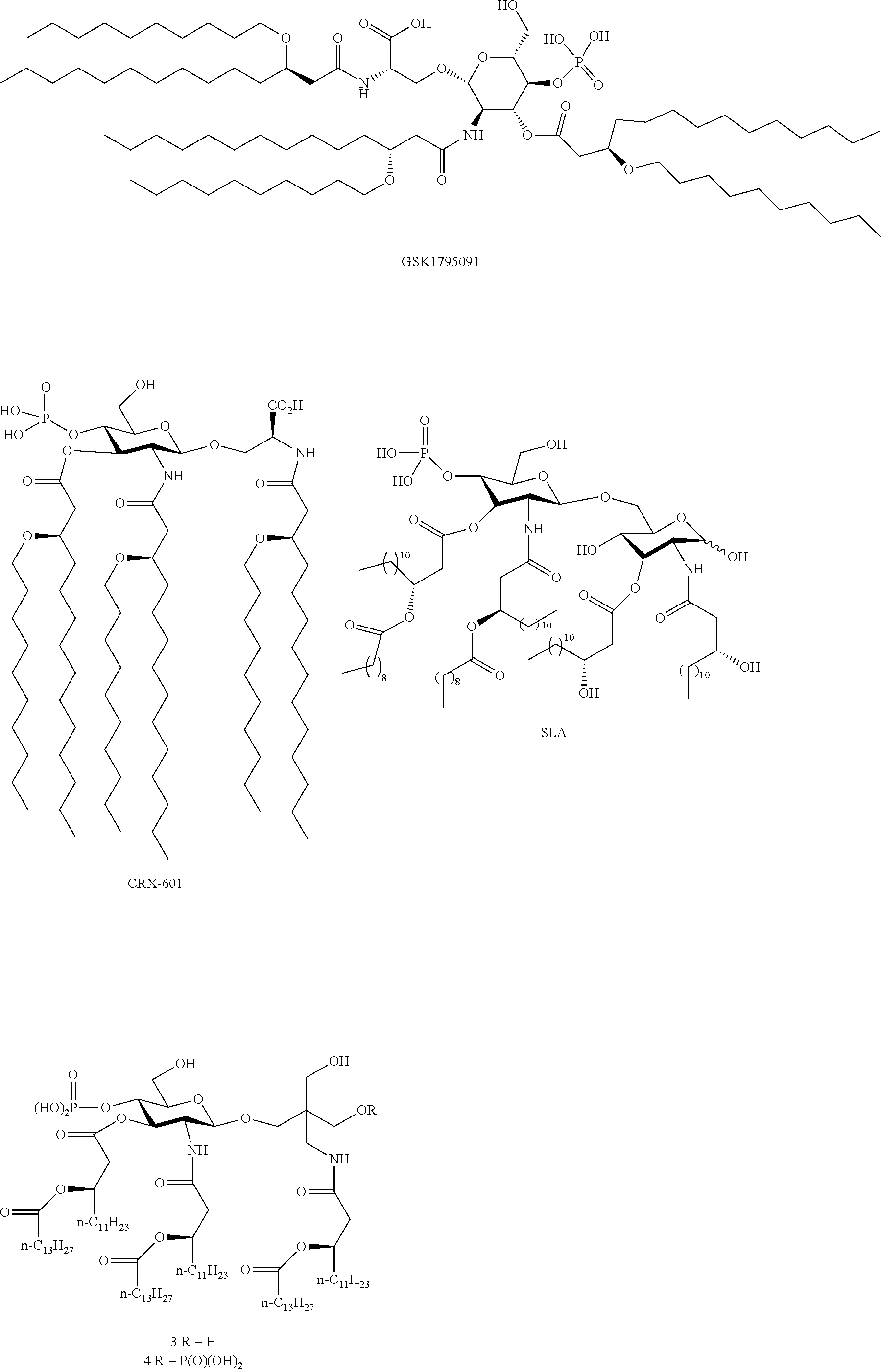

### PET-Lipid A (Containing an Amine-Substituted Pentaerythritol Instead of a Glucosamine Unit Found in Natural Lipid A and Retains Only One Phosphoryl Group)

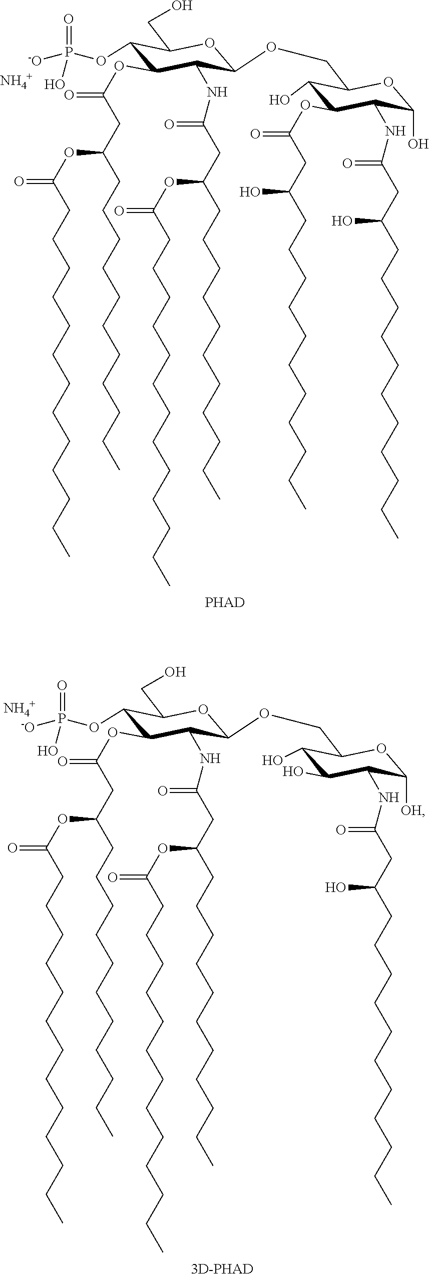

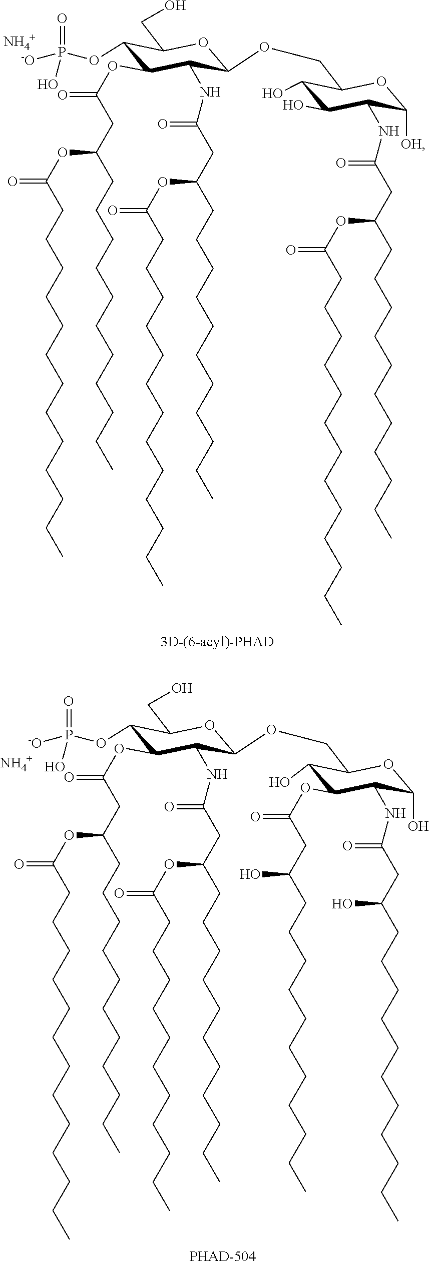

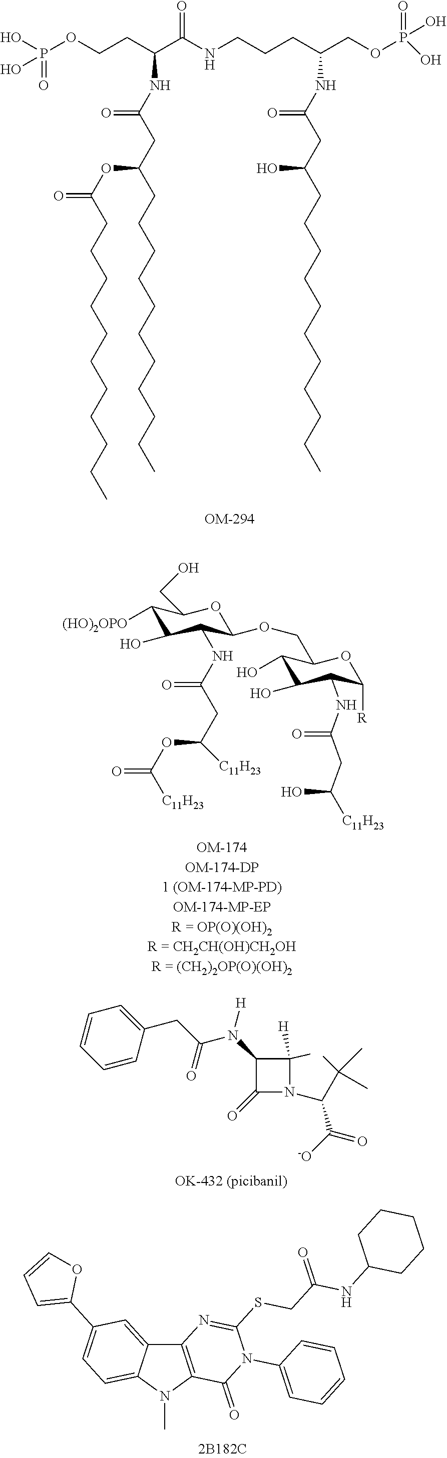

In another aspect, the present application provides E. coli derived monophosphoryl lipid A (EcML). EcML is a mixture of 4′-monophosphoryl lipids A (MPLAs) produced directly by an engineered Escherichia coli strain. In some embodiments, EcML comprises a mixture of hexa-acylated and penta-acylated MPLA congeners. In some embodiments, EcML comprises a mixture of hexa-acylated, penta-acylated, and tetra-acylated MPLA

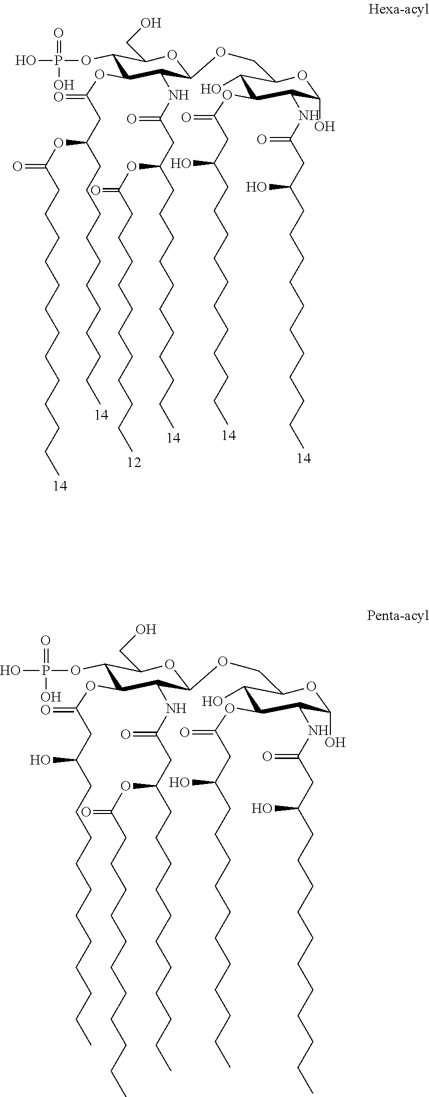

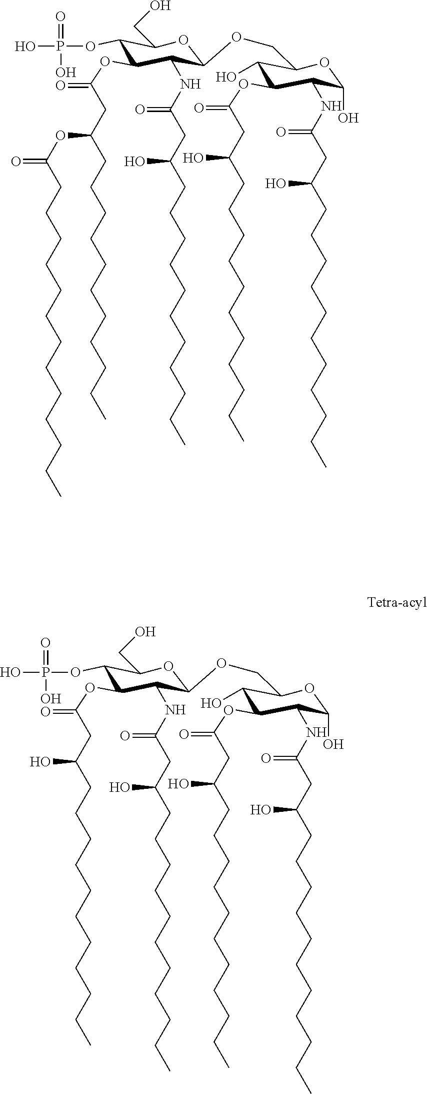

In some embodiments, EcML comprises at least 60% of the hexa-acyl congener and up to 40% of the penta-acyl congeners. In some embodiments, EcML comprises approximately 70% of the hexa-acyl congener, approximately 22% of the penta-acyl congeners, and approximately 8% of the tetra-acyl congeners.

In another aspect, the present application provides the following compounds:

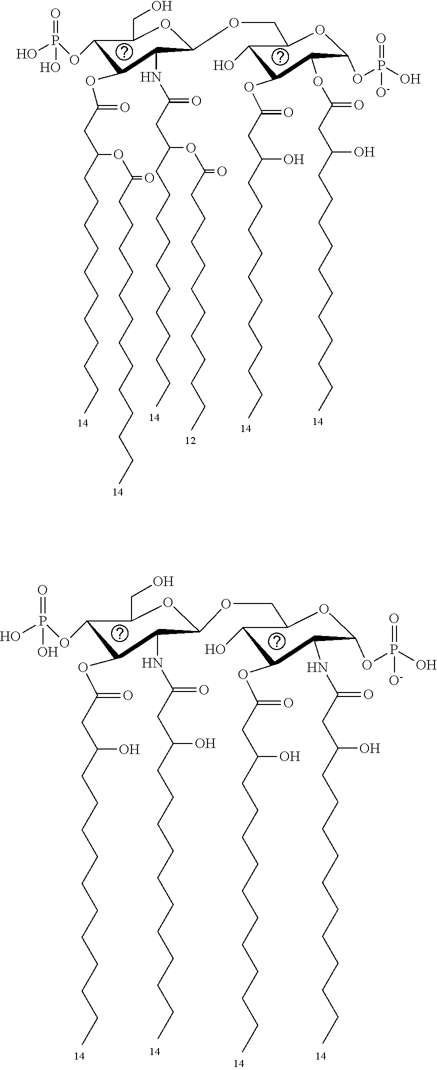

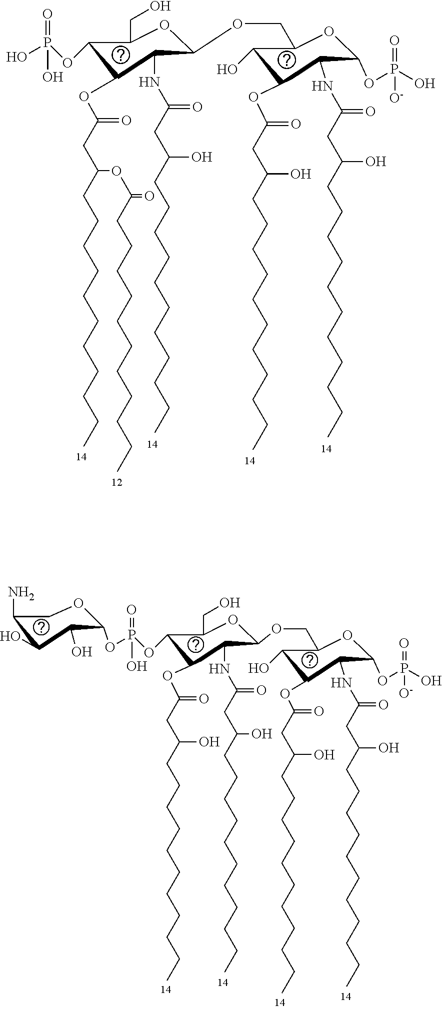

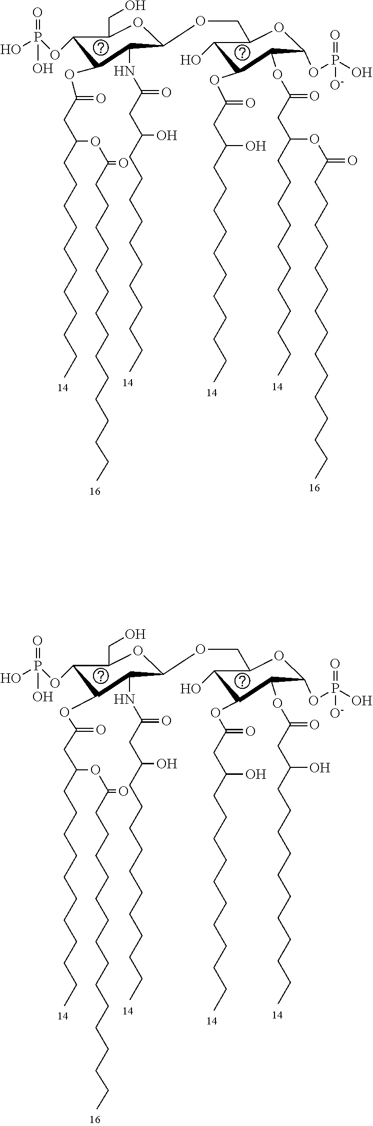

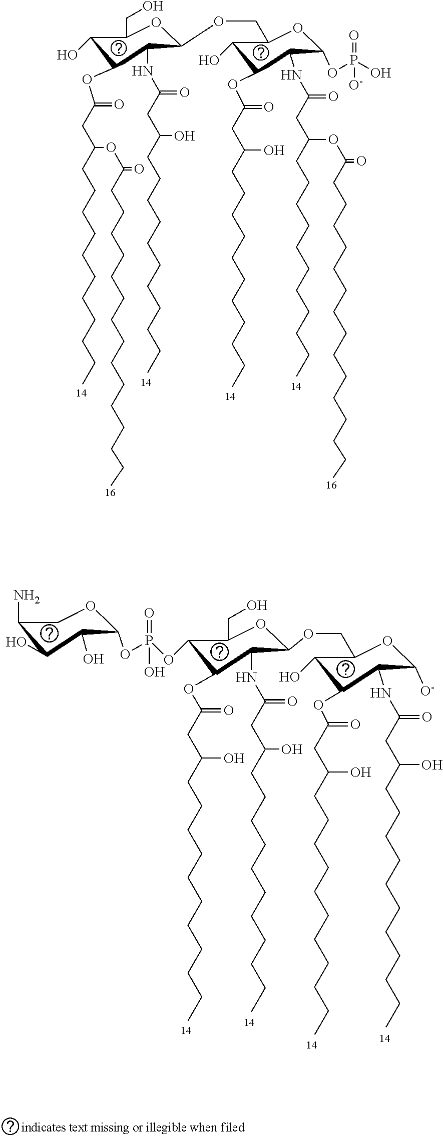

### TLR9 Agonists

In another aspect, the present application provides TLR9 agonists for combination with a saponin derivative. In some embodiments, the TLR9 agonist is an immunostimulatory sequence (“ISS”). In some embodiments, the ISS is selected from the group consisting of CpG oligodeoxynucleotides class A, B and C, or an immunostimulatory peptide mimicking any of the CpG oligodeoxynucleotides. In some embodiments, the ISS is CpG DNA or a synthetic oligonucleotide containing CpG motifs. In some embodiments, the ISS is CpG 1018, SD-101, or CpG 2006. In some embodiments, the ISS is ODN 1585, ODN 1668, ODN 1826, ODN 2006, ODN 2007, ODN 2216, ODN 2336, ODN 2395, or ODN M362.

In some embodiments, the ISS comprises the sequence 5′-purine, purine, C. G, pyrimidine, pyrimidine, C, G-3′ or 5′-purine, purine, C, G, pyrimidine, pyrimidine, C, C-3′. In some embodiments, the ISS comprises a sequence selected from the group consisting of AACGTTCC, AACGTTCG, GACGTTCC and GACGTTCG. In some embodiments, the ISS comprises the sequence 5′-T, C, G-3′. In some embodiments of the methods and kits of the invention, the ISS comprises the sequence 5′-TGACTGTGAACGTTCGAGATGA-3′ (SEQ ID NO: 10). In some embodiments, the ISS comprises the sequence 5′-TCGAACGTTCGAACGTTCGAACGTTCGAAT-3′ (SEQ ID NO: 11).

In some embodiments, the TLR9 agonist is a CpG-C ON or a CpG-C type oligonucleotide. “CpG-C ONs” or “CpG-C type oligonucleotides” are oligonucleotides from 12 to 100 bases in length, which have one or more 5′-TCG trinucleotides wherein the 5′-T is positioned 0, 1, 2, or 3 bases from the 5′-end of the oligonucleotide, and at least one palindromic sequence of at least 8 bases in length comprising one or more unmethylated CG dinucleotides. The one or more 5′-TCG trinucleotide sequence may be separated from the 5′-end of the palindromic sequence by 0, 1, or 2 bases or the palindromic sequence may contain all or part of the one or more 5′-TCG trinucleotide sequence. In one embodiment, the oligonucleotide is an oligodeoxynucleotide (ODN). In one embodiment, the oligonucleotide is a 2′-oligodeoxynucleotide. CpG-C ODNs have the ability to stimulate B cells, induce plasmacytoid dendritic cell (PDC) maturation and cause secretion of high levels of type I interferons (e.g., IFN-α, IFN-γ, etc.). In some embodiments, the CpG-C ODNs are 12 to 100 bases in length, preferably 12 to 50 bases in length, preferably 12 to 40 bases in length, or preferably 12-30 bases in length. In some embodiments, the ODN is at least (lower limit) 12, 13, 14, 15, 16, 17, 18, 19, 20, 21, 22, 23, 24, 25, 26, 27, 28, 29, 30, 32, 34, 36, 38, 40, 50, 60, 70, 80, or 90 bases in length. In some embodiments, the ODN is at most (upper limit) 100, 90, 80, 70, 60, 50, 49, 48, 47, 46, 45, 44, 43, 42, 41, 40, 39, 38, 37, 36, 35, 34, 33, 32, 31, or 30 bases in length. In some embodiments, the at least one palindromic sequence is 8 to 97 bases in length, preferably 8 to 50 bases in length, or preferably 8 to 32 bases in length. In some embodiments, the at least one palindromic sequence is at least (lower limit) 8, 10, 12, 14, 16, 18, 20, 22, 24, 26, 28, or 30 bases in length. In some embodiments, the at least one palindromic sequence is at most (upper limit) 50, 48, 46, 44, 42, 40, 38, 36, 34, 32, 30, 28, 26, 24, 22, 20, 18, 16, 14, 12 or 10 bases in length. In one embodiment, the oligonucleotide is an oligodeoxynucleotide. In one embodiment, one or more of the internucleotide linkages of the CpG-C ODN are modified linkages. In one embodiment, one or more of the internucleotide linkages of CpG-C ODN are phosphorothioate (PS) linkages. In one embodiment, all of the internucleotide linkages of CpG-C ODN are phosphorothioate (PS) linkages. A phosphorothioate backbone refers to all of the internucleotide linkages of CpG-C ODN being phosphorothioate (PS) linkages.

In one embodiment, the CpG-C ODNs of the present disclosure comprise:
(a) 5′-Nₓ(TCG(N_{q}))_{y}N_{w}(X₁X₂CGX₂′X₁′(CG)ₚ)_{z},Nᵥ (SEQ ID NO:22) wherein N are nucleosides, x=0, 1, 2 or 3, y=1, 2, 3 or 4, w=0, 1 or 2, p=0 or 1, q=0, 1 or 2, v=0 to 89 and z=1 to 20, X₁ and X₁′ are self-complementary nucleosides, X₂ and X₂′ are self-complementary nucleosides, and wherein the 5′-T of the (TCG(N_{q}))_{y}, sequence is 0-3 bases from the 5′ end of the oligonucleotide; and
(b) a palindromic sequence at least 8 bases in length wherein the palindromic sequence comprises the first (X₁X₂CGX₂X₁′) (SEQ ID NO:25) of the (X₁X₂CGX₂′X₁′(CG)ₚ)_{z} (SEQ ID NO:26) sequences, wherein the ODN is from 12 to 100 bases in length. In some embodiments, x=0, y=1, w=0, p=0 or 1, q=0, 1 or 2, v=0 to 20 and z=1, 2, 3 or 4. In some embodiments, X₁ and X₂ are each either A or T. In some embodiments, the palindromic sequence has a base composition of more than one-third As and Ts. In some embodiments, the CpG-C ODN comprises a sequence selected from the group consisting of SEQ ID NOs: 10-24.

In some embodiments, the CpG-C ODNs of the present disclosure consist of TCGN_{q}(X₁X₂CGX₂′X₁′CG)_{z}Nᵥ (SEQ ID NO:23), wherein N are nucleosides, q=0, 1, 2, 3, 4, or 5, v=0 to 20, z=1 to 4, X₁ and X₁′ are self-complementary nucleosides, X₂ and X₂′ are self-complementary nucleosides, and wherein the ODN is at least 12 bases in length. In some embodiments, the CpG-C ODN consists of a sequence selected from the group consisting of SEQ ID NOs: 10-24.

In some embodiments, the CpG-C ODNs of the present disclosure consist of 5′-TCGN_{q}TTCGAACGTTCGAACGTTNₛ-3′ (SEQ ID NO:24), wherein N are nucleosides, q=0, 1, 2, 3, 4, or 5, s=0 to 20, and wherein the ODN is at least 12 bases in length. In one embodiment, s=0, 1, 2, 3, 4, or 5. In some embodiments, the CpG-C ODN consists of a sequence selected from the group consisting of

(SEQ ID NO: 13)
5′-TCGTTCGAACGTTCGAACGTTCGAA-3′
q = 0 and s = 4,
(SEQ ID NO: 14)
5′-TCGAACGTTCGAACGTTCGAACGTT-3′
q = 4 and s = 0,
(SEQ ID NO: 11)
5′-TCGAACGTTCGAACGTTCGAACGTTCGAAT-3′
q = 4 and s = 5,
(SEQ ID NO: 16)
5′-TCGTAACGTTCGAACGTTCGAACGTTA-3′
q = 5 and s = 1,
and
(SEQ ID NO: 17)
5′-TCGTAACGTTCGAACGTTCGAACGTT-3′
q = 5 and s = 0.

In one embodiment, the TLR9 agonist is a CpG-C ODN consisting of the sequence 5′-TCGAACGTTCGAACGTTCGAACGTTCGAAT-3′ (SEQ ID NO:11). In another embodiment, the CpG-C ODN is the sodium salt of 5′-TCGAACGTTCGAACGTTCGAACGTTCGAAT-3′ (SEQ ID NO:11). In a further embodiment, the CpG-C type oligonucleotide has a sequence that consists of 5′-TCGTTCGAACGTTCGAACGTTCGAA-3′ (SEQ ID NO:13). In a further embodiment, the CpG-C type oligonucleotide is a sodium salt of 5′-TCGTTCGAACGTTCGAACGTTCGAA-3′ (SEQ ID NO:13).

In another embodiment, the TLR9 agonist CpG-C type oligonucleotide is selected from the group consisting of:

(SEQ ID NO: 12)
5′-TCGTCGAACGTTCGAGATGAT-3′;
(SEQ ID NO: 13)
5′-TCGTTCGAACGTTCGAACGTTCGAA-3′;
(SEQ ID NO: 14)
5′-TCGAACGTTCGAACGTTCGAACGTT-3′;
(SEQ ID NO: 15)
5′-TCGAACGTTCGAACGTTCGAATTTT-3′;
(SEQ ID NO: 11)
5′-TCGAACGTTCGAACGTTCGAACGTTCGAAT-3′;
(SEQ ID NO: 16)
5′-TCGTAACGTTCGAACGTTCGAACGTTA-3′;
(SEQ ID NO: 17)
5′-TCGTAACGTTCGAACGTTCGAACGTT-3′;
(SEQ ID NO: 18)
5′-TCGTAACGTTCGAACGTTCGAACGT-3′;
(SEQ ID NO: 19)
5′-TCGTAACGTTCGAACGTTCGAACG-3′;
(SEQ ID NO: 20)
5′-TCGTAACGTTCGAACGTTCGAAC-3′;
and
(SEQ ID NO: 21)
5′-TCGTAACGTTCGAACGTTCGAA-3′.

### Synthesis

Certain compounds of the present application may be synthesized as provided in PCT/US2009/039954, PCT/US2015/33567, PCT/US2016/67530, PCT/US2016/60564, and/or PCT/US2018/027462. Certain compounds of the present application may be synthesized as provided in PCT/US2019/017669. Certain compounds of the present application are commercially available. Based on the disclosures in the foregoing documents, those of ordinary skill in the art would understand how to synthesize the compounds disclosed in the present application.

### Adjuvants

The present application encompasses the recognition that synthetic access to and structural modification of QS-21 and related Quillaja saponins may afford compounds with high adjuvant potency and low toxicity, as well as having more stability and being more cost-effective. Accordingly, compounds of the present application, including TQL-1055, have industrial applicability and are useful as adjuvants, in free form, acid or base form, or pharmaceutically acceptable salt form.

The present application also encompasses the recognition that certain TLR4 agonists and antagonists may be potent immunomodulatory agents. Accordingly, compounds of the present application, have industrial applicability and are useful as adjuvants, in free form, acid or base form, or pharmaceutically acceptable salt form.

The inventors of the present application have invented combinations of certain saponin adjuvants, including preferably TQL-1055, and certain TLR4 agonists, which together will produce surprising, beneficial, and/or synergistic effects. Thus, the present application encompasses combinations of the compounds described herein.

### Antigens

The present application also provides antigens associated with certain pathogens where CD4 responses are thought to control infection directly, including, for example, HIV, all herpes viruses (e.g. HSV-1/2, varicella zoster virus (VZV), EBV, CMV, and HHV6/7/8, and their related disorders such as herpes zoster and post herpetic neuralgia (PHN)), Plasmodium falciparum and other Plasmodium species, Mycobacterium tuberculosis, M. leprae other Mycobacteria species, Salmonella typhi and other Salmonella species, Leishmania major and other Leishmania species, Necator americanus, Ancylostoma duodenale, Giardia lamblia and other Giardia species, Cryptosporidium parvum and other Cryptosporidium species, Coccidioides immitis and other Coccidiodes species, and Histoplasma capsulatum and other Histoplasma species.

The present application also provides antigens associated with certain pathogens where the antibody response may be improved in quality or duration by a helper CD4 response, including, for example, Influenza A and B viruses, Bordetella pertussis, Coronaviruses (including SARS-COV1, MERS-COV, and SARS-Cov2), vesicular stomatitis virus (VSV), Vaccinia viruses, respiratory syncytial virus (RSV), Dengue virus, Zika virus, West Nile Virus, Yellow Fever virus (YFV), and alphaviruses including equine encephalitis (VEE/EEE/WEE), Chikungunya, Ross River, and O'Nyong-nyong viruses.

In certain embodiments, the antigen is a protein, e.g. a glycoprotein, associated with a specific pathogen, or a recombinant version thereof. In certain embodiments, the antigen is DNA/RNA molecule associated with a specific pathogen, or a recombinant version thereof. In some embodiments, the antigen is a polysaccharide associated with a pathogen, or a recombinant version thereof. In some embodiments, the antigen is a live, live-attenuated, or inactivated pathogen, or a recombinant version thereof.

In some embodiments, the antigen may be expressed in a vector-based system.

### Varicella Zoster Antigens

The VZV antigen for use in the present application is an optionally truncated VZV glycoprotein, a fragment thereof, or an immunogenic derivative thereof. The optionally truncated VZV glycoprotein may be VZV glycoprotein E (gE) (VZV gE is also known as gp1), a fragment thereof, or an immunogenic derivative thereof, and may have the sequence of SEQ ID No. 1.

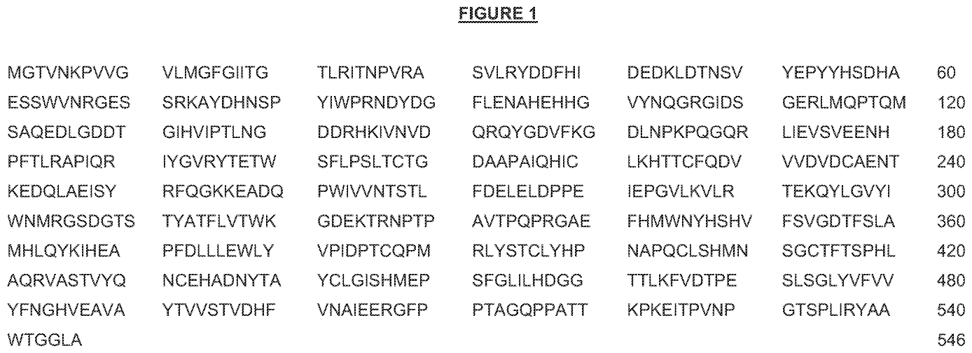

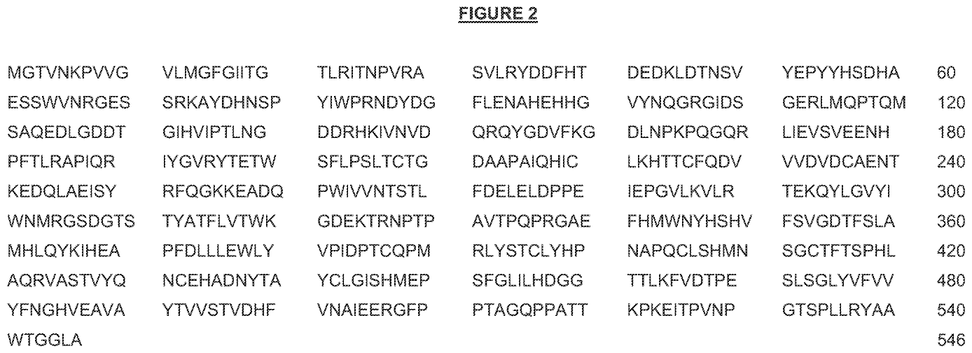

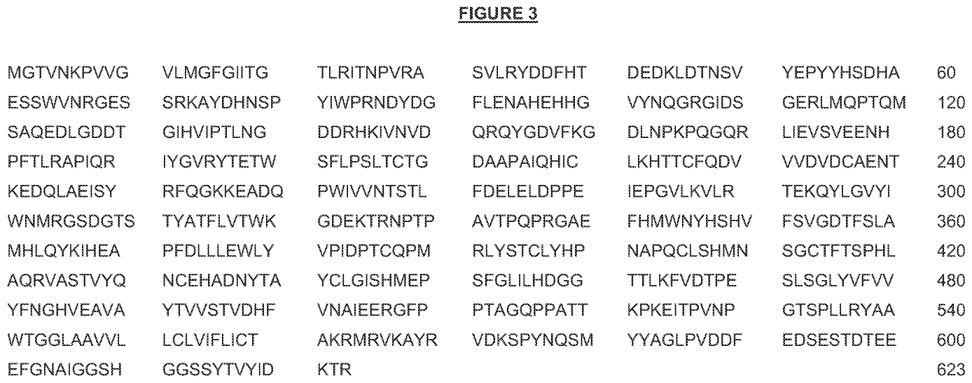

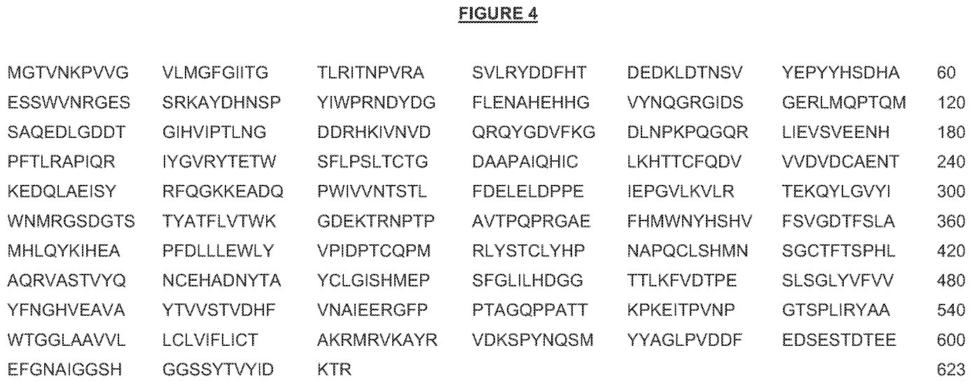

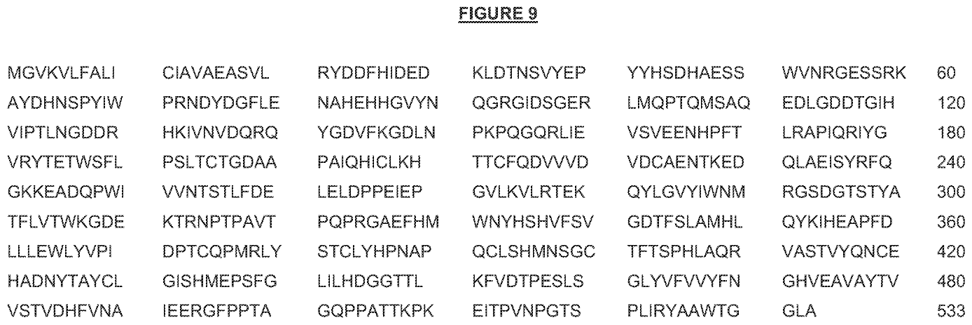

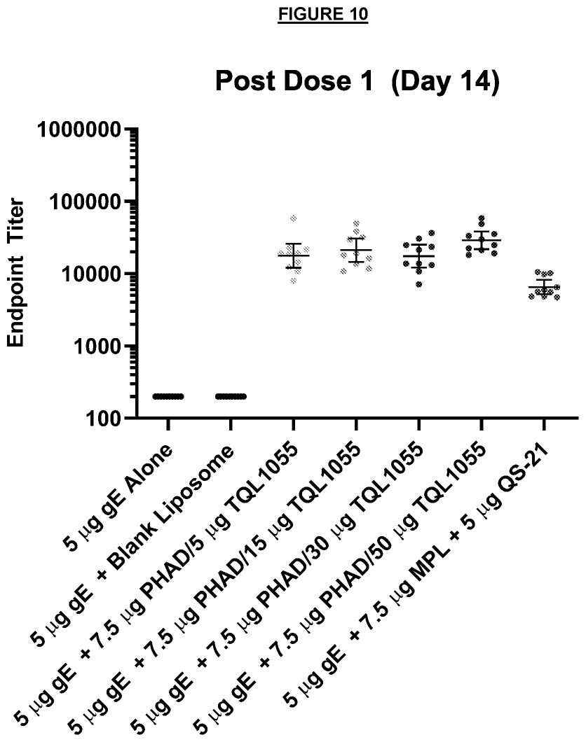

In one aspect the VZV gE is a truncated gE having the sequence of SEQ ID No. 1 (FIG. 1), and as disclosed in Virus research, vol 40, 1996 p 199 ff, herein incorporated fully by reference. In another aspect, the VZV gE is a truncated gE having the sequence of SEQ ID No. 2 (FIG. 2). In another aspect, the VZV gE is a truncated gE having the sequence of SEQ ID No. 9 (FIG. 9). In another aspect, the VZV gE is gE having the sequence of SEQ ID No. 3 (FIG. 3). In another aspect, the VZV gE is gE having the sequence of SEQ ID No. 4 (FIG. 4). In another aspect, the VZV gE is a truncated version of SEQ ID No. 3 or 4.

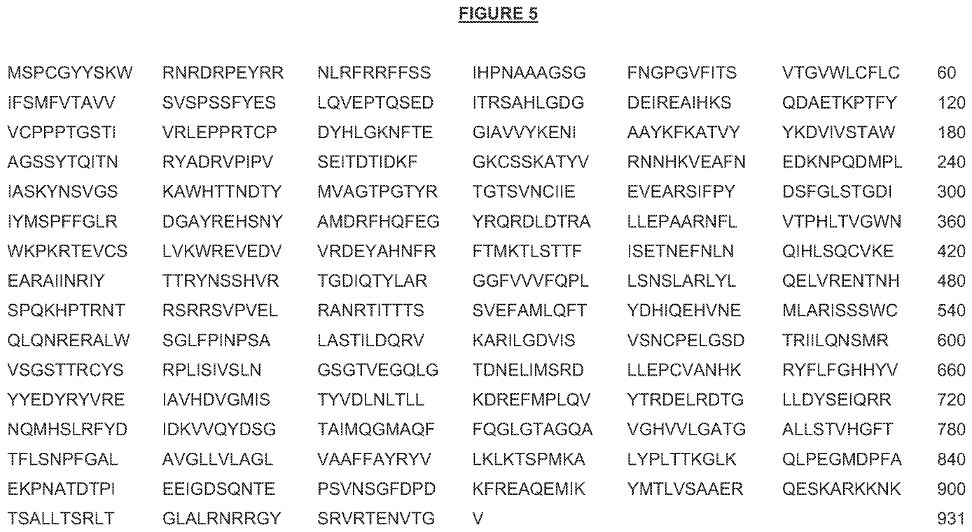

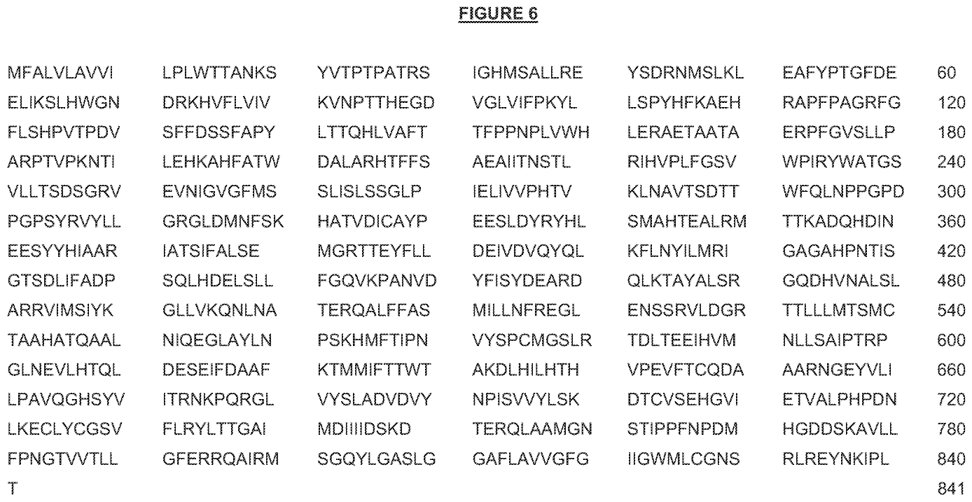

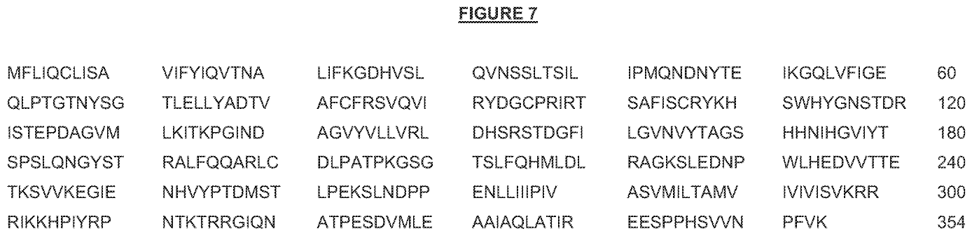

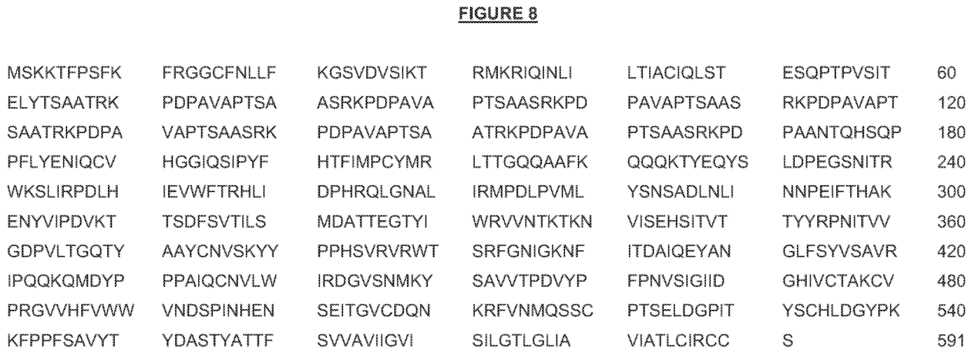

In another aspect, the VZV antigen may include, by way of example, VZV gB (SEQ ID No. 5. FIG. 5), VZV gH (SEQ ID No. 6, FIG. 6). VZV gC (SEQ ID No. 8, FIG. 8), VZV gI (SEQ ID No. 7, FIG. 7), IE63 (e.g. see, Huang et al. J. Virol. 1992, 66:2664, Sharp et al. J. Inf. Dis. 1992, 165:852, Debrus, J. Virol. 1995 May: 69 (5): 3240-5 and references therein), IE62 (e.g. see Arvin et al. J. Immunol. 1991 146:257, Sabella J. Virol. 1993 December; 67 (12): 7673-6 and references therein) ORF 4 or ORF 10 (Arvin et al. Viral Immunol. 2002 15:507.). In another aspect, the VZV antigen may be a truncated version of any of the foregoing.

The present application herein also contemplates that antigen combinations may be used with the live attenuated or killed VZV, and in one aspect a truncated gE as discussed above (SEQ ID Nos. 1, 2, or 9) may be included in any such combination. In one aspect the present application relates to combinations of truncated gE (SEQ ID Nos. 1, 2, or 9) with IE63 and truncated gE (SEQ ID Nos. 1, 2, or 9) with IE62, for example.

Embodiments of a gE antigen, derivatives thereof, and production thereof are described in EP0405867 and references therein (see also Vafai A. Antibody binding sites on truncated forms of varicella-zoster virus gpI(gE) glycoprotein Vaccine 1994 12:1265-9). EP0192902 also discloses embodiments of gE and production thereof.

### Vaccine Compositions

Vaccine preparation is generally described in New Trends and Developments in Vaccines, Voller et al. (eds), University Park Press, Baltimore, Md., 1978.

In an embodiment of the present application, a composition that induces an immune response against Varicella zoster virus comprises an optionally truncated VZV glycoprotein or a fragment thereof in combination with a triterpene glycoside saponin-derived adjuvant. The optionally truncated VZV glycoprotein may be VZV glycoprotein E or a fragment thereof or may have the sequence of any of SEQ ID Nos. 1-4, and 9 (FIGS. 1-4, and 9). The saponin-derived adjuvant may be a compound according to Formula I as described previously.

In a preferred embodiment of the present application, a composition that induces an immune response against Varicella zoster virus comprises a VZV glycoprotein E truncated to remove the carboxy terminal anchor region, which gE is not in the form of a fusion protein, in combination with an adjuvant comprising a compound of Formula II (or a pharmaceutically acceptable salt thereof, e.g. a choline salt), a TLR4 agonist (e.g. MPLA, PHAD, MPL) and/or a TLR9 agonist (e.g., CPG-1018, SD-101), and a liposome-forming compound (e.g. DOPC, DMPC, DMPG, cholesterol, and/or combinations of the foregoing).

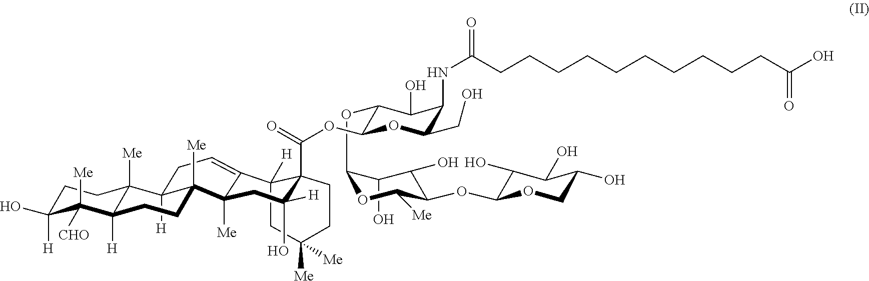

In some of the preferred embodiments, the VZV glycoprotein contains a non-native signal peptide on the N-terminus to improve cleavage. In the context of the present application, such non-native signal peptides together with truncated or untruncated glycoproteins are not considered fusion proteins.

In another preferred embodiment, the present application utilizes an emulsion-based technology instead of or in conjunction with a liposome-forming compound.

Vaccine compositions, VZV antigens, and derivatives of VZV antigens can be tested for suitable immunogenic activity by use in the model systems by clinical trials in humans. One or more of the following indicators of activity are suitable for consideration in the assessment of immunogenic activity: (1) Increased CD4 or CD8 T cell responses to VZV or antigen derivatives; (2) Elevation in VZV or antigens derivative specific antibodies: (3) Enhanced production of cytokines such as interferon γ or IL-2 or TNF α, (4) Enhanced expression of CD40L on CD4 and CD8 T cells; and/or (5) Reduction in the incidence of zoster below the incidence found in the general population of similarly at-risk individuals, and likewise reduced disease severity and/or associated pain below the incidence found in the general population of similarly at-risk individuals.

In another aspect, the present application relates to vaccine compositions comprising VZV antigen in combination with live attenuated or killed VZV. Suitable combinations of antigens include, for example, optionally truncated gE (SEQ ID No. 1, 2, 9), fragments thereof, or immunogenic derivatives thereof. The combined composition or either or both of the individual components may additionally comprise an adjuvant composition as set forth in the present application.

Where a live attenuated strain is used, in one aspect the live attenuated VZV strain is the OKA strain, a strain well known in the art, for example as disclosed in Arbeter et al. (Journal of Pediatrics, vol 100, No 6, p 886 ff), WO9402596, and references therein, such as U.S. Pat. No. 3,985,615, all incorporated herein by reference. Any other suitable live attenuated strain may also be used in the present application. For example, the VARILRIX and VARIVAX strains are both appropriate and commercially available and could be employed. VZV-Dumas (either attenuated or inactivated) could also be employed. Whole inactivated VZV strains, such as inactivated VZV OKA are also suitable for use in the subject matter of the present application.

The amount of VZV antigen used in vaccine compositions of the present application is selected as an amount that induces an immunoprotective response without significant, adverse side effects. Such amount will vary depending upon which specific immunogen is employed and how it is presented. Generally, it is expected that each dose will comprise 1-1000 μg of protein, such as 2-100 μg, or 5-60 μg. Where gE (SEQ ID No 1, 2, 3, 4, or 9) is used then in one aspect 25-100 μg of gE (SEQ ID No 1, 2, 3, 4, or 9) may be used in humans, such as 40-100 μg of gE (SEQ ID No 1, 2, 3, 4, or 9) for human use, in one aspect about 25 μg, about 50 μg or about 100 μg of gE (SEQ ID No 1, 2, 3, 4, or 9), suitably 25 μg, 50 μg or 100 μg gE (SEQ ID No 1, 2, 3, 4, or 9). For the OKA strain, for example, a suitable dose is 500-50000 pfu/0.5 ml, such as 2000-6000 pfu/0.5 ml, with a suitable dose of the Oka strain for example being 6000-25,000 per dose, for example, 10,000 pfu/dose. Higher doses such as 30,000 pfu, 40000 pfu, 50,000 pfu 60,000 pfu, 70000 pfu, 80000 pfu, 90000 pfu, or even 100000 pfu may be employed.

An optimal amount for a particular vaccine can be ascertained by standard studies involving observation of appropriate immune responses in subjects. Following an initial vaccination, subjects may receive one or several booster immunizations adequately spaced. The composition(s) of the present application may be formulated for any appropriate manner of administration, including for example, topical, oral, nasal, mucosal, intravenous, intradermal, intraperitoneal, subcutaneous, and intramuscular administration. Delivery of the OKA strain is, in one aspect, by subcutaneous delivery.

In another embodiment, a gE antigen (SEQ ID No. 1, 2, 3, 4, or 9), or immunogenic derivative or immunogenic fragment thereof, may be used with an adjuvant composition of the present application to provide an immunogenic composition or vaccine. That is, the gE antigen (SEQ ID No. 1, 2, 3, 4, or 9) or immunogenic derivative or immunogenic fragment thereof may be used in a vaccination schedule in the absence of a live attenuated strain or whole inactivated strain. Thus, the application relates to an immunogenic composition or vaccine comprising gE (SEQ ID No. 1, 2, 3, 4, or 9) or immunogenic derivative or immunogenic fragment thereof in combination with an adjuvant composition according to the present application.

In one aspect of the present application, a gE truncate is used in which gE has a C terminal truncation. In one aspect the truncation removes from 4 to 20 percent of the total amino acid residues at the carboxy terminal end. In one aspect the gE is lacking the carboxy terminal anchor region (suitably approximately amino acids 547-623 of the wild type sequence). In one aspect gE is a truncated gE having the sequence of SEQ ID No. 1 (FIG. 1) and as disclosed in Virus research, (Haumont et al Vol 40, 1996 p 199-204), herein incorporated fully by reference. In one aspect, with respect to SEQ ID No. 1, Thr 40 is substituted for Ile 40 (i.e. p.Ile40Thr). In one aspect, with respect to SEQ ID No. 1, Leu 536 is substituted for Ile 536 (i.e. p.Ile536Leu). In one aspect, both substitutions are made.

In one aspect gE is a truncated gE having the sequence of SEQ ID No. 2 (FIG. 2). In one aspect gE is a truncated gE having the sequence of SEQ ID No. 9 (FIG. 9). In another aspect of the present application, the composition comprises full-length gE (SEQ ID Nos. 3 or 4, FIG. 3 or 4).

In another aspect, the composition comprises a truncated gE having a portion of SEQ ID Nos. 3 or 4. In one aspect, with respect to SEQ ID No. 3, Ile 40 is substituted for Thr 40 (i.e. p. Thr40Ile). In one aspect, with respect to SEQ ID No. 3, Ile 536 is substituted for Leu 536 (i.e. p.Leu536Ile). In one aspect, both substitutions are made.

In another aspect the gE or derivative or fragment thereof is lyophilized. In another aspect, the gE or derivative or fragment thereof is reconstituted in a solution containing an adjuvant composition according to the present application (such as an adjuvant containing Formula II, cholesterol, DOPC, and a TLR4 agonist) before delivery.

In one embodiment the composition or vaccine comprises gE and an adjuvant composition according to the present application and does not comprise an IE63 antigen or portion thereof. In one embodiment the composition or vaccine comprises gE (SEQ ID No. 1, 2, 3, 4, or 9) and an adjuvant composition according to the present application and does not comprise any other VZV antigen. In one embodiment the composition or vaccine comprises gE (SEQ ID No. 1, 2, 3, 4, or 9) and an adjuvant according to the present application and does not comprise any other viral antigen.

In one aspect the gE or immunogenic fragment thereof is not in the form of a fusion protein. In the context of the present application, non-native signal peptides together with truncated or untruncated glycoproteins are not considered fusion proteins.

In one aspect the composition or vaccine consists essentially of the compound of Formula II, a truncated VZV gE antigen and liposomes comprising DOPC, DMPC, DMPG, cholesterol, and/or combinations of the foregoing, and a TLR4 agonist.

In one aspect the composition or vaccine consists of the compound of Formula II, a truncated VZV gE antigen and liposomes comprising cholesterol and a TLR4 agonist, and a pharmaceutically acceptable carrier.

The term ‘immunogenic derivative’ encompasses any molecule which retains the ability to induce an immune response to VZV following administration to man. Immunogenic compounds herein are suitably capable of reacting detectably within an immunoassay (such as an ELISA or T-cell stimulation assay) with antisera and/or T-cells from a patient with VZV. Screening for immunogenic activity can be performed using techniques well known to the skilled artisan. For example, such screens can be performed using methods such as those described in Harlow and Lane, Antibodies: A Laboratory Manual, Cold Spring Harbor Laboratory, 1988.

Immunogenic fragments as described herein are immunogenic derivatives that retain the ability to induce an immune response to VZV following administration to man.

Suitable methods for the generation of derivatives are well known in the art and include standard molecular biology techniques as disclosed, for example, in Sambrook et al [Molecular Cloning: A Laboratory Manual, third edition, 2000, Cold Spring Harbor Laboratory Press], such as techniques for the addition, deletion, substitution or rearrangement of amino acids or chemical modifications thereof. In one aspect derivatives include, for example, truncations or other fragments.

In one aspect derivatives in the context of this application are amino acid sequences comprising epitopes, i.e., antigenic determinants substantially responsible for the immunogenic properties of a polypeptide and being capable of eliciting an immune response, in one aspect being T cell epitopes.

In one aspect, the level of immunogenic activity of the immunogenic derivative is at least about 50%, in one aspect at least about 70% and in one aspect at least or greater than about 90% of the immunogenicity for the polypeptide from which it is derived, suitably as assessed by immunoassay techniques described above. In some aspects of the present application, immunogenic portions may be identified that have a level of immunogenic activity greater than that of the corresponding full-length polypeptide, e.g., having greater than about 100% or 150% or more immunogenic activity.

### Vaccines

Compositions in this application and their pharmaceutically acceptable salts are useful as vaccines to induce active immunity towards antigens in subjects. Any animal that may experience the beneficial effects of the compositions of the present application is within the scope of subjects that may be treated. In some embodiments, the subjects are mammals. In some embodiments, the subjects are humans.

The vaccines of the present application may be used to confer resistance to infection by either passive or active immunization. When the vaccines of the present application are used to confer resistance through active immunization, a vaccine of the present application is administered to an animal to elicit a protective immune response that either prevents or attenuates a proliferative or infectious disease. When the vaccines of the present application are used to confer resistance to infection through passive immunization, the vaccine is provided to a host animal (e.g., human, dog, or mouse), and the antisera elicited by this vaccine is recovered and directly provided to a recipient suspected of having an infection or disease or exposed to a causative organism.

The present application thus concerns and provides a means for preventing or attenuating a proliferative disease resulting from organisms that have antigens that are recognized and bound by antisera produced in response to the immunogenic antigens included in vaccines of the present application. As used herein, a vaccine is said to prevent or attenuate a disease if its administration to an animal results either in the total or partial attenuation (i.e., suppression) of a symptom or condition of the disease or in the total or partial immunity of the animal to the disease.

The administration of the vaccine (or the antisera which it elicits) may be for either a “prophylactic” or “therapeutic” purpose. When provided prophylactically, the vaccine(s) are provided in advance of any symptoms of proliferative disease. The prophylactic administration of the vaccine(s) serves to prevent or attenuate any subsequent presentation of the disease. When provided therapeutically, the vaccine(s) is provided upon or after the detection of symptoms that indicate that an animal may be infected with a pathogen. The therapeutic administration of the vaccine(s) serves to attenuate any actual disease presentation. Thus, the vaccines may be provided either before the onset of disease proliferation (to prevent or attenuate an anticipated infection) or after the initiation of an actual proliferation.

One of ordinary skill in the art will appreciate that vaccines may optionally include a pharmaceutically acceptable excipient or carrier. Thus, according to another aspect, provided vaccines may comprise one or more antigens that are optionally conjugated to a pharmaceutically acceptable excipient or carrier. In some embodiments, said one or more antigens are conjugated covalently to a pharmaceutically acceptable excipient. In other embodiments, said one or more antigens are non-covalently associated with a pharmaceutically acceptable excipient.

As described above, adjuvants may be used to increase the immune response to an antigen. According to the present application, provided vaccines may be used to invoke an immune response when administered to a subject. In certain embodiments, an immune response to an antigen may be potentiated by administering to a subject a provided vaccine in an effective amount to potentiate the immune response of said subject to said antigen.

### Formulations

The compounds of the present application and/or their salts may be combined with a pharmaceutically acceptable excipient to form a pharmaceutical composition. In certain embodiments, formulations of the present application include injectable formulations. In certain embodiments, the pharmaceutical composition includes a pharmaceutically acceptable amount of a compound of the present application. In certain embodiments, the compounds of the application and an antigen form an active ingredient. In certain embodiments, the compound of the present application alone forms an active ingredient.

The amount of active ingredient(s) which can be combined with a carrier material to produce a single dosage form will vary depending upon the host being treated, and the particular mode of administration. The amount of active ingredient(s) that can be combined with a carrier material to produce a single dosage form will generally be that amount of the compound which produces a therapeutic effect. Generally, this amount will range from about 1% to about 99% of active ingredient, preferably from about 5% to about 70%, most preferably from about 10% to about 30%, or from about 1% to 99%, preferably from 10% to 90%, 20% to 80%, 30% to 70%, 40% to 60%, 45% to 55%, or about 50%.

Wetting agents, emulsifiers, and lubricants, such as sodium lauryl sulfate and magnesium stearate, as well as coloring agents, release agents, coating agents, sweetening, flavoring, and perfuming agents, preservatives, and antioxidants can also be present in the compositions.

Thus, in one aspect the present application provides formulations comprising a liposome formulation of a TLR4 agonist or antagonist according to the present application and Compound 1-4. In another aspect, the present application provides formulations comprising a TLR4 agonist or antagonist according to the present application, Compound 1-4, and a squalene emulsion. In another aspect, the present application provides formulations comprising a TLR4 agonist or antagonist according to the present application, Compound 1-4, and CpG 7909 or CpG 1018.

Liposomal formulations of a TLR4 agonist or antagonist according to the present application and naturally occurring QS-21 are formulated, for example, by first producing liposomes by mixing methanol and a cholesterol. Liposomes are closed bilayer membranes containing an entrapped aqueous volume. Liposomes may also be uni-lamellar vesicles possessing a single membrane bilayer or multi-lamellar vesicles with multiple membrane bilayers, each separated from the next by an aqueous layer. The structure of the resulting membrane bilayer is such that the hydrophobic (non-polar) tails of the lipid are oriented toward the center of the bilayer while the hydrophilic (polar) heads orient towards the aqueous phase. Suitable hydrophilic polymers for surrounding the liposomes include, without limitation, PEG, polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxethylacrylate, hydroxymethylcellulose, hydroxyethylcellulose, polyethyleneglycol, polyaspartamide and hydrophilic peptide sequences as described in U.S. Pat. Nos. 6,316,024; 6,126,966; 6,056,973; and 6,043,094. Liposomes can be made without hydrophilic polymers. Therefore, liposome formulations may or may not contain hydrophilic polymers.

Liposomes may be comprised of any lipid or lipid combination known in the art. For example, the vesicle-forming lipids may be naturally-occurring or synthetic lipids, including phospholipids, such as phosphatidylcholine, phosphatidylethanolamine, phosphatide acid, phosphatidylserine, phosphatidylglycerol, phosphatidylinositol, and sphingomyelin as disclosed in U.S. Pat. Nos. 6,056,973 and 5,874,104.

The vesicle-forming lipids may also be glycolipids, cerebrosides, or cationic lipids, such as 1,2-dioleyloxy-3-(trimethylamino) propane (DOTAP); N—[I-(2,3, -ditetradecyloxy)propyl]-N,N-dimethyl-N-hydroxyethylammonium bromide (DMRIE); N—[I [(2,3,-dioleyloxy)propyl]-N,N-dimethyl-N-hydroxy ethylammonium bromide (DORIE); N—[I-(2,3-dioleyloxy)propyl]-N,N,N-trimethylammonium chloride (DOTMA); 3 [N—(N′,N′-dimethylaminoethane) carbamoly] cholesterol (DCChol); or dimethyldioctadecylammonium (DDAB) also as disclosed in U.S. Pat. No. 6,056,973. Cholesterol may also be present in the proper range to impart stability to the liposome vesicle, as disclosed in U.S. Pat. Nos. 5,916,588 and 5,874,104. Additional liposomal technologies are described in U.S. Pat. Nos. 6,759,057; 6,406,713; 6,352,716; 6,316,024; 6,294,191; 6,126,966; 6,056,973; 6,043,094; 5,965,156; 5,916,588; 5,874,104; 5,215,680; and 4,684,479. These described liposomes and lipid-coated microbubbles, and methods for their manufacture. Thus, one skilled in the art, considering both the present disclosure and the disclosures of these other patents could produce a liposome for the purposes of the present embodiments. Liposomes may comprise phospholipid or nonphospholipid bilayers. Phospholipid bilayers may comprise hydrocarbon chains, optionally having a melting temperature in water of at least 23C. Such phospholipids may comprise, for example, dimyristoyl phosphatidylcholine (DMPC), dimyristoyl phosphatidylglycerol (DMPG), cholesterol (Chol), or similar molecules, and mixtures thereof. The liposome may optionally comprise a neutral lipid that is non-crystalline at room temperatures, such as dioleoyl phosphatidylcholine or similar compounds. See U.S. Published Patent Application No. 2011/0206758.

During the manufacture of liposomal formulations containing, for example, QS-21, small unicellular liposomal vesicles (SUV) are first created. The SUV is then added to an aqueous environment having QS-21 or another saponin and the SUV takes up QS-21 or the saponin from the aqueous environment. The liposomal composition also may have certain optional ingredients, such as MPL, synthetic MPL such as MPLA, CpG 7909, or CpG 1018, or similar substances.

However, formulation of liposomal formulations containing other saponin derivatives such as Compound 1-4 cannot be accomplished using procedures known in the art, because the SUV or liposomal formulations do not take up such saponin derivatives, resulting in SUV or liposomes without the saponin derivative molecule. Thus, another aspect of the present application provides a novel method of producing liposomal formulations of saponin derivates that cannot be formulated using traditional methods. In such a method, the SUVs or liposomes are first formulated with the presence of a saponin derivative such as Compound 1-4. For example, the SUV may be formulated by combining a lipid such as a cholesterol and methanol in the presence of Compound 1-4. The SUV may also be formulated according to the traditional method as set forth above; however, the SUV is formulated in the presence of a saponin derivative such as QS-21. These SUV or liposomes form with Compound I-4 incorporated therein. Such SUV or liposomes are then added to an aqueous environment having, for example, MPL or other compositions as set forth above.

Non-limiting examples of pharmaceutically-acceptable antioxidants include water-soluble antioxidants, such as ascorbic acid, cysteine hydrochloride, sodium bisulfate, sodium metabisulfite, sodium sulfite, and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Suspensions, in addition to the active compounds, may contain suspending agents such as, for example, ethoxylated isostearyl alcohols, polyoxyethylene sorbitol, and sorbitan esters, microcrystalline cellulose, aluminum metahydroxide, bentonite, agar-agar and tragacanth, and mixtures thereof.

Non-limiting examples of suitable aqueous and nonaqueous carriers, which may be employed in the pharmaceutical compositions of the present application include water, alcohols (including but not limited to methanol, ethanol, butanol, etc.), polyols (including but not limited to glycerol, propylene glycol, polyethylene glycol, etc.), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain additives such as preservatives, wetting agents, emulsifying agents, and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, to prolong the effect of a formulation, it is desirable to slow the absorption of the drug from a subcutaneous or intramuscular injection. This may be accomplished by the use of a liquid suspension of crystalline or amorphous material having poor water solubility. The rate of absorption of the drug then depends upon its rate of dissolution, which in turn, may depend upon crystal size and crystalline form.

Regardless of the route of administration selected, the compounds of the present application, which may be used in a suitable hydrated form, and/or the pharmaceutical compositions of the present application, are formulated into pharmaceutically-acceptable dosage forms by conventional methods known to those of skill in the art.

Actual dosage levels of the active ingredients in the pharmaceutical compositions of the present application may be varied to obtain an amount of the active ingredient that is effective to achieve the desired therapeutic response for a particular patient, composition, and mode of administration, without being toxic to the patient.

The selected dosage level will depend upon a variety of factors including the activity of the particular compound of the present application employed, or the ester, salt, or amide thereof, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the present application employed in the pharmaceutical composition at levels lower than that required to achieve the desired therapeutic effect and then gradually increase the dosage until the desired effect is achieved.

In some embodiments, a compound or pharmaceutical composition of the present application is provided to a subject chronically. Chronic treatments include any form of repeated administration for an extended period, such as repeated administrations for one or more months, between a month and a year, one or more years, or longer. In many embodiments, a chronic treatment involves administering a compound or pharmaceutical composition of the present application repeatedly over the life of the subject. Preferred chronic treatments involve regular administrations, for example, one or more times a day, one or more times a week, or one or more times a month. In general, a suitable dose, such as a daily dose of a compound of the present application, will be that amount of the compound that is the lowest dose effective to produce a therapeutic effect. Such an effective dose will generally depend upon the factors described above.

Generally, doses of the compounds of the present application for a patient, when used for the indicated effects, will range from about 0.0001 to about 100 mg per kg of body weight per day. Preferably the daily dosage will range from 0.001 to 50 mg of compound per kg of body weight, and even more preferably from 0.01 to 10 mg of compound per kg of body weight. However, lower or higher doses can be used. In some embodiments, the dose administered to a subject may be modified as the physiology of the subject changes due to age, disease progression, weight, or other factors.

In some embodiments, provided adjuvant compounds of the present application are administered as pharmaceutical compositions or vaccines. In certain embodiments, it is contemplated that the amount of adjuvant compound administered will be 1-2000 μg. In certain embodiments, it is contemplated that the amount of adjuvant compound administered will be 1-1000 μg. In certain embodiments, it is contemplated that the amount of adjuvant compound administered will be 1-500 μg. In certain embodiments, it is contemplated that the amount of adjuvant compound administered will be 1-250 μg. In certain embodiments, it is contemplated that the amount of adjuvant compound administered will be 100-1000 μg. In certain embodiments, it is contemplated that the amount of adjuvant compound administered will be 100-500 μg. In certain embodiments, it is contemplated that the amount of adjuvant compound administered will be 100-200 μg. In certain embodiments, it is contemplated that the amount of adjuvant compound administered will be 250-500 μg. In certain embodiments, it is contemplated that the amount of adjuvant compound administered will be 10-1000 μg. In certain embodiments, it is contemplated that the amount of adjuvant compound administered will be 500-1000 μg. In certain embodiments, it is contemplated that the amount of adjuvant compound administered will be 50-250 μg. In certain embodiments, it is contemplated that the amount of adjuvant compound administered will be 50-500 g.

In some embodiments, provided adjuvant compounds of the present application are administered as pharmaceutical compositions or vaccines. In certain embodiments, it is contemplated that the amount of adjuvant compound administered will be 1-2000 mg. In certain embodiments, it is contemplated that the amount of adjuvant compound administered will be 1-1000 mg. In certain embodiments, it is contemplated that the amount of adjuvant compound administered will be 1-500 mg. In certain embodiments, it is contemplated that the amount of adjuvant compound administered will be 1-250 mg. In certain embodiments, it is contemplated that the amount of adjuvant compound administered will be 100-1000 mg. In certain embodiments, it is contemplated that the amount of adjuvant compound administered will be 100-500 mg. In certain embodiments, it is contemplated that the amount of adjuvant compound administered will be 100-200 mg. In certain embodiments, it is contemplated that the amount of adjuvant compound administered will be 250-500 mg. In certain embodiments, it is contemplated that the amount of adjuvant compound administered will be 10-1000 mg. In certain embodiments, it is contemplated that the amount of adjuvant compound administered will be 500-1000 mg. In certain embodiments, it is contemplated that the amount of adjuvant compound administered will be 50-250 mg. In certain embodiments, it is contemplated that the amount of adjuvant compound administered will be 50-500 mg. In certain embodiments, it is contemplated that the amount of adjuvant compound administered will be 0.01-215.4 mg.

In certain embodiments, it is contemplated that the amount of adjuvant administered will be 1000-5000 μg/kg. In certain embodiments, it is contemplated that the amount of adjuvant administered will be 1000-4000 μg/kg. In certain embodiments, it is contemplated that the amount of adjuvant administered will be 1000-3000 μg/kg. In certain embodiments, it is contemplated that the amount of adjuvant administered will be 1000-2000 g/kg. In certain embodiments, it is contemplated that the amount of adjuvant administered will be 2000-5000 g/kg. In certain embodiments, it is contemplated that the amount of adjuvant administered will be 2000-4000 μg/kg. In certain embodiments, it is contemplated that the amount of adjuvant administered will be 2000-3000 μg/kg. In certain embodiments, it is contemplated that the amount of adjuvant administered will be 3000-5000 μg/kg. In certain embodiments, it is contemplated that the amount of adjuvant administered will be 3000-4000 μg/kg. In certain embodiments, it is contemplated that the amount of adjuvant administered will be 4000-5000 μg/kg. In certain embodiments, it is contemplated that the amount of adjuvant administered will be 1-500 μg/kg. In certain embodiments, it is contemplated that the amount of adjuvant administered will be 500-1000 μg/kg. In certain embodiments, it is contemplated that the amount of adjuvant administered will be 1000-1500 μg/kg. In certain embodiments, it is contemplated that the amount of adjuvant administered will be 1 mg/kg. In certain embodiments, it is contemplated that the amount of adjuvant administered will be 2 mg/kg. In certain embodiments, it is contemplated that the amount of adjuvant administered will be 3 mg/kg. In certain embodiments, it is contemplated that the amount of adjuvant administered will be 4 mg/kg. In certain embodiments, it is contemplated that the amount of adjuvant administered will be 5 mg/kg. In certain embodiments, it is contemplated that the amount of adjuvant administered will be 0.0029-5 mg/kg. In certain embodiments, the amount of adjuvant administered in females is less than the amount of adjuvant administered in males. In certain embodiments, the amount of adjuvant administered to infants is less than the amount of adjuvant administered to adults. In certain embodiments, the amount of adjuvant administered to pediatric recipients is less than the amount of adjuvant administered to adults. In certain embodiments, the amount of adjuvant administered to immunocompromised recipients is more than the amount of adjuvant administered to healthy recipients. In certain embodiments, the amount of adjuvant administered to elderly recipients is more than the amount of adjuvant administered to non-elderly recipients.

If desired, the effective dose of the active compound may be administered as two, three, four, five, six, or more sub-doses administered separately at appropriate intervals throughout the day, optionally, in unit dosage forms.

While a compound of the present application can be administered alone, in certain embodiments the compound is administered as a pharmaceutical formulation or composition as described above.

The compounds according to the present application may be formulated for administration in any convenient way for use in human or veterinary medicine, by analogy with other pharmaceuticals.

The present application provides kits comprising pharmaceutical formulations or compositions of a compound of the present application. In certain embodiments, such kits include the combination of a compound of formulae I and/or II and an antigen. The agents may be packaged separately or together. The kit optionally includes instructions for prescribing the medication. In certain embodiments, the kit includes multiple doses of each agent. The kit may include sufficient quantities of each component to treat one or more subjects for a week, two weeks, three weeks, four weeks, or multiple months. The kit may include a full cycle of immunotherapy. In some embodiments, the kit includes a vaccine comprising one or more bacterial or viral-associated antigens, and one or more provided compounds.

### Vaccine Regimes

In one aspect the present application relates to a prime boost regime wherein a VZV antigen, in one aspect an adjuvanted antigen, is delivered first, after which the immune system is boosted with delivery of an attenuated VZV.

A prime boost regime in humans comprises, in one aspect, priming with 25-100 μg gE (SEQ ID No. 1, 2, 3, 4, or 9), in one aspect 40-100 μg gE (SEQ ID No. 1, 2, 3, 4, or 9), such as 50 or about 50 μg gE (SEQ ID No. 1, 2, 3, 4, or 9), or an immunogenic derivative thereof, adjuvanted with an adjuvant of Formula II, and boosting with the OKA strain of VZV.

Where prime boost regimes are used, or where multiple vaccination regimes are used, then 2, 3, 4 or more immunizations may be employed. Suitable regimes for prime boost include 1, 2, 3, 4, 5 or 6 months between individual immunizations. A prime boost schedule comprises, in one aspect, delivery of a VZV antigen or immunogenic derivative thereof, suitably an adjuvanted VZV antigen or derivative, at 0 months and boosting with a live attenuated VZV at 2 months.

In an alternative delivery schedule, there is concomitant delivery of both of the two individual components (VZV antigen or derivative and live attenuated VZV) at both 0 and 2 months.

In an alternative delivery schedule, there is delivery of VZV antigen or derivative thereof (no live attenuated or killed VZV) at both 0 and 2 months. The VZV antigen may be, for example, VZV gE (SEQ ID No. 1, 2, 3, 4, or 9).

In an alternative delivery schedule, there is delivery of a VZV antigen or a derivate thereof in a single dose. The VZV antigen may be, for example, VZV gE (SEQ ID No. 1, 2, 3, 4, or 9).

The composition or vaccine is suitably used in the population of people 50 or older than 50. Suitably the population is the population of those older than 55, 60, 65, 70, 75, 80, or older than 80. Suitably the population is 50-70 years.

In one aspect the population of individuals are those who have had varicella or who have had a live varicella vaccine.

Thus, the present application relates to use of a composition as described above in the preparation of a medicament for the prevention or amelioration of herpes zoster reactivation and/or post herpetic neuralgia in a population of people 50 or above.

The present application thus also relates to a method for the prevention or amelioration of herpes zoster reactivation and/or post herpetic neuralgia, the method comprising delivering to an individual in need thereof a composition of the present application.

In one aspect the composition of the first and second aspects of the present application are used in those individuals in whom the varicella zoster virus has not reactivated.

The composition may be used at doses and delivery routes as outlined above for the first aspect of the invention. Specifically, the amount of gE antigen (SEQ ID No. 1, 2, 3, 4, or 9) is selected as an amount which induces an immunoprotective response without significant, adverse side effects in typical vaccines. Such amount will vary depending upon which specific immunogen is employed and how it is presented. Generally, it is expected that each dose will comprise 1-1000 μg of protein, such as 2-100 μg, or 5-60 μg. Where gE (SEQ ID No. 1, 2, 3, 4, or 9) is used then suitably 25-100 μg gE (SEQ ID No. 1, 2, 3, 4, or 9) is used, in one aspect 40-100 μg of gE (SEQ ID No. 1, 2, 3, 4, or 9), such as about 25 μg, 50 μg or about 100 μg of gE (SEQ ID No. 1, 2, 3, 4, or 9), suitably 25 μg, 50 μg or 100 μg gE (SEQ ID No. 1, 2, 3, 4, or 9). An optimal amount for a particular vaccine can be ascertained by standard studies involving observation of appropriate immune responses in subjects. Following an initial vaccination, subjects may receive one or several booster immunization adequately spaced.

In one aspect the gE (SEQ ID No. 1, 2, 3, 4, or 9) and adjuvant composition or vaccine is used in a one dose delivery regime. In one aspect the gE (SEQ ID No. 1, 2, 3, 4, or 9) and adjuvant composition or vaccine is used in a two-dose delivery regime. In one aspect the composition or vaccine of the invention is used in a 2 dose regime with a 2 month spacing between doses.

### Vaccine Kits and Medicaments

In another embodiment, the present application relates to a kit comprising a live attenuated VZV or inactivated whole VZV and a VZV antigen.

In another aspect, the present application relates to a kit comprising, as separate components, an adjuvant composition according to the present application and a gE antigen or immunogenic fragment thereof, as described above, suitable for extemporaneous preparation of a vaccine composition. In one aspect both components are liquids. In one aspect one component is lyophilized and is suitable for reconstitution with the other component. In one aspect the kit comprises a gE antigen having the sequence SEQ ID No. 1 and an adjuvant comprising the compound of Formula II and liposomes comprising cholesterol and a TLR4 agonist.

In yet another embodiment, the present application relates to use of a VZV antigen, including a composition comprising gE (SEQ ID No. 1, 2, 3, 4, or 9), or an immunogenic derivative or immunogenic fragment thereof in combination with an adjuvant composition according to the present application, in the preparation of a medicament for the prevention or amelioration of herpes zoster reactivation and/or post herpetic neuralgia.

### Methods

The present application also encompasses methods of conferring immune resistance to an individual. Such methods include administering to an individual a vaccine comprising a therapeutically effective amount of a compound of Formula I or Formula II, or any of the individual compounds disclosed herein, alone or in combination with one another, in free form or pharmaceutically acceptable salt form, together with an antigen.

### Further Embodiments

In a series of further specific or alternate embodiments, the present application also provides:
1.1 An immunogenic composition comprising:
   a varicella zoster virus antigen, and
   a compound of Formula I:

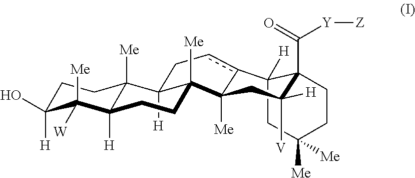

or a pharmaceutically acceptable salt thereof, wherein
[symbol] is a single or double bond;
W is —CHO;
V is hydrogen or OR^{x};
Y is CH₂, —O—, —NR—, or —NH—;
   Z is hydrogen; a cyclic or acyclic, optionally substituted moiety selected from the group consisting of acyl, aliphatic, heteroaliphatic, aryl, arylalkyl, heteroacyl, and heteroaryl; or a carbohydrate domain having the structure:

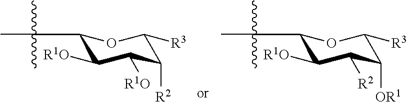

wherein each occurrence of R¹ is R^{x} or a carbohydrate domain having the structure:

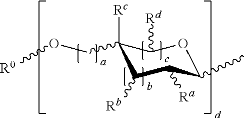

wherein:
each occurrence of a, b, and c is independently 0, 1, or 2;
d is an integer from 1-5, wherein each d bracketed structure may be the same or different; with the proviso that the d bracketed structure represents a furanose or a pyranose moiety, and the sum of b and c is 1 or 2;
R⁰ is hydrogen; an oxygen protecting group selected from the group consisting of alkyl ethers, benzyl ethers, silyl ethers, acetals, ketals, esters, carbamates, and carbonates; or an optionally substituted moiety selected from the group consisting of acyl, C₁₋₁₀ aliphatic, C₁₋₆ heteroaliphatic, 6-10-membered aryl, arylalkyl, 5-10 membered heteroaryl having 1-4 heteroatoms independently selected from nitrogen, oxygen, or sulfur, 4-7 membered heterocyclyl having 1-2 heteroatoms independently selected from the group consisting of nitrogen, oxygen, and sulfur;
each occurrence of R^{a}, R^{b}, R^{c}, and R^{d} is independently hydrogen, halogen, OH, OR, OR^{x}, NR₂, NHCOR, or an optionally substituted group selected from acyl, C₁₋₁₀ aliphatic, C₁₋₆ heteroaliphatic, 6-10-membered aryl, arylalkyl, 5-10-membered heteroaryl having 1-4 heteroatoms independently selected from nitrogen, oxygen, sulfur; 4-7-membered heterocyclyl having 1-2 heteroatoms independently selected from the group consisting of nitrogen, oxygen, and sulfur;
   R² is hydrogen, halogen, OH, OR, OC(O)R⁴, OC(O)OR⁴, OC(O)NHR⁴, OC(O)NRR⁴, OC(O)SR⁴, NHC(O)R⁴, NRC(O)R⁴, NHC(O)OR⁴, NHC(O)NHR⁴, NHC(O)NRR⁴, NHR⁴, N(R⁴)₂, NHR⁴, NRR⁴, N₃, or an optionally substituted group selected from C₁₋₁₀ aliphatic, C₁₋₆ heteroaliphatic, 6-10-membered aryl, arylalkyl, 5-10 membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur;
   R³ is hydrogen, halogen, CH₂OR¹, or an optionally substituted group selected from the group consisting of acyl, C₁₋₁₀ aliphatic, C₁₋₆ heteroaliphatic, 6-10-membered aryl, arylalkyl, 5-10-membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur,
   R⁴ is -T-R^{z}, —C(O)-T-R^{z}, —NH-T-R^{z}, —O-T-R^{z}, —S-T-R^{z}, —C(O)NH-T-R^{z} C(O)O-T-R^{z}, C(O)S-T-R^{z}, C(O)NH-T-O-T-R^{z}, —O-T-R^{z}, -T-O-T-R^{z}, -T-S-T-R^{z}, or

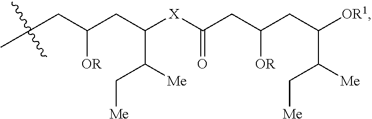

wherein
X is —O—, —NR—, or T-R^{z};
T is a covalent bond or a bivalent C₁₋₂₆ saturated or unsaturated, straight or branched, aliphatic or heteroaliphatic chain; and
R^{z} is hydrogen, halogen, —OR, —OR^{x}, —OR¹, —SR, NR₂, —C(O)OR, —C(O)R, —NHC(O)R, —NHC(O)OR, NC(O)OR, or an optionally substituted group selected from acyl, arylalkyl, heteroarylalkyl, C₁₋₆ aliphatic, 6-10-membered aryl, 5-10-membered heteroaryl having 1-4 heteroatoms independently selected from nitrogen, oxygen, or sulfur, 4-7-membered heterocyclyl having 1-2 heteroatoms independently selected from the group consisting of nitrogen, oxygen, and sulfur;
   each occurrence of R^{x} is independently hydrogen or an oxygen protecting group selected from the group consisting of alkyl ethers, benzyl ethers, silyl ethers, acetals, ketals, esters, carbamates, and carbonates;
   each occurrence of R is independently hydrogen, an optionally substituted group selected from acyl, arylalkyl, 6-10-membered aryl, C₁₋₆ aliphatic, or C₁₋₆ heteroaliphatic having 1-2 heteroatoms independently selected from the group consisting of nitrogen, oxygen, and sulfur, or:
   two R on the same nitrogen atom are taken with the nitrogen atom to form a 4-7-membered heterocyclic ring having 1-2 heteroatoms independently selected from the group consisting of nitrogen, oxygen, and sulfur.
1.2. The immunogenic composition of 1.1,
   wherein the compound of Formula I is:

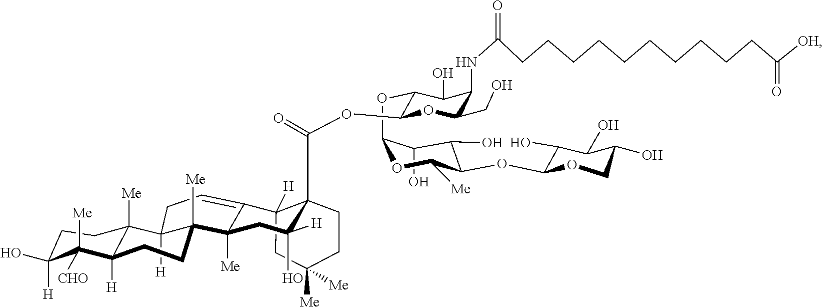

or
a pharmaceutically acceptable salt thereof.
1.3. The immunogenic composition of any of 1.1 to 1.2,
   wherein the compound of Formula I is in free acid form.
1.4. The immunogenic composition of any of 1.1 to 1.2,
   wherein the compound of Formula I is in choline salt form.
1.5. The immunogenic composition of any of 1.1 to 1.4,
   wherein the varicella zoster virus antigen is a varicella zoster virus gE antigen truncated to remove the carboxy terminal anchor region.
1.6. The immunogenic composition of any of 1.1 to 1.5,
   wherein the varicella zoster virus gE antigen is a truncate.
1.7. The immunogenic composition of any of 1.1 to 1.6,
   wherein the varicella zoster virus gE antigen is a C-terminal truncate.
1.8. The immunogenic composition of any of 1.1 to 1.7,
   wherein the varicella zoster virus antigen has the sequence of SEQ ID No. 1, SEQ ID No. 2, or SEQ ID No. 9.
1.9. The immunogenic composition of any of 1.1 to 1.7,
   wherein the varicella zoster virus antigen has the sequence of SEQ ID No. 1.
1.10. The immunogenic composition of any of 1.1 to 1.7,
   wherein the varicella zoster virus antigen has the sequence of SEQ ID No. 9.
1.11. The immunogenic composition of any of 1.1 to 1.10,
   further comprising a TLR4 or a TLR9 agonist.
1.12. The immunogenic composition of any of 1.1 to 1.11,
   further comprising a liposome-forming compound.
1.13. The immunogenic composition of 1.12,
   wherein the liposome-forming compound forms liposomes containing the TLR4 or TLR9 agonist.
1.14. The immunogenic composition of any of 1.12 to 1.13,
   wherein the liposome-forming compound is selected from the group consisting of DOPC, DMPC, DMPG, cholesterol, and combinations thereof.
1.15. The immunogenic composition according to any of 1.1 to 1.11.
   further comprising an emulsion.
1.16. The immunogenic composition according to 1.15,
   wherein the emulsion is an oil-in-water emulsion.
1.17. The immunogenic composition according to any of 1.15 to 1.16,
   wherein the emulsion contains the compound of Formula I or pharmaceutically acceptable salt thereof.
1.18. The immunogenic composition according to any of 1.15 to 1.17,
   wherein the emulsion contains the TLR4 or TLR9 agonist.
1.19. The immunogenic composition according to any of 1.1 to 1.13,
   further comprising an emulsion.
1.20. The immunogenic composition according to 1.19,
   wherein the emulsion is an oil-in-water emulsion.
1.21. The immunogenic composition according to any of 1.19 to 1.20,
   wherein the emulsion contains the compound of Formula I or pharmaceutically acceptable salt thereof.
1.22. The immunogenic composition according to any of 1.1 to 1.11,
   further comprising a cyclodextrin.
2.1. A method of increasing cell-mediated immunity in a patient, said method comprising administering to said patient an effective amount of an immunogenic composition comprising a varicella zoster virus antigen and a compound of Formula I:

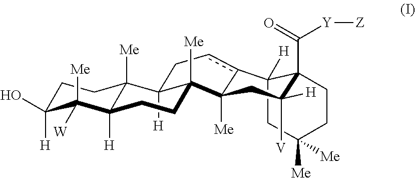

or a pharmaceutically acceptable salt thereof, wherein
is a single or double bond;
W is —CHO;
V is hydrogen or OR^{x};
Y is CH₂, —O—, —NR—, or —NH—;
   Z is hydrogen; a cyclic or acyclic, optionally substituted moiety selected from the group consisting of acyl, aliphatic, heteroaliphatic, aryl, arylalkyl, heteroacyl, and heteroaryl; or a carbohydrate domain having the structure:

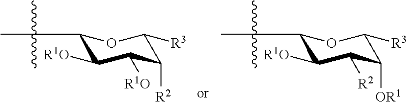

wherein each occurrence of R¹ is R^{x} or a carbohydrate domain having the structure:

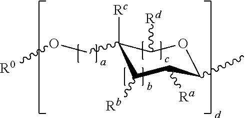

wherein:
each occurrence of a, b, and c is independently 0, 1, or 2;
d is an integer from 1-5, wherein each d bracketed structure may be the same or different; with the proviso that the d bracketed structure represents a furanose or a pyranose moiety, and the sum of b and c is 1 or 2;
R⁰ is hydrogen; an oxygen protecting group selected from the group consisting of alkyl ethers, benzyl ethers, silyl ethers, acetals, ketals, esters, carbamates, and carbonates; or an optionally substituted moiety selected from the group consisting of acyl, C₁₋₁₀ aliphatic, C₁₋₆ heteroaliphatic, 6-10-membered aryl, arylalkyl, 5-10 membered heteroaryl having 1-4 heteroatoms independently selected from nitrogen, oxygen, or sulfur, 4-7 membered heterocyclyl having 1-2 heteroatoms independently selected from the group consisting of nitrogen, oxygen, and sulfur;
each occurrence of R^{a}, R^{b}, R^{c}, and R^{d} is independently hydrogen, halogen, OH, OR, OR^{x}, NR₂, NHCOR, or an optionally substituted group selected from acyl, C₁₋₁₀ aliphatic, C₁₋₆ heteroaliphatic, 6-10-membered aryl, arylalkyl, 5-10-membered heteroaryl having 1-4 heteroatoms independently selected from nitrogen, oxygen, sulfur; 4-7-membered heterocyclyl having 1-2 heteroatoms independently selected from the group consisting of nitrogen, oxygen, and sulfur;
   R² is hydrogen, halogen, OH, OR, OC(O)R⁴, OC(O)OR⁴, OC(O)NHR⁴, OC(O)NRR⁴, OC(O)SR⁴, NHC(O)R⁴, NRC(O)R⁴, NHC(O)OR⁴, NHC(O)NHR⁴, NHC(O)NRR⁴, NHR⁴, N(R⁴)₂, NHR⁴, NRR⁴, N₃, or an optionally substituted group selected from C₁₋₁₀ aliphatic, C₁₋₆ heteroaliphatic, 6-10-membered aryl, arylalkyl, 5-10 membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur;
   R³ is hydrogen, halogen, CH₂OR¹, or an optionally substituted group selected from the group consisting of acyl, C₁₋₁₀ aliphatic, C₁₋₆ heteroaliphatic, 6-10-membered aryl, arylalkyl, 5-10-membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur,
   R⁴ is -T-R^{z}, —C(O)-T-R^{z}, —NH-T-R^{z}, —O-T-R^{z}, —S-T-R^{z}, —C(O)NH-T-R^{z}, C(O)O-T-R^{z}, C(O)S-T-R^{z}, C(O)NH-T-O-T-R^{z}, —O-T-R^{z}, -T-O-T-R^{z}, -T-S-T-R^{z}, or

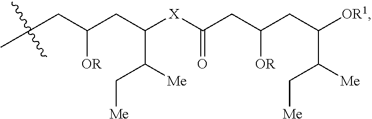

wherein
X is —O—, —NR—, or T-R^{z};
T is a covalent bond or a bivalent C₁₋₂₆ saturated or unsaturated, straight or branched, aliphatic or heteroaliphatic chain; and
R^{z} is hydrogen, halogen, —OR, —OR^{x}, —OR¹, —SR, NR₂, —C(O)OR, —C(O)R, —NHC(O)R, —NHC(O)OR, NC(O)OR, or an optionally substituted group selected from acyl, arylalkyl, heteroarylalkyl, C₁₋₆ aliphatic, 6-10-membered aryl, 5-10-membered heteroaryl having 1-4 heteroatoms independently selected from nitrogen, oxygen, or sulfur, 4-7-membered heterocyclyl having 1-2 heteroatoms independently selected from the group consisting of nitrogen, oxygen, and sulfur;
   each occurrence of R^{x} is independently hydrogen or an oxygen protecting group selected from the group consisting of alkyl ethers, benzyl ethers, silyl ethers, acetals, ketals, esters, carbamates, and carbonates;
   each occurrence of R is independently hydrogen, an optionally substituted group selected from acyl, arylalkyl, 6-10-membered aryl, C₁₋₆ aliphatic, or C₁₋₆ heteroaliphatic having 1-2 heteroatoms independently selected from the group consisting of nitrogen, oxygen, and sulfur, or:
   two R on the same nitrogen atom are taken with the nitrogen atom to form a 4-7-membered heterocyclic ring having 1-2 heteroatoms independently selected from the group consisting of nitrogen, oxygen, and sulfur,
wherein the cell mediated immunity prevents herpes zoster reactivation in the patient.
2.2. The method of 2.1,
   wherein the compound of Formula I is:

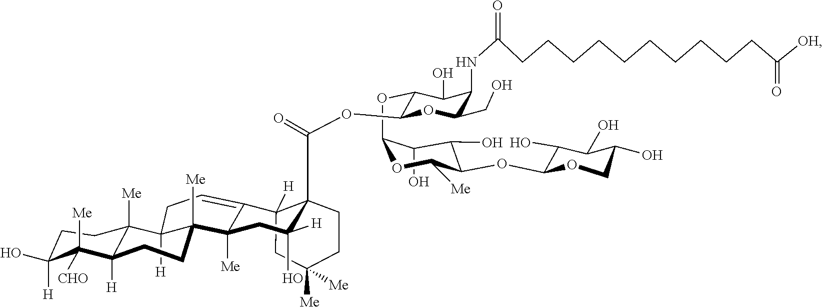

or
a pharmaceutically acceptable salt thereof.
2.3. The method of any of 2.1 to 2.2,
   wherein the compound of Formula I is in free acid form.
2.4. The method of any of 2.1 to 2.2,
   wherein the compound of Formula I is in choline salt form.
2.5. The method of any of 2.1 to 2.4,
   wherein the varicella zoster virus antigen is a varicella zoster virus gE antigen truncated to remove the carboxy terminal anchor region.
2.6. The method of any of 2.1 to 2.5,
   wherein the varicella zoster virus gE antigen is a truncate.
2.7. The method of any of 2.1 to 2.6,
   wherein the varicella zoster virus gE antigen is a C-terminal truncate.
2.8. The method of any of 2.1 to 2.7,
   wherein the varicella zoster virus antigen has the sequence of SEQ ID No. 1, SEQ ID No. 2, or SEQ ID No. 9.
2.9. The method of any of 2.1 to 2.7,
   wherein the varicella zoster virus antigen has the sequence of SEQ ID No. 1.
2.10. The method of any of 2.1 to 2.7,
   wherein the varicella zoster virus antigen has the sequence of SEQ ID No. 9.
2.11. The method of any of 2.1 to 2.10,
   further comprising a TLR4 or TLR9 agonist.
2.12. The method of any of 2.1 to 2.11,
   further comprising a liposome-forming compound.
2.13. The method of 2.12,
   wherein the liposome-forming compound forms liposomes containing the TLR4 agonist.
2.14. The method of any of 2.12 to 2.13,
   wherein the liposome-forming compound is selected from the group consisting of DOPC, DMPC, DMPG, cholesterol, and combinations thereof.
2.15. The method according to any of 2.1 to 2.11,
   further comprising an emulsion.
2.16. The method according to 2.15,
   wherein the emulsion is an oil-in-water emulsion.
2.17. The method according to any of 2.15 to 2.16,
   wherein the emulsion contains the compound of Formula I or pharmaceutically acceptable salt thereof.
2.18. The method according to any of 2.15 to 2.17,
   wherein the emulsion contains the TLR4 or TLR9 agonist.
2.19. The method according to any of 2.1 to 2.13,
   further comprising an emulsion.
2.20. The method according to 2.19,
   wherein the emulsion is an oil-in-water emulsion.
2.21. The method according to any of 2.19 to 2.20,
   wherein the emulsion contains the compound of Formula I or pharmaceutically acceptable salt thereof.
2.22. The method according to any of 2.1 to 2.11,
   further comprising a cyclodextrin.

### EXAMPLES

### Example 1—Liposome PHAD+Compound I-4 Free Acid Form

The impact of TQL-1055 free acid (Compound I-4 free acid) on antibody titers induced by gE antigen was tested. Mice were immunized with gE (5 mcg) alone, gE (5 mcg) with blank liposomes, gE (5 mcg) with liposomes containing PHAD (7.5 mcg), gE (5 mcg) with liposomes containing PHAD (7.5 mcg) and TQL-1055 free acid (four groups: 5 mcg, 15 mcg, 30 mcg, 50 mcg), and gE (5 mcg) with liposomes containing MPL (7.5 mcg) and QS-21 (5 mcg). Mice were immunized at Day 0 and Day 14. Groups were bled at Day 14 (post Dose 1) and Day 28 (post Dose 2) for serum analysis for anti-gE specific antibody response. The results are shown below.

gE-Specific Total IgG Titers Post Dose 1

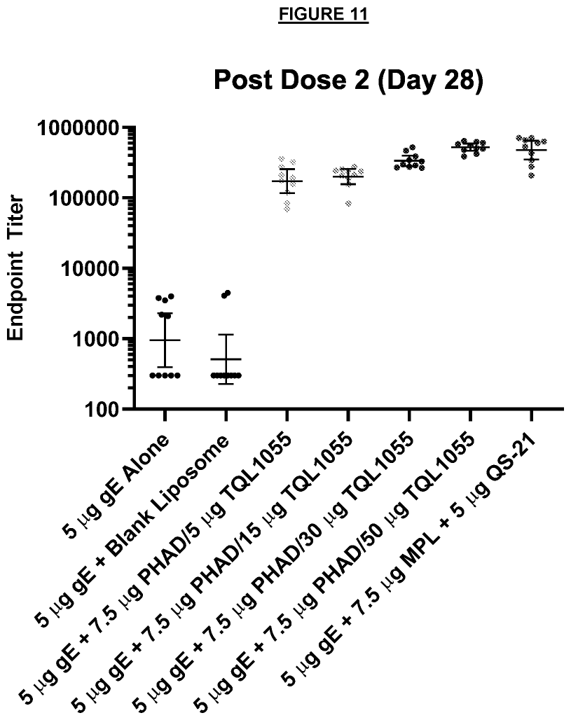

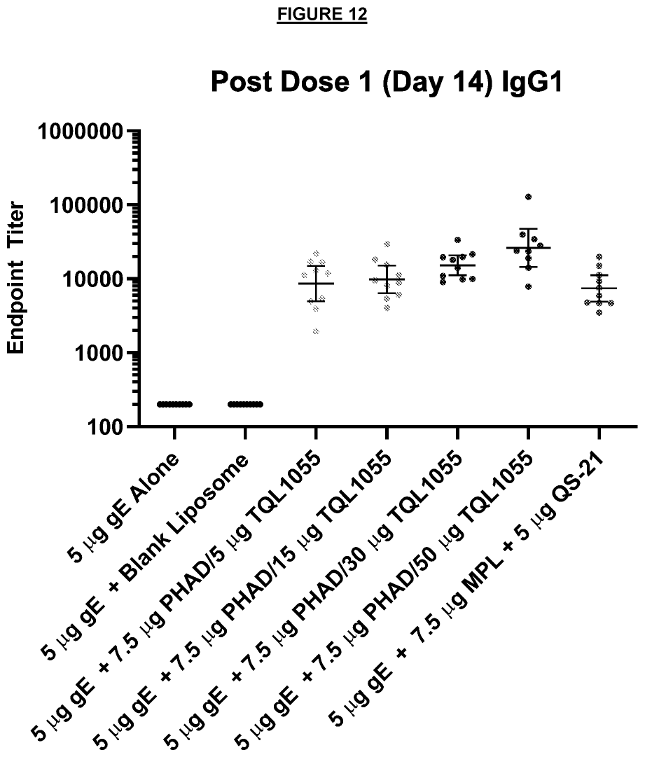

FIG. 10 is a graph depicting gE-specific total IgG titers post Dose 1 for the groups described above (Groups 1 to 7). For each group shown in FIG. 10, Tables 1.1 and 1.2 below contain geometric mean titer (GMT) values, 95% confidence intervals (95% Cl) for titer values, and adjusted P-values comparing the GMT for Groups 3-6 vs. Group 7.

TABLE 1.1
gE-specific total IgG titers post dose 1

| | | | | Adjusted P-value |
|---|---|---|---|---|
| Group | | | | (compared to |
| # | Group | GMT | 95% CI | Group 7) |
| 1 | 5 mcg gE alone | Below | N/A | — |
| | | Limit of | | |
| | | Detection | | |
| 2 | 5 mcg gE + blank | Below | N/A | — |
| | liposome | Limit of | | |
| | | Detection | | |
| 3 | 5 mcg gE + | 17,667 | [12,027; | 0.0002 |
| | 7.5 mcg liposomal | | 25,953] | |
| | PHAD/5 mcg 1055 | | | |
| 4 | 5 mcg gE + | 21,077 | [14,534; | <0.0001 |
| | 7.5 mcg liposomal | | 30566] | |
| | PHAD/15 mcg 1055 | | | |
| 5 | 5 mcg gE + | 17,457 | [12,117; | 0.0002 |
| | 7.5 mcg liposomal | | 25,150] | |
| | PHAD/30 mcg 1055 | | | |
| 6 | 5 mcg gE + | 28,885 | [21,778; | <0.0001 |
| | 7.5 mcg liposomal | | 38,312] | |
| | PHAD/50 mcg 1055 | | | |
| 7 | 5 mcg gE + | 6,519 | [5,176; | — |
| | 7.5 mcg liposomal | | 8,210] | |
| | MPL + 5 mcg QS21 | | | |

TABLE 1.2
Group-wise comparisons between groups in Table 1.1

| Comparison (Group vs Group) | GMT | Adjusted P-value |
|---|---|---|
| 3 (5 mcg) vs 4 (15 mcg) | 17,667 vs 21,077 | 0.9135 |
| 3 (5 mcg) vs 5 (30 mcg) | 17,667 vs 17,457 | >0.999 |
| 3 (5 mcg) vs 6 (50 mcg) | 17,667 vs 28,885 | 0.1436 |
| 4 (15 mcg) vs 5 (30 mcg) | 21,077 vs 17,457 | 0.8927 |
| 4 (15 mcg) vs 6 (50 mcg) | 21,077 vs 28,885 | 0.5574 |
| 5 (30 mcg) vs 6 (50 mcg) | 17,457 vs 28,885 | 0.1276 |

Log 10 transformed GMT were compared using One-way ANOVA. Groupwise comparisons were made and p-values adjusted for multiple comparisons with Tukey's post-hoc test. Groups 1 and 2 were excluded from the analysis. Family-wise α=0.05. The data demonstrate post dose 1, Liposomal PHAD+TQL-1055 free acid significantly enhanced gE-specific IgG GMT compared to Group 7 at all TQL-1055 free acid doses. There was a trend for increasing GMT with increasing TQL-1055 free acid dose, however, not statistically significant between any TQL-1055 free acid dose groups.

gE-Specific Total IgG Titers Post Dose 2

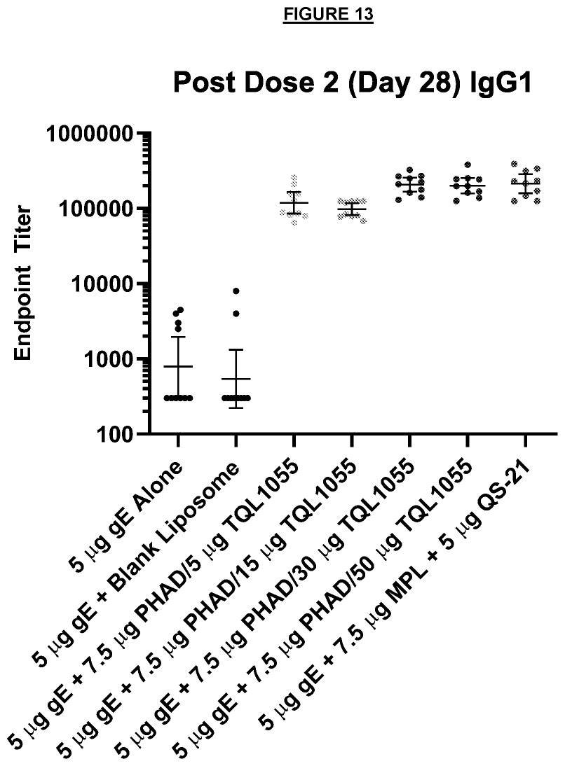

FIG. 11 is a graph depicting gE-specific total IgG titers post Dose 2 for the groups described above (Groups 1 to 7). For each group shown in FIG. 11, Tables 1.3 and 1.4 below contain geometric mean titer (GMT) values, 95% confidence intervals (95% Cl) for titer values, and adjusted P-values comparing the GMT for Groups 3-6 vs. Group 7.

TABLE 1.3
gE-specific total IgG titers post dose 2

| | | | | Adjusted |
|---|---|---|---|---|
| | | | | P-value |
| Group | | | | (compared to |
| # | Group | GMT | 95% CI | Group 7) |
| 1 | 5 μg gE alone | 949.8 | 393.3; | — |
| | | | 2293 | |
| 2 | 5 μg gE + blank | 510.9 | 228.9; | — |
| | liposome | | 1140 | |
| 3 | 5 μg gE + | 173,150 | 117,302; | <0.0001 |
| | 7.5 mcg liposomal | | 255,587 | |
| | PHAD/5 μg 1055 | | | |
| 4 | 5 μg gE + | 200,974 | 156,245; | <0.0001 |
| | 7.5 mcg liposomal | | 258,509 | |
| | PHAD/15 μg 1055 | | | |
| 5 | 5 μg gE + | 336,323 | 283,817; | 0.2442 |
| | 7.5 mcg liposomal | | 398,543 | |
| | PHAD/30 μg 1055 | | | |
| 6 | 5 μg gE + | 524,413 | 464,592; | 0.9762 |
| | 7.5 mcg liposomal | | 591,936 | |
| | PHAD/50 μg 1055 | | | |
| 7 | 5 μg gE + | 475,612 | 349,596; | — |
| | 7.5 μg liposomal | | 647,051 | |
| | MPL + 5 μg QS21 | | | |

TABLE 1.4
Group-wise comparisons between groups in Table 1.3

| Comparison (Group vs Group) | GMT | Adjusted P-value |
|---|---|---|
| 3 (5 μg) vs 4 (15 μg) | 173,150 vs 200,974 | 0.8966 |
| 3 (5 μg) vs 5 (30 μg) | 173,150 vs 336,323 | 0.0021 |
| 3 (5 μg) vs 6 (50 μg) | 173,150 vs 524,413 | <0.0001 |
| 4 (15 μg) vs 5 (30 μg) | 200,974 vs 336,323 | 0.0263 |
| 4 (15 μg) vs 6 (50 μg) | 200,974 vs 524,413 | <0.0001 |
| 5 (30 μg) vs 6 (50 μg) | 336,323 vs 524,413 | 0.0741 |

Log 10 transformed GMT were compared using One-way ANOVA. Groupwise comparisons were made and p-values adjusted for multiple comparisons with Tukey's post-hoc test. Groups 1 and 2 were excluded from the analysis. Family-wise α=0.05.

The data demonstrate after dose 2, GMT for all PHAD liposome+1055 groups increased by up to 18-fold compared to post dose 1. Post dose 2, Group 7 resulted in significantly higher GMT compared to PHAD liposomes+TQL-1055 free acid at 5 and 15 mcg doses. GMTs for Group 7 trended higher than PHAD liposomes+TQL-1055 free acid at 30 mcg dose, although not statistically significant. GMT for the 50 mcg TQL-1055 free acid dose trended higher than Group 7, although not statistically significant. There was a trend for increasing GMT with increasing TQL-1055 free acid dose. Increasing the TQL-1055 free acid dose from 5 to 30 mcg and from 15 to 30 mcg significantly increased GMTs.

gE-Specific Total IgG1 Titers Post Dose 1

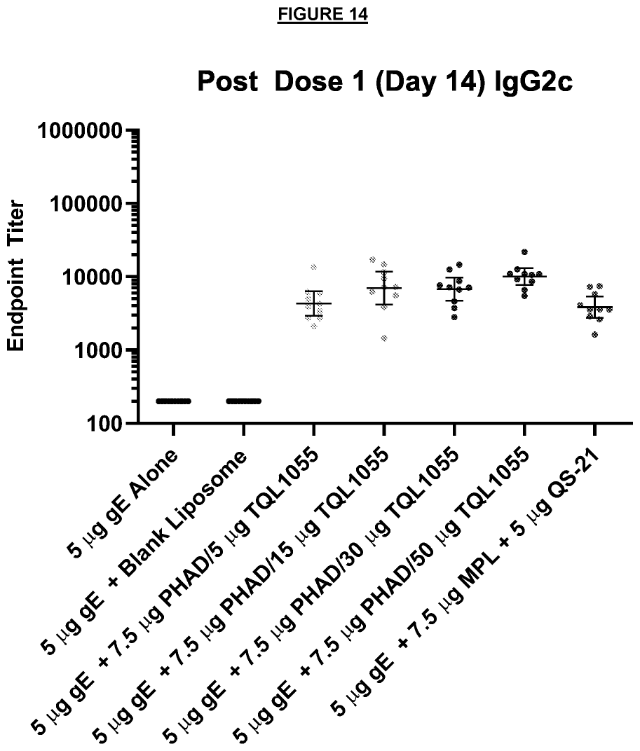

FIG. 12 is a graph depicting gE-specific total IgG1 titers post Dose 1 for the groups described above (Groups 1 to 7). For each group shown in FIG. 12, Tables 1.5 and 1.6 below contain geometric mean titer (GMT) values, 95% confidence intervals (95% Cl) for titer values, and adjusted P-values comparing the GMT for Groups 3-6 vs. Group 7.

TABLE 1.5
gE-specific total IgG1 titers post dose 1

| | | | | Adjusted |
|---|---|---|---|---|
| | | | | P-value |
| Group | | | | (compared to |
| # | Group | GMT | 95% CI | Group 7) |
| 1 | 5 mcg gE alone | Below | N/A | — |
| | | Limit of | | |
| | | Detection | | |
| 2 | 5 mcg gE + blank | Below | N/A | — |
| | liposome | Limit of | | |
| | | Detection | | |
| 3 | 5 mcg gE + | 8,591 | 4,943; | 0.9854 |
| | 7.5 mcg liposomal | | 14,932 | |
| | PHAD/5 mcg 1055 | | | |
| 4 | 5 mcg gE + | 9,801 | 6,379; | 0.8641 |
| | 7.5 mcg liposomal | | 15,061 | |
| | PHAD/15 mcg 1055 | | | |
| 5 | 5 mcg gE + | 15,240 | 11,211; | 0.1038 |
| | 7.5 mcg liposomal | | 20,716 | |
| | PHAD/30 mcg 1055 | | | |
| 6 | 5 mcg gE + | 26,180 | 14,493; | 0.0008 |
| | 7.5 mcg liposomal | | 47,291 | |
| | PHAD/50 mcg 1055 | | | |
| 7 | 5 mcg gE + | 7,416 | 4,935; | — |
| | 7.5 mcg liposomal | | 11,144 | |
| | MPL + 5 mcg QS21 | | | |

TABLE 1.6
Group-wise comparisons between groups in Table 1.5

| Comparison (Group vs Group) | GMT | Adjusted P-value |
|---|---|---|
| 3 (5 mcg) vs 4 (15 mcg) | 8,591 vs 9,801 | 0.9903 |
| 3 (5 mcg) vs 5 (30 mcg) | 8,591 vs 15,240 | 0.2793 |
| 3 (5 mcg) vs 6 (50 mcg) | 8,591 vs 26,180 | 0.0039 |
| 4 (15 mcg) vs 5 (30 mcg) | 9,801 vs 15,240 | 0.5389 |
| 4 (15 mcg) vs 6 (50 mcg) | 9,801 vs 26,180 | 0.0137 |
| 5 (30 mcg) vs 6 (50 mcg) | 15,240 vs 26,180 | 0.3618 |

Log 10 transformed GMT were compared using One-way ANOVA. Groupwise comparisons were made and p-values adjusted for multiple comparisons with Tukey's post-hoc test. Groups 1 and 2 were excluded from the analysis. Family-wise α=0.05. The data demonstrate that after dose 1, gE-specific IgG1 GMT trended higher in all groups having PHAD Liposomes+TQL-1055 free acid compared to Group 7. GMT for the 50 mcg TQL-1055 free acid dose was significantly higher compared to Group 7. There was a trend for increasing GMT with increasing TQL-1055 free acid dose. Increasing TQL-1055 free acid dose from 5 or 15 mcg to 50 mcg resulted in significantly higher GMTs.

gE-Specific Total IgG1 Titers Post Dose 2

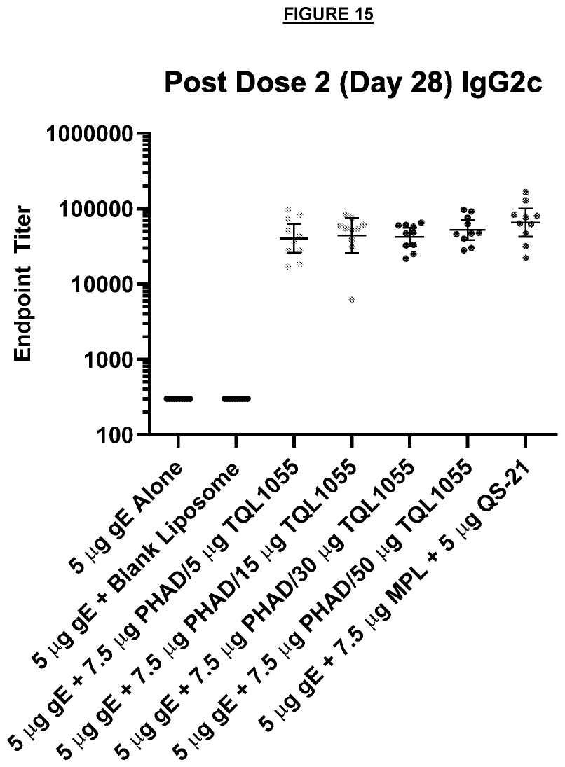

FIG. 13 is a graph depicting gE-specific total IgG1 titers post Dose 2 for the groups described above (Groups 1 to 7). For each group shown in FIG. 13, Tables 1.7 and 1.8 below contain geometric mean titer (GMT) values, 95% confidence intervals (95% Cl) for titer values, and adjusted P-values comparing the GMT for Groups 3-6 vs. Group 7.

TABLE 1.7
gE-specific total IgG1 titers post dose 2

| | | | | Adjusted |
|---|---|---|---|---|
| | | | | P-value |
| Group | | | | (compared to |
| # | Group | GMT | 95% CI | Group 7) |
| 1 | 5 mcg gE alone | 793.1 | 321; | — |
| | | | 1,960 | |
| 2 | 5 mcg gE + blank | 539.8 | 220; | — |
| | liposome | | 1,319 | |
| 3 | 5 mcg gE + | 118,756 | 85,120; | 0.0063 |
| | 7.5 mcg liposomal | | 165,684 | |
| | PHAD/5 mcg 1055 | | | |
| 4 | 5 mcg gE + | 97,959 | 81,845; | 0.0002 |
| | 7.5 mcg liposomal | | 117,245 | |
| | PHAD/15 mcg 1055 | | | |
| 5 | 5 mcg gE + | 206,466 | 166,154; | 0.9997 |
| | 7.5 mcg liposomal | | 256,558 | |
| | PHAD/30 mcg 1055 | | | |
| 6 | 5 mcg gE + | 200,551 | 158,328; | 0.9959 |
| | 7.5 mcg liposomal | | 254,034 | |
| | PHAD/50 mcg 1055 | | | |
| 7 | 5 mcg gE + | 212,822 | 158,986; | — |
| | 7.5 mcg liposomal | | 284,890 | |
| | MPL + 5 mcg QS21 | | | |

TABLE 1.8
Group-wise comparisons between groups in Table 1.7

| Comparison (Group vs Group) | GMT | Adjusted P-value |
|---|---|---|
| 3 (5 mcg) vs 4 (15 mcg) | 118,756 vs 97,959 | 0.7534 |
| 3 (5 mcg) vs 5 (30 mcg) | 118,756 vs 206,466 | 0.0107 |
| 3 (5 mcg) vs 6 (50 mcg) | 118,756 vs 200,551 | 0.0174 |
| 4 (15 mcg) vs 5 (30 mcg) | 97,959 vs 206,466 | 0.0003 |
| 4 (15 mcg) vs 6 (50 mcg) | 97,959 vs 200,551 | 0.0005 |
| 5 (30 mcg) vs 6 (50 mcg) | 206,466 vs 200,551 | 0.9998 |

Log 10 transformed GMT were compared using One-way ANOVA. Groupwise comparisons were made and p-values adjusted for multiple comparisons with Tukey's post-hoc test. Groups 1 and 2 were excluded from the analysis. Family-wise α=0.05. The data demonstrate after a second dose, IgG1 GMT for all PHAD liposome+TQL-1055 free acid groups increased compared to post dose 1. Post dose 2, Group 7 resulted in significantly higher GMT compared to PHAD liposomes+TQL-1055 free acid at 5 and 15 mcg doses. GMT titers for Group 7 trended higher than PHAD liposomes+30 mcg and 50 mcg TQL-1055 free acid, although not statistically significant. GMTs for the 30 and 50 mcg TQL-1055 free acid dose were significantly higher than both the 5 and 15 mcg 1055 dose.

gE-Specific Total IgG2c Titers Post Dose 1

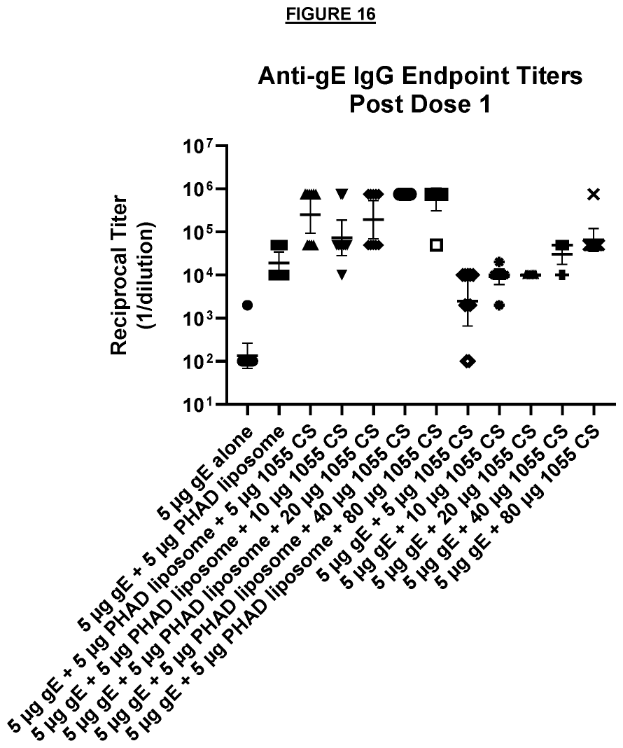

FIG. 14 is a graph depicting gE-specific total IgG2c titers post Dose 1 for the groups described above (Groups 1 to 7). For each group shown in FIG. 14, Tables 1.9 and 1.10 below contain geometric mean titer (GMT) values, 95% confidence intervals (95% Cl) for titer values, and adjusted P-values comparing the GMT for Groups 3-6 vs. Group 7.

TABLE 1.9
gE-specific total IgG2c titers post dose 1

| | | | | Adjusted |
|---|---|---|---|---|
| | | | | P-value |
| Group | | | | (compared to |
| # | Group | GMT | 95% CI | Group 7) |
| 1 | 5 mcg gE alone | Below | N/A | — |
| | | Limit of | | |
| | | Detection | | |
| 2 | 5 mcg gE + blank | Below | N/A | — |
| | liposome | Limit of | | |
| | | Detection | | |
| 3 | 5 mcg gE + | 4,313 | 2,943; | 0.9892 |
| | 7.5 mcg liposomal | | 6,321 | |
| | PHAD/5 mcg 1055 | | | |
| 4 | 5 mcg gE + | 7,010 | 4,197; | 0.1058 |
| | 7.5 mcg liposomal | | 11,706 | |
| | PHAD/15 mcg 1055 | | | |
| 5 | 5 mcg gE + | 6,762 | 4,694; | 0.1448 |
| | 7.5 mcg liposomal | | 9,738 | |
| | PHAD/30 mcg 1055 | | | |
| 6 | 5 mcg gE + | 10,072 | 7,714; | 0.0019 |
| | 7.5 mcg liposomal | | 13,150 | |
| | PHAD/50 mcg 1055 | | | |
| 7 | 5 mcg gE + | 3,851 | 2,750; | — |
| | 7.5 mcg liposomal | | 5,393 | |
| | MPL + 5 mcg QS21 | | | |

TABLE 1.10
Group-wise comparisons between groups in Table 1.9

| Comparison (Group vs Group) | GMT | Adjusted P-value |
|---|---|---|
| 3 (5 mcg) vs 4 (15 mcg) | 4,313 vs 7,010 | 0.2652 |
| 3 (5 mcg) vs 5 (30 mcg) | 4,313 vs 6,762 | 0.3390 |
| 3 (5 mcg) vs 6 (50 mcg) | 4,313 vs 10,072 | 0.0076 |
| 4 (15 mcg) vs 5 (30 mcg) | 7,010 vs 6,762 | 0.9999 |
| 4 (15 mcg) vs 6 (50 mcg) | 7,010 vs 10,072 | 0.5548 |
| 5 (30 mcg) vs 6 (50 mcg) | 6,762 vs 10,072 | 0.4614 |

Log 10 transformed GMT were compared using One-way ANOVA. Groupwise comparisons were made and p-values adjusted for multiple comparisons with Tukey's post-hoc test. Groups 1 and 2 were excluded from the analysis. Family-wise α=0.05. The data demonstrate post dose 1, gE-specific IgG2c GMT for PHAD Liposomes+TQL-1055 free acid groups trended higher compared to Group 7. GMT for the 50 mcg TQL-1055 free acid dose were significantly higher compared to Group 7. There was a trend for increasing GMT with increasing TQL-1055 free acid dose. Increasing TQL-1055 free acid dose from 5 to 50 mcg resulted in significantly higher GMTs.

gE-Specific Total IgG2c Titers Post Dose 2

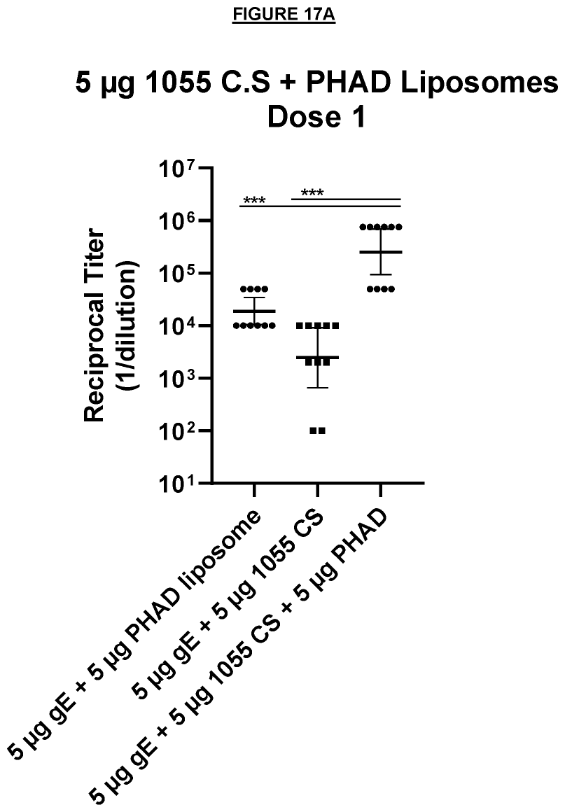

FIG. 15 is a graph depicting gE-specific total IgG2c titers post Dose 2 for the groups described above (Groups 1 to 7). For each group shown in FIG. 15, Tables 1.11 and 1.12 below contain geometric mean titer (GMT) values, 95% confidence intervals (95% Cl) for titer values, and adjusted P-values comparing the GMT for Groups 3-6 vs. Group 7.

TABLE 1.11
gE-specific total IgG2c titers post dose 2

| | | | | Adjusted |
|---|---|---|---|---|
| | | | | P-value |
| Group | | | | (compared to |
| # | Group | GMT | 95% CI | Group 7) |
| 1 | 5 mcg gE alone | Below | N/A | — |
| | | Limit of | | |
| | | Detection | | |
| 2 | 5 mcg gE + blank | Below | N/A | — |
| | liposome | Limit of | | |
| | | Detection | | |
| 3 | 5 mcg gE + | 40,409 | 26,051; | 0.3380 |
| | 7.5 mcg liposomal | | 62,679 | |
| | PHAD/5 mcg 1055 | | | |
| 4 | 5 mcg gE + | 44,104 | 25,874; | 0.5384 |
| | 7.5 mcg liposomal | | 75,181 | |
| | PHAD/15 mcg 1055 | | | |
| 5 | 5 mcg gE + | 42,295 | 31,856; | 0.4380 |
| | 7.5 mcg liposomal | | 56,154 | |
| | PHAD/30 mcg 1055 | | | |
| 6 | 5 mcg gE + | 52,170 | 38,323; | 0.8990 |
| | 7.5 mcg liposomal | | 71,020 | |
| | PHAD/50 mcg 1055 | | | |
| 7 | 5 mcg gE + | 65,517 | 42,621; | — |
| | 7.5 mcg liposomal | | 100,715 | |
| | MPL + 5 mcg QS21 | | | |

TABLE 1.12
Group-wise comparisons between groups in Table 1.11

| Comparison (Group vs Group) | GMT | Adjusted P-value |
|---|---|---|
| 3 (5 mcg) vs 4 (15 mcg) | 40,409 vs 44,104 | 0.9969 |
| 3 (5 mcg) vs 5 (30 mcg) | 40,409 vs 42,295 | 0.9998 |
| 3 (5 mcg) vs 6 (50 mcg) | 40,409 vs 52,170 | 0.8547 |
| 4 (15 mcg) vs 5 (30 mcg) | 44,104 vs 42,295 | 0.9998 |
| 4 (15 mcg) vs 6 (50 mcg) | 44,104 vs 52,170 | 0.9645 |
| 5 (30 mcg) vs 6 (50 mcg) | 42,295 vs 52,170 | 0.9230 |

Log 10 transformed GMT were compared using One-way ANOVA. Groupwise comparisons were made and p-values adjusted for multiple comparisons with Tukey's post-hoc test. Groups 1 and 2 were excluded from the analysis. Family-wise α=0.05. The data demonstrate after a second dose, IgG2c GMT for all PHAD liposome+TQL-1055 free acid groups increased compared to post dose 1. Post dose 2, Group 7 IgG2c GMT trended higher compared to PHAD liposomes+TQL-1055 free acid at all doses of TQL-1055 free acid, although not statistically significant. IgG2c GMT for the 50 mcg TQL-1055 free acid dose trended slightly higher compared to lower TQL-1055 free acid doses.

### Example 2—Liposome PHAD+Compound I-4 Choline Salt Form

The impact of TQL-1055 choline salt (Compound 1-4 choline salt) on antibody titers induced by gE antigen was tested. Mice were immunized with gE (5 mcg) alone, gE (5 mcg) with liposomes containing PHAD (5 mcg), gE (5 mcg) with liposomes containing PHAD (5 mcg) and TQL-1055 choline salt (five groups: 5 mcg, 10 mcg, 20 mcg, 40 mcg, 80 mcg), and gE (5 mcg) with TQL-1055 choline salt (five groups: 5 mcg, 10 mcg, 20 mcg, 40 mcg, 80 mcg). Mice were immunized at Day 0 and Day 14. Groups were bled at Day 13 (post Dose 1) and Day 28 (post Dose 2) for serum analysis. The results are shown below.

### Anti-gE IgG Endpoint Titers Post Dose 1

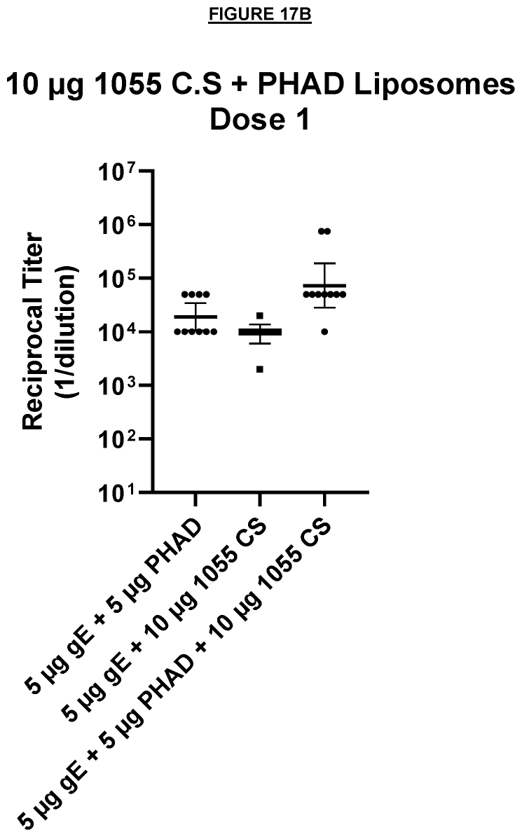

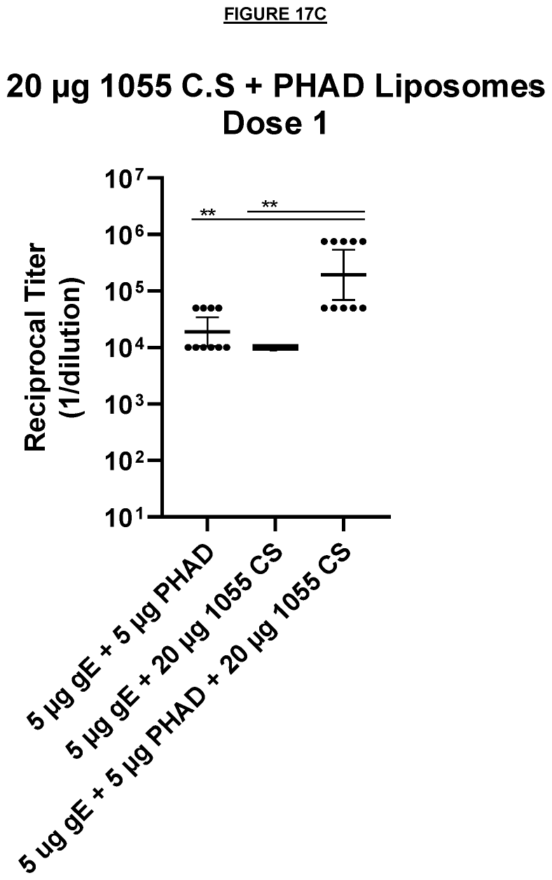

FIG. 16 is a graph depicting gE-specific total IgG titers post Dose 1 for the groups described above. FIGS. 17A-E depict subsets of the data shown in FIG. 16, in which the synergistic effects of PHAD and TQL-1055 choline salt become apparent. For groups shown in FIG. 16, Table 2.1 below contains geometric mean titer (GMT) values and adjusted P-values comparing various groups.

TABLE 2.1
gE-specific total IgG titers post dose 1

| | | | Day 13 | | | | Day 28 | |
|---|---|---|---|---|---|---|---|---|
| | Day 13 Anti-gE | | Comparison | | Day 28 Anti-gE | | Comparison | |
| | GMT titer | | Stat | | GMT titer | | Stat | |
| | without | 5 mcg | Combo | Stats | without | 5 mcg | Combo vs | Stats |
| | PHAD | PHAD in | vs PHAD | Combo vs | PHAD | PHAD in | PHAD | Combo vs |
| | liposomes | liposomes | alone | CS alone | liposomes | liposomes | alone | CS alone |
| 5 mcg | 135 | 19,037 | Not | Not | 12,311 | 689,865 | Not | (40: p = 0.025) |
| gE | | | statistically | statistically | | | statistically | (80: p < 0.0001) |
| | | | significant | significant | | | significant | |
| 5 mcg | 2,456 | 253,878 | P = 0.0006 | P = 0.0002 | 61,703 | 2,128,350 | P = 0.0005 | P < 0.0001 |
| gE + | | | | | | | | |
| 5 mcg | | | | | | | | |
| 1055 | | | | | | | | |
| CS | | | | | | | | |
| 5 mcg | 9,124 | 73,163 | P = 0.9423 | P = 0.8836 | 425,670 | 1,313,264 | P = 0.319 | P < 0.0001 |
| gE + | | | | | | | | |
| 10 mcg | | | | | | | | |
| 1055 | | | | | | | | |
| CS | | | | | | | | |
| 5 mcg | 10,000 | 193,649 | P = 0.0099 | P = 0.0054 | 500,000 | 1,811,949 | P = 0.0066 | P = 0.04 |
| gE + | | | | | | | | |
| 20 mcg | | | | | | | | |
| 1055 | | | | | | | | |
| CS | | | | | | | | |
| 5 mcg | 30,852 | 750,022 | P < 0.0001 | P < 0.0001 | 810,328 | 2,128,350 | P = 0.0005 | P < 0.0001 |
| gE + | | | | | | | | |
| 40 mcg | | | | | | | | |
| 1055 | | | | | | | | |
| CS | | | | | | | | |
| 5 mcg | 65,561 | 572,074 | P < 0.0001 | P < 0.0001 | 2,128,350 | >2,500,000 | P < 0.0001 | P = 0.0066 |
| gE + | | | | | | | | |
| 80 mcg | | | | | | | | |
| 1055 | | | | | | | | |
| CS | | | | | | | | |
| +6 h | 2,091 | 85,939 | vs T = 0 | | 117,462 | 1,313,264 | vs T = 0 | |
| 5 mcg | | | Not statistically | | | | Not statistically | |
| gE + | | | significant | | | | significant | |
| 5 mcg | | | | | | | | |
| 1055 | | | | | | | | |
| CS | | | | | | | | |

| +6 h | 332,839 | 572,074 | vs T = 0 | 1,811,949 | 2,128,350 | vs T = 0 |
|---|---|---|---|---|---|---|
| 5 mcg | | | with PHAD: | | | Not statistically |
| gE + | | | Not statistically | | | significant |
| 80 mcg | | | significant | | | |
| 1055 | | | Without PHAD: | | | |
| CS | | | P = 0.0016 | | | |

The data demonstrate after the first dose, gE-specific IgG for TQL-1055 choline salt+PHAD in liposome exhibits as synergistic effect as compared to either compound alone.

### Anti-gE IgG Endpoint Titers Post Dose 2

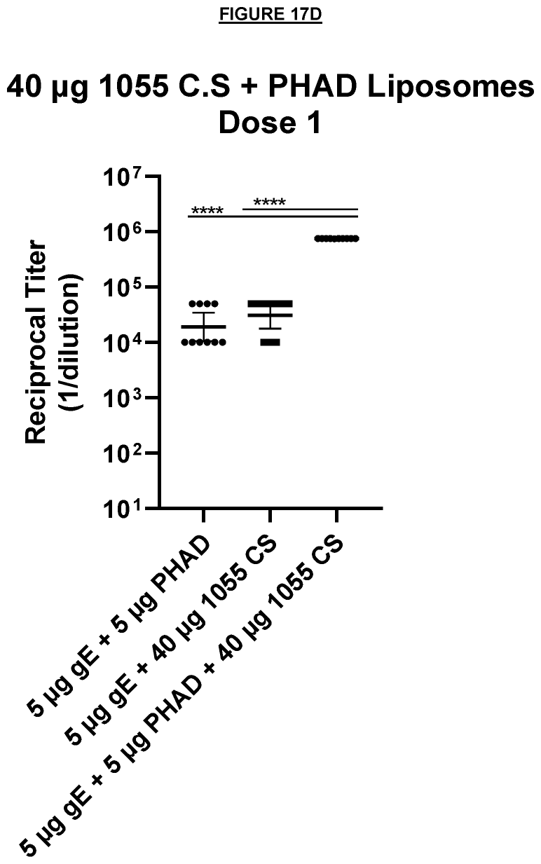

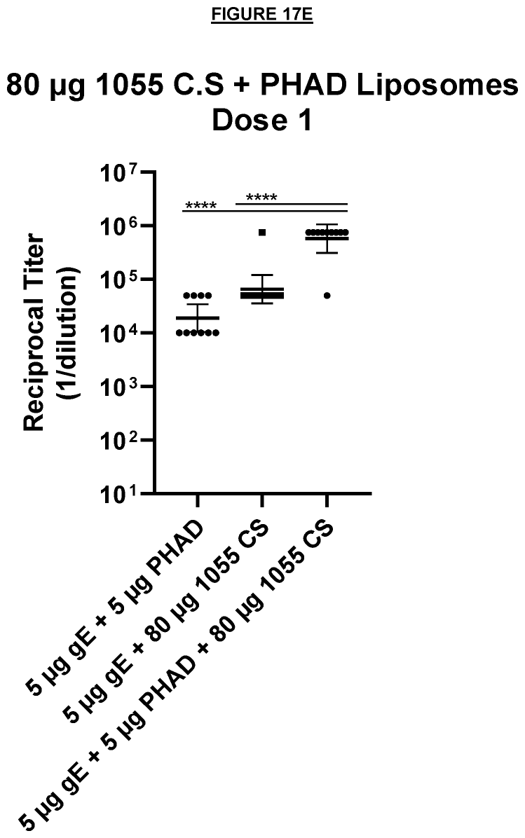

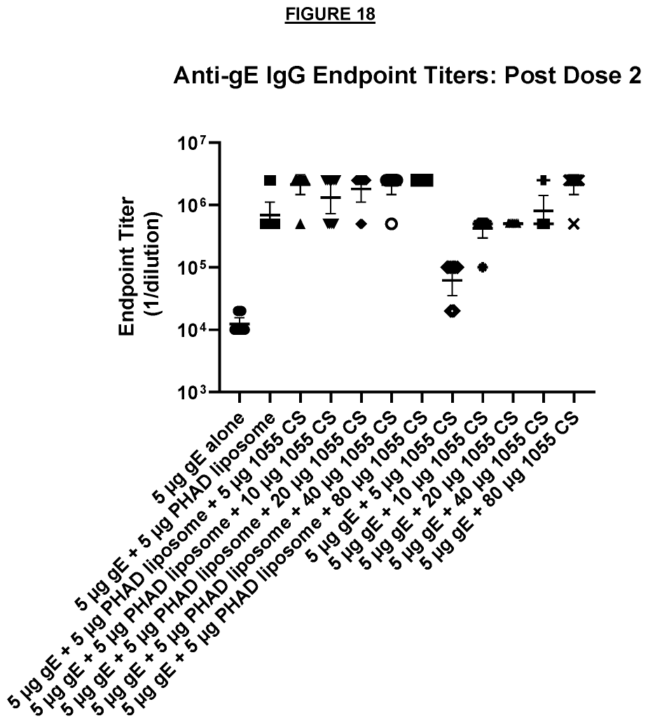

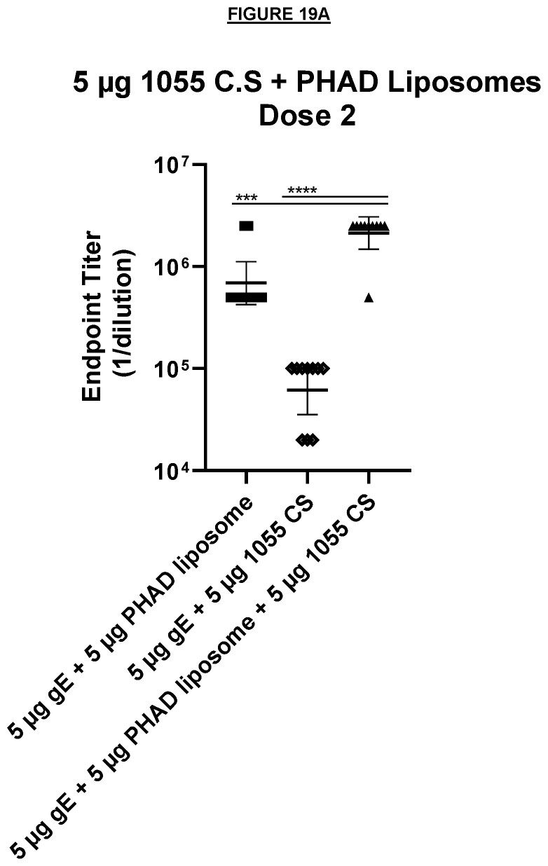

FIG. 18 is a graph depicting gE-specific total IgG titers post Dose 2 for the groups described above. FIGS. 19A-E depict subsets of the data shown in FIG. 18, in which the synergistic effects of PHAD and TQL-1055 choline salt once again become apparent. For groups shown in FIG. 16, Table 2.1 above contains geometric mean titer (GMT) values and adjusted P-values comparing various groups.

The data demonstrate after the second dose, gE-specific IgG for TQL-1055 choline salt+PHAD in liposome exhibits as synergistic effect as compared to either compound alone.

### Example 3—Oil in Water Emulsions+Compound I-4 Choline Salt Form

The impact of an oil-in-water emulsion containing TQL-1055 free acid (Compound 1-4 free acid), TQL-1055 choline salt (Compound I-4 choline salt), PHAD liposomes, and combinations thereof on antibody titers induced by gE antigen was tested. Mice were immunized with PBS alone, gE (5 mcg) alone, gE (5 mcg) in an oil-in-water emulsion (L2), gE (5 mcg) and TQL-1055 choline salt (three groups: 5 mcg, 30 mcg, 100 mcg), gE (5 mcg) in an oil-in-water emulsion (L2) with TQL-1055 free acid (three groups: 5 mcg, 30 mcg, 100 mcg), gE (5 mcg) in an oil-in-water emulsion (L2) with TQL-1055 free acid (three groups: 5 mcg, 30 mcg, 100 mcg) and PHAD liposomes (20 mcg), and gE (5 mcg) with PHAD liposomes (20 mcg). Mice were immunized at Day 0 and Day 14. Groups were bled at Day 13 (post Dose 1) and Day 28 (post Dose 2) for serum analysis. The results are shown below.

### Anti-gE IgG Endpoint Titers Post Dose 1

FIG. 20 is a graph depicting gE-specific total IgG titers post Dose 1 for the groups described above. For groups shown in FIG. 20, Table 3.1 below contains geometric mean titer (GMT) values and confidence intervals. Table 3.2 below shows adjusted P-values comparing various groups.

TABLE 3.1
gE-specific total IgG titers post dose 1

| | | | | 5 μg | 5 μg | 5 μg | 5 μg |
|---|---|---|---|---|---|---|---|
| | | | | gE + | gE + | gE + | gE + L2 + |
| | | 5 μg | | 5 μg | 30 μg | 100 μg | 5 μg |
| | PBS | gE | 5 μg | 1055 | 1055 | 1055 | 1055 |
| | Alone | Alone | gE + L2 | C.S | C.S | C.S | F.A |
| Number of | 10 | 10 | 10 | 10 | 10 | 10 | 10 |
| values | | | | | | | |
| Geometric | Below | 138.0 | 190.4 | 388.4 | 6295 | 19905 | 5000 |
| mean | Limit of | | | | | | |
| | Detection | | | | | | |
| Geometric | 1.000 | 1.971 | 2.296 | 3.240 | 2.071 | 3.284 | 1.000 |
| SD factor | | | | | | | |
| Lower 95% | 100.0 | 84.91 | 105.0 | 167.5 | 3739 | 8503 | 5000 |
| CI of geo. | | | | | | | |
| mean | | | | | | | |
| Upper 95% | 100.0 | 224.2 | 345.0 | 900.4 | 10597 | 46599 | 5000 |
| CI of geo. | | | | | | | |
| mean | | | | | | | |

| | | | | | 5 μg | |
|---|---|---|---|---|---|---|
| | | | 5 μg | 5 μg | gE + | |
| | | | gE + L2 + | gE + L2 + | L2 + | |
| | 5 μg | 5 μg | 5 μg | 30 μg | 100 μg | |
| | gE + L2 + | gE + L2 + | 1055 | 1055 | 1055 | 5 μg |
| | 30 μg | 100 μg | F.A + | F.A + | F.A + | gE + |
| | 1055 | 1055 | 20 μg | 20 μg | 20 μg | 20 μg |
| | F.A | F.A | PHAD | PHAD | PHAD | PHAD |
| Number of | 10 | 10 | 10 | 10 | 10 | 10 |
| values | | | | | | |
| Geometric | Below | 25059 | 50000 | 50000 | 1233844 | 50000 |
| mean | Limit of | | | | | |
| | Detection | | | | | |
| Geometric SD | 1.000 | 3.041 | 1.000 | 1.000 | 5.418 | 1.000 |
| factor | | | | | | |
| Lower 95% CI | 5000 | 11309 | 50000 | 50000 | 368409 | 50000 |
| of geo. mean | | | | | | |
| Upper 95% CI | 5000 | 55529 | 50000 | 50000 | 4132283 | 50000 |
| of geo. mean | | | | | | |

TABLE 3.2
Group-wise comparisons between groups in Table 3.1

| Tukey's multiple | | | Adjusted |
|---|---|---|---|
| comparisons test | Significant? | Summary | P Value |
| PBS Alone vs. 5 μg | No | ns | >0.9999 |
| gE Alone | | | |
| PBS Alone vs. 5 μg | No | ns | >0.9999 |
| gE + L2 | | | |
| PBS Alone vs. 5 μg | No | ns | >0.9999 |
| gE + 5 μg 1055 CS | | | |
| PBS Alone vs. 5 μg | No | ns | >0.9999 |
| gE + 30 μg 1055 | | | |
| CS | | | |
| PBS Alone vs. 5 μg | No | ns | >0.9999 |
| gE + 100 μg 1055 | | | |
| CS | | | |
| PBS Alone vs. 5 μg | No | ns | >0.9999 |
| gE + L2 + 5 μg | | | |
| 1055 FA | | | |
| PBS Alone vs. 5 μg | No | ns | >0.9999 |
| gE + L2 + 30 μg | | | |
| 1055 FA | | | |
| PBS Alone vs. 5 μg | No | ns | >0.9999 |
| gE + L2 + 100 μg | | | |
| 1055 FA | | | |
| PBS Alone vs. 5 μg | No | ns | >0.9999 |
| gE + L2 + 5 μg | | | |
| 1055 FA + 20 μg | | | |
| PHAD liposomes | | | |
| PBS Alone vs. 5 μg | No | ns | >0.9999 |
| gE + L2 + 30 μg | | | |
| 1055 FA + 20 μg | | | |
| PHAD liposomes | | | |
| PBS Alone vs. 5 μg | Yes | **** | <0.0001 |
| gE + L2 + 100 μg | | | |
| 1055 FA + 20 μg | | | |
| PHAD liposomes | | | |
| PBS Alone vs. 5 μg | No | ns | >0.9999 |
| gE + 20 μg PHAD | | | |
| liposomes | | | |
| 5 μg gE Alone vs. | No | ns | >0.9999 |
| 5 μg gE + L2 | | | |
| 5 μg gE Alone vs. | No | ns | >0.9999 |
| 5 μg gE + 5 | | | |
| μg 1055 CS | | | |
| 5 μg gE Alone vs. | No | ns | >0.9999 |
| 5 μg gE + 30 | | | |
| μg 1055 CS | | | |
| 5 μg gE Alone vs. | No | ns | >0.9999 |
| 5 μg gE + 100 | | | |
| μg 1055 CS | | | |
| 5 μg gE Alone vs. | No | ns | >0.9999 |
| 5 μg gE + L2 + | | | |
| 5 μg 1055 FA | | | |
| 5 μg gE Alone vs. | No | ns | >0.9999 |
| 5 μg gE + L2 + | | | |
| 30 μg 1055 FA | | | |
| 5 μg gE Alone vs. | No | ns | >0.9999 |
| 5 μg gE + L2 + | | | |
| 100 μg 1055 FA | | | |
| 5 μg gE Alone vs. | No | ns | >0.9999 |
| 5 μg gE + L2 + | | | |
| 5 μg 1055 FA + | | | |
| 20 μg PHAD liposomes | | | |
| 5 μg gE Alone vs. | No | ns | >0.9999 |
| 5 μg gE + L2 + | | | |
| 30 μg 1055 FA + | | | |
| 20 μg PHAD liposomes | | | |
| 5 μg gE Alone vs. | Yes | **** | <0.0001 |
| 5 μg gE + L2 + | | | |
| 100 μg 1055 FA + | | | |
| 20 μg PHAD liposomes | | | |
| 5 μg gE Alone vs. | No | ns | >0.9999 |
| 5 μg gE + 20 μg | | | |
| PHAD liposomes | | | |
| 5 μg gE + L2 vs. | No | ns | >0.9999 |
| 5 μg gE + 5 μg | | | |
| 1055 CS | | | |
| 5 μg gE + L2 vs. | No | ns | >0.9999 |
| 5 μg gE + 30 μg | | | |
| 1055 CS | | | |
| 5 μg gE + L2 vs. | No | ns | >0.9999 |
| 5 μg gE + 100 μg | | | |
| 1055 CS | | | |
| 5 μg gE + L2 vs. | No | ns | >0.9999 |
| 5 μg gE + L2 + | | | |
| 5 μg 1055 FA | | | |
| 5 μg gE + L2 vs. | No | ns | >0.9999 |
| 5 μg gE + L2 + | | | |
| 30 μg 1055 FA | | | |
| 5 μg gE + L2 vs. | No | ns | >0.9999 |
| 5 μg gE + L2 + | | | |
| 100 μg 1055 FA | | | |
| 5 μg gE + L2 vs. | No | ns | >0.9999 |
| 5 μg gE + L2 + | | | |
| 5 μg 1055 FA + 20 | | | |
| μg PHAD liposomes | | | |
| 5 μg gE + L2 vs. | No | ns | >0.9999 |
| 5 μg gE + L2 + | | | |
| 30 μg 1055 FA + | | | |
| 20 μg PHAD liposomes | | | |
| 5 μg gE + L2 vs. | Yes | **** | <0.0001 |
| 5 μg gE + L2 + | | | |
| 100 μg 1055 FA + | | | |
| 20 μg PHAD liposomes | | | |
| 5 μg gE + L2 vs. | No | ns | >0.9999 |
| 5 μg gE + 20 μg | | | |
| PHAD liposomes | | | |
| 5 μg gE + 5 μg | No | ns | >0.9999 |
| 1055 CS vs. 5 μg gE + 30 | | | |
| μg 1055 CS | | | |
| 5 μg gE + 5 μg | No | ns | >0.9999 |
| 1055 CS vs. 5 μg gE + 100 | | | |
| μg 1055 CS | | | |
| 5 μg gE + 5 μg | No | ns | >0.9999 |
| 1055 CS vs. 5 μg gE + L2 + | | | |
| 5 μg 1055 FA | | | |
| 5 μg gE + 5 μg | No | ns | >0.9999 |
| 1055 CS vs. 5 μg gE + L2 + | | | |
| 30 μg 1055 FA | | | |
| 5 μg gE + 5 μg | No | ns | >0.9999 |
| 1055 CS vs. 5 μg gE + L2 + | | | |
| 100 μg 1055 FA | | | |
| 5 μg gE + 5 μg | No | ns | >0.9999 |
| 1055 CS vs. 5 μg gE + L2 + | | | |
| 5 μg 1055 FA + 20 | | | |
| μg PHAD liposomes | | | |
| 5 μg gE + 5 μg | No | ns | >0.9999 |
| 1055 CS vs. 5 μg gE + L2 + | | | |
| 30 μg 1055 FA + 20 | | | |
| μg PHAD liposomes | | | |
| 5 μg gE + 5 μg | Yes | **** | <0.0001 |
| 1055 CS vs. 5 μg gE + L2 + | | | |
| 100 μg 1055 FA + 20 | | | |
| μg PHAD liposomes | | | |
| 5 μg gE + 5 μg | No | ns | >0.9999 |
| 1055 CS vs. 5 μg gE + 20 | | | |
| μg PHAD liposomes | | | |
| 5 μg gE + 30 μg | No | ns | >0.9999 |
| 1055 CS vs. 5 μg | | | |
| gE + 100 μg 1055 | | | |
| CS | | | |
| 5 μg gE + 30 μg | No | ns | >0.9999 |
| 1055 CS vs. 5 μg | | | |
| gE + L2 + 5 μg | | | |
| 1055 FA | | | |
| 5 μg gE + 30 μg | No | ns | >0.9999 |
| 1055 CS vs. 5 μg | | | |
| gE + L2 + 30 μg | | | |
| 1055 FA | | | |
| 5 μg gE + 30 μg | No | ns | >0.9999 |
| 1055 CS vs. 5 μg | | | |
| gE + L2 + 100 μg | | | |
| 1055 FA | | | |
| 5 μg gE + 30 μg | No | ns | >0.9999 |
| 1055 CS vs. 5 μg | | | |
| gE + L2 + 5 μg | | | |
| 1055 FA + 20 μg | | | |
| PHAD liposomes | | | |
| 5 μg gE + 30 μg | No | ns | >0.9999 |
| 1055 CS vs. 5 μg | | | |
| gE + L2 + 30 μg | | | |
| 1055 FA + 20 μg | | | |
| PHAD liposomes | | | |
| 5 μg gE + 30 μg | Yes | **** | <0.0001 |
| 1055 CS vs. 5 μg | | | |
| gE + L2 + 100 μg | | | |
| 1055 FA + 20 μg | | | |
| PHAD liposomes | | | |
| 5 μg gE + 30 μg | No | ns | >0.9999 |
| 1055 CS vs. 5 μg | | | |
| gE + 20 μg PHAD | | | |
| liposomes | | | |
| 5 μg gE + 100 μg | No | ns | >0.9999 |
| 1055 CS vs. 5 μg gE + | | | |
| L2 + 5 μg 1055 FA | | | |
| 5 μg gE + 100 μg | No | ns | >0.9999 |
| 1055 CS vs. 5 μg gE + | | | |
| L2 + 30 μg 1055 FA | | | |
| 5 μg gE + 100 μg | No | ns | >0.9999 |
| 1055 CS vs. 5 μg gE + | | | |
| L2 + 100 μg 1055 FA | | | |
| 5 μg gE + 100 μg | No | ns | >0.9999 |
| 1055 CS vs. 5 μg gE + | | | |
| L2 + 5 μg 1055 FA + | | | |
| 20 μg PHAD liposomes | | | |
| 5 μg gE + 100 μg | No | ns | >0.9999 |
| 1055 CS vs. 5 μg gE + | | | |
| L2 + 30 μg 1055 FA + | | | |
| 20 μg PHAD liposomes | | | |
| 5 μg gE + 100 μg | Yes | **** | <0.0001 |
| 1055 CS vs. 5 μg gE + | | | |
| L2 + 100 μg 1055 FA + | | | |
| 20 μg PHAD liposomes | | | |
| 5 μg gE + 100 μg | No | ns | >0.9999 |
| 1055 CS vs. 5 μg gE + | | | |
| 20 μg PHAD liposomes | | | |
| 5 μg gE + L2 + 5 | No | ns | >0.9999 |
| μg 1055 FA vs. 5 μg | | | |
| gE + L2 + 30 μg 1055 FA | | | |
| 5 μg gE + L2 + 5 | No | ns | >0.9999 |
| μg 1055 FA vs. 5 μg | | | |
| gE + L2 + 100 μg 1055 FA | | | |
| 5 μg gE + L2 + 5 | No | ns | >0.9999 |
| μg 1055 FA vs. 5 μg | | | |
| gE + L2 + 5 μg 1055 | | | |
| FA + 20 μg PHAD | | | |
| liposomes | | | |
| 5 μg gE + L2 + 5 | No | ns | >0.9999 |
| μg 1055 FA vs. 5 μg | | | |
| gE + L2 + 30 μg | | | |
| 1055 FA + 20 μg PHAD | | | |
| liposomes | | | |
| 5 μg gE + L2 + 5 | Yes | **** | <0.0001 |
| μg 1055 FA vs. 5 μg | | | |
| gE + L2 + 100 μg | | | |
| 1055 FA + 20 μg PHAD | | | |
| liposomes | | | |
| 5 μg gE + L2 + 5 | No | ns | >0.9999 |
| μg 1055 FA vs. 5 μg | | | |
| gE + 20 μg PHAD | | | |
| liposomes | | | |
| 5 μg gE + L2 + 30 | No | ns | >0.9999 |
| μg 1055 FA vs. 5 μg | | | |
| gE + L2 + 100 μg | | | |
| 1055 FA | | | |
| 5 μg gE + L2 + 30 | No | ns | >0.9999 |
| μg 1055 FA vs. 5 μg | | | |
| gE + L2 + 5 μg | | | |
| 1055 FA + 20 μg PHAD | | | |
| liposomes | | | |
| 5 μg gE + L2 + 30 | No | ns | >0.9999 |
| μg 1055 FA vs. 5 μg | | | |
| gE + L2 + 30 μg | | | |
| 1055 FA + 20 μg PHAD | | | |
| liposomes | | | |
| 5 μg gE + L2 + 30 | Yes | **** | <0.0001 |
| μg 1055 FA vs. 5 μg | | | |
| gE + L2 + 100 μg | | | |
| 1055 FA + 20 μg PHAD | | | |
| liposomes | | | |
| 5 μg gE + L2 + 30 | No | ns | >0.9999 |
| μg 1055 FA vs. 5 μg | | | |
| gE + 20 μg PHAD | | | |
| liposomes | | | |
| 5 μg gE + L2 + 100 | No | ns | >0.9999 |
| μg 1055 FA vs. 5 μg | | | |
| gE + L2 + 5 μg | | | |
| 1055 FA + 20 μg PHAD | | | |
| liposomes | | | |
| 5 μg gE + L2 + 100 | No | ns | >0.9999 |
| μg 1055 FA vs. 5 μg | | | |
| gE + L2 + 30 μg | | | |
| 1055 FA + 20 μg PHAD | | | |
| liposomes | | | |
| 5 μg gE + L2 + 100 | Yes | **** | <0.0001 |
| μg 1055 FA vs. 5 μg | | | |
| gE + L2 + 100 μg | | | |
| 1055 FA + 20 μg PHAD | | | |
| liposomes | | | |
| 5 μg gE + L2 + 5 μg | No | ns | >0.9999 |
| 1055 FA + 20 μg | | | |
| PHAD liposomes | | | |
| vs. 5 μg gE + L2 + | | | |
| 30 μg 1055 FA + 20 | | | |
| μg PHAD | | | |
| liposomes | | | |
| 5 μg gE + L2 + 5 μg | Yes | **** | <0.0001 |
| 1055 FA + 20 μg | | | |
| PHAD liposomes | | | |
| vs. 5 μg gE + L2 + | | | |
| 100 μg 1055 FA + | | | |
| 20 μg PHAD | | | |
| liposomes | | | |
| 5 μg gE + L2 + 5 μg | No | ns | >0.9999 |
| 1055 FA + 20 μg | | | |
| PHAD liposomes | | | |
| vs. 5 μg gE + 20 μg | | | |
| PHAD liposomes | | | |
| 5 μg gE + L2 + 30 | Yes | **** | <0.0001 |
| μg 1055 FA + 20 | | | |
| μg PHAD | | | |
| liposomes vs. 5 μg | | | |
| gE + L2 + 100 μg | | | |
| 1055 FA + 20 μg | | | |
| PHAD liposomes | | | |
| 5 μg gE + L2 + 30 | No | ns | >0.9999 |
| μg 1055 FA + 20 | | | |
| μg PHAD | | | |
| liposomes vs. 5 μg | | | |
| gE + 20 μg PHAD | | | |
| liposomes | | | |
| 5 μg gE + L2 + 100 | Yes | **** | <0.0001 |
| μg 1055 FA + 20 | | | |
| μg PHAD | | | |
| liposomes vs. 5 μg | | | |
| gE + 20 μg PHAD | | | |
| liposomes | | | |

### Anti-gE IgG Endpoint Titers Post Dose 2

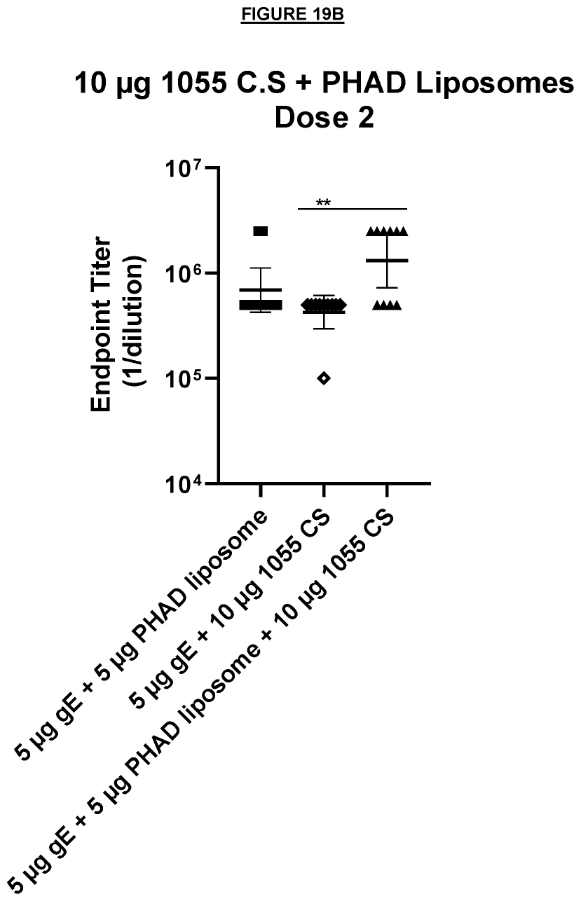

FIG. 21 is a graph depicting gE-specific total IgG titers post Dose 2 for the groups described above. For groups shown in FIG. 21, Table 3.3 below contains geometric mean titer (GMT) values and confidence intervals. Table 3.4 below shows adjusted P-values comparing various groups.

TABLE 3.3
gE-specific total IgG titers post dose 2

| | | | | 5 μg | 5 μg | 5 μg | 5 μg |
|---|---|---|---|---|---|---|---|
| | | | | gE + | gE + | gE + | gE + L2 + |
| | | 5 μg | | 5 μg | 30 μg | 100 μg | 5 μg |
| | PBS | gE | 5 μg | 1055 | 1055 | 1055 | 1055 |
| | Alone | Alone | gE + L2 | C.S | C.S | C.S | F.A |
| Number of | 10 | 10 | 10 | 10 | 10 | 10 | 10 |
| values | | | | | | | |
| Geometric | 125.9 | 5012 | 19953 | 39811 | 1000001 | 794329 | 125893 |
| mean | | | | | | | |
| Geometric | 2.071 | 8.886 | 3.041 | 3.284 | 1 | 2.071 | 2.071 |
| SD factor | | | | | | | |
| Lower 95% | 74.78 | 1050 | 9004 | 17006 | 1000001 | 471829 | 74780 |
| CI of geo. | | | | | | | |
| mean | | | | | | | |
| Upper 95% | 211.9 | 23914 | 44213 | 93198 | 1000001 | 1337261 | 211941 |
| CI of geo. | | | | | | | |
| mean | | | | | | | |

| | | | 5 μg | 5 μg | 5 μg | |
|---|---|---|---|---|---|---|
| | | | gE + L2 + | gE + L2 + | gE + L2 + | |
| | 5 μg | 5 μg | 5 μg | 30 μg | 100 μg | |
| | gE + L2 + | gE + L2 + | 1055 | 1055 | 1055 | 5 μg |
| | 30 μg | 100 μg | F.A + | F.A + | F.A + | gE + |
| | 1055 | 1055 | 20 μg | 20 μg | 20 μg | 20 μg |
| | F.A | F.A | PHAD | PHAD | PHAD | PHAD |
| Number of | 10 | 10 | 10 | 10 | 10 | 10 |
| values | | | | | | |
| Geometric | 125893 | 501188 | 1000001 | 1000001 | 1000001 | 1000001 |
| mean | | | | | | |
| Geometric | 2.071 | 3.041 | 1 | 1 | 1 | 1 |
| SD factor | | | | | | |
| Lower 95% | 74780 | 226178 | 1000001 | 1000001 | 1000001 | 1000001 |
| CI of geo. | | | | | | |
| mean | | | | | | |
| Upper 95% | 211941 | 1110582 | 1000001 | 1000001 | 1000001 | 1000001 |
| CI of geo. | | | | | | |
| mean | | | | | | |

TABLE 3
Group-wise comparisons between groups in Table 3.3

| | | | Adjusted P |
|---|---|---|---|
| Tukey's multiple comparisons test | Significant? | Summary | Value |
| PBS Alone vs. 5 μg gE Alone | No | ns | >0.9999 |
| PBS Alone vs. 5 μg gE + L2 | No | ns | >0.9999 |
| PBS Alone vs. 5 μg gE + 5 μg 1055 CS | No | ns | >0.9999 |
| PBS Alone vs. 5 μg gE + 30 μg 1055 CS | Yes | **** | <0.0001 |
| PBS Alone vs. 5 μg gE + 100 μg 1055 CS | Yes | **** | <0.0001 |
| PBS Alone vs. 5 μg gE + L2 + 5 μg 1055 | No | ns | 0.5482 |
| FA | | | |
| PBS Alone vs. 5 μg gE + L2 + 30 μg 1055 | No | ns | 0.5482 |
| FA | | | |
| PBS Alone vs. 5 μg gE + L2 + 100 μg 1055 | Yes | **** | <0.0001 |
| FA | | | |
| PBS Alone vs. 5 μg gE + L2 + 5 μg 1055 | Yes | **** | <0.0001 |
| FA + 20 μg PHAD liposomes | | | |
| PBS Alone vs. 5 μg gE + L2 + 30 μg 1055 | Yes | **** | <0.0001 |
| FA + 20 μg PHAD liposomes | | | |
| PBS Alone vs. 5 μg gE + L2 + 100 μg 1055 | Yes | **** | <0.0001 |
| FA + 20 μg PHAD liposomes | | | |
| PBS Alone vs. 5 μg gE + 20 μg PHAD | Yes | **** | <0.0001 |
| liposomes | | | |
| 5 μg gE Alone vs. 5 μg gE + L2 | No | ns | >0.9999 |
| 5 μg gE Alone vs. 5 μg gE + 5 μg 1055 CS | No | ns | >0.9999 |
| 5 μg gE Alone vs. 5 μg gE + 30 μg 1055 | Yes | **** | <0.0001 |
| CS | | | |
| 5 μg gE Alone vs. 5 μg gE + 100 μg 1055 | Yes | **** | <0.0001 |
| CS | | | |
| 5 μg gE Alone vs. 5 μg gE + L2 + 5 μg | No | ns | 0.7461 |
| 1055 FA | | | |
| 5 μg gE Alone vs. 5 μg gE + L2 + 30 μg | No | ns | 0.7461 |
| 1055 FA | | | |
| 5 μg gE Alone vs. 5 μg gE + L2 + 100 μg | Yes | **** | <0.0001 |
| 1055 FA | | | |
| 5 μg gE Alone vs. 5 μg gE + L2 + 5 μg | Yes | **** | <0.0001 |
| 1055 FA + 20 μg PHAD liposomes | | | |
| 5 μg gE Alone vs. 5 μg gE + L2 + 30 μg | Yes | **** | <0.0001 |
| 1055 FA + 20 μg PHAD liposomes | | | |
| 5 μg gE Alone vs. 5 μg gE + L2 + 100 μg | Yes | **** | <0.0001 |
| 1055 FA + 20 μg PHAD liposomes | | | |
| 5 μg gE Alone vs. 5 μg gE + 20 μg PHAD | Yes | **** | <0.0001 |
| liposomes | | | |
| 5 μg gE + L2 vs. 5 | No | ns | >0.9999 |
| μg gE + 5 μg 1055 | | | |
| CS | | | |
| 5 μg gE + L2 vs. 5 | Yes | **** | <0.0001 |
| μg gE + 30 μg 1055 | | | |
| CS | | | |
| 5 μg gE + L2 vs. 5 | Yes | **** | <0.0001 |
| μg gE + 100 μg | | | |
| 1055 CS | | | |
| 5 μg gE + L2 vs. 5 | No | ns | 0.8333 |
| μg gE + L2 + 5 μg | | | |
| 1055 FA | | | |
| 5 μg gE + L2 vs. 5 | No | ns | 0.8333 |
| μg gE + L2 + 30 μg | | | |
| 1055 FA | | | |
| 5 μg gE + L2 vs. 5 | Yes | **** | <0.0001 |
| μg gE + L2 + 100 | | | |
| μg 1055 FA | | | |
| 5 μg gE + L2 vs. 5 | Yes | **** | <0.0001 |
| μg gE + L2 + 5 μg | | | |
| 1055 FA + 20 μg | | | |
| PHAD liposomes | | | |
| 5 μg gE + L2 vs. 5 | Yes | **** | <0.0001 |
| μg gE + L2 + 30 μg | | | |
| 1055 FA + 20 μg | | | |
| PHAD liposomes | | | |
| 5 μg gE + L2 vs. 5 | Yes | **** | <0.0001 |
| μg gE + L2 + 100 | | | |
| μg 1055 FA + 20 | | | |
| μg PHAD | | | |
| liposomes | | | |
| 5 μg gE + L2 vs. 5 | Yes | **** | <0.0001 |
| μg gE + 20 μg | | | |
| PHAD liposomes | | | |
| 5 μg gE + 5 μg 1055 CS vs. 5 μg gE + 30 | Yes | **** | <0.0001 |
| μg 1055 CS | | | |
| 5 μg gE + 5 μg 1055 CS vs. 5 μg gE + 100 | Yes | **** | <0.0001 |
| μg 1055 CS | | | |
| 5 μg gE + 5 μg 1055 CS vs. 5 μg gE + L2 + | No | ns | 0.9527 |
| 5 μg 1055 | | | |
| FA | | | |
| 5 μg gE + 5 μg 1055 CS vs. 5 μg gE + L2 + | No | ns | 0.9527 |
| 30 μg 1055 FA | | | |
| 5 μg gE + 5 μg 1055 CS vs. 5 μg gE + L2 + | Yes | **** | <0.0001 |
| 100 μg 1055 | | | |
| FA | | | |
| 5 μg gE + 5 μg 1055 CS vs. 5 μg gE + L2 + | Yes | **** | <0.0001 |
| 5 μg 1055 FA + 20 μg PHAD liposomes | | | |
| 5 μg gE + 5 μg 1055 CS vs. 5 μg gE + L2 + | Yes | **** | <0.0001 |
| 30 μg 1055 | | | |
| FA + 20 μg PHAD liposomes | | | |
| 5 μg gE + 5 μg 1055 CS vs. 5 μg gE + L2 + | Yes | **** | <0.0001 |
| 100 μg 1055 FA + 20 μg PHAD liposomes | | | |
| 5 μg gE + 5 μg 1055 CS vs. 5 μg gE + 20 | Yes | **** | <0.0001 |
| μg PHAD liposomes | | | |
| 5 μg gE + 30 μg 1055 CS vs. 5 μg gE + | No | ns | 0.9972 |
| 100 μg 1055 CS | | | |
| 5 μg gE + 30 μg 1055 CS vs. 5 μg gE + | Yes | **** | <0.0001 |
| L2 + 5 μg 1055 FA | | | |
| 5 μg gE + 30 μg 1055 CS vs. 5 μg gE + | Yes | **** | <0.0001 |
| L2 + 30 μg 1055 FA | | | |
| 5 μg gE + 30 μg 1055 CS vs. 5 μg gE + | No | ns | 0.0792 |
| L2 + 100 μg 1055 FA | | | |
| 5 μg gE + 30 μg 1055 CS vs. 5 μg gE + | No | ns | >0.9999 |
| L2 + 5 μg 1055 | | | |
| FA + 20 μg PHAD liposomes | | | |
| 5 μg gE + 30 μg 1055 CS vs. 5 μg gE + L2 + | No | ns | >0.9999 |
| 30 μg 1055 FA + 20 μg PHAD liposomes | | | |
| 5 μg gE + 30 μg 1055 CS vs. 5 μg gE + L2 + | No | ns | >0.9999 |
| 100 μg 1055 FA + 20 μg PHAD | | | |
| liposomes | | | |
| 5 μg gE + 30 μg 1055 CS vs. 5 μg gE + 20 | No | ns | >0.9999 |
| μg PHAD liposomes | | | |
| 5 μg gE + 100 μg 1055 CS vs. 5 μg gE + | Yes | **** | <0.0001 |
| L2 + 5 μg 1055 FA | | | |
| 5 μg gE + 100 μg 1055 CS vs. 5 μg gE + | Yes | **** | <0.0001 |
| L2 + 30 μg 1055 FA | | | |
| 5 μg gE + 100 μg 1055 CS vs. 5 μg gE + | No | ns | 0.6314 |
| L2 + 100 μg 1055 FA | | | |
| 5 μg gE + 100 μg 1055 CS vs. 5 μg gE + | No | ns | 0.9996 |
| L2 + 5 μg 1055 FA + 20 μg PHAD | | | |
| liposomes | | | |
| 5 μg gE + 100 μg 1055 CS vs. 5 μg gE + | No | ns | 0.9972 |
| L2 + 30 μg 1055 FA + 20 μg PHAD | | | |
| liposomes | | | |
| 5 μg gE + 100 μg 1055 CS vs. 5 μg gE + | No | ns | 0.9972 |
| L2 + 100 μg 1055 FA + 20 μg PHAD | | | |
| liposomes | | | |
| 5 μg gE + 100 μg 1055 CS vs. 5 μg gE + | No | ns | 0.9972 |
| 20 μg PHAD liposomes | | | |
| 5 μg gE + L2 + 5 μg 1055 FA vs. 5 μg gE + | No | ns | >0.9999 |
| L2 + | | | |
| 30 μg 1055 FA | | | |
| 5 μg gE + L2 + 5 μg 1055 FA vs. 5 μg gE + | Yes | **** | <0.0001 |
| L2 + | | | |
| 100 μg 1055 FA | | | |
| 5 μg gE + L2 + 5 μg 1055 FA vs. 5 μg gE + | Yes | **** | <0.0001 |
| L2 + 5 μg 1055 FA + 20 μg PHAD | | | |
| liposomes | | | |
| 5 μg gE + L2 + 5 μg 1055 FA vs. 5 μg gE + | Yes | **** | <0.0001 |
| L2 + | | | |
| 30 μg 1055 FA + 20 μg PHAD | | | |
| liposomes | | | |
| 5 μg gE + L2 + 5 μg 1055 FA vs. 5 μg gE + | Yes | **** | <0.0001 |
| L2 + | | | |
| 100 μg 1055 FA + 20 μg PHAD | | | |
| liposomes | | | |
| 5 μg gE + L2 + 5 μg 1055 FA vs. 5 μg gE + | Yes | **** | <0.0001 |
| 20 μg | | | |
| PHAD liposomes | | | |
| 5 μg gE + L2 + 30 μg 1055 FA vs. 5 μg | Yes | **** | <0.0001 |
| gE + L2 + 100 μg 1055 FA | | | |
| 5 μg gE + L2 + 30 μg 1055 FA vs. 5 μg | Yes | **** | <0.0001 |
| gE + L2 + 5 μg 1055 FA + 20 μg PHAD | | | |
| liposomes | | | |
| 5 μg gE + L2 + 30 μg 1055 FA vs. 5 μg | Yes | **** | <0.0001 |
| gE + L2 + 30 μg 1055 FA + 20 μg PHAD | | | |
| liposomes | | | |
| 5 μg gE + L2 + 30 μg 1055 FA vs. 5 μg | Yes | **** | <0.0001 |
| gE + L2 + 100 μg 1055 FA + 20 μg PHAD | | | |
| liposomes | | | |
| 5 μg gE + L2 + 30 μg 1055 FA vs. 5 μg | Yes | **** | <0.0001 |
| gE + 20 μg PHAD liposomes | | | |
| 5 μg gE + L2 + 100 μg 1055 FA vs. 5 μg | No | ns | 0.3062 |
| gE + L2 + 5 μg 1055 FA + 20 μg PHAD | | | |
| liposomes | | | |
| 5 μg gE + L2 + 100 μg 1055 FA vs. 5 μg | No | ns | 0.0792 |
| gE + L2 + 30 μg 1055 FA + 20 μg PHAD | | | |
| liposomes | | | |
| 5 μg gE + L2 + 100 μg 1055 FA vs. 5 μg | No | ns | 0.0792 |
| gE + L2 + 100 μg 1055 FA + 20 μg PHAD | | | |
| liposomes | | | |
| 5 μg gE + L2 + 100 μg 1055 FA vs. 5 μg | No | ns | 0.0792 |
| gE + 20 μg PHAD liposomes | | | |
| 5 μg gE + L2 + 5 μg 1055 FA + 20 μg | No | ns | >0.9999 |
| PHAD liposomes vs. 5 μg gE + L2 + 30 μg | | | |
| 1055 FA + 20 μg PHAD liposomes | | | |
| 5 μg gE + L2 + 5 μg 1055 FA + 20 μg | No | ns | >0.9999 |
| PHAD liposomes vs. 5 μg gE + L2 + 100 | | | |
| μg 1055 FA + 20 μg PHAD liposomes | | | |
| 5 μg gE + L2 + 5 μg 1055 FA + 20 μg | No | ns | >0.9999 |
| PHAD liposomes vs. 5 μg gE + 20 μg | | | |
| PHAD liposomes | | | |
| 5 μg gE + L2 + 30 μg 1055 FA + 20 μg | No | ns | >0.9999 |
| PHAD liposomes vs. 5 μg gE + L2 + 100 | | | |
| μg 1055 FA + 20 μg PHAD liposomes | | | |
| 5 μg gE + L2 + 30 μg 1055 FA + 20 μg | No | ns | >0.9999 |
| PHAD liposomes vs. 5 μg gE + 20 μg | | | |
| PHAD liposomes | | | |
| 5 μg gE + L2 + 100 μg 1055 FA + 20 μg | No | ns | >0.9999 |
| PHAD liposomes vs. 5 μg gE + 20 μg | | | |
| PHAD liposomes | | | |

### Example 4—Efficacy Evaluation of TQL-1055 In Vivo

The impact of TQL-1055 choline salt (in situ salt) at various dosages, a TLR9 or TLR4 agonist, and VZV gE (expressed by CHO cells), as compared to AS-01B (5 mcg MPL+5 mcg QS-21) was tested. In Experiment #1, mice were immunized with PBS alone, gE (5 mcg)+TLR9 ligand (50 mcg)+TQL-1055 (four groups: 5 mcg, 15 mcg, 25 mcg, 50 mcg), and AS-01B. In Experiment #2, mice were immunized with PBS alone, gE (5 mcg)+TLR4 ligand (5 mcg)+TQL-1055 (four groups: 5 mcg, 15 mcg, 25 mcg, 50 mcg), and AS-01B.

Mice were immunized at Day 0 and Day 21. Groups were bled at Day 31 (post Dose 1) for serum analysis. The results are shown below.

### ICS (CD4+ T-Cell) Results

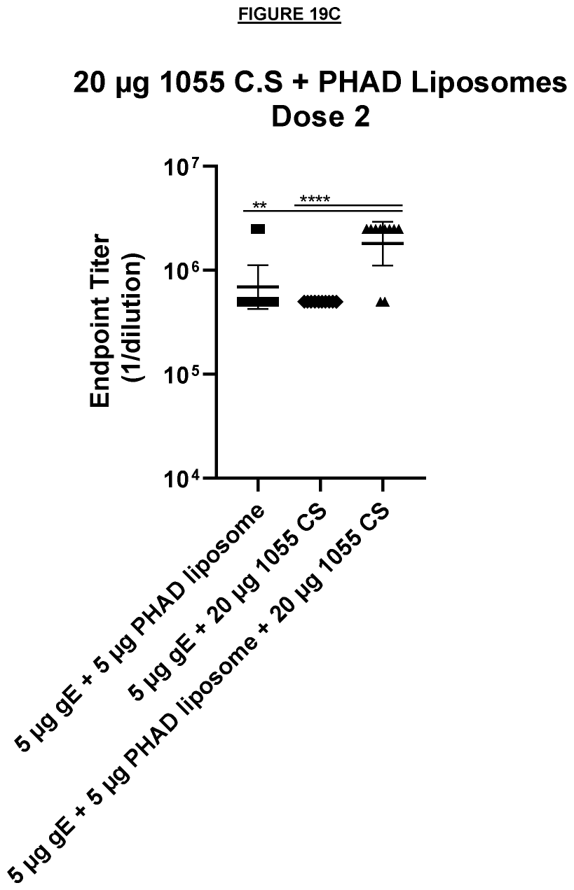

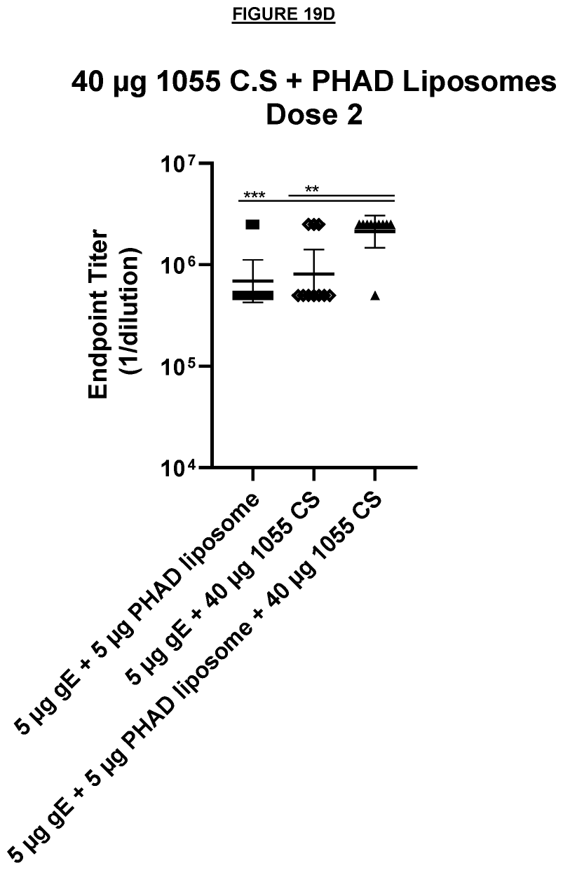

FIG. 22 is a graph depicting the results of a T-cell intracellular cytokine staining (ICS) assay (CD4+ T-cell) showing the percentage of CD4+ T cells expressing one or more cytokines in response to stimulation with VZV gE for Experiment 1. FIG. 23 is a graph depicting the results of T-cell intracellular cytokine staining (ICS) assays (CD4+ T-cell) showing the percentage of CD4+ T cells expressing one or more cytokines in response to stimulation with VZV gE for Experiment 2.

### FluoroSpot (T-Cell) Results

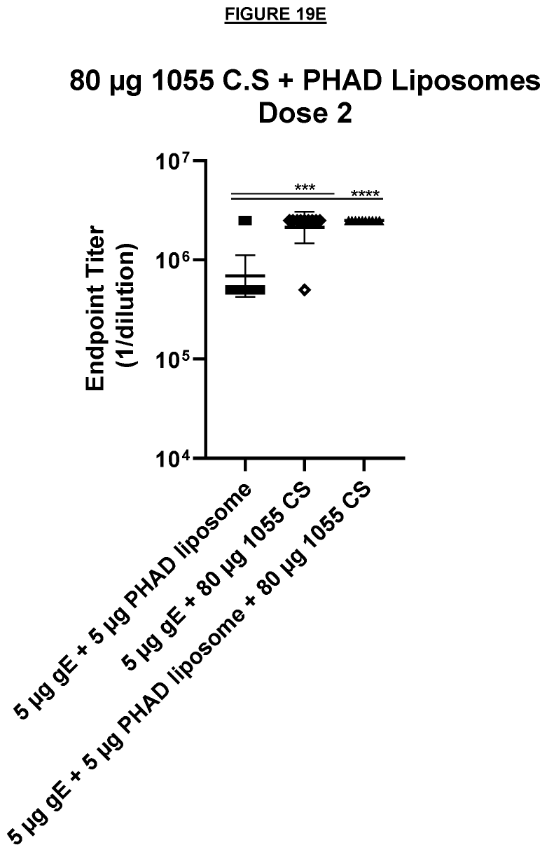

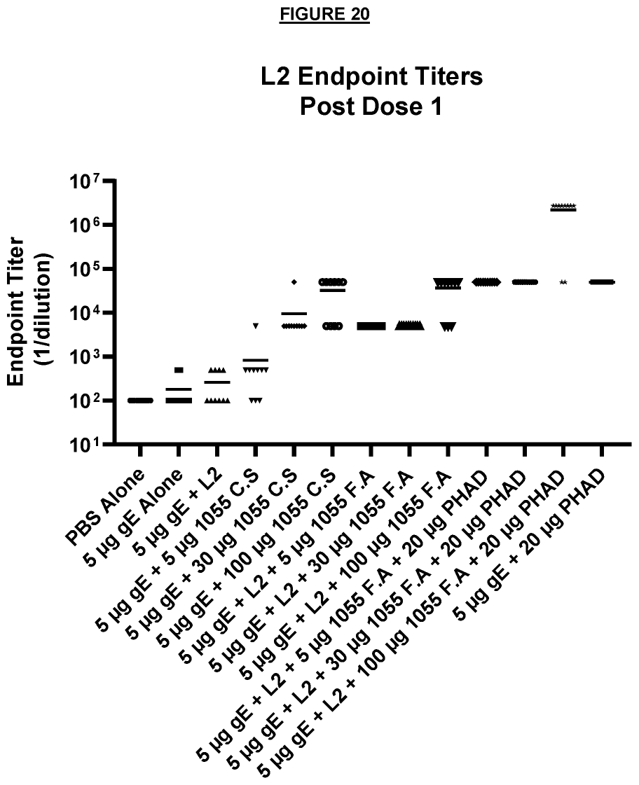

FIG. 24 is a graph depicting the results of a FluroSpot (T-cell) assay showing spot forming cells per million spleen lymphocytes for Experiment 1. FIG. 25 is a graph depicting the results of a FluroSpot (T-cell) assay showing spot forming cells per million spleen lymphocytes for Experiment 2.

### ELISA (Antibody) Results

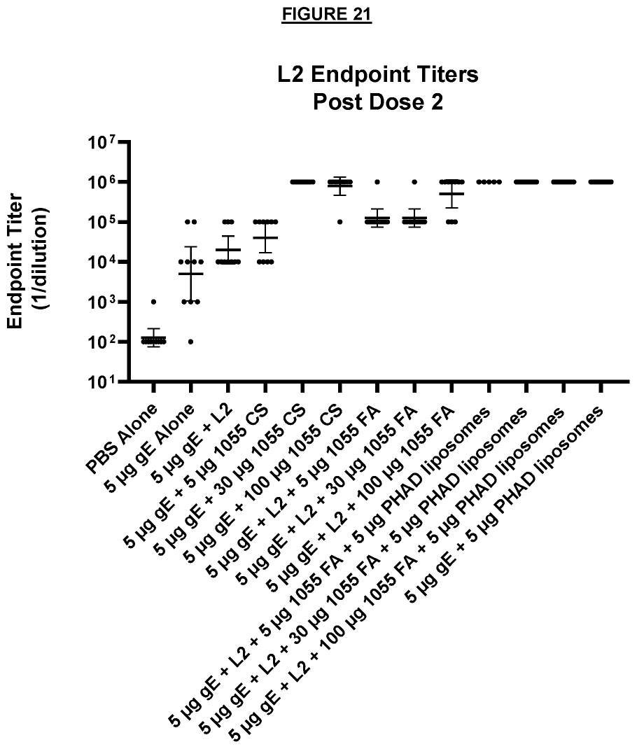

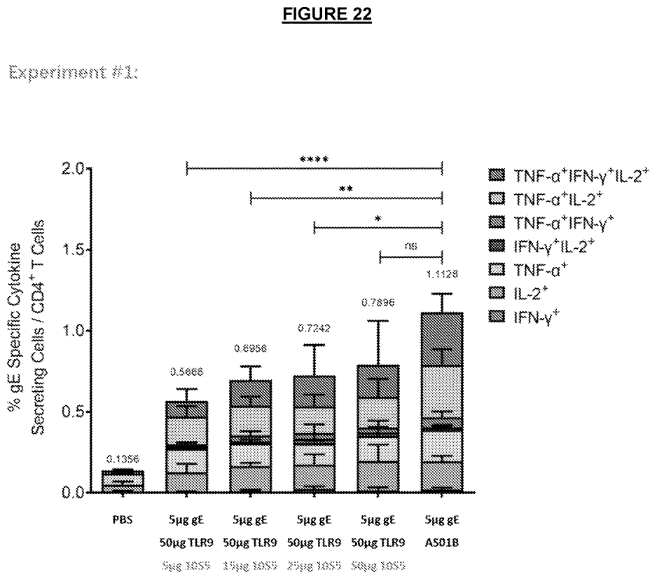

FIG. 26 is a graph depicting the results of an enzyme-linked immunosorbent assay (ELISA) showing antibody titers for Experiment 1. FIG. 27 is a graph depicting the results of an enzyme-linked immunosorbent assay (ELISA) showing antibody titers for Experiment 1.

### Discussion

The data shown in FIGS. 22 to 27 demonstrate (1) TQL-1055, when combined with TLR9 or TLR4 ligand, induces dose-dependent T-cell and antibody responses in mice; and (2) TQL-1055 at 5 mcg dose, when combined with TLR4 ligand, can replace AS-01B without any significant difference in T-cell and antibody responses in mice.

### Example 5—Immunogenicity of TQL-1055+TLR4 Agonist Candidates in CD-1 Mice

The tolerability and immunogenicity of formulations containing (TQL-1055 free acid HP-β-CD (2-hydroxypropyl beta-cyclodextrin) and TLR4 agonist candidates were studied. CD-1 mice were immunized as shown in the table below. PHAD liposomes were obtained from Avanti. EcML was obtained from EuBiologics. CRX-527 was obtained from InvivoGen. TLR4a-1 is an undisclosed TLR4 compound. AS01 is a combination of 5 mcg QS-21 and 5 mcg MPL used in SHINGRIX.

| Group | Dose |
|---|---|
| 1 | 5 mcg gE |
| 2 | 5 mcg gE + 40 mcg TQL-1055 FA HPβCD + 1 mcg PHAD |
| 3 | 5 mcg gE + 40 mcg TQL-1055 FA HPβCD + 5 mcg PHAD |
| 4 | 5 mcg gE + 40 mcg TQL-1055 FA HPβCD + 1 mcg EcML |
| 5 | 5 mcg gE + 40 mcg TQL-1055 FA HPβCD + 5 mcg EcML |
| 6 | 5 mcg gE + 40 mcg TQL-1055 FA HPβCD + 0.5 mcg CRX-527 |
| 7 | 5 mcg gE + 40 mcg TQL-1055 FA HPβCD + 1 mcg CRX-527 |
| 8 | 5 mcg gE + 40 mcg TQL-1055 FA HPβCD + 1 mcg TLR4a-1 |
| 9 | 5 mcg gE + 40 mcg TQL-1055 FA HPβCD + 5 mcg TLR4a-1 |
| 10 | 5 mcg gE + 5 mcg AS01 |

Mice were immunized via I.M. on Day 0 and Day 14. Groups were bled at Day-1, Day 13 (post Dose 1), and Day 28 (post Dose 2) for serum analysis for anti-gE specific antibody response and anti-gE specific T cell response. Weight measurements were also obtained on Days 0, 1, 2, 14, 15, 16, and 17. The results are shown below.

### Weight Loss

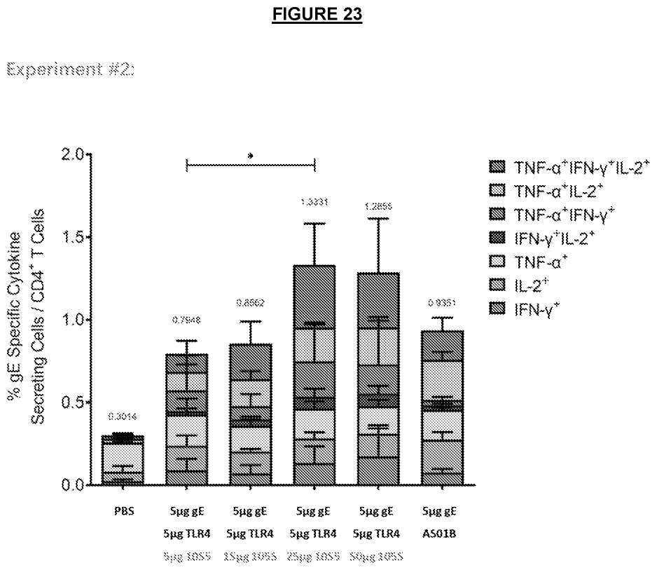

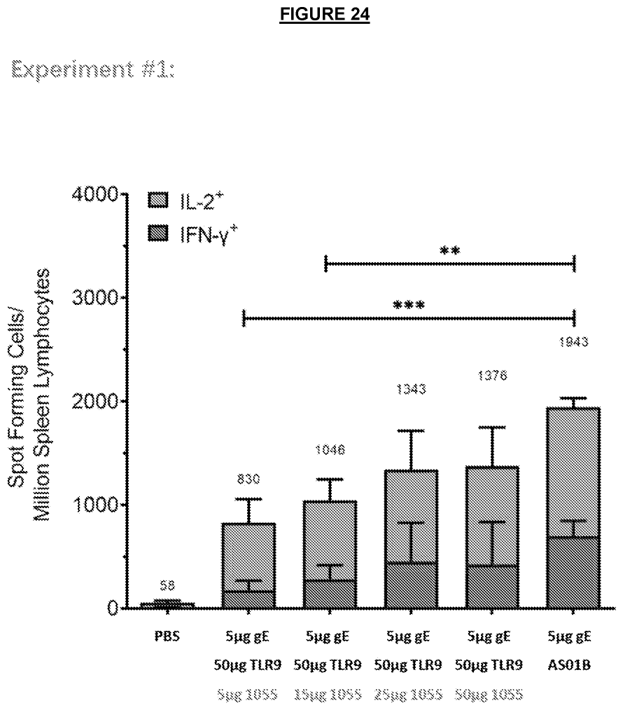

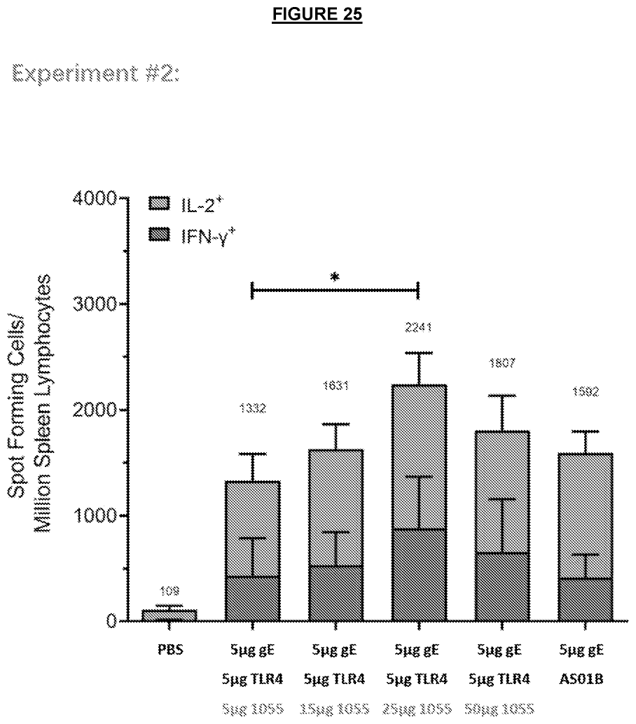

Post dose 1 and dose 2 mean percent (%) initial weight change for each group is depicted in FIGS. 28 and 29. Fisher's least significant difference test values are shown in the tables below. Post dose 1, only AS01 adjuvanted mice were significantly lower than unadjuvanted control. Post dose 2, only high dose PHAD and high dose EcML were significantly lower than AS01 unadjuvanted on D15/D16.

| Post Dose 1 Fisher's LSD | | |
|---|---|---|
| Compared to AS01 adjuvanted | D1 | D2 |
| 5 μg gE | 0.0059 | 0.0273 |
| 5 μg gE + 40 μg | 0.0124 | 0.1210 |
| TQL1055 + 1 μg PHAD | | |
| 5 μg gE + 40 μg | 0.0029 | 0.0454 |
| TQL1055 + 5 μg PHAD | | |
| 5 μg gE + 40 μg | 0.0193 | 0.1718 |
| TQL1055 + 1 μg EcML | | |
| 5 μg gE + 40 μg | 0.0423 | 0.0941 |
| TQL1055 + 5 μg EcML | | |
| 5 μg gE + 40 μg | 0.0021 | 0.0578 |
| TQL1055 + 1 μg TLR4a-1 | | |
| 5 μg gE + 40 μg | 0.0176 | 0.0246 |
| TQL1055 + 5 μg TLR4a-1 | | |
| 5 μg gE + 40 μg | 0.0547 | 0.2483 |
| TQL1055 + 0.5 μg CRX-527 | | |
| 5 μg gE + 40 μg | 0.0034 | 0.1422 |
| TQL1055 + 1 μg CRX-527 | | |

| Post Dose 2 Fisher's LSD | | | |
|---|---|---|---|
| Compared to AS01 adjuvanted | D15 | D16 | D17 |
| 5 μg gE | 0.7862 | 0.3474 | 0.6646 |
| 5 μg gE + 40 μg | 0.7986 | 0.3673 | 0.6483 |
| TQL1055 + 1 μg PHAD | | | |
| 5 μg gE + 40 μg | 0.0017 | 0.0131 | 0.4607 |
| TQL1055 + 5 μg PHAD | | | |
| 5 μg gE + 40 μg | 0.1567 | 0.0549 | 0.4473 |
| TQL1055 + 1 μg EcML | | | |
| 5 μg gE + 40 μg | 0.0103 | 0.0129 | 0.3682 |
| TQL1055 + 5 μg EcML | | | |
| 5 μg gE + 40 μg | 0.3662 | 0.2867 | 0.4508 |
| TQL1055 + 1 μg TLR4a-1 | | | |
| 5 μg gE + 40 μg | 0.1476 | 0.0888 | 0.5915 |
| TQL1055 + 5 μg TLR4a-1 | | | |
| 5 μg gE + 40 μg | 0.8931 | 0.1801 | 0.4515 |
| TQL1055 + 0.5 μg CRX-527 | | | |
| 5 μg gE + 40 μg | 0.9934 | 0.8737 | 0.8700 |
| TQL1055 + 1 μg CRX-527 | | | |

| Post Dose 1 Fisher's LSD | | |
|---|---|---|
| Compared to gE alone | D1 | D2 |
| 5 μg gE + 40 μg TQL1055 + 1 μg PHAD | 0.3981 | 0.5998 |
| 5 μg gE + 40 μg TQL1055 + 5 μg PHAD | 0.9612 | 0.8778 |
| 5 μg gE + 40 μg TQL1055 + 1 μg EcML | 0.9275 | 0.4103 |
| 5 μg gE + 40 μg TQL1055 + 5 μg EcML | 0.1450 | 0.3023 |
| 5 μg gE + 40 μg TQL1055 + 1 μg TLR4a-1 | 0.6176 | 0.6535 |
| 5 μg gE + 40 μg TQL1055 + 5 μg TLR4a-1 | 0.5731 | 0.7829 |
| 5 μg gE + 40 μg TQL1055 + 0.5 μg CRX-527 | 0.5548 | 0.4023 |
| 5 μg gE + 40 μg TQL1055 + 1 μg CRX-527 | 0.7412 | 0.3647 |
| | 0.0059 | 0.0273 |

| 5 μg gE + 5 μg AS01 | | | |
|---|---|---|---|
| Compared to gE alone | D15 | D16 | D17 |
| 5 μg gE + 40 μg | 0.9514 | 0.9358 | 0.8859 |
| TQL1055 + 1 μg PHAD | | | |
| 5 μg gE + 40 μg | 0.0022 | 0.1511 | 0.6477 |
| TQL1055 + 5 μg PHAD | | | |
| 5 μg gE + 40 μg | 0.1675 | 0.5402 | 0.6710 |
| TQL1055 + 1 μg EcML | | | |
| 5 μg gE + 40 μg | 0.0165 | 0.2893 | 0.5405 |
| TQL1055 + 5 μg EcML | | | |
| 5 μg gE + 40 μg | 0.3140 | 0.8706 | 0.6409 |
| TQL1055 + 1 μg TLR4a-1 | | | |
| 5 μg gE + 40 μg | 0.1521 | 0.4906 | 0.8450 |
| TQL1055 + 5 μg TLR4a-1 | | | |
| 5 μg gE + 40 μg | 0.9245 | 0.7166 | 0.6671 |
| TQL1055 + 0.5 μg CRX-527 | | | |
| 5 μg gE + 40 μg | 0.8269 | 0.5201 | 0.6017 |
| TQL1055 + 1 μg CRX-527 | | | |
| 5 μg gE + 5 μg AS01 | 0.7862 | 0.3474 | 0.6646 |

### Anti-gE ELISA

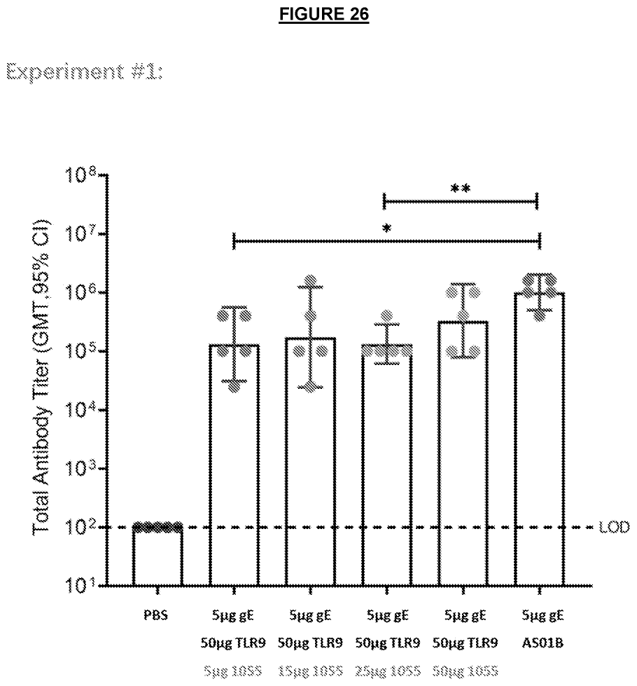

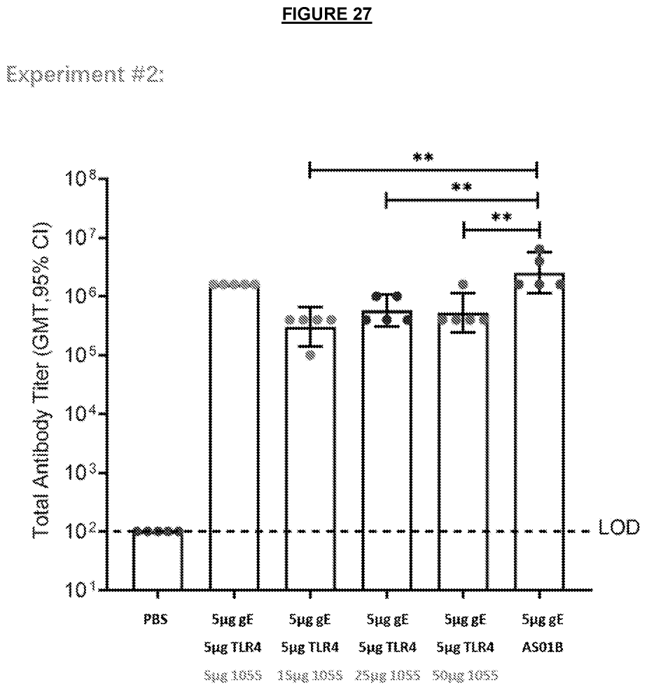

D28 (post dose 2) total IgG endpoint titers are shown in FIG. 30. D28 (post dose 2 IgG2a subclass endpoint titers are shown in FIG. 31. The combination of TQL 1055 and any TLR4 at all doses significantly increased total IgG endpoint titers by roughly 2 logs over unadjuvanted control. There were no large differences seen between different TLR4 agonists. Increasing the TLR4 dose had no effect on endpoint titers for EcML and CRX-527. In contrast, high dose of TLR4a for PHAD and TLR4a-1 approximately doubled titers of the low dose counterparts.

The combination of TQL1055 and any TLR4 at all doses significantly increased IgG2a endpoint titers by 2-3 logs over unadjuvanted control. Similar responses were observed from all 4 TLR4 agonists with CRX-527 having the lowest IgG2a titers. There was a more pronounced dose effect of TLR4a on IgG2a than total IgG. PHAD had the largest difference between low and high dose at 8 times higher. At the low TLR4a dose, EcML had the highest IgG2a.

### D26 CD4 T-Cell ICS

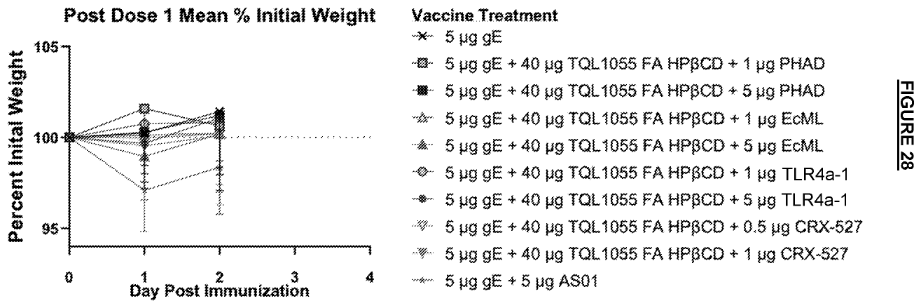

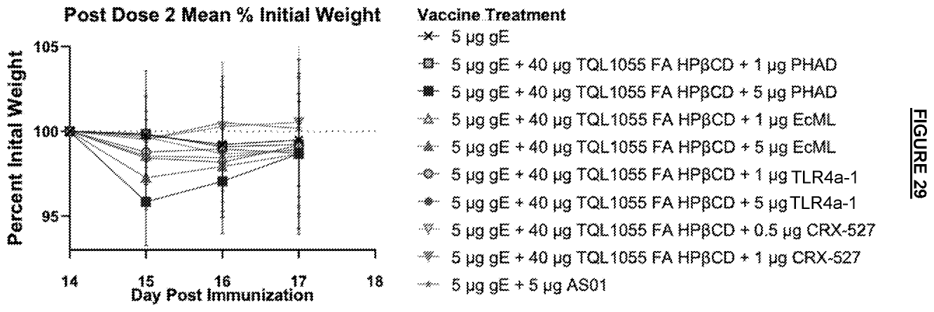

D26 CD4 T cell ICS results are shown in FIG. 32. D26 CD4 T cell ICS polyfunctional results are shown in FIG. 33. Fisher's least significant difference test values are shown in the tables below. AS01 significantly increased total cytokine⁺ CD4⁺ T cells compared to unadjuvanted, as did high dose PHAD, high dose CRX-527, and low dose TLR4a-1. The increases were driven largely by higher single cytokine positive T cells for IFN-γ or IL-2. Total polyfunctional CD4⁺ T cells were significantly higher in high dose PHAD, high dose CRX-527, and low dose TLR4a-1 groups compared to unadjuvanted control. Only the total polyfunctional CD4+ T cells were significantly different. No double or triple positive groups were significantly different

| Fisher's LSD | | | | | | | IFNg+ |
|---|---|---|---|---|---|---|---|
| Compared to gE | | | | IFNg+ | IFNg+ | IL2+ | IL2+ |
| alone | IFNg+ | IL2+ | TNFa+ | IL2+ | TNFa+ | TNFa+ | TNFa+ |
| 5 μg gE + 40 μg | 0.7309 | 0.9758 | 0.6948 | 0.9840 | 0.9763 | 0.9554 | 0.9885 |
| TQL1055 + 1 μg | | | | | | | |
| PHAD | | | | | | | |
| 5 μg gE + 40 μg | <0.0001 | <0.0001 | 0.3561 | 0.9515 | 0.8968 | 0.0618 | 0.8546 |
| TQL1055 + 5 μg | | | | | | | |
| PHAD | | | | | | | |
| 5 μg gE + 40 μg | 0.9760 | 0.9863 | 0.9969 | >0.9999 | 0.9904 | 0.9502 | 0.9790 |
| TQL1055 + 1 μg | | | | | | | |
| EcML | | | | | | | |
| 5 μg gE + 40 μg | 0.9569 | 0.9649 | 0.8840 | 0.9967 | 0.9776 | 0.9867 | 0.9889 |
| TQL1055 + 5 μg | | | | | | | |
| EcML | | | | | | | |
| 5 μg gE + 40 μg | 0.0080 | 0.1211 | 0.1938 | 0.9651 | 0.9306 | 0.1822 | 0.8870 |
| TQL1055 + 1 μg | | | | | | | |
| TLR4a-1 | | | | | | | |
| 5 μg gE + 40 μg | 0.9954 | 0.9582 | 0.9874 | 0.9947 | 0.9713 | 0.9251 | 0.9718 |
| TQL1055 + 5 μg | | | | | | | |
| TLR4a-1 | | | | | | | |
| 5 μg gE + 40 μg | 0.7862 | <0.0001 | 0.9310 | 0.9935 | 0.9751 | 0.4636 | 0.9851 |
| TQL1055 + 0.5 μg | | | | | | | |
| CRX-527 | | | | | | | |
| 5 μg gE + 40 μg | 0.0003 | <0.0001 | 0.2843 | 0.9691 | 0.9623 | 0.3459 | 0.9357 |
| TQL1055 + 1 μg | | | | | | | |
| CRX-527 | | | | | | | |
| 5 μg gE + 5 μg | 0.0672 | 0.0587 | 0.3842 | 0.9847 | 0.8616 | 0.6759 | 0.9607 |
| AS01 | | | | | | | |

| Fisher's LSD | | | | | | | IFNg+ |
|---|---|---|---|---|---|---|---|
| Compared to gE + | | | | IFNg+ | IFNg+ | IL2+ | IL2+ |
| AS01 adjuvanted | IFNg+ | IL2+ | TNFa+ | IL2+ | TNFa+ | TNFa+ | TNFa+ |
| 5 μg gE | 0.0672 | 0.0587 | 0.3842 | 0.9847 | 0.8616 | 0.6759 | 0.9607 |
| 5 μg gE + 40 μg | 0.1365 | 0.0628 | 0.6324 | 0.9992 | 0.8849 | 0.7172 | 0.9721 |
| TQL1055 + 1 μg | | | | | | | |
| PHAD | | | | | | | |
| 5 μg gE + 40 μg | 0.0152 | <0.0001 | 0.9580 | 0.9668 | 0.9643 | 0.1464 | 0.8934 |
| TQL1055 + 5 μg | | | | | | | |
| PHAD | | | | | | | |
| 5 μg gE + 40 μg | 0.0718 | 0.0610 | 0.3821 | 0.9847 | 0.8711 | 0.6309 | 0.9397 |
| TQL1055 + 1 μg | | | | | | | |
| EcML | | | | | | | |
| 5 μg gE + 40 μg | 0.0756 | 0.0647 | 0.3097 | 0.9881 | 0.8837 | 0.6638 | 0.9496 |
| TQL1055 + 5 μg | | | | | | | |
| EcML | | | | | | | |
| 5 μg gE + 40 μg | 0.4049 | 0.7312 | 0.6668 | 0.9803 | 0.9304 | 0.3590 | 0.9260 |
| TQL1055 + 1 μg | | | | | | | |
| TLR4a-1 | | | | | | | |
| 5 μg gE + 40 μg | 0.0681 | 0.0659 | 0.3929 | 0.9901 | 0.8899 | 0.6086 | 0.9325 |
| TQL1055 + 5 μg | | | | | | | |
| TLR4a-1 | | | | | | | |
| 5 μg gE + 40 μg | 0.1185 | 0.0369 | 0.3388 | 0.9912 | 0.8862 | 0.7527 | 0.9458 |
| TQL1055 + 0.5 μg | | | | | | | |
| CRX-527 | | | | | | | |
| 5 μg gE + 40 μg | 0.0688 | <0.0001 | 0.8407 | 0.9843 | 0.8989 | 0.5996 | 0.9750 |
| TQL1055 + 1 μg | | | | | | | |
| CRX-527 | | | | | | | |

### D26 CD8 T-Cell

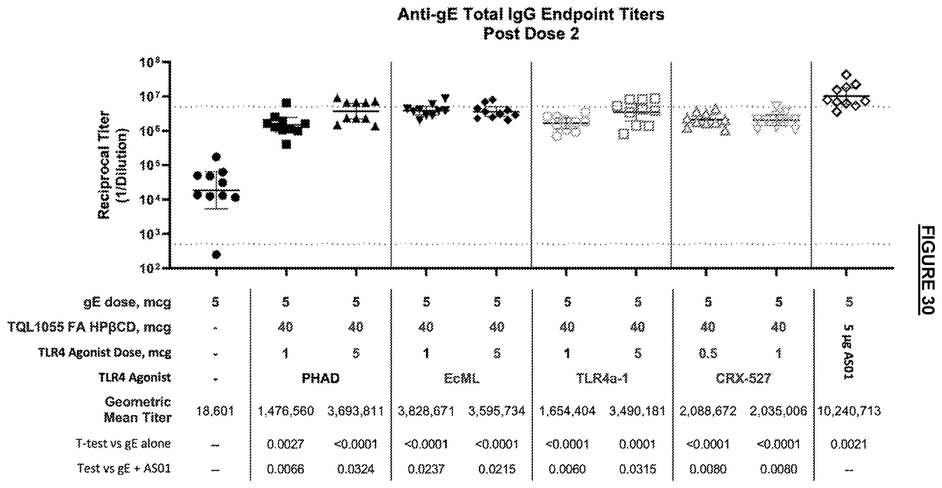

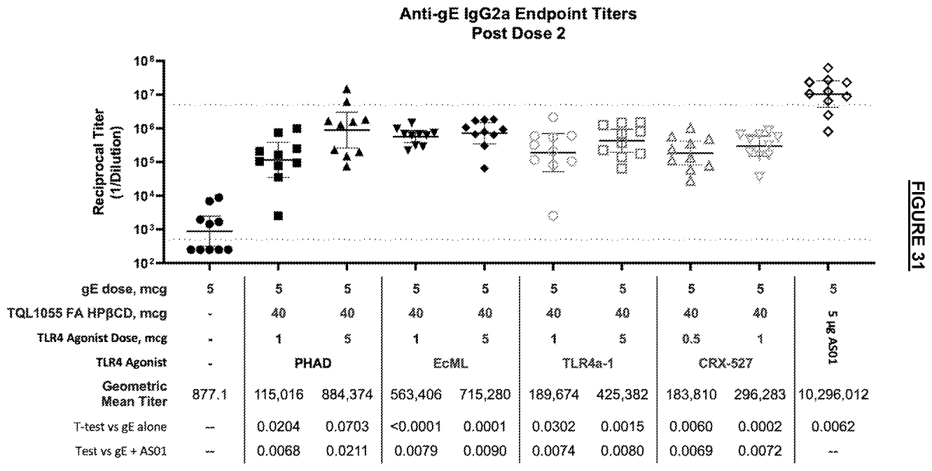

D26 CD8 T cell ICS results are shown in FIG. 34. D26 CD8 T cell ICS polyfunctional results are shown in FIG. 35. Fisher's least significant difference test values are shown in the tables below. The results show CD8 activation closely mirrored CD4 activation. High dose PHAD and both doses of CRX-527 had significantly higher activated CD8 T cells than the unadjuvanted control group. High dose TLR4a-1 trends toward higher, while AS01 has a higher point estimate but does not trend higher than unadjuvanted.

| Fisher's LSD | | | | | | | IFNg+ |
|---|---|---|---|---|---|---|---|
| Compared to gE | | | | IFNg+ | IFNg+ | IL2+ | IL2+ |
| alone | IFNg+ | IL2+ | TNFa+ | IL2+ | TNFa+ | TNFa+ | TNFa+ |
| 5 μg gE + 40 μg | 0.7731 | 0.7522 | 0.7713 | 0.9969 | 0.9980 | 0.7141 | 0.5836 |
| TQL1055 + 1 μg | | | | | | | |
| PHAD | | | | | | | |
| 5 μg gE + 40 μg | <0.0001 | 0.1288 | 0.9850 | 0.7673 | 0.9776 | 0.8584 | 0.3967 |
| TQL1055 + 5 μg | | | | | | | |
| PHAD | | | | | | | |
| 5 μg gE + 40 μg | 0.9906 | 0.9671 | 0.9856 | 0.9928 | 0.9943 | 0.9297 | 0.8277 |
| TQL1055 + 1 μg | | | | | | | |
| EcML | | | | | | | |
| 5 μg gE + 40 μg | 0.8614 | 0.6906 | 0.7970 | 0.9959 | 0.9873 | 0.8064 | 0.9007 |
| TQL1055 + 5 μg | | | | | | | |
| EcML | | | | | | | |
| 5 μg gE + 40 μg | 0.2963 | 0.5525 | 0.9104 | 0.9432 | 0.9861 | 0.7837 | 0.0056 |
| TQL1055 + 1 μg | | | | | | | |
| TLR4a-1 | | | | | | | |
| 5 μg gE + 40 μg | 0.8831 | 0.9859 | 0.9300 | 0.9992 | 0.9971 | 0.9886 | 0.7704 |
| TQL1055 + 5 μg | | | | | | | |
| TLR4a-1 | | | | | | | |
| 5 μg gE + 40 μg | 0.7912 | <0.0001 | 0.8688 | 0.9454 | 0.9945 | 0.7644 | 0.9245 |
| TQL1055 + 0.5 μg | | | | | | | |
| CRX-527 | | | | | | | |
| 5 μg gE + 40 μg | 0.0163 | 0.0007 | 0.9312 | 0.9001 | 0.9557 | 0.7915 | 0.5005 |
| TQL1055 + 1 μg | | | | | | | |
| CRX-527 | | | | | | | |
| 5 μg gE + 5 μg | 0.4182 | 0.1170 | 0.9584 | 0.9317 | 0.9929 | 0.8130 | 0.6458 |
| AS01 | | | | | | | |

| Fisher's LSD | | | | | | | IFNg+ |
|---|---|---|---|---|---|---|---|
| Compared to gE + | | | | IFNg+ | IFNg+ | IL2+ | IL2+ |
| AS01 adjuvanted | IFNg+ | IL2+ | TNFa+ | IL2+ | TNFa+ | TNFa+ | TNFa+ |
| 5 μg gE | 0.4182 | 0.1170 | 0.9584 | 0.9317 | 0.9929 | 0.8130 | 0.6458 |
| 5 μg gE + 40 μg | 0.6020 | 0.2101 | 0.8115 | 0.9348 | 0.9949 | 0.8967 | 0.9294 |
| TQL1055 + 1 μg | | | | | | | |
| PHAD | | | | | | | |
| 5 μg gE + 40 μg | <0.0001 | 0.9609 | 0.9434 | 0.8335 | 0.9847 | 0.9537 | 0.6978 |
| TQL1055 + 5 μg | | | | | | | |
| PHAD | | | | | | | |
| 5 μg gE + 40 μg | 0.4250 | 0.1269 | 0.9728 | 0.9245 | 0.9872 | 0.8821 | 0.8088 |
| TQL1055 + 1 μg EcML | | | | | | | |
| 5 μg gE + 40 μg | 0.5253 | 0.2415 | 0.8375 | 0.9277 | 0.9944 | 0.9931 | 0.7377 |
| TQL1055 + 5 μg EcML | | | | | | | |
| 5 μg gE + 40 μg | 0.8140 | 0.3291 | 0.9518 | 0.9884 | 0.9790 | 0.9697 | 0.0203 |
| TQL1055 + 1 μg | | | | | | | |
| TLR4a-1 | | | | | | | |
| 5 μg gE + 40 μg | 0.5076 | 0.1130 | 0.9715 | 0.9325 | 0.9900 | 0.8242 | 0.8667 |
| TQL1055 + 5 μg | | | | | | | |
| TLR4a-1 | | | | | | | |
| 5 μg gE + 40 μg | 0.5857 | 0.0007 | 0.9101 | 0.9862 | 0.9874 | 0.9496 | 0.7152 |
| TQL1055 + 0.5 μg | | | | | | | |
| CRX-527 | | | | | | | |
| 5 μg gE + 40 μg | 0.1095 | 0.0658 | 0.9728 | 0.9683 | 0.9627 | 0.9778 | 0.8304 |
| TQL1055 + 1 μg | | | | | | | |
| CRX-527 | | | | | | | |

### D42 CD4 T-Cell

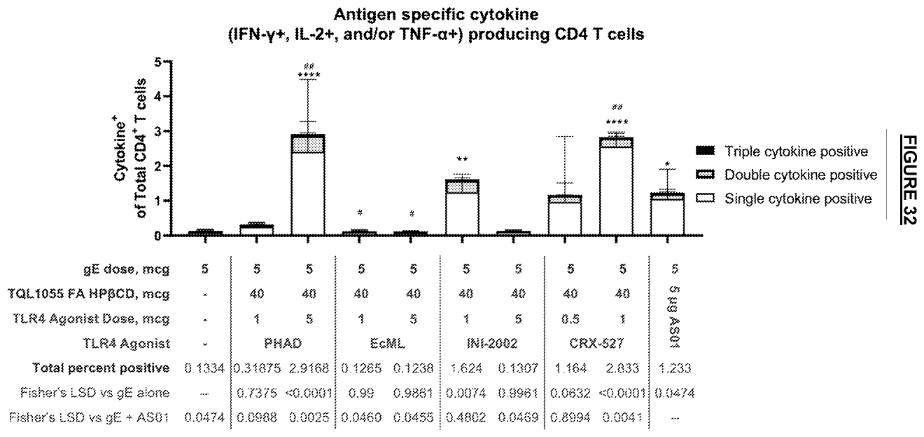

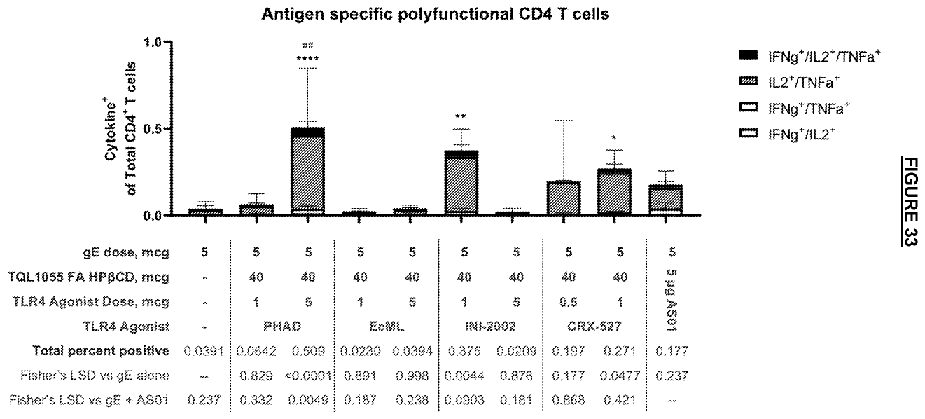

D42 CD4 T cell ICS results are shown in FIG. 36. D42 CD4 T cell ICS polyfunctional results are shown in FIG. 37. Fisher's least significant difference test values are shown in the tables below. AS01 significantly increased total cytokine⁺ CD4⁺ T cells compared to unadjuvanted, as did both PHAD doses, high dose TLR4a-1, and high dose CRX-527. Overall, levels of activation much lower than D26 T cell data. The inventors believe the data indicate D42 is too long after dose 2 to observe good activation of CD4 T cells.

| Fisher's LSD | | | | | | | IFNg+ |
|---|---|---|---|---|---|---|---|
| Compared to gE | | | | IFNg+ | IFNg+ | IL2+ | IL2+ |
| alone | IFNg+ | IL2+ | TNFa+ | IL2+ | TNFa+ | TNFa+ | TNFa+ |
| 5 μg gE + 40 μg | 0.9857 | 0.0514 | 0.3020 | 0.4483 | 0.6068 | 0.0790 | 0.0199 |
| TQL1055 + 1 μg | | | | | | | |
| PHAD | | | | | | | |
| 5 μg gE + 40 μg | 0.3156 | 0.3663 | 0.3087 | 0.9986 | 0.8633 | 0.1875 | 0.4056 |
| TQL1055 + 5 μg | | | | | | | |
| PHAD | | | | | | | |
| 5 μg gE + 40 μg | 0.1470 | 0.6669 | 0.3798 | 0.9457 | 0.9900 | 0.5637 | 0.7879 |
| TQL1055 + 1 μg | | | | | | | |
| EcML | | | | | | | |
| 5 μg gE + 40 μg | 0.1392 | 0.8562 | 0.5059 | 0.8902 | 0.6998 | 0.7495 | 0.8212 |
| TQL1055 + 5 μg | | | | | | | |
| EcML | | | | | | | |
| 5 μg gE + 40 μg | 0.4733 | 0.4867 | 0.3935 | 0.8794 | 0.6410 | 0.5481 | 0.2713 |
| TQL1055 + 1 μg | | | | | | | |
| TLR4a-1 | | | | | | | |
| 5 μg gE + 40 μg | 0.7468 | 0.0160 | 0.5025 | 0.7265 | 0.7482 | 0.4957 | 0.1647 |
| TQL1055 + 5 μg | | | | | | | |
| TLR4a-1 | | | | | | | |
| 5 μg gE + 40 μg | 0.4733 | 0.2287 | 0.9314 | 0.5186 | 0.5303 | 0.3182 | 0.4689 |
| TQL1055 + 0.5 μg | | | | | | | |
| CRX-527 | | | | | | | |
| 5 μg gE + 40 μg | 0.8324 | 0.0926 | 0.0409 | 0.3866 | 0.5613 | 0.4472 | 0.8282 |
| TQL1055 + 1 μg | | | | | | | |
| CRX-527 | | | | | | | |
| 5 μg gE + 5 μg AS01 | 0.3243 | 0.0059 | 0.0017 | 0.6206 | 0.6630 | 0.2314 | 0.5002 |

| Fisher's LSD | | | | | | | IFNg+ |
|---|---|---|---|---|---|---|---|
| Compared to gE + | | | | IFNg+ | IFNg+ | IL2+ | IL2+ |
| AS01 adjuvanted | IFNg+ | IL2+ | TNFa+ | IL2+ | TNFa+ | TNFa+ | TNFa+ |
| 5 μg gE | 0.3243 | 0.0059 | 0.0017 | 0.6206 | 0.6630 | 0.2314 | 0.5002 |
| 5 μg gE + 40 μg | 0.3156 | 0.4137 | 0.0335 | 0.7920 | 0.9371 | 0.5734 | 0.0968 |
| TQL1055 + 1 μg | | | | | | | |
| PHAD | | | | | | | |
| 5 μg gE + 40 μg | 0.9857 | 0.0624 | 0.0324 | 0.6194 | 0.7920 | 0.9029 | 0.8746 |
| TQL1055 + 5 μg | | | | | | | |
| PHAD | | | | | | | |
| 5 μg gE + 40 μg | 0.0152 | 0.0197 | 0.0227 | 0.6695 | 0.6539 | 0.5350 | 0.3458 |
| TQL1055 + 1 μg EcML | | | | | | | |
| 5 μg gE + 40 μg | 0.0141 | 0.0100 | 0.0002 | 0.7212 | 0.4116 | 0.3798 | 0.3682 |
| TQL1055 + 5 μg EcML | | | | | | | |
| 5 μg gE + 40 μg | 0.0892 | 0.0385 | 0.0213 | 0.7314 | 0.3673 | 0.5504 | 0.6695 |
| TQL1055 + 1 μg | | | | | | | |
| TLR4a-1 | | | | | | | |
| 5 μg gE + 40 μg | 0.5071 | 0.7252 | 0.0130 | 0.8845 | 0.4493 | 0.6055 | 0.4733 |
| TQL1055 + 5 μg | | | | | | | |
| TLR4a-1 | | | | | | | |
| 5 μg gE + 40 μg | 0.0892 | 0.1178 | 0.0013 | 0.8802 | 0.2880 | 0.8422 | 0.9599 |
| TQL1055 + 0.5 μg | | | | | | | |
| CRX-527 | | | | | | | |
| 5 μg gE + 40 μg | 0.2314 | 0.2776 | 0.2651 | 0.7105 | 0.8845 | 0.6617 | 0.6475 |
| TQL1055 + 1 μg | | | | | | | |
| CRX-527 | | | | | | | |

### D42 CD8 T-Cell

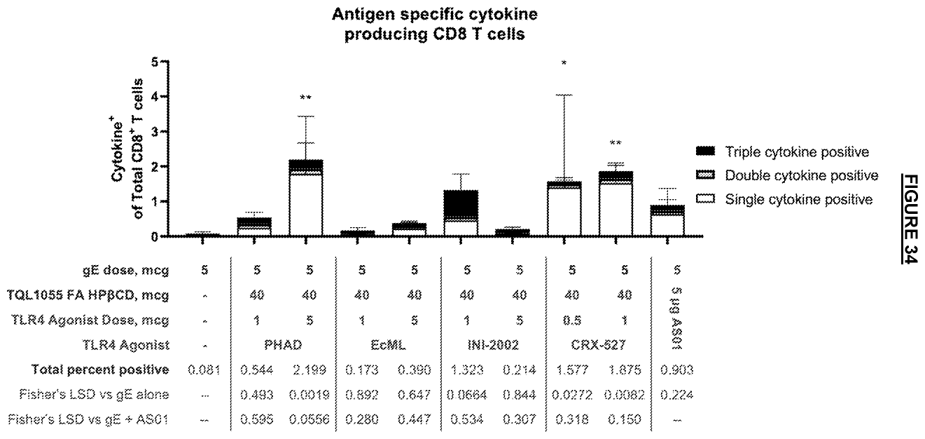

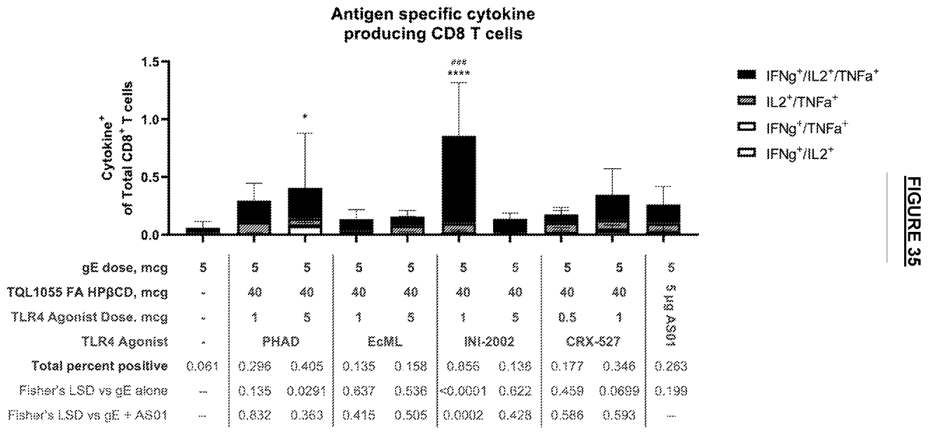

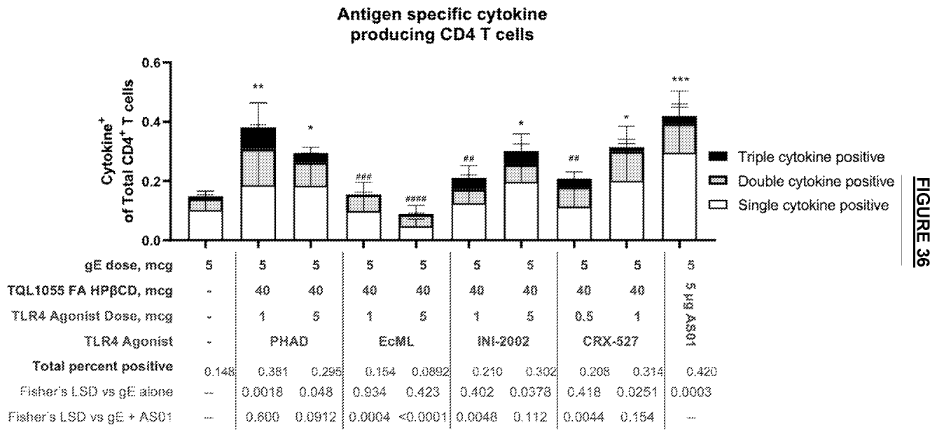

D42 CD8 T cell ICS results are shown in FIG. 38. D42 CD8 T cell ICS polyfunctional results are shown in FIG. 39. Fisher's least significant difference test values are shown in the tables below.

| Fisher's LSD | | | | | | | IFNg+ |
|---|---|---|---|---|---|---|---|
| Compared to gE | | | | IFNg+ | IFNg+ | IL2+ | IL2+ |
| alone | IFNg+ | IL2+ | TNFa+ | IL2+ | TNFa+ | TNFa+ | TNFa+ |
| 5 μg gE + 40 μg | 0.6083 | 0.8582 | 0.8102 | 0.7507 | 0.7651 | 0.5396 | 0.7898 |
| TQL1055 + 1 μg | | | | | | | |
| PHAD | | | | | | | |
| 5 μg gE + 40 μg | 0.6926 | 0.7696 | 0.9160 | 0.8204 | 0.8238 | 0.5119 | 0.9463 |
| TQL1055 + 5 μg | | | | | | | |
| PHAD | | | | | | | |
| 5 μg gE + 40 μg | 0.5195 | 0.4293 | 0.9393 | 0.8398 | 0.9021 | 0.8000 | 0.0899 |
| TQL1055 + 1 μg | | | | | | | |
| EcML | | | | | | | |
| 5 μg gE + 40 μg | 0.8375 | 0.2948 | 0.2764 | 0.9033 | 0.8605 | 0.8456 | 0.0722 |
| TQL1055 + 5 μg | | | | | | | |
| EcML | | | | | | | |
| 5 μg gE + 40 μg | 0.2921 | 0.9801 | 0.0485 | >0.9999 | 0.3887 | 0.8559 | 0.0874 |
| TQL1055 + 1 μg | | | | | | | |
| TLR4a-1 | | | | | | | |
| 5 μg gE + 40 μg | 0.5981 | <0.0001 | 0.6991 | 0.5900 | 0.4651 | 0.2315 | 0.1541 |
| TQL1055 + 5 μg | | | | | | | |
| TLR4a-1 | | | | | | | |
| 5 μg gE + 40 μg | 0.4041 | 0.5007 | 0.7977 | >0.9999 | 0.9708 | 0.7252 | 0.0150 |
| TQL1055 + 0.5 μg | | | | | | | |
| CRX-527 | | | | | | | |
| 5 μg gE + 40 μg | 0.5386 | 0.3269 | 0.1730 | 0.7418 | 0.5223 | 0.7696 | 0.2757 |
| TQL1055 + 1 μg | | | | | | | |
| CRX-527 | | | | | | | |
| 5 μg gE + 5 μg | 0.8375 | 0.2455 | 0.8813 | 0.9009 | 0.7296 | 0.7932 | 0.0601 |
| AS01 | | | | | | | |

| Fisher's LSD | | | | | | | IFNg+ |
|---|---|---|---|---|---|---|---|
| Compared to gE + | | | | IFNg+ | IFNg+ | IL2+ | IL2+ |
| AS01 adjuvanted | IFNg+ | IL2+ | TNFa+ | IL2+ | TNFa+ | TNFa+ | TNFa+ |
| 5 μg gE | 0.8375 | 0.2455 | 0.8813 | 0.9009 | 0.7296 | 0.7932 | 0.0601 |
| 5 μg gE + 40 μg | 0.4732 | 0.1804 | 0.9277 | 0.8467 | 0.9626 | 0.7252 | 0.1061 |
| TQL1055 + 1 μg | | | | | | | |
| PHAD | | | | | | | |
| 5 μg gE + 40 μg | 0.8490 | 0.1462 | 0.7989 | 0.9183 | 0.9021 | 0.6936 | 0.0697 |
| TQL1055 + 5 μg | | | | | | | |
| PHAD | | | | | | | |
| 5 μg gE + 40 μg | 0.3959 | 0.7099 | 0.8216 | 0.9381 | 0.8238 | 0.9930 | 0.8525 |
| TQL1055 + 1 μg | | | | | | | |
| EcML | | | | | | | |
| 5 μg gE + 40 μg | >0.9999 | 0.0277 | 0.3475 | 0.9977 | 0.6022 | 0.9463 | 0.9335 |
| TQL1055 + 5 μg | | | | | | | |
| EcML | | | | | | | |
| 5 μg gE + 40 μg | 0.2085 | 0.2355 | 0.0339 | 0.9009 | 0.6053 | 0.9358 | 0.8628 |
| TQL1055 + 1 μg | | | | | | | |
| TLR4a-1 | | | | | | | |
| 5 μg gE + 40 μg | 0.4642 | 0.0004 | 0.5920 | 0.6786 | 0.7001 | 0.3497 | 0.6467 |
| TQL1055 + 5 μg | | | | | | | |
| TLR4a-1 | | | | | | | |
| 5 μg gE + 40 μg | 0.2989 | 0.0671 | 0.9149 | 0.9009 | 0.7023 | 0.9288 | 0.5770 |
| TQL1055 + 0.5 μg | | | | | | | |
| CRX-527 | | | | | | | |
| 5 μg gE + 40 μg | 0.6818 | 0.0327 | 0.1307 | 0.8375 | 0.7685 | 0.9755 | 0.4267 |
| TQL1055 + 1 μg | | | | | | | |
| CRX-527 | | | | | | | |

### Example 6—Immunogenicity of TQL-1055+TLR4 Agonist Candidates in C57BL/6J Mice

The tolerability and immunogenicity of formulations containing (TQL-1055 free acid HP-β-CD (2-hydroxypropyl beta-cyclodextrin) and TLR4 agonist candidates were studied. CD-1 mice were immunized as shown in the table below. PHAD liposomes were obtained from Avanti. EcML was obtained from EuBiologics. CRX-527 was obtained from InvivoGen. TLR4a-1 is an undisclosed TLR4 compound. AS01 is a combination of 5 mcg QS-21 and 5 mcg MPL used in SHINGRIX.

The study was conducted in two parts with staggered start dates. Part A involved testing TQL1055 in combination with PHAD and EcML. Part B involved testing TQL1055 in combination with CRX-527 and TR4a-1.

Part A

| Group | BL/6 Mice | gE | TQL1055 FA HPβCD | PHAD | EcML |
|---|---|---|---|---|---|
| 1 | 10 | 5 μg | — | — | — |
| 2 | 10 | 5 μg | 15 μg | — | — |
| 3 | 10 | 5 μg | 40 μg | — | — |
| 4 | 10 | 5 μg | 80 μg | — | — |
| 5 | 10 | 5 μg | — | 0.5 μg | — |
| 6 | 10 | 5 μg | — | 1 μg | — |
| 7 | 10 | 5 μg | — | 5 μg | — |
| 8 | 10 | 5 μg | 15 μg | 0.5 μg | — |
| 9 | 10 | 5 μg | 40 μg | 0.5 μg | — |
| 10 | 10 | 5 μg | 80 μg | 0.5 μg | — |
| 11 | 10 | 5 μg | 15 μg | 1 μg | — |
| 12 | 10 | 5 μg | 40 μg | 1 μg | — |
| 13 | 10 | 5 μg | 80 μg | 1 μg | — |
| 14 | 10 | 5 μg | 15 μg | 5 μg | — |
| 15 | 10 | 5 μg | 40 μg | 5 μg | — |
| 16 | 10 | 5 μg | 80 μg | 5 μg | — |
| 17 | 10 | 5 μg | — | — | 0.5 μg |
| 18 | 10 | 5 μg | — | — | 1 μg |
| 19 | 10 | 5 μg | — | — | 5 μg |
| 20 | 10 | 5 μg | 15 μg | — | 0.5 μg |
| 21 | 10 | 5 μg | 40 μg | — | 0.5 μg |
| 22 | 10 | 5 μg | 80 μg | — | 0.5 μg |
| 23 | 10 | 5 μg | 15 μg | — | 1 μg |
| 24 | 10 | 5 μg | 40 μg | — | 1 μg |
| 25 | 10 | 5 μg | 80 μg | — | 1 μg |
| 26 | 10 | 5 μg | 15 μg | — | 5 μg |
| 27 | 10 | 5 μg | 40 μg | — | 5 μg |
| 28 | 10 | 5 μg | 80 μg | — | 5 μg |

| | | | |
|---|---|---|---|
| 29 | 10 | 5 μg | % μg AS01 (5 μg MPL + 5 μg QS-21) |

Part B

| | | | TQL1055 FA | | |
|---|---|---|---|---|---|
| Group | BL/6 Mice | gE | HPβCD | CRX-527 | TLR4a-1 |
| 1 | 10 | 5 μg | — | — | — |
| 2 | 10 | 5 μg | 15 μg | — | — |
| 3 | 10 | 5 μg | 40 μg | — | — |
| 4 | 10 | 5 μg | 80 μg | — | — |
| 5 | 10 | 5 μg | — | 0.5 μg | — |
| 6 | 10 | 5 μg | — | 1 μg | — |
| 7 | 10 | 5 μg | — | 5 μg | — |
| 8 | 10 | 5 μg | 15 μg | 0.5 μg | — |
| 9 | 10 | 5 μg | 40 μg | 0.5 μg | — |
| 10 | 10 | 5 μg | 80 μg | 0.5 μg | — |
| 11 | 10 | 5 μg | 15 μg | 1 μg | — |
| 12 | 10 | 5 μg | 40 μg | 1 μg | — |
| 13 | 10 | 5 μg | 80 μg | 1 μg | — |
| 14 | 10 | 5 μg | 15 μg | 5 μg | — |
| 15 | 10 | 5 μg | 40 μg | 5 μg | — |
| 16 | 10 | 5 μg | 80 μg | 5 μg | — |
| 17 | 10 | 5 μg | — | — | 0.5 μg |
| 18 | 10 | 5 μg | — | — | 1 μg |
| 19 | 10 | 5 μg | — | — | 5 μg |
| 20 | 10 | 5 μg | 15 μg | — | 0.5 μg |
| 21 | 10 | 5 μg | 40 μg | — | 0.5 μg |
| 22 | 10 | 5 μg | 80 μg | — | 0.5 μg |
| 23 | 10 | 5 μg | 15 μg | — | 1 μg |
| 24 | 10 | 5 μg | 40 μg | — | 1 μg |
| 25 | 10 | 5 μg | 80 μg | — | 1 μg |
| 26 | 10 | 5 μg | 15 μg | — | 5 μg |
| 27 | 10 | 5 μg | 40 μg | — | 5 μg |
| 28 | 10 | 5 μg | 80 μg | — | 5 μg |

| | | | |
|---|---|---|---|
| 29 | 10 | 5 μg | 5 μg AS01 (5 μg MPL + 5 μg QS-21) |

Mice were immunized via I.M. on Day 0 and Day 14. Groups were bled at Day-1, Day 13 (post Dose 1), and Day 28 (post Dose 2) for serum analysis for anti-gE specific antibody response. Weight measurements were also obtained on Days 0, 1, 2, 14, 15, 16, and 17. The results are shown below.

### Weight Loss

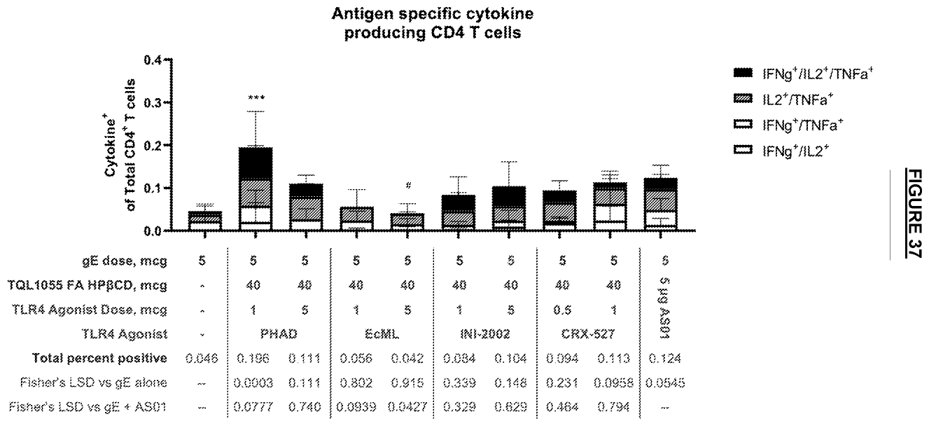

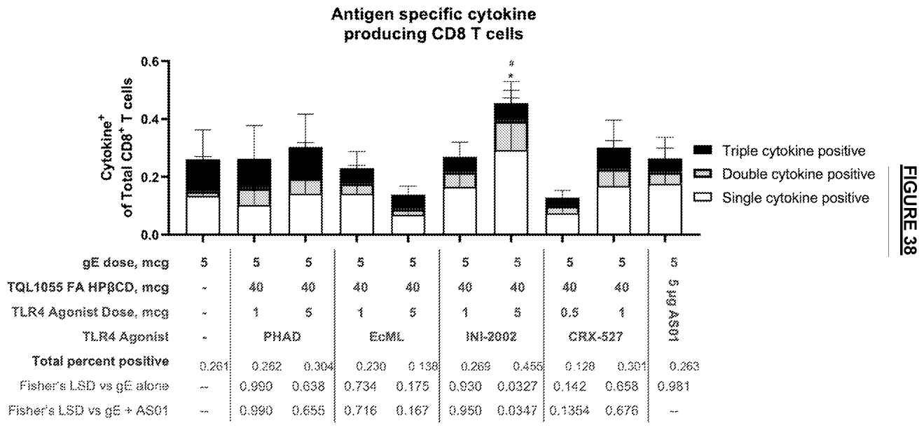

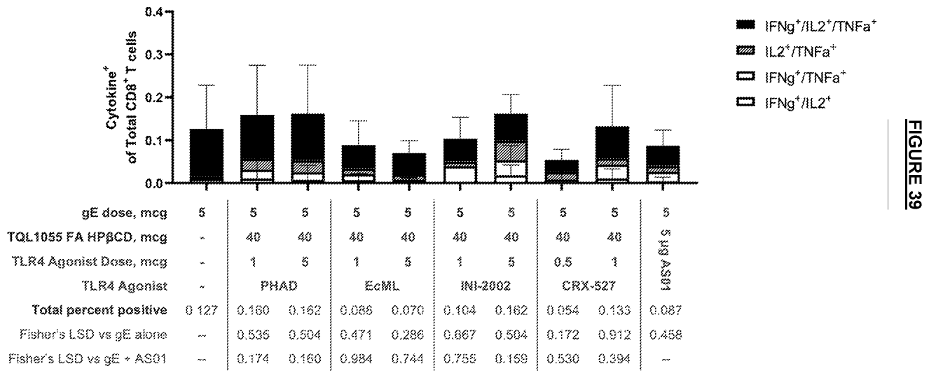

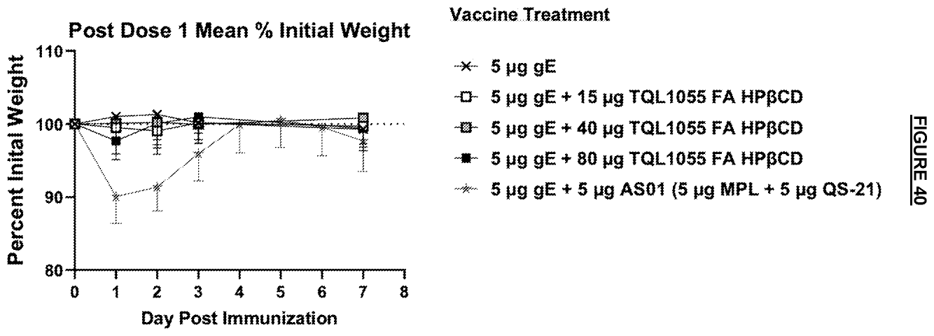

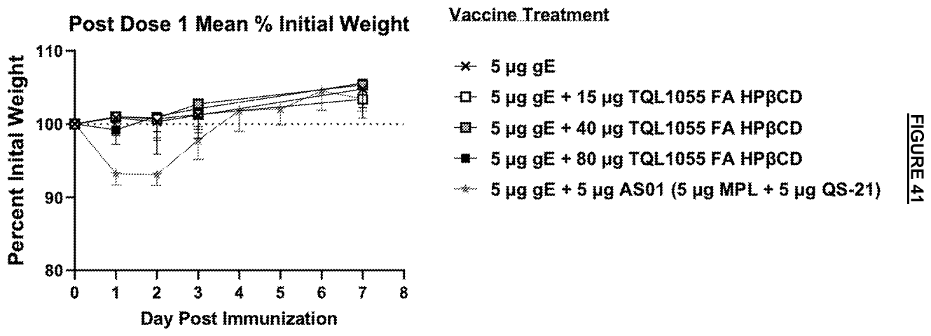

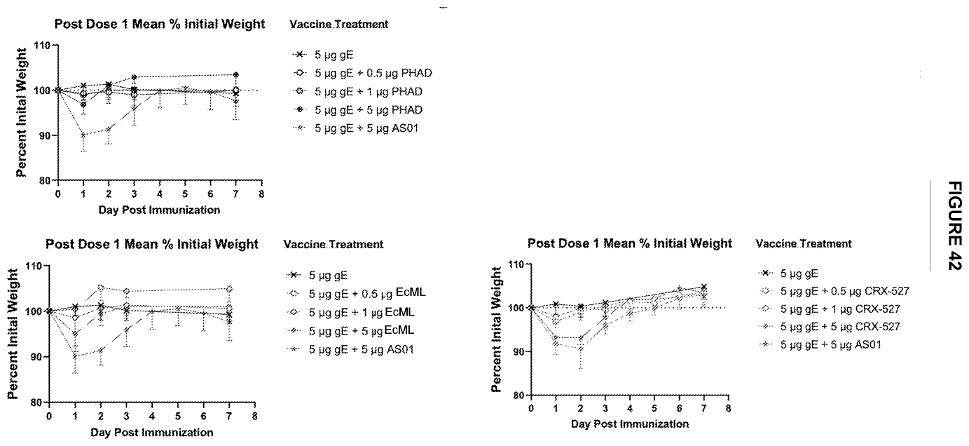

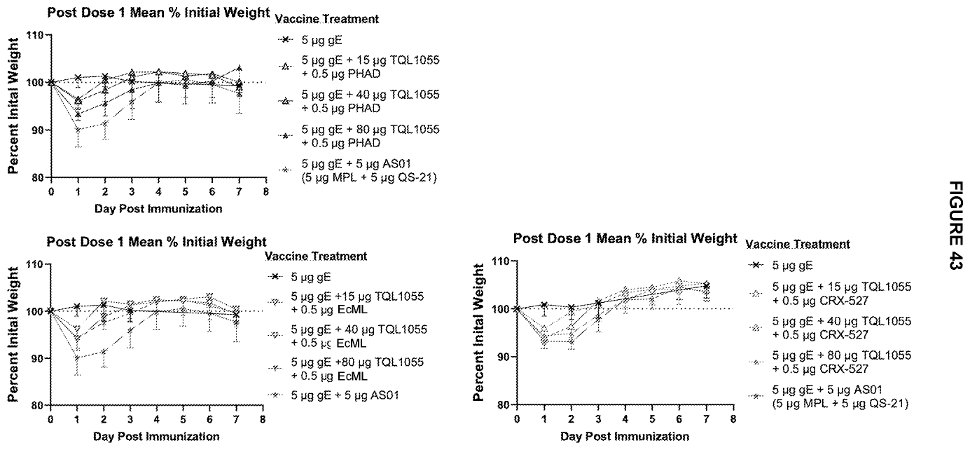

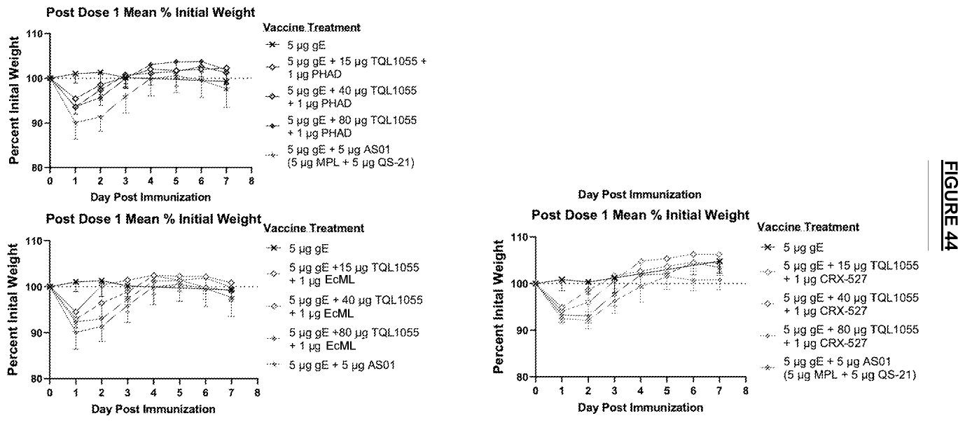

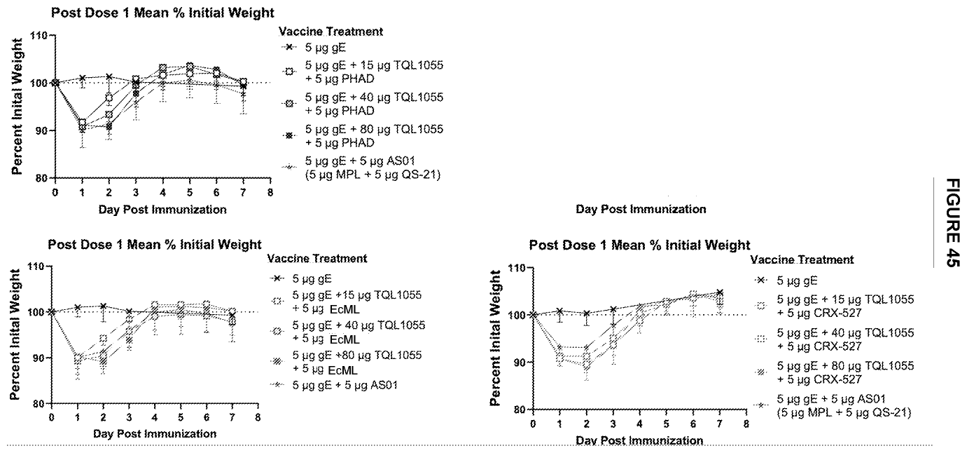

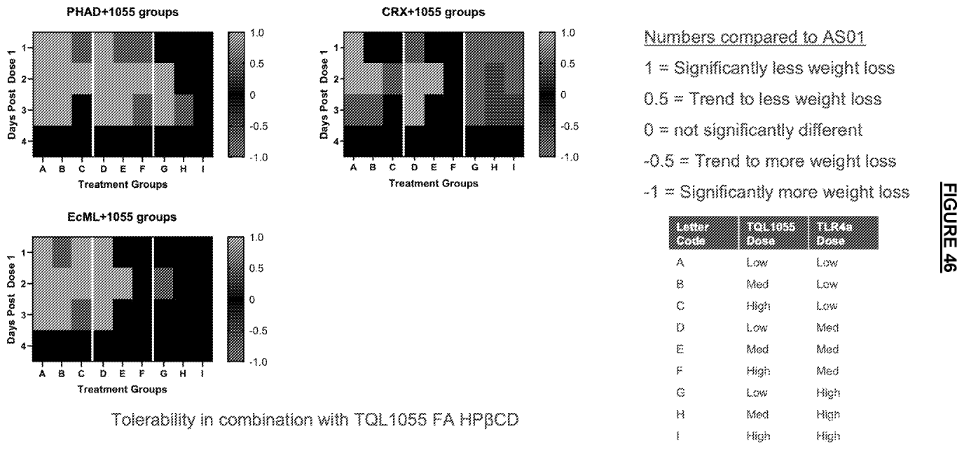

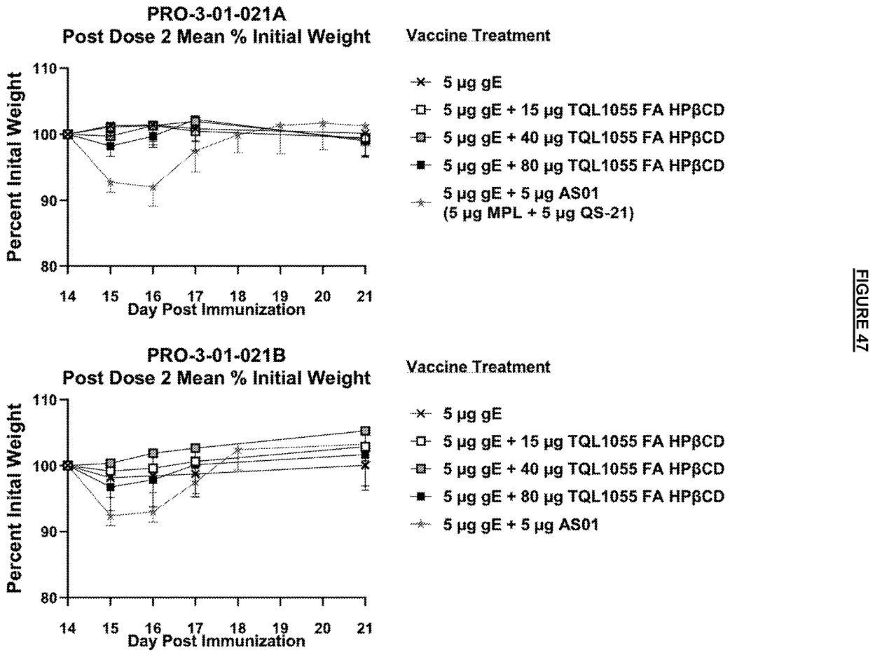

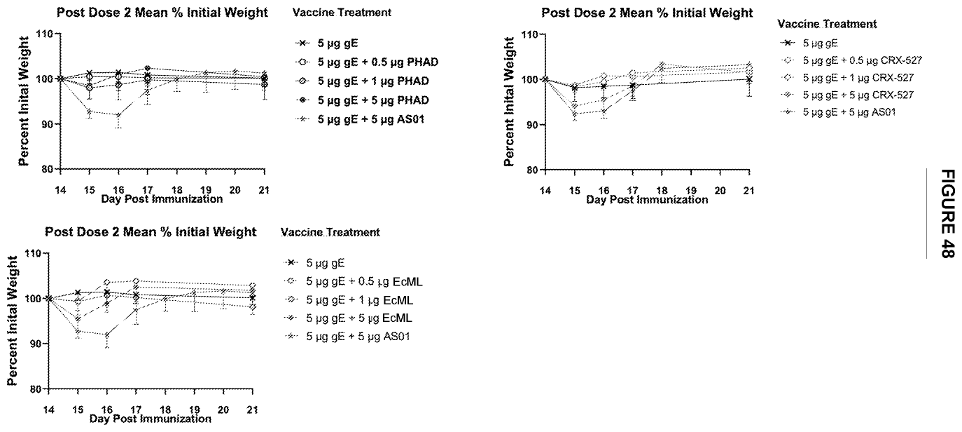

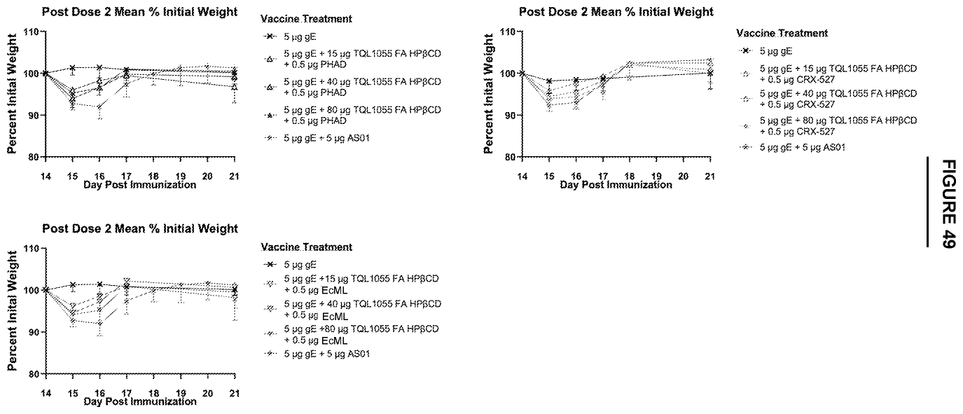

Post dose 1 mean percent (%) initial weight change for each group is depicted in FIGS. 40-45. Post dose 1 weight loss heatmaps are depicted in FIG. 46. Post dose 2 mean percent (%) initial weight change for each group is depicted in FIGS. 47-51. Two-way ANOVA (analysis of variance) test results are shown below. TQL 1055 alone induced no significant weight loss compared to unadjuvanted. Weight loss appeared to be driven mostly by TLR4 dose.

| 2 way ANOVA of Day 1 | P-value compared to gE on Day 1 |
|---|---|
| 5 μg gE | — |
| 5 μg gE + 0.5 μg PHAD | 0.3266 |
| 5 μg gE + 1 μg PHAD | 0.1586 |
| 5 μg gE + 5 μg PHAD | 0.0039 |
| 5 μg gE + 0.5 μg EcML | 0.9574 |
| 5 μg gE + 1 μg EcML | 0.9997 |
| 5 μg gE + 5 μg EcML | 0.0003 |
| 5 μg gE + 0.5 μg CRX-527 | 0.0652 |
| 5 μg gE + 1 μg CRX-527 | 0.0069 |
| 5 μg gE + 5 μg CRX-527 | <0.0001 |

| 2 way ANOVA compared to gE + AS01 | D1 | D2 | D3 | D4 |
|---|---|---|---|---|
| 5 μg gE + 15 μg TQL1055 + 0.5 μg PHAD | 0.0030 | 0.0004 | 0.0205 | 0.5807 |
| 5 μg gE + 40 μg TQL1055 + 0.5 μg PHAD | 0.0018 | <0.0001 | 0.0027 | 0.4834 |
| 5 μg gE + 80 μg TQL1055 + 0.5 μg PHAD | 0.1209 | 0.0410 | 0.6418 | >0.9999 |
| 5 μg gE + 15 μg TQL1055 + 0.5 μg EcML | 0.0030 | <0.0001 | 0.0125 | 0.4631 |
| 5 μg gE + 40 μg TQL1055 + 0.5 μg EcML | 0.0655 | 0.0002 | 0.0090 | 0.6494 |
| 5 μg gE + 80 μg TQL1055 + 0.5 μg EcML | 0.0451 | 0.0008 | 0.0960 | 0.6113 |
| 5 μg gE + 15 μg TQL1055 + 0.5 μg CRX | 0.0249 | 0.0001 | 0.1207 | 0.9242 |
| 5 μg gE + 40 μg TQL1055 + 0.5 μg CRX | 0.8367 | 0.0180 | 0.0597 | 0.6281 |
| 5 μg gE + 80 μg TQL1055 + 0.5 μg CRX | 0.4771 | 0.0786 | 0.9501 | 0.9999 |
| 5 μg gE + 15 μg TQL1055 + 1 μg PHAD | 0.0086 | 0.0002 | 0.0414 | 0.6109 |
| 5 μg gE + 40 μg TQL1055 + 1 μg PHAD | 0.0913 | 0.0015 | 0.0196 | 0.9644 |
| 5 μg gE + 80 μg TQL1055 + 1 μg PHAD | 0.0913 | 0.0175 | 0.0703 | 0.2232 |
| 5 μg gE + 15 μg TQL1055 + 1 μg EcML | 0.0433 | <0.0001 | 0.0082 | 0.3477 |
| 5 μg gE + 40 μg TQL1055 + 1 μg EcML | 0.2176 | 0.0116 | 0.2459 | 0.3894 |
| 5 μg gE + 80 μg TQL1055 + 1 μg EcML | 0.3895 | 0.6717 | 0.9309 | 0.9554 |
| 5 μg gE + 15 μg TQL1055 + 1 μg CRX | 0.0836 | <0.0001 | 0.0439 | 0.9962 |
| 5 μg gE + 40 μg TQL1055 + 1 μg CRX | 0.8593 | 0.0165 | 0.2464 | 0.1628 |
| 5 μg gE + 80 μg TQL1055 + 1 μg CRX | 0.8113 | 0.8311 | 0.8056 | 0.4890 |

| 2 way ANOVA | | | | |
|---|---|---|---|---|
| compared to gE + | | | | |
| AS01 | D1 | D2 | D3 | D4 |
| 5 μg gE + 15 μg | 0.7143 | 0.0025 | 0.0167 | 0.8222 |
| TQL1055 + 5 μg | | | | |
| PHAD | | | | |
| 5 μg gE + 40 μg | 0.9927 | 0.4461 | 0.1246 | 0.2409 |
| TQL1055 + 5 μg | | | | |
| PHAD | | | | |
| 5 μg gE + 80 μg | 0.9619 | 0.9968 | 0.7745 | 0.8248 |
| TQL1055 + 5 μg | | | | |
| PHAD | | | | |
| 5 μg gE + 15 μg | >0.9999 | 0.1358 | 0.3666 | 0.7698 |
| TQL1055 + 5 μg | | | | |
| EcML | | | | |
| 5 μg gE + 40 μg | 0.9976 | 0.9943 | 0.9999 | 0.9971 |
| TQL1055 + 5 μg | | | | |
| EcML | | | | |
| 5 μg gE + 80 μg | 0.9999 | 0.3448 | 0.5334 | 0.9694 |
| TQL1055 + 5 g | | | | |
| EcML | | | | |
| 5 μg gE + 15 μg | 0.0141 | 0.0019 | 0.0146 | 0.1525 |
| TQL1055 + 5 μg | | | | |
| CRX | | | | |
| 5 μg gE + 40 μg | 0.0419 | 0.0804 | 0.1423 | 0.8285 |
| TQL1055 + 5 μg | | | | |
| CRX | | | | |
| 5 μg gE + 80 μg | 0.0251 | 0.0189 | 0.0979 | 0.9995 |
| TQL1055 + 5 μg | | | | |
| CRX | | | | |

| 2 way ANOVA of Day 15 | P-value compared to gE on Day 15 |
|---|---|
| 5 μg gE | — |
| 5 μg gE + 0.5 μg PHAD | 0.9197 |
| 5 μg gE + 1 μg PHAD | 0.0290 |
| 5 μg gE + 5 μg PHAD | 0.0050 |
| 5 μg gE + 0.5 μg EcML | 0.2645 |
| 5 μg gE + 1 μg EcML | 0.4896 |
| 5 μg gE + 5 μg EcML | 0.0011 |
| 5 μg gE + 0.5 μg CRX-527 | 0.9997 |
| 5 μg gE + 1 μg CRX-527 | 0.9993 |
| 5 μg gE + 5 μg CRX-527 | 0.0313 |

| 2 way ANOVA compared to gE + AS01 | D15 | D16 | D17 |
|---|---|---|---|
| 5 μg gE + 15 μg TQL1055 + 0.5 μg PHAD | 0.0007 | 0.0004 | 0.6950 |
| 5 μg gE + 40 μg TQL1055 + 0.5 μg PHAD | 0.7355 | 0.0065 | 0.3599 |
| 5 μg gE + 80 μg TQL1055 + 0.5 μg PHAD | 0.0130 | 0.0084 | 0.0666 |
| 5 μg gE + 15 μg TQL1055 + 0.5 μg EcML | 0.0085 | 0.0007 | 0.2317 |
| 5 μg gE + 40 μg TQL1055 + 0.5 μg EcML | 0.1774 | 0.0033 | 0.0202 |
| 5 μg gE + 80 μg TQL1055 + 0.5 μg EcML | 0.3059 | 0.1264 | 0.1756 |
| 5 μg gE + 15 μg TQL1055 + 0.5 μg CRX | 0.0998 | 0.0653 | 0.9038 |
| 5 μg gE + 40 μg TQL1055 + 0.5 μg CRX | 0.0694 | 0.0242 | 0.9991 |
| 5 μg gE + 80 μg TQL1055 + 0.5 μg CRX | 0.2654 | 0.8161 | 0.9997 |

| 2 way ANOVA | | | |
|---|---|---|---|
| compared to gE + | | | |
| AS01 | D15 | D16 | D17 |
| 5 μg gE + 15 μg | 0.2355 | 0.0639 | 0.9396 |
| TQL1055 + 1 μg | | | |
| PHAD | | | |
| 5 μg gE + 40 μg | 0.8508 | 0.0558 | 0.9957 |
| TQL1055 + 1 μg | | | |
| PHAD | | | |
| 5 μg gE + 80 μg | 0.8859 | 0.1683 | >0.9999 |
| TQL1055 + 1 μg | | | |
| PHAD | | | |
| 5 μg gE + 15 μg | 0.0371 | 0.0016 | 0.0658 |
| TQL1055 + 1 μg | | | |
| EcML | | | |
| 5 μg gE + 40 μg | 0.1801 | 0.0328 | 0.3221 |
| TQL1055 + 1 μg | | | |
| EcML | | | |
| 5 μg gE + 80 μg | 0.9911 | 0.9998 | 0.9999 |
| TQL1055 + 1 μg | | | |
| EcML | | | |
| 5 μg gE + 15 μg | 0.0243 | 0.0068 | 0.7038 |
| TQL1055 + 1 μg | | | |
| CRX | | | |
| 5 μg gE + 40 μg | 0.0317 | 0.0035 | 0.1080 |
| TQL1055 + 1 μg | | | |
| CRX | | | |
| 5 μg gE + 80 μg | 0.5932 | >0.9999 | 0.9960 |
| TQL1055 + 1 μg | | | |
| CRX | | | |

| 2 way ANOVA | | | |
|---|---|---|---|
| compared to gE + | | | |
| AS01 | D15 | D16 | D17 |
| 5 μg gE + 15 μg | 0.9161 | 0.3662 | 0.9074 |
| TQL1055 + 5 μg | | | |
| PHAD | | | |
| 5 μg gE + 40 μg | 0.9903 | 0.9997 | 0.9993 |
| TQL1055 + 5 μg | | | |
| PHAD | | | |
| 5 μg gE + 80 μg | 0.0213 | 0.1202 | 0.2027 |
| TQL1055 + 5 μg | | | |
| PHAD | | | |
| 5 μg gE + 15 μg | 0.0631 | 0.3031 | 0.9276 |
| TQL1055 + 5 μg | | | |
| EcML | | | |
| 5 μg gE + 40 μg | 0.9631 | 0.9998 | 0.9662 |
| TQL1055 + 5 μg | | | |
| EcML | | | |
| 5 μg gE + 80 μg | 0.0920 | 0.0488 | 0.0164 |
| TQL1055 + 5 μg | | | |
| EcML | | | |
| 5 μg gE + 15 μg | 0.0100 | 0.0216 | 0.4180 |
| TQL1055 + 5 μg CRX | | | |
| 5 μg gE + 40 μg | 0.5046 | 0.9957 | 0.9999 |
| TQL1055 + 5 μg CRX | | | |
| 5 μg gE + 80 μg | 0.5791 | 0.0004 | 0.0020 |
| TQL1055 + 5 μg CRX | | | |

### Anti-gE ELISA

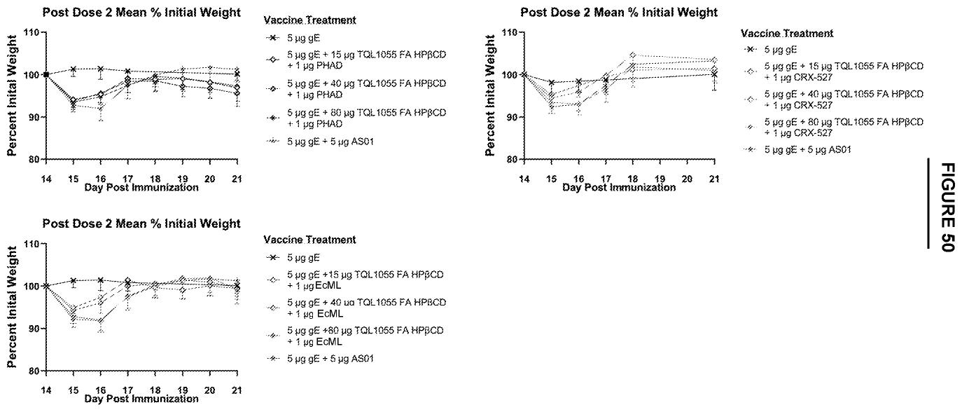

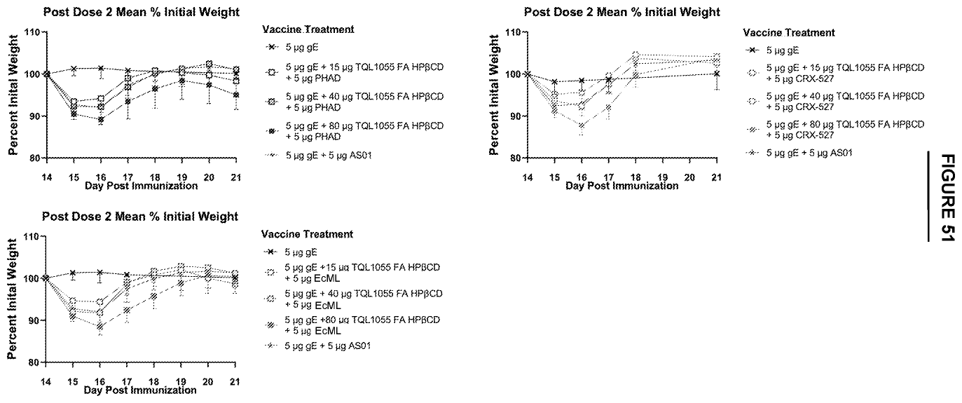

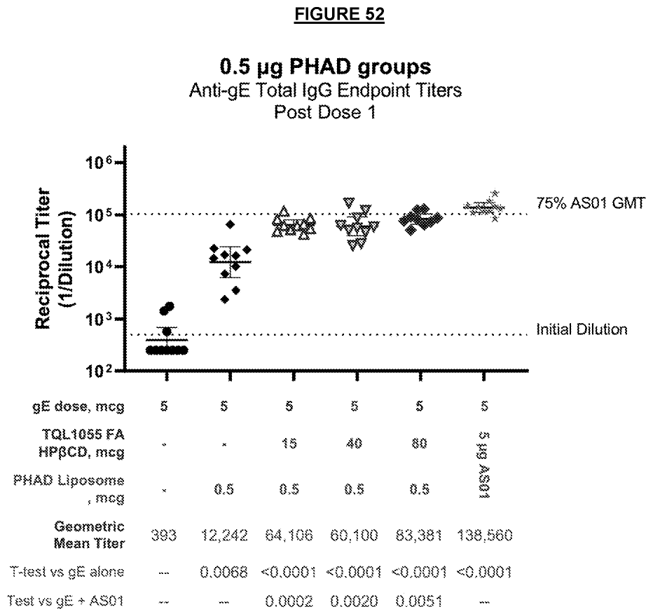

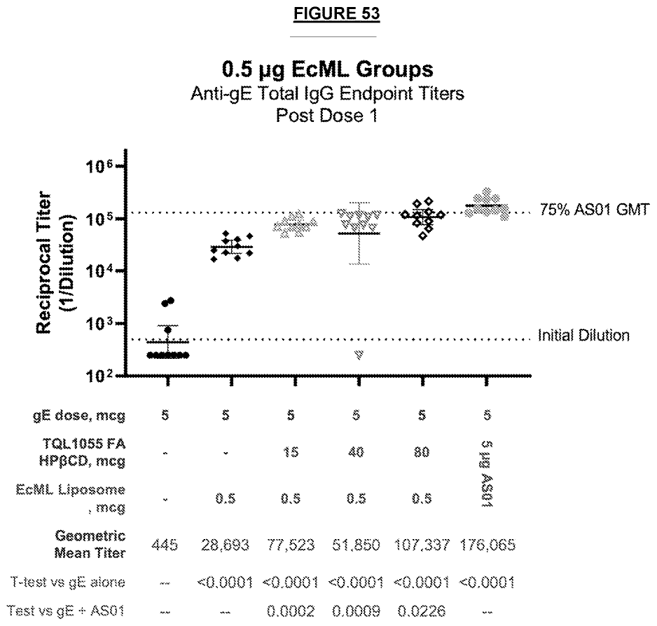

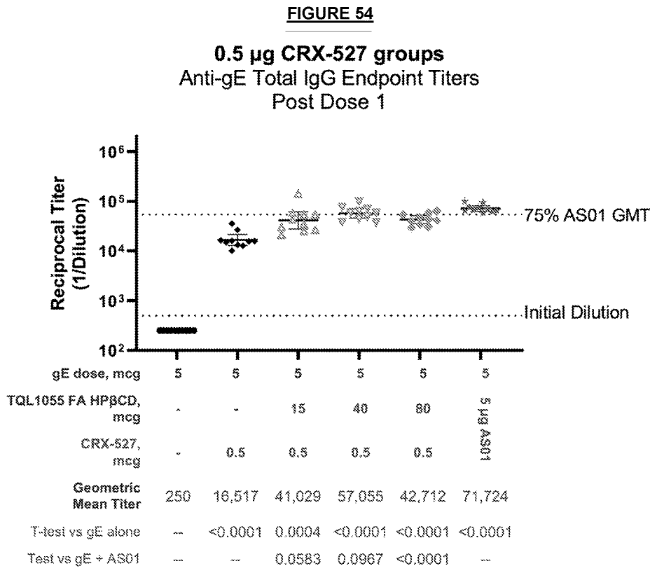

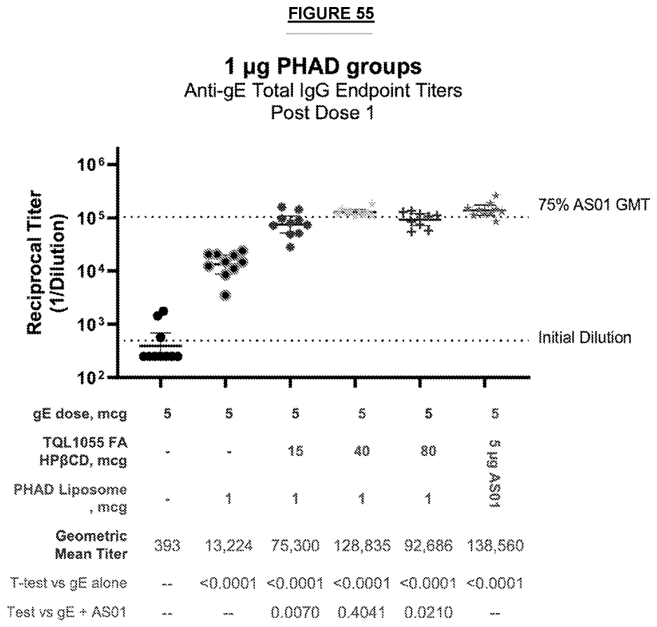

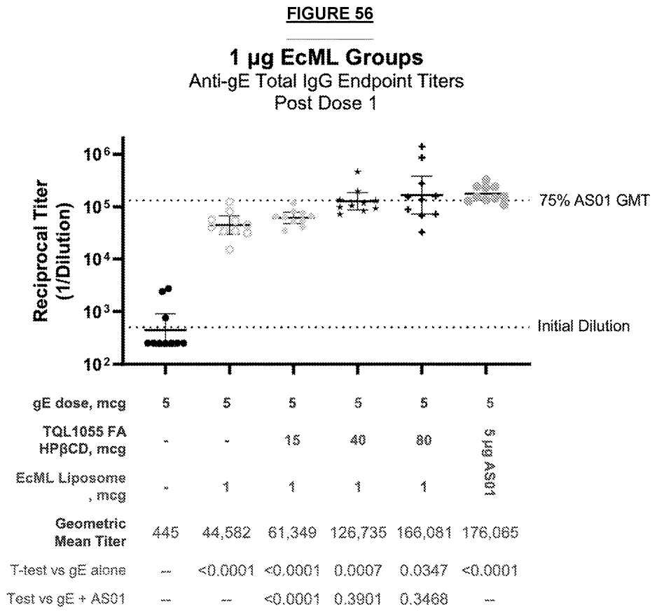

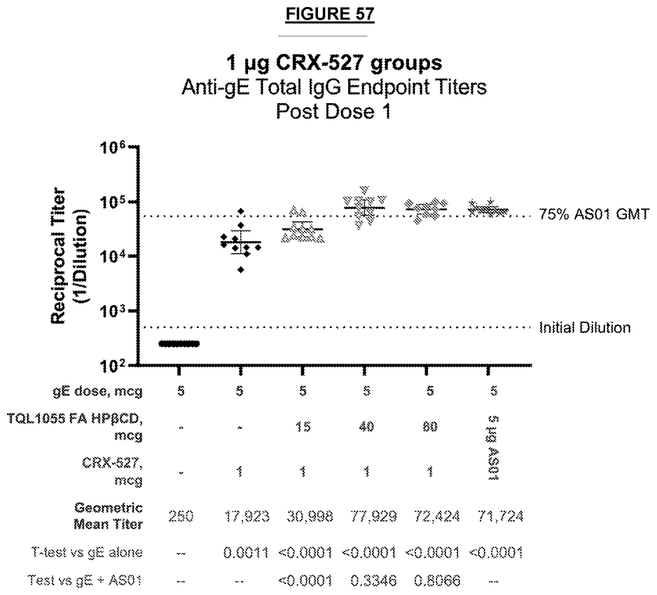

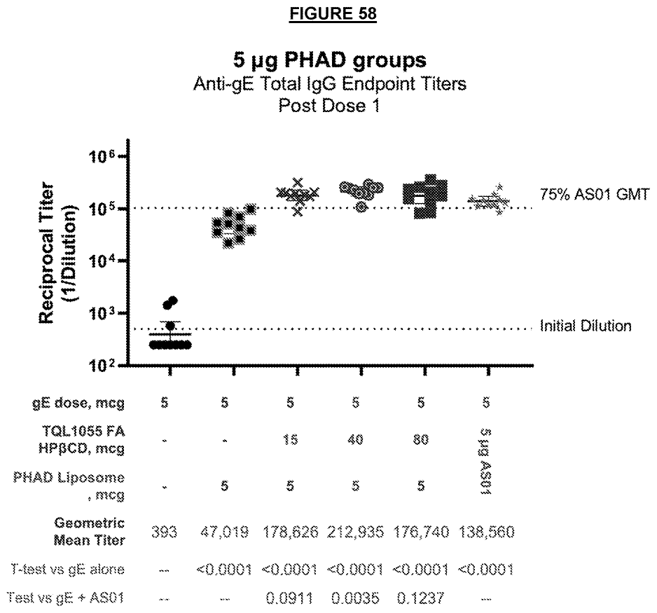

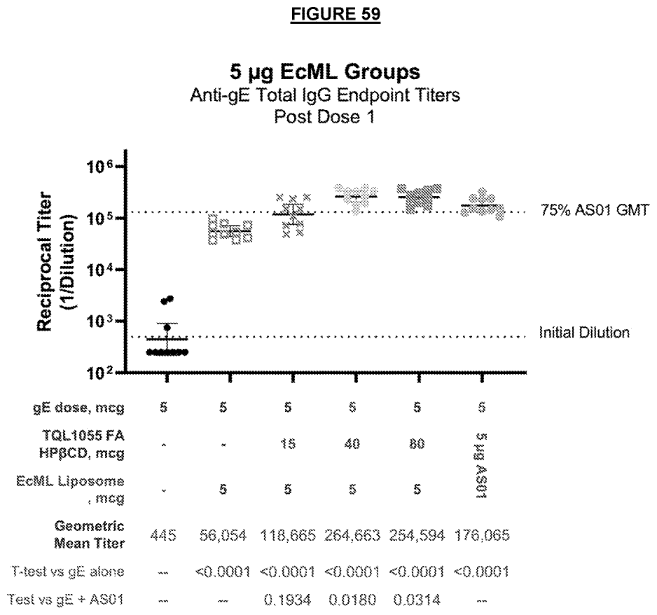

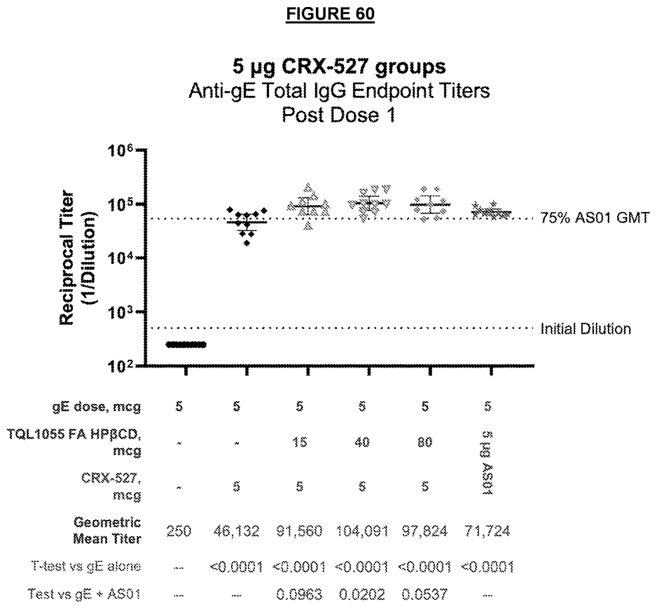

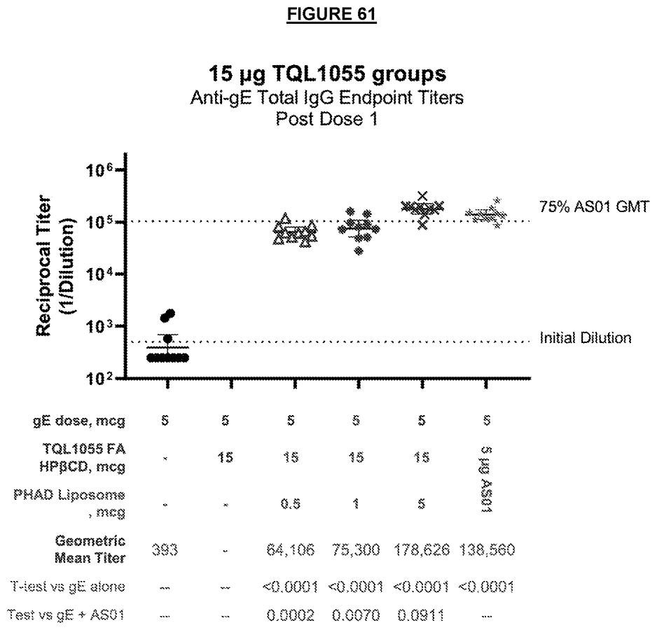

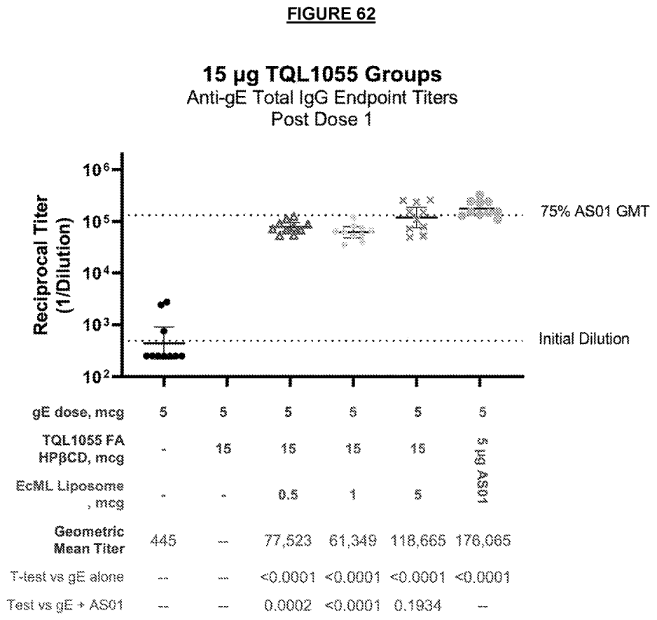

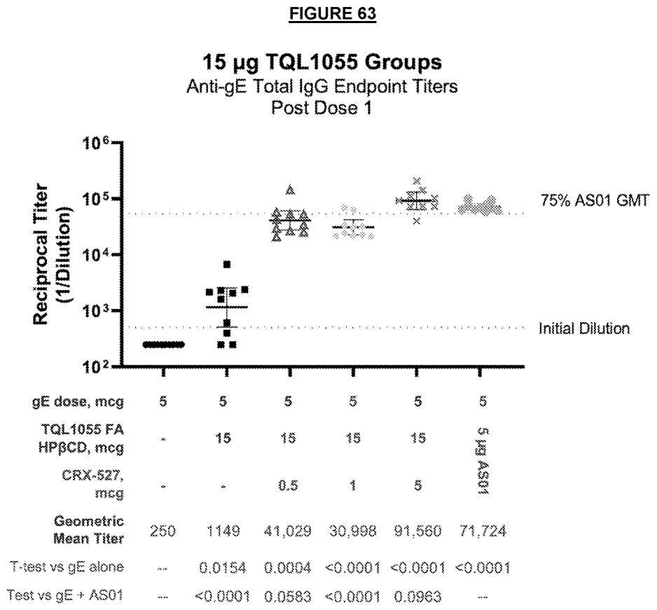

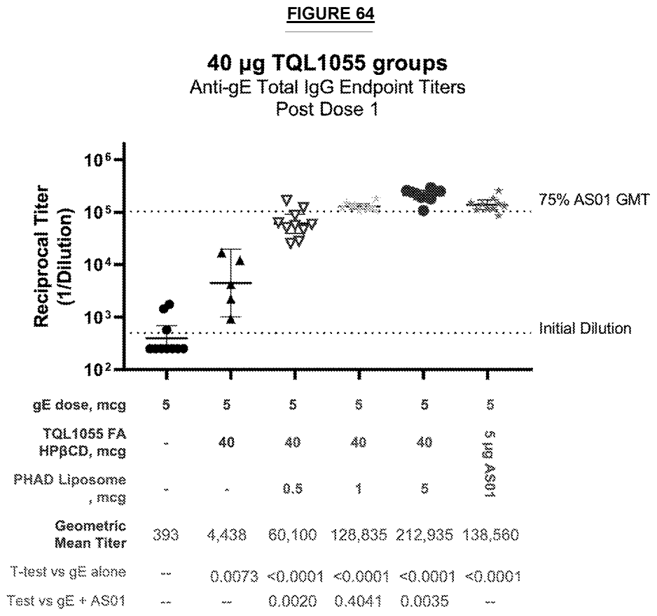

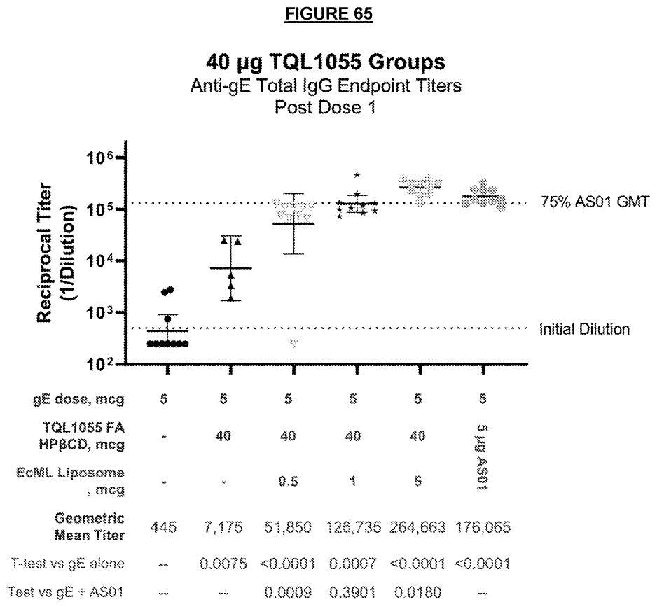

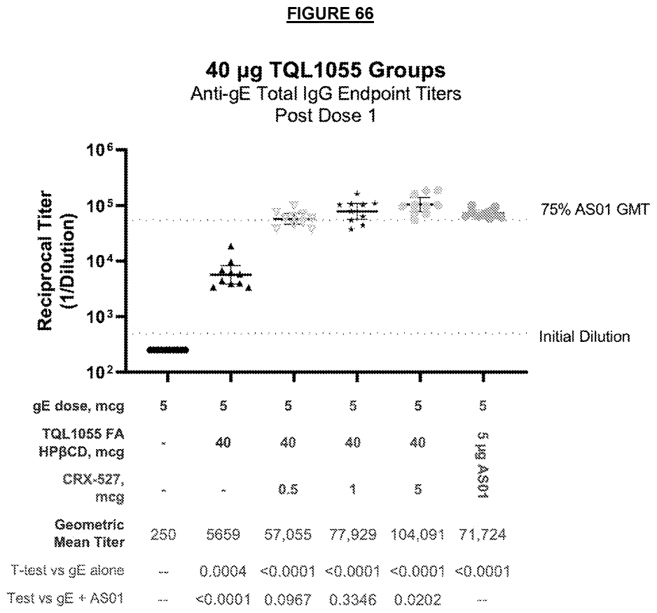

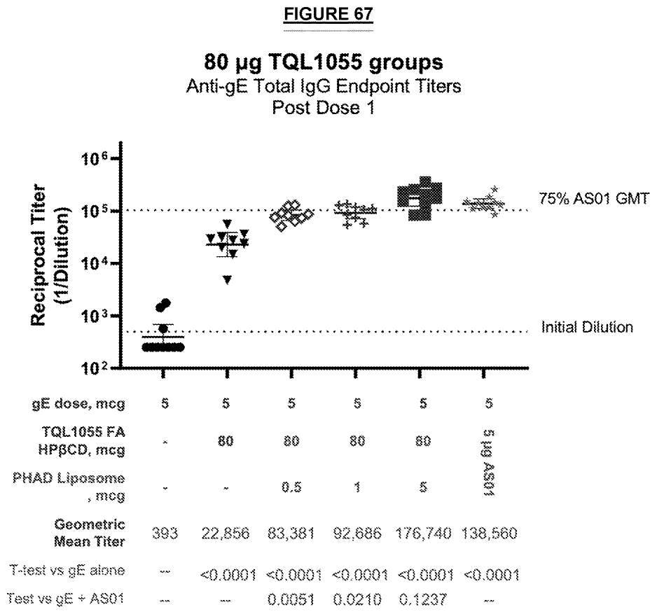

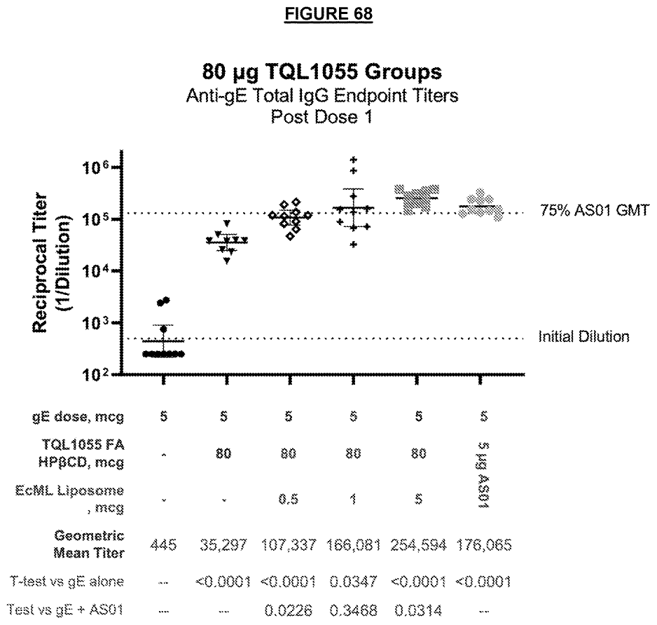

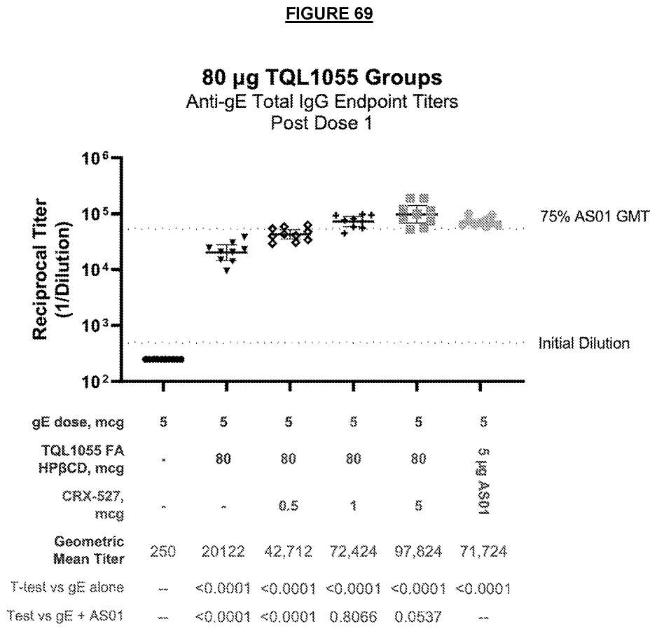

D13 (post dose 1) total IgG endpoint titers are shown in FIGS. 52-69. TQL 1055+PHAD boosts titers over either adjuvant alone. 80 μg TQL 1055 dose did not consistently improve titers over 40 μg dose when in combination with PHAD-maximal synergy may be achieved at TQL1055 doses at or below 40 μg.

TQL1055+EcML significantly boosts titers over either adjuvant alone. TQL1055 had a dose dependent increase in titers in combination with 0.5 or 1 μg EcML. At 5 μg EcML+TQL1055, there is no added benefit between 40 and 80 μg TQL1055. Maximal synergy may be achieved at TQL1055 doses at or below 40 μg.

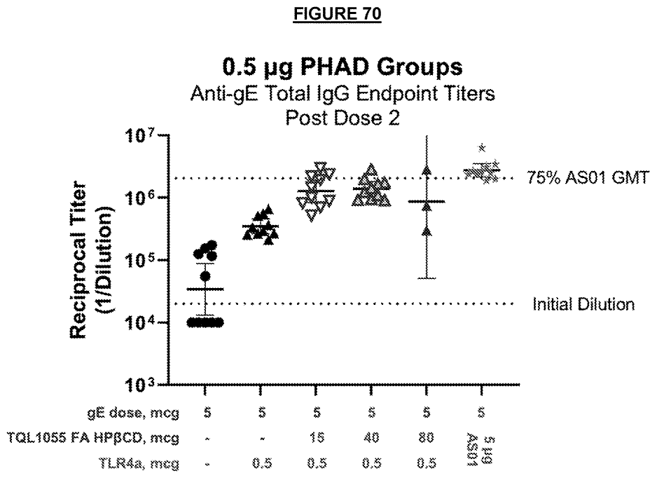

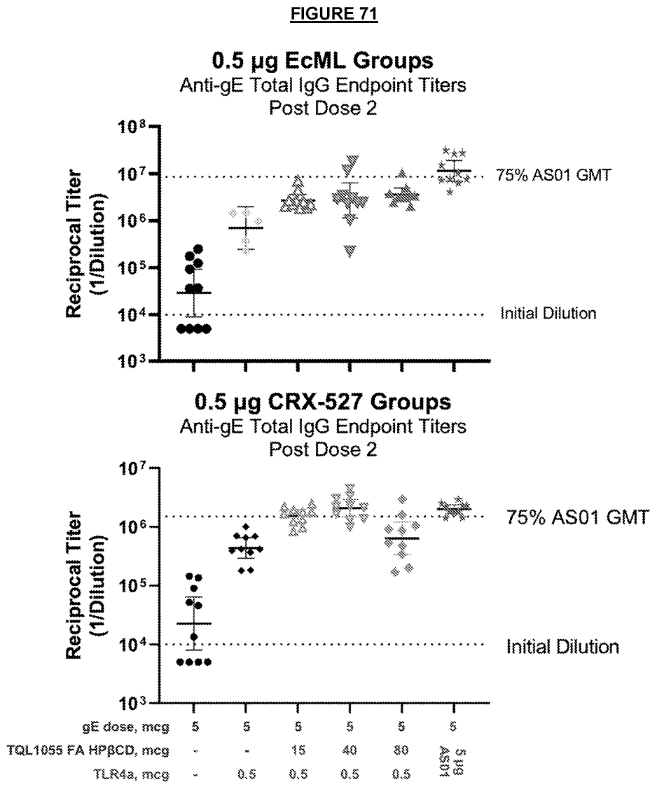

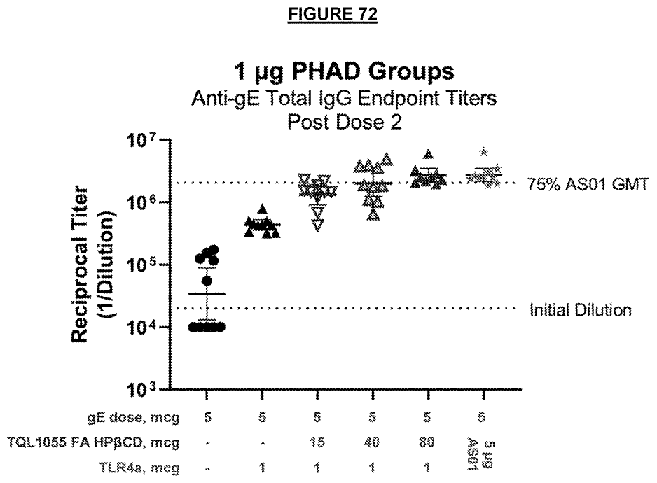

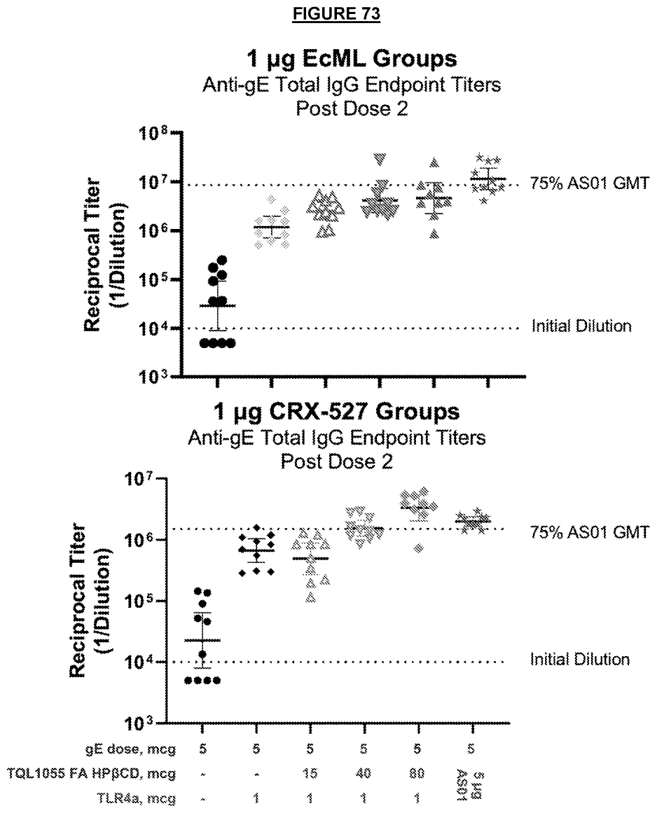

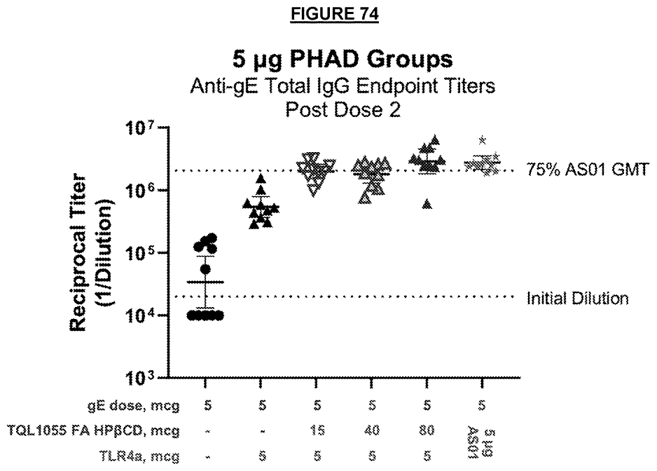

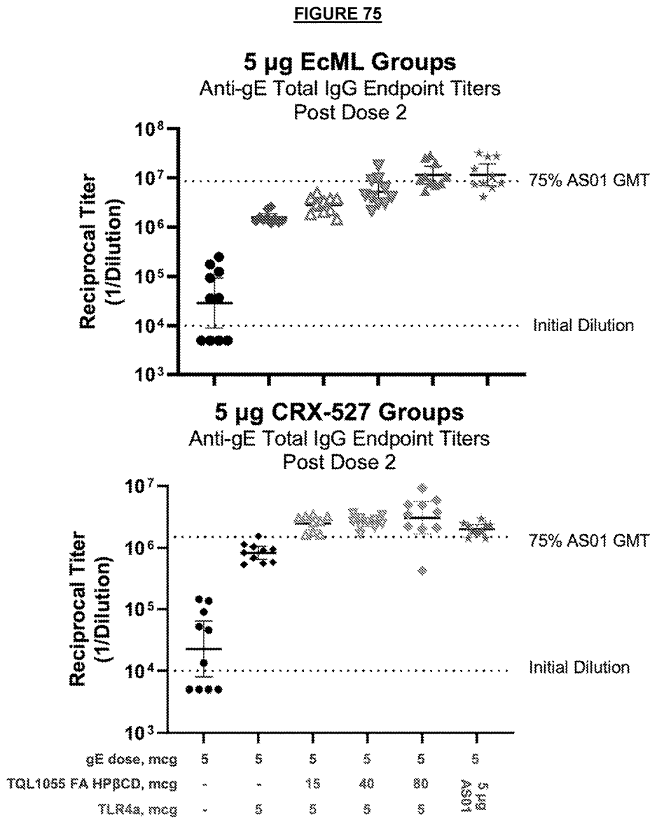

D28 (post dose 2) total IgG endpoint titers are shown in FIGS. 70-75. D28 T-test results vs. unadjuvanted are shown below.

| T-test v unadjuvanted | PHAD | EcML | CRX-527 |
|---|---|---|---|
| 5 μg gE + 15 μg TQL1055 | 0.0155 | 0.0242 | 0.0057 |
| 5 μg gE + 40 μg TQL1055 | <0.0001 | 0.0010 | <0.0001 |
| 5 μg gE + 80 μg TQL1055 | 0.0001 | <0.0001 | <0.0001 |
| 5 μg gE + 0.5 μg TLR4a | <0.0001 | 0.0005 | <0.0001 |
| 5 μg gE + 1 μg TLR4a | <0.0001 | 0.0014 | <0.0001 |
| 5 μg gE + 5 μg TLR4a | 0.0004 | <0.0001 | <0.0001 |
| 5 μg gE + 15 μg | <0.0001 | <0.0001 | <0.0001 |
| TQL1055 + 0.5 μg TLR4a | | | |
| 5 μg gE + 40 μg | <0.0001 | 0.0177 | <0.0001 |
| TQL1055 + 0.5 μg TLR4a | | | |
| 5 μg gE + 80 μg | 0.0086 | <0.0001 | 0.0045 |
| TQL1055 + 0.5 μg TLR4a | | | |
| 5 μg gE + 15 μg | <0.0001 | <0.0001 | 0.0004 |
| TQL1055 + 1 μg TLR4a | | | |
| 5 μg gE + 40 μg | <0.0001 | 0.0272 | <0.0001 |
| TQL1055 + 1 μg TLR4a | | | |
| 5 μg gE + 80 μg | <0.0001 | 0.0090 | <0.0001 |
| TQL1055 + 1 μg TLR4a | | | |
| 5 μg gE + 15 μg | <0.0001 | <0.0001 | <0.0001 |
| TQL1055 + 5 μg TLR4a | | | |
| 5 μg gE + 40 μg | <0.0001 | 0.0006 | <0.0001 |
| TQL1055 + 5 μg TLR4a | | | |
| 5 μg gE + 80 μg | <0.0001 | <0.0001 | 0.0001 |
| TQL1055 + 5 μg TLR4a | | | |
| 5 μg gE + 5 μg AS01 | <0.0001 | 0.0003 | <0.0001 |
| 5 μg gE + 15 μg | 0.0075 | 0.0026 | 0.0717 |
| TQL1055 + 0.5 μg TLR4a | | | |
| 5 μg gE + 40 μg | 0.0045 | 0.0185 | 0.5239 |
| TQL1055 + 0.5 μg TLR4a | | | |
| 5 μg gE + 80 μg | 0.0824 | 0.0058 | 0.0014 |
| TQL1055 + 0.5 μg TLR4a | | | |
| 5 μg gE + 15 μg | 0.0038 | 0.0024 | <0.0001 |
| TQL1055 + 1 μg TLR4a | | | |
| 5 μg gE + 40 μg | 0.4261 | 0.0609 | 0.1932 |
| TQL1055 + 1 μg TLR4a | | | |
| 5 μg gE + 80 μg | 0.92 | 0.0853 | 0.0046 |
| TQL1055 + 1 μg TLR4a | | | |
| 5 μg gE + 15 μg | 0.0906 | 0.0025 | 0.0765 |
| TQL1055 + 5 μg TLR4a | | | |
| 5 μg gE + 40 μg | 0.0508 | 0.0362 | 0.0176 |
| TQL1055 + 5 μg TLR4a | | | |
| 5 μg gE + 80 μg | 0.5584 | 0.7932 | 0.0343 |
| TQL1055 + 5 μg TLR4a | | | |

1. A pharmaceutical composition comprising
(a) a compound of Formula I 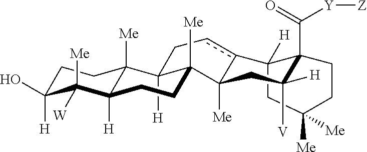
wherein [symbol]
is a single or double bond;
W is —CHO;
V is hydrogen or OR^{x};
Y is CH₂, —O—, —NR—, or —NH—;
Z is hydrogen; a cyclic or acyclic, optionally substituted moiety selected from the group consisting of acyl, aliphatic, heteroaliphatic, aryl, arylalkyl, heteroacyl, and heteroaryl; or a carbohydrate domain having the structure: 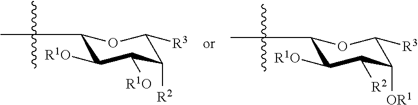
wherein each occurrence of R¹ is R^{x} or a carbohydrate domain having the structure: 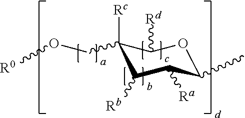
wherein:
each occurrence of a, b, and c is independently 0, 1, or 2;
d is an integer from 1-5, wherein each d bracketed structure may be the same or different; with the proviso that the d bracketed structure represents a furanose or a pyranose moiety, and the sum of b and c is 1 or 2;
R⁰ is hydrogen; an oxygen protecting group selected from the group consisting of alkyl ethers, benzyl ethers, silyl ethers, acetals, ketals, esters, carbamates, and carbonates; or an optionally substituted moiety selected from the group consisting of acyl, C₁₋₁₀ aliphatic, C₁₋₆ heteroaliphatic, 6-10-membered aryl, arylalkyl, 5-10 membered heteroaryl having 1-4 heteroatoms independently selected from nitrogen, oxygen, or sulfur, 4-7 membered heterocyclyl having 1-2 heteroatoms independently selected from the group consisting of nitrogen, oxygen, and sulfur;
each occurrence of R^{a}, R^{b}, R^{c}, and R^{d} is independently hydrogen, halogen, OH, OR, OR^{x}, NR₂, NHCOR, or an optionally substituted group selected from acyl, C₁₋₁₀ aliphatic, C₁₋₆ heteroaliphatic, 6-10-membered aryl, arylalkyl, 5-10-membered heteroaryl having 1-4 heteroatoms independently selected from nitrogen, oxygen, sulfur; 4-7-membered heterocyclyl having 1-2 heteroatoms independently selected from the group consisting of nitrogen, oxygen, and sulfur;
R² is hydrogen, halogen, OH, OR, OC(O)R⁴, OC(O)OR⁴, OC(O)NHR⁴, OC(O)NRR⁴, OC(O)SR⁴, NHC(O)R⁴, NRC(O)R⁴, NHC(O)OR⁴, NHC(O)NHR⁴, NHC(O)NRR⁴, NHR⁴, N(R⁴)₂, NHR⁴, NRR⁴, N₃, or an optionally substituted group selected from C₁₋₁₀ aliphatic, C₁₋₆ heteroaliphatic, 6-10-membered aryl, arylalkyl, 5-10 membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur;
R³ is hydrogen, halogen, CH₂OR¹, or an optionally substituted group selected from the group consisting of acyl, C₁₋₁₀ aliphatic, C₁₋₆ heteroaliphatic, 6-10-membered aryl, arylalkyl, 5-10-membered heteroaryl having 1-4 heteroatoms independently selected from the group consisting of nitrogen, oxygen, and sulfur, 4-7-membered heterocyclyl having 1-2 heteroatoms independently selected from the group consisting of nitrogen, oxygen, and sulfur,
R⁴ is -T-R^{z}, —C(O)-T-R^{z}, —NH-T-R^{z}, —O-T-R^{z}, —S-T-R^{z}, —C(O)NH-T-R^{z}, C(O)O-T-R^{z}, C(O)S-T-R^{z}, C(O)NH-T-O-T-R^{z}, —O-T-R^{z}, -T-O-T-R^{z}, -T-S-T-R^{z}, or 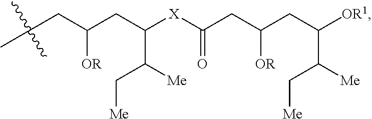
wherein
X is —O—, —NR—, or T-R^{z};
T is a covalent bond or a bivalent C₁₋₂₆ saturated or unsaturated, straight or branched, aliphatic or heteroaliphatic chain; and
R^{z} is hydrogen, halogen, —OR, —OR^{x}, —OR¹, —SR, NR₂, —C(O)OR, —C(O)R, —NHC(O)R, —NHC(O)OR, NC(O)OR, or an optionally substituted group selected from acyl, arylalkyl, heteroarylalkyl, C₁₋₆ aliphatic, 6-10-membered aryl, 5-10-membered heteroaryl having 1-4 heteroatoms independently selected from nitrogen, oxygen, or sulfur, 4-7-membered heterocyclyl having 1-2 heteroatoms independently selected from the group consisting of nitrogen, oxygen, and sulfur;
each occurrence of R^{x} is independently hydrogen or an oxygen protecting group selected from the group consisting of alkyl ethers, benzyl ethers, silyl ethers, acetals, ketals, esters, carbamates, and carbonates;
each occurrence of R is independently hydrogen, an optionally substituted group selected from acyl, arylalkyl, 6-10-membered aryl, C₁₋₆ aliphatic, or C₁₋₆ heteroaliphatic having 1-2 heteroatoms independently selected from the group consisting of nitrogen, oxygen, and sulfur, or:
two R on the same nitrogen atom are taken with the nitrogen atom to form a 4-7-membered heterocyclic ring having 1-2 heteroatoms independently selected from the group consisting of nitrogen, oxygen, and sulfur; and
(b) a TLR4 agonist selected from the group consisting of Lipid A, MPL, 3D-MPL, GLA, LPS, RC-529, E6020, ONO-4007, CRX-527, CRX-547, GSK1795091, CRX-601, SLA, PET-lipid A, PHAD, 3D-PHAD, 3D-(6-acyl)-PHAD, OM-294, OM-174, OK-432, 2B182C, and EcML.

2. The pharmaceutical composition of claim 1,
wherein the compound of Formula I is Compound I-4:

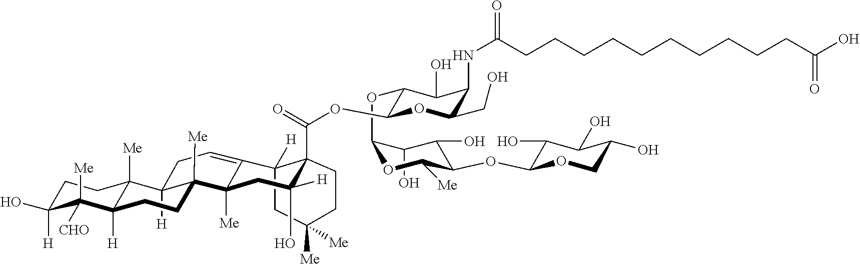

3. The pharmaceutical composition of claim 2,
further comprising an antigen.

4. The pharmaceutical composition of claim 3,
wherein the antigen is associated with a bacteria or virus.

5. The pharmaceutical composition of claim 4,
wherein the antigen is associated with the SARS-COV-2 virus.

6. The pharmaceutical composition of claim 4,
wherein the antigen is associated with Varicella Zoster.

7. A method of conferring resistance to an infection, the method comprising administering an antigen in combination with a pharmaceutical composition of any of claims 1 to 6.


SEQUENCE LISTING (21 sequences)

SEQ ID NO: 1 - protein, 546 aa, Varicella zoster
MGTVNKPVVGVLMGFGIITGTLRITNPVRASVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLIRYAAWTGGLA

SEQ ID NO: 2 - protein, 546 aa, Varicella zoster
MGTVNKPVVGVLMGFGIITGTLRITNPVRASVLRYDDFHTDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLLRYAAWTGGLA

SEQ ID NO: 3 - protein, 623 aa, Varicella zoster
MGTVNKPVVGVLMGFGIITGTLRITNPVRASVLRYDDFHTDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLLRYAAWTGGLAAVVLLCLVIFLICTAKRMRVKAYRVDKSPYNQSMYYAGLPVDDFEDSESTDTEEEFGNAIGGSHGGSSYTVYIDKTR

SEQ ID NO: 4 - protein, 623 aa, Varicella zoster
MGTVNKPVVGVLMGFGIITGTLRITNPVRASVLRYDDFHTDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLIRYAAWTGGLAAVVLLCLVIFLICTAKRMRVKAYRVDKSPYNQSMYYAGLPVDDFEDSESTDTEEEFGNAIGGSHGGSSYTVYIDKTR

SEQ ID NO: 5 - protein, 931 aa, Varicella zoster
MSPCGYYSKWRNRDRPEYRRNLRFRRFFSSIHPNAAAGSGFNGPGVFITSVTGVWLCFLCIFSMFVTAVVSVSPSSFYESLQVEPTQSEDITRSAHLGDGDEIREAIHKSQDAETKPTFYVCPPPTGSTIVRLEPPRTCPDYHLGKNFTEGIAVVYKENIAAYKFKATVYYKDVIVSTAWAGSSYTQITNRYADRVPIPVSEITDTIDKFGKCSSKATYVRNNHKVEAFNEDKNPQDMPLIASKYNSVGSKAWHTTNDTYMVAGTPGTYRTGTSVNCIIEEVEARSIFPYDSFGLSTGDIIYMSPFFGLRDGAYREHSNYAMDRFHQFEGYRQRDLDTRALLEPAARNFLVTPHLTVGWNWKPKRTEVCSLVKWREVEDVVRDEYAHNFRFTMKTLSTTFISETNEFNLNQIHLSQCVKEEARAIINRIYTTRYNSSHVRTGDIQTYLARGGFVVVFQPLLSNSLARLYLQELVRENTNHSPQKHPTRNTRSRRSVPVELRANRTITTTSSVEFAMLQFTYDHIQEHVNEMLARISSSWCQLQNRERALWSGLFPINPSALASTILDQRVKARILGDVISVSNCPELGSDTRIILQNSMRVSGSTTRCYSRPLISIVSLNGSGTVEGQLGTDNELIMSRDLLEPCVANHKRYFLFGHHYVYYEDYRYVREIAVHDVGMISTYVDLNLTLLKDREFMPLQVYTRDELRDTGLLDYSEIQRRNQMHSLRFYDIDKVVQYDSGTAIMQGMAQFFQGLGTAGQAVGHVVLGATGALLSTVHGFTTFLSNPFGALAVGLLVLAGLVAAFFAYRYVLKLKTSPMKALYPLTTKGLKQLPEGMDPFAEKPNATDTPIEEIGDSQNTEPSVNSGFDPDKFREAQEMIKYMTLVSAAERQESKARKKNKTSALLTSRLTGLALRNRRGYSRVRTENVTGV

SEQ ID NO: 6 - protein, 841 aa, Varicella zoster
MFALVLAVVILPLWTTANKSYVTPTPATRSIGHMSALLREYSDRNMSLKLEAFYPTGFDEELIKSLHWGNDRKHVFLVIVKVNPTTHEGDVGLVIFPKYLLSPYHFKAEHRAPFPAGRFGFLSHPVTPDVSFFDSSFAPYLTTQHLVAFTTFPPNPLVWHLERAETAATAERPFGVSLLPARPTVPKNTILEHKAHFATWDALARHTFFSAEAIITNSTLRIHVPLFGSVWPIRYWATGSVLLTSDSGRVEVNIGVGFMSSLISLSSGLPIELIVVPHTVKLNAVTSDTTWFQLNPPGPDPGPSYRVYLLGRGLDMNFSKHATVDICAYPEESLDYRYHLSMAHTEALRMTTKADQHDINEESYYHIAARIATSIFALSEMGRTTEYFLLDEIVDVQYQLKFLNYILMRIGAGAHPNTISGTSDLIFADPSQLHDELSLLFGQVKPANVDYFISYDEARDQLKTAYALSRGQDHVNALSLARRVIMSIYKGLLVKQNLNATERQALFFASMILLNFREGLENSSRVLDGRTTLLLMTSMCTAAHATQAALNIQEGLAYLNPSKHMFTIPNVYSPCMGSLRTDLTEEIHVMNLLSAIPTRPGLNEVLHTQLDESEIFDAAFKTMMIFTTWTAKDLHILHTHVPEVFTCQDAAARNGEYVLILPAVQGHSYVITRNKPQRGLVYSLADVDVYNPISVVYLSKDTCVSEHGVIETVALPHPDNLKECLYCGSVFLRYLTTGAIMDIIIIDSKDTERQLAAMGNSTIPPFNPDMHGDDSKAVLLFPNGTVVTLLGFERRQAIRMSGQYLGASLGGAFLAVVGFGIIGWMLCGNSRLREYNKIPLT

SEQ ID NO: 7 - protein, 354 aa, Varicella zoster
MFLIQCLISAVIFYIQVTNALIFKGDHVSLQVNSSLTSILIPMQNDNYTEIKGQLVFIGEQLPTGTNYSGTLELLYADTVAFCFRSVQVIRYDGCPRIRTSAFISCRYKHSWHYGNSTDRISTEPDAGVMLKITKPGINDAGVYVLLVRLDHSRSTDGFILGVNVYTAGSHHNIHGVIYTSPSLQNGYSTRALFQQARLCDLPATPKGSGTSLFQHMLDLRAGKSLEDNPWLHEDVVTTETKSVVKEGIENHVYPTDMSTLPEKSLNDPPENLLIIIPIVASVMILTAMVIVIVISVKRRRIKKHPIYRPNTKTRRGIQNATPESDVMLEAAIAQLATIREESPPHSVVNPFVK

SEQ ID NO: 8 - protein, 591 aa, Varicella zoster
MSKKTFPSFKFRGGCFNLLFKGSVDVSIKTRMKRIQINLILTIACIQLSTESQPTPVSITELYTSAATRKPDPAVAPTSAASRKPDPAVAPTSAASRKPDPAVAPTSAASRKPDPAVAPTSAATRKPDPAVAPTSAASRKPDPAVAPTSAATRKPDPAVAPTSAASRKPDPAANTQHSQPPFLYENIQCVHGGIQSIPYFHTFIMPCYMRLTTGQQAAFKQQQKTYEQYSLDPEGSNITRWKSLIRPDLHIEVWFTRHLIDPHRQLGNALIRMPDLPVMLYSNSADLNLINNPEIFTHAKENYVIPDVKTTSDFSVTILSMDATTEGTYIWRVVNTKTKNVISEHSITVTTYYRPNITVVGDPVLTGQTYAAYCNVSKYYPPHSVRVRWTSRFGNIGKNFITDAIQEYANGLFSYVSAVRIPQQKQMDYPPPAIQCNVLWIRDGVSNMKYSAVVTPDVYPFPNVSIGIIDGHIVCTAKCVPRGVVHFVWWVNDSPINHENSEITGVCDQNKRFVNMQSSCPTSELDGPITYSCHLDGYPKKFPPFSAVYTYDASTYATTFSVVAVIIGVISILGTLGLIAVIATLCIRCCS

SEQ ID NO: 9 - protein, 533 aa, Varicella zoster
MGVKVLFALICIAVAEASVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLIRYAAWTGGLA

SEQ ID NO: 10 - DNA, 22 bp, Artificial Sequence
  Sequence is synthesized
tgactgtgaacgttcgagatga

SEQ ID NO: 11 - DNA, 30 bp, Artificial Sequence
  Sequence is synthesized
tcgaacgttcgaacgttcgaacgttcgaat

SEQ ID NO: 12 - DNA, 21 bp, Artificial Sequence
  Sequence is synthesized
tcgtcgaacgttcgagatgat

SEQ ID NO: 13 - DNA, 25 bp, Artificial Sequence
  Sequence is synthesized
tcgttcgaacgttcgaacgttcgaa

SEQ ID NO: 14 - DNA, 25 bp, Artificial Sequence
  Sequence is synthesized
tcgaacgttcgaacgttcgaacgtt

SEQ ID NO: 15 - DNA, 25 bp, Artificial Sequence
  Sequence is synthesized
tcgaacgttcgaacgttcgaatttt

SEQ ID NO: 16 - DNA, 27 bp, Artificial Sequence
  Sequence is synthesized
tcgtaacgttcgaacgttcgaacgtta

SEQ ID NO: 17 - DNA, 26 bp, Artificial Sequence
  Sequence is synthesized
tcgtaacgttcgaacgttcgaacgtt

SEQ ID NO: 18 - DNA, 25 bp, Artificial Sequence
  Sequence is synthesized
tcgtaacgttcgaacgttcgaacgt

SEQ ID NO: 19 - DNA, 24 bp, Artificial Sequence
  Sequence is synthesized
tcgtaacgttcgaacgttcgaacg

SEQ ID NO: 20 - DNA, 23 bp, Artificial Sequence
  Sequence is synthesized
tcgtaacgttcgaacgttcgaac

SEQ ID NO: 21 - DNA, 22 bp, Artificial Sequence
  Sequence is synthesized
tcgtaacgttcgaacgttcgaa